(19) United States
(12) Patent Application Publication
(10) Pub. No.: US 2008/0058521 A1
(43) Pub. Date: Mar. 6, 2008

(54) Processes for the preparation of compounds

(75) Inventors: Lalitha Krishnan, Suffern, NY (US); David Blum, Upper Saddle River, NJ (US); Anja Susanne Dilley, Blooming Grove, NY (US); Sherry Pan, Asburn, VA (US); John Potoski, West Nyack, NY (US); Uresh Shah, Cranbury, NJ (US); Archana Sharma, Congers, NY (US); Henry Lee Strong, Somerset, NJ (US); Yanzhong Wu, Bardonia, NY (US); Mei-Yi Zhang, Princeton, NJ (US); Cynthia Blum, Upper Saddle River, NJ (US)

Correspondence Address: ROPES & GRAY LLP;PATENT DOCKETING 39/41, ONE INTERNATIONAL PLACE, BOSTON, MA, 02110-2624, US

(73) Assignee: Wyeth, Madison, NJ (US)

(21) Appl. No.: 11/698,258

(22) Filed: Jan. 25, 2007

Related U.S. Application Data
(60) Provisional application No. 60/763,154, filed on Jan. 26, 2006.
(60) Provisional application No. 60/856,865, filed on Nov. 2, 2006.

(51) Int. Cl. C07D 239/02; C07D 213/02; C07D 241/02; C07D 333/52

(52) U.S. Cl. 544/331; 544/405; 546/281.1; 549/57

(57) ABSTRACT

The present invention provides improved synthetic methods for the preparation of compounds that modulate proliferation or differentiation in a cell or tissue.

### CROSS REFERENCE TO RELATED APPLICATIONS

This application claims priority to and claims the benefit of U.S. Provisional Patent Application Nos. 60/856,865, filed Nov. 2, 2006 and 60/763,154, filed Jan. 26, 2006, the specifications of each of which are incorporated herein by reference.

### BACKGROUND OF THE INVENTION

International application WO 01/74344 presented compounds for modulating proliferation or differentiation in a cell or tissue. These compounds can be employed to correct or inhibit an aberrant or unwanted growth state. There remains a need for improved methods of synthesizing these compounds.

### SUMMARY OF THE INVENTION

The invention relates to a process for the preparation of a compound of Formula I, or a pharmaceutically acceptable salt thereof:

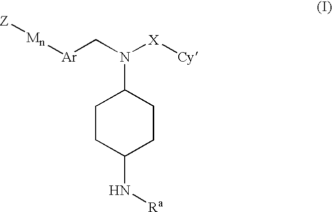

Ar is a substituted or unsubstituted aryl or heteroaryl, such as a substituted or unsubstituted phenyl;

M, independently for each occurrence, represents a substituted or unsubstituted methylene group (e.g., substituted with lower alkyl, oxo, hydroxyl, etc.), NR⁴ (wherein R⁴ is H, substituted or unsubstituted alkyl, such as substituted or unsubstituted lower alkyl, or an amine protecting group), O, S, S(O), or S(O)₂, preferably selected such that no two heteroatoms are adjacent to each other;

n is an integer from 0-4, such as 0, 1, or 2, particularly 0;

Z is H, a substituted or unsubstituted aryl, carbocyclyl, heterocyclyl, or heteroaryl, or a nitro, cyano, or halogen,

R^{a} is H, a substituted or unsubstituted alkyl, such as a substituted or unsubstituted lower alkyl, for example, methyl, or an amine protecting group;

X is —C(═O)—, —C(═S)—, —S(O₂)—, —S(O)—, —C(═NCN)—, or —P(═O)(OR)—, particularly —C(═O)—, —C(═S)—, or —S(O₂)—; and

Cy′ is a substituted or unsubstituted benzothiophene, such as a substituted or unsubstituted 2-benzothiophene,

and for the preparation of various intermediates useful in the preparation of such a compound.

### DETAILED DESCRIPTION OF THE INVENTION

### 1. Definitions

The term “acyl” is art-recognized and refers to a group represented by the general formula hydrocarbylC(O)—, preferably alkylC(O)—.

The term “acylamino” is art-recognized and refers to an amino group substituted with an acyl group and may be represented, for example, by the formula hydrocarbylC(O)NH—, preferably alkylC(O)NH—.

The term “acyloxy” is art-recognized and refers to a group represented by the general formula hydrocarbylC(O)O—, preferably alkylC(O)O—.

The term “aliphatic” as used herein includes straight, chained, branched or cyclic hydrocarbons which are completely saturated or contain one or more units of unsaturation. Aliphatic groups may be substituted or unsubstituted.

The term “alkoxy” refers to an alkyl group having an oxygen attached thereto. Representative alkoxy groups include methoxy, ethoxy, propoxy, tert-butoxy and the like.

The term “alkoxyalkyl” refers to an alkyl group substituted with an alkoxy group and may be represented by the general formula alkyl-O-alkyl.

The term “alkenyl”, as used herein, refers to an aliphatic group containing at least one double bond and is intended to include both “unsubstituted alkenyls” and “substituted alkenyls”, the latter of which refers to alkenyl moieties having substituents replacing a hydrogen on one or more carbons of the alkenyl group. Such substituents may occur on one or more carbons that are included or not included in one or more double bonds. Moreover, such substituents include all those contemplated for alkyl groups, as discussed below, except where stability is prohibitive. For example, substitution of alkenyl groups by one or more alkyl, carbocyclyl, aryl, heterocyclyl, or heteroaryl groups is contemplated. In preferred embodiments, a straight chain or branched chain alkenyl has 1-12 carbons in its backbone, preferably 1-8 carbons in its backbone, and more preferably 1-6 carbons in its backbone. Exemplary alkenyl groups include allyl, propenyl, butenyl, 2-methyl-2-butenyl, and the like.

The term “alkyl” refers to the radical of saturated aliphatic groups, including straight-chain alkyl groups, and branched-chain alkyl groups. In preferred embodiments, a straight chain or branched chain alkyl has 30 or fewer carbon atoms in its backbone (e.g., C₁-C₃₀ for straight chains, C₃-C₃₀ for branched chains), and more preferably 20 or fewer. In certain embodiments, alkyl groups are lower alkyl groups, e.g. methyl, ethyl, n-propyl, i-propyl, n-butyl and n-pentyl.

Moreover, the term “alkyl” (or “lower alkyl”) as used throughout the specification, examples, and claims is intended to include both “unsubstituted alkyls” and “substituted alkyls”, the latter of which refers to alkyl moieties having substituents replacing a hydrogen on one or more carbons of the hydrocarbon backbone. Such substituents can include, for example, a halogen, a hydroxyl, a carbonyl (such as a carboxyl, an alkoxycarbonyl, a formyl, or an acyl), a thiocarbonyl (such as a thioester, a thioacetate, or a thioformate), an alkoxyl, a phosphoryl, a phosphate, a phosphonate, a phosphinate, an amino, an amido, an amidine, an imine, a cyano, a nitro, an azido, a sulfhydryl, an alkylthio, a sulfate, a sulfonate, a sulfamoyl, a sulfonamido, a sulfonyl, a heterocyclyl, an aralkyl, or an aryl or heteroaryl moiety.

The term “C_{x-y}” when used in conjunction with a chemical moiety, such as, acyl, acyloxy, alkyl, alkenyl, alkynyl, or alkoxy is meant to include groups that contain from x to y carbons in the chain. For example, the term “C_{x-y}alkyl” refers to substituted or unsubstituted saturated hydrocarbon groups, including straight-chain alkyl and branched-chain alkyl groups that contain from x to y carbons in the chain, including haloalkyl groups such as trifluoromethyl and 2,2,2-tirfluoroethyl, etc. C₀ alkyl indicates a hydrogen where the group is in a terminal position, a bond if internal. The terms “C_{2-y}alkenyl” and “C_{2-y}alkynyl” refer to substituted or unsubstituted unsaturated aliphatic groups analogous in length and possible substitution to the alkyls described above, but that contain at least one double or triple bond respectively.

The term “alkylamino”, as used herein, refers to an amino group substituted with at least one alkyl group.

The term “alkylthio”, as used herein, refers to a thiol group substituted with an alkyl group and may be represented by the general formula alkylS—.

The term “alkynyl”, as used herein, refers to an aliphatic group containing at least one triple bond and is intended to include both “unsubstituted alkynyls” and “substituted alkynyls”, the latter of which refers to alkynyl moieties having substituents replacing a hydrogen on one or more carbons of the alkynyl group. Such substituents may occur on one or more carbons that are included or not included in one or more triple bonds. Moreover, such substituents include all those contemplated for alkyl groups, as discussed above, except where stability is prohibitive. For example, substitution of alkynyl groups by one or more alkyl, carbocyclyl, aryl, heterocyclyl, or heteroaryl groups is contemplated. In preferred embodiments, an alkynyl has 1-12 carbons in its backbone, preferably 1-8 carbons in its backbone, and more preferably 1-6 carbons in its backbone. Exemplary alkynyl groups include propynyl, butynyl, 3-methylpent-1-ynyl, and the like.

The term “amide”, as used herein, refers to a group 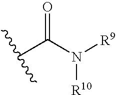
wherein R⁹ and R¹⁰ each independently represent a hydrogen or hydrocarbyl group, or R⁹ and R¹⁰ taken together with the N atom to which they are attached complete a heterocycle having from 4 to 8 atoms in the ring structure.

The terms “amine” and “amino” are art-recognized and refer to both unsubstituted and substituted amines and salts thereof, e.g., a moiety that can be represented by 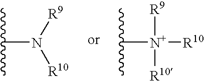
wherein R⁹, R¹⁰, and R^{10′} each independently represent a hydrogen or a hydrocarbyl group, or R⁹ and R¹⁰ taken together with the N atom to which they are attached complete a heterocycle having from 4 to 8 atoms in the ring structure.

The term “aminoalkyl”, as used herein, refers to an alkyl group substituted with an amino group.

The term “aralkyl”, as used herein, refers to an alkyl group substituted with an aryl group.

The term “aryl” as used herein include substituted or unsubstituted single-ring aromatic groups in which each atom of the ring is carbon. Preferably the ring is a 5- to 7-membered ring, more preferably a 6-membered ring. Aryl groups include phenyl, phenol, aniline, and the like.

The term “aryloxy” refers to an aryl group having an oxygen attached thereto. Representative aryloxy groups include phenoxy, naphthoxyl, and the like.

The term “carbamate” is art-recognized and refers to a group 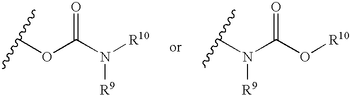
wherein R⁹ and R¹⁰ independently represent hydrogen or a hydrocarbyl group, such as an alkyl group.

The terms “carbocycle”, “carbocyclyl”, and “carbocyclic”, as used herein, refers to a non-aromatic saturated or unsaturated ring in which each atom of the ring is carbon. Preferably a carbocycle ring contains from 3 to 10 atoms, more preferably from 5 to 7 atoms.

The term “carbocyclylalkyl”, as used herein, refers to an alkyl group substituted with a carbocycle group.

The term “carbonate” is art-recognized and refers to a group —OCO₂—R⁹, wherein R⁹ represents a hydrocarbyl group, such as an alkyl group.

The term “carboxy”, as used herein, refers to a group represented by the formula —CO₂H.

The term “cycloalkyl”, as used herein, refers to the radical of a saturated aliphatic ring. In preferred embodiments, cycloalkyls have from 3-10 carbon atoms in their ring structure, and more preferably from 5-7 carbon atoms in the ring structure. Suitable cycloalkyls include cycloheptyl, cyclohexyl, cyclopentyl, cyclobutyl and cyclopropyl.

The term “ester”, as used herein, refers to a group —C(O)OR⁹ wherein R⁹ represents a hydrocarbyl group, such as an alkyl group or an aralkyl group.

The term “ether”, as used herein, refers to a hydrocarbyl group linked through an oxygen to another hydrocarbyl group. Accordingly, an ether substituent of a hydrocarbyl group may be hydrocarbyl-O—. Ethers may be either symmetrical or unsymmetrical. Examples of ethers include, but are not limited to, heterocycle-O-heterocycle and aryl-O-heterocycle. Ethers include “alkoxyalkyl” groups, which may be represented by the general formula alkyl-O-alkyl.

The terms “halo” and “halogen” as used herein means halogen and includes chloro, fluoro, bromo, and iodo.

The terms “hetaralkyl” and “heteroaralkyl”, as used herein, refers to an alkyl group substituted with a hetaryl group.

The terms “heteroaryl” and “hetaryl” include substituted or unsubstituted aromatic single ring structures, preferably 5- to 7-membered rings, more preferably 5- to 6-membered rings, whose ring structures include at least one heteroatom (e.g., O, N, or S), preferably one to four or one to 3 heteroatoms, more preferably one or two heteroatoms. When two or more heteroatoms are present in a heteroaryl ring, they may be the same or different. The terms “heteroaryl” and “hetaryl” also include polycyclic ring systems having two or more cyclic rings in which two or more carbons are common to two adjoining rings wherein at least one of the rings is heteroaromatic, e.g., the other cyclic rings can be cycloalkyls, cycloalkenyls, cycloalkynyls, aryls, heteroaryls, and/or heterocyclyls. Preferred polycyclic ring systems have two cyclic rings in which both of the rings are aromatic. Heteroaryl groups include, for example, pyrrole, furan, thiophene, imidazole, oxazole, thiazole, pyrazole, pyridine, pyrazine, pyridazine, quinoline, and pyrimidine, and the like.

The term “heteroaryloxy” refers to a heteroaryl group having an oxygen attached thereto. Representative heteroaryloxy groups include pyridoxy and the like.

The term “heteroatom” as used herein means an atom of any element other than carbon or hydrogen. Preferred heteroatoms are nitrogen, oxygen, and sulfur.

The terms “heterocyclyl”, “heterocycle”, and “heterocyclic” refer to substituted or unsubstituted non-aromatic ring structures, preferably 3- to 10-membered rings, more preferably 3- to 7-membered rings, whose ring structures include at least one heteroatom, preferably one to four heteroatoms, more preferably one or two heteroatoms. Heterocyclyl groups include, for example, piperidine, piperazine, pyrrolidine, morpholine, lactones, lactams, and the like.

The term “heterocyclylalkyl”, as used herein, refers to an alkyl group substituted with a heterocycle group.

The term “hydrocarbyl”, as used herein, refers to a group that is bonded through a carbon atom that does not have a ═O or ═S substituent, and typically has at least one carbon-hydrogen bond and a primarily carbon backbone, but may optionally include heteroatoms. Thus, groups like methyl, ethoxyethyl, 2-pyridyl, and trifluoromethyl are considered to be hydrocarbyl for the purposes of this application, but substituents such as acetyl (which has a ═O substituent on the linking carbon) and ethoxy (which is linked through oxygen, not carbon) are not. Hydrocarbyl groups include, but are not limited to aryl, heteroaryl, carbocycle, heterocycle, alkyl, alkenyl, alkynyl, and combinations thereof.

The term “hydroxyalkyl”, as used herein, refers to an alkyl group substituted with a hydroxy group.

The term “lower” when used in conjunction with a chemical moiety, such as, acyl, acyloxy, alkyl, alkenyl, alkynyl, or alkoxy is meant to include groups where there are ten or fewer non-hydrogen atoms in the substituent, preferably six or fewer. A “lower alkyl”, for example, refers to an alkyl group that contains ten or fewer carbon atoms, preferably six or fewer. Examples of straight chain or branched chain lower alkyl include methyl, ethyl, isopropyl, propyl, butyl, tertiary-butyl, and the like. In certain embodiments, acyl, acyloxy, alkyl, alkenyl, alkynyl, or alkoxy substituents defined herein are respectively lower acyl, lower acyloxy, lower alkyl, lower alkenyl, lower alkynyl, or lower alkoxy, whether they appear alone or in combination with other substituents, such as in the recitations hydroxyalkyl and aralkyl (in which case, for example, the atoms within the aryl group are not counted when counting the carbon atoms in the alkyl substituent).

The terms “polycyclyl”, “polycycle”, and “polycyclic” refer to two or more rings (e.g., cycloalkyls, cycloalkenyls, cycloalkynyls, aryls, heteroaryls, and/or heterocyclyls) in which two or more atoms are common to two adjoining rings, e.g., the rings are “fused rings”. Preferred polycycles have 2-3 rings. Each of the rings of the polycycle can be substituted or unsubstituted. In certain embodiments, each ring of the polycycle contains from 3 to 10 atoms in the ring, preferably from 5 to 7.

The term “substituted” refers to moieties having substituents replacing a hydrogen on one or more carbons of the backbone. It will be understood that “substitution” or “substituted with” includes the implicit proviso that such substitution is in accordance with permitted valence of the substituted atom and the substituent, and that the substitution results in a stable compound, e.g., which does not spontaneously undergo transformation such as by rearrangement, cyclization, elimination, etc. As used herein, the term “substituted” is contemplated to include all permissible substituents of organic compounds. In a broad aspect, the permissible substituents include acyclic and cyclic, branched and unbranched, carbocyclic and heterocyclic, aromatic and non-aromatic substituents of organic compounds. The permissible substituents can be one or more and the same or different for appropriate organic compounds. For purposes of this invention, the heteroatoms such as nitrogen may have hydrogen substituents and/or any permissible substituents of organic compounds described herein which satisfy the valences of the heteroatoms. Substituents can include any substituents described herein, for example, a halogen, a hydroxyl, a carbonyl (such as a carboxyl, an alkoxycarbonyl, a formyl, or an acyl), a thiocarbonyl (such as a thioester, a thioacetate, or a thioformate), an alkoxyl, a phosphoryl, a phosphate, a phosphonate, a phosphinate, an amino, an amido, an amidine, an imine, a cyano, a nitro, an azido, a sulfhydryl, an alkylthio, a sulfate, a sulfonate, a sulfamoyl, a sulfonamido, a sulfonyl, a heterocyclyl, an aralkyl, or an aromatic or heteroaromatic moiety.

Unless specifically stated as “unsubstituted,” references to chemical moieties herein are understood to include substituted variants. For example, reference to an “aryl” group or moiety implicitly includes both substituted and unsubstituted variants.

The term “sulfate” is art-recognized and refers to the group —OSO₃H, or a pharmaceutically acceptable salt thereof.

The term “sulfonamide” is art-recognized and refers to the group represented by the general formulae 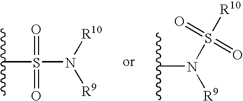
wherein R⁹ and R¹⁰ independently represents hydrogen or hydrocarbyl, such as alkyl.

The term “sulfoxide” is art-recognized and refers to the group —S(O)—R⁹, wherein R⁹ represents a hydrocarbyl, such as alkyl, aryl, or heteroaryl.

The term “sulfonate” is art-recognized and refers to the group SO₃H, or a pharmaceutically acceptable salt thereof.

The term “sulfone” is art-recognized and refers to the group —S(O)₂—R⁹, wherein R⁹ represents a hydrocarbyl, such as alkyl, aryl, or heteroaryl.

The term “thioalkyl”, as used herein, refers to an alkyl group substituted with a thiol group.

The term “thioester”, as used herein, refers to a group —C(O)SR⁹ or —SC(O)R⁹ wherein R⁹ represents a hydrocarbyl, such as alkyl.

The term “thioether”, as used herein, is equivalent to an ether, wherein the oxygen is replaced with a sulfur.

The term “urea” is art-recognized and may be represented by the general formula 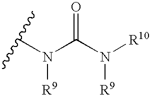
wherein R⁹ and R¹⁰ independently represent hydrogen or a hydrocarbyl, such as alkyl.

For a number qualified by the term “about”, a variance of 2%, 5%, 10% or even 20% is within the ambit of the qualified number.

### 2. Processes

In certain embodiments, the present invention provides a method for the preparation of a compound of Formula D, or a salt thereof: 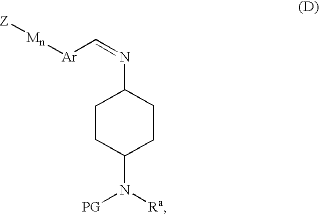
comprising treating a compound of Formula A, or a salt thereof, with a compound of Formula B, or a salt thereof: 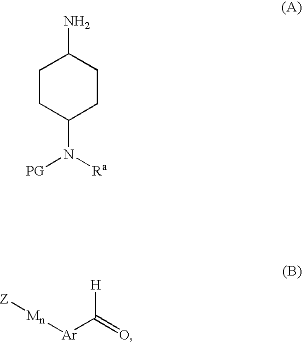
under imine formation conditions, wherein, as valence and stability permit,

Ar is a substituted or unsubstituted aryl or heteroaryl, such as a substituted or unsubstituted phenyl;

M, independently for each occurrence, represents a substituted or unsubstituted methylene group (e.g., substituted with lower alkyl, oxo, hydroxyl, etc.), NR⁴ (wherein R⁴ is H or substituted or unsubstituted alkyl, such as substituted or unsubstituted lower alkyl, or an amine protecting group), O, S, S(O), or S(O)₂, preferably selected such that no two heteroatoms are adjacent to each other;

n is an integer from 0-4, such as 0, 1, or 2, particularly 0;

Z is H, a substituted or unsubstituted aryl, carbocyclyl, heterocyclyl, or heteroaryl, or a nitro, cyano, or halogen;

R^{a} is H, substituted or unsubstituted alkyl, such as a substituted or unsubstituted lower alkyl, for example, methyl, or an amine protecting group; and

PG is H or an amine protecting group.

In some embodiments, the compound of Formula A and the compound of Formula B are combined in about a 1:0.9 to 1 ratio, respectively.

In certain embodiments, the imine formation conditions comprise maintaining a temperature between about 20 and about 50° C., such as between about 25 or 30 and about 40° C., particularly, between about 30 and about 35° C. In certain embodiments, the imine formation conditions comprise maintaining a temperature above room temperature, for example, about 2, 5, 7, 10, 15, 20, 30, or 40° C. above room temperature.

In some embodiments, the imine formation conditions comprise one or more organic or aqueous solvent systems or combinations thereof. For example, in certain embodiments, the imine formation conditions comprise an alkyl acetate, such as isopropyl acetate, wherein the alkyl acetate may represent part of the solvent system, for example, about 10, 20, 30, 50, 70, or 90% of the solvent system, or substantially all of the solvent system, for example about 95% or greater of the solvent system.

In certain embodiments, n is 0.

In certain embodiments, Z is H. In other embodiments, Z is a substituted or unsubstituted aryl, carbocyclyl, heterocyclyl, or heteroaryl. For example, in some embodiments Z is a substituted or unsubstituted thiophene, thianthrene, furan, pyran, isobenzofuran, chromene, xanthene, phenoxathiin, pyrrole, imidazole, pyrazole, isothiazole, isoxazole, pyridine, pyrazine, pyrimidine, pyridazine, indolizine, isoindole, indole, indazole, purine, quinolizine, isoquinoline, quinoline, phthalazine, naphthyridine, quinoxaline, quinazoline, cinnoline, pteridine, carbazole, carboline, phenanthridine, acridine, phenanthroline, phenazine, phenarsazine, phenothiazine, furazan, phenoxazine, pyrrolidine, oxolane, thiolane, oxazole, piperidine, piperazine, morpholine, lactone, lactam, azetidinone, pyrrolidinone, sultam, or sultone. In particular embodiments, Z is a substituted or unsubstituted aryl or heteroaryl, such as a substituted or unsubstituted phenyl. In other embodiments, Z is a substituted or unsubstituted carbocyclyl or heterocyclyl.

In some embodiments, Z is a substituted or unsubstituted aryl ring, such as a phenyl ring.

In other embodiments, Z is a substituted or unsubstituted heteroaryl ring, such as a substituted or unsubstituted pyridine, pyrimidine, pyrazine, pyridazine, triazine, tetrazine, pyrrole, pyrazole, or imidazole ring or N-oxide thereof. In specific embodiments, Z is a substituted or unsubstituted pyridine, or pyrazine ring or N-oxide thereof, particularly a substituted or unsubstituted pyridine ring or N-oxide thereof.

In some examples, Z is a substituted or unsubstituted pyridine ring or N-oxide thereof where connection of the pyridine ring to the phenyl ring bearing R¹ and R² can occur at any location on the pyridine ring, for example, at a 2-, 3-, or 4-position relative to the nitrogen of the pyridine ring, i.e., at an ortho-, meta-, or para-position relative to the nitrogen of the pyridine ring. In certain embodiments, Z is a substituted or unsubstituted pyridine N-oxide.

In other embodiments, Z is a substituted or unsubstituted pyrimidine ring or N-oxide thereof where connection of the pyrimidine ring to the phenyl ring bearing R¹ and R² can occur at any location on the pyrimidine ring, for example, at a 2-, 3-, 4-, 5-, or 6-position relative to N1 of the pyrimidine ring.

In some embodiments, Z is unsubstituted. In other embodiments, Z is substituted with one or more groups selected from halogen, lower alkyl, lower alkenyl, —CN, azido, —NR^{x}R^{x}, —CO₂OR^{x}, —C(O)—NR^{x}R^{x}, —C(O)—R^{x}, —NR^{x}—C(O)—R^{x}, —NR^{x}SO₂R^{x}, —SR^{x}, —S(O)R^{x}, —SO₂R^{x}, —SO₂NR^{x}R^{x}, (C(R^{x})₂)ₙ—OR^{x}, (C(R^{x})₂)ₙ—NR^{x}R^{x}, and —(C(R^{x})₂)ₙ—SO₂R^{x}; wherein R^{x} are, independently for each occurrence, H or lower alkyl; and n is, independently for each occurrence, an integer from 0 to 2. For example, in some embodiments, Z is substituted with halogen, such as fluoro.

In certain embodiments, Z is substituted with one or more electron withdrawing groups. For example, sometimes Z is substituted with one or more groups selected from halogen, —CN, azido, —CO₂OR^{x}, —C(O)—NR^{x}R^{x}, and —C(O)—R^{x}.

Combinations of the various values for Z are contemplated. In some instances, suitable compounds include those where Z is a pyridine ring or N-oxide thereof and Z is substituted by fluoro at any carbon position on the pyridine ring, such as a 2-, 3-, or 4-position relative to the nitrogen of the pyridine ring.

In certain embodiments, the compound of Formula B has the structure: 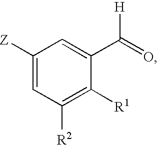
wherein R¹ and R² are, independently, H, halogen, hydroxyl, lower alkyl, or lower alkoxy, provided that at least one of R¹ and R² is not H.

In certain embodiments, R¹ and R² are, independently, H, fluorine, hydroxyl, lower alkyl, methoxy, or ethoxy, provided that at least one of R¹ and R² is not H.

In particular embodiments, R¹ is hydroxyl, ethyl, propyl, methoxy, or ethoxy; and R² is H, fluorine, hydroxyl, lower alkyl, methoxy, or ethoxy. In certain such embodiments, R² is H.

In certain embodiments, one or both of R¹ or R² is methoxy. For example, in some embodiments, R¹ is methoxy and R² is H, while in others, R² is methoxy and R¹ is H. In some instances, both R¹ and R² are methoxy.

In certain embodiments, one or both of R¹ or R² is lower alkyl. For example, sometimes R¹ is lower alkyl, such as methyl, ethyl, or propyl.

In certain embodiments, one or both of R¹ or R² is hydroxyl. For example, sometimes R¹ is hydroxyl.

In certain embodiments, Z is a substituted or unsubstituted aryl or heteroaryl ring; R^{a} is methyl; R¹ is halogen, such as fluoro or chloro, methoxy, or ethoxy; and R² is H. In some embodiments, R¹ is methoxy. In other embodiments, R¹ is fluoro. In some instances, R¹ is ethoxy. In some instances, Z is not a substituted or unsubstituted pyridine N-oxide ring or a pyridine ring substituted with one or more halogens. In certain embodiments, Z is substituted with one or more electron withdrawing groups.

In certain embodiments, Z is a substituted or unsubstituted aryl or heteroaryl ring; R^{a} is H or methyl; R¹ is H; and R² is halogen, such as fluoro or chloro, hydroxyl, methyl, ethyl, methoxy, or ethoxy. In some embodiments, R² is methoxy. In certain embodiments, R^{a} is methyl. In certain embodiments, Z is substituted with one or more electron withdrawing groups.

In certain embodiments, Z is a substituted or unsubstituted aryl or heteroaryl ring; R^{a} is H or methyl; R¹ is hydroxyl, methyl, or ethyl; and R² is H, halogen, such as fluoro or chloro, hydroxyl, methyl, ethyl, methoxy, or ethoxy. In certain embodiments, R^{a} is methyl. In certain embodiments, R² is H. In certain embodiments, R¹ is hydroxyl. In certain embodiments, R¹ is methyl. In certain embodiments, R¹ is ethyl. In some embodiments, one or both of R¹ or R² is hydroxyl; for example, sometimes R¹ is hydroxyl. In certain embodiments, Z is substituted with one or more electron withdrawing groups.

In certain embodiments, Z is a substituted or unsubstituted pyridine N-oxide ring; R^{a} is H or methyl; and R¹ and R² are, independently, H, halogen, such as fluoro or chloro, hydroxyl, methyl, ethyl, methoxy, or ethoxy, provided that at least one of R¹ and R² is not H. In certain embodiments, one or both of R¹ or R² is methoxy; for example, sometimes R¹ is methoxy, sometimes R² is methoxy, and sometimes both R¹ and R² are methoxy. In certain embodiments, one or both of R¹ or R² is fluoro; for example, sometimes R¹ is fluoro, sometimes R² is fluoro, and sometimes both R¹ and R² are fluoro. In certain embodiments, R² is H. In certain embodiments, R¹ is hydroxyl. In certain embodiments, R¹ is methyl. In certain embodiments, R¹ is ethyl. In some embodiments, one or both of R¹ or R² is hydroxyl; for example, sometimes R¹ is hydroxyl. In certain embodiments, Z is substituted with one or more electron withdrawing groups.

In certain embodiments, Z is a pyridine ring substituted with one or more halogens, such as fluoro and/or chloro, and optionally further substituted; R^{a} is H or methyl; and R¹ and R² are, independently, H, halogen, such as fluoro or chloro, hydroxyl, methyl, ethyl, methoxy, or ethoxy, provided that at least one of R¹ and R² is not H. In certain embodiments, one or both of R¹ or R² is methoxy; for example, sometimes R¹ is methoxy, sometimes R² is methoxy, and sometimes both R¹ and R² are methoxy. In certain embodiments, one or both of R¹ or R² is fluoro; for example, sometimes R¹ is fluoro, sometimes R² is fluoro, and sometimes both R¹ and R² are fluoro. In certain embodiments, one or both of R¹ or R² is fluoro; for example, sometimes R¹ is fluoro, sometimes R² is fluoro, and sometimes both R¹ and R² are fluoro. In certain embodiments, R² is H. In certain embodiments, R¹ is hydroxyl. In certain embodiments, R¹ is methyl. In certain embodiments, R¹ is ethyl. In some embodiments, one or both of R¹ or R² is hydroxyl; for example, sometimes R¹ is hydroxyl. In certain embodiments, Z is substituted with one or more electron withdrawing groups.

In certain embodiments, Z is a substituted or unsubstituted aryl or heteroaryl ring; R^{a} is H; and R¹ and R² are, independently, H, halogen, such as fluoro or chloro, hydroxyl, methyl, ethyl, methoxy, or ethoxy, provided that at least one of R¹ and R² is not H. In certain embodiments, one or both of R¹ or R² is methoxy; for example, sometimes R¹ is methoxy, sometimes R² is methoxy, and sometimes both R¹ and R² are methoxy. In certain embodiments, R² is H. In certain embodiments, R¹ is hydroxyl. In certain embodiments, R¹ is methyl. In certain embodiments, R¹ is ethyl. In some embodiments, one or both of R¹ or R² is hydroxyl; for example, sometimes R¹ is hydroxyl. In certain embodiments, Z is substituted with one or more electron withdrawing groups.

In some embodiments, the compound of Formula A has the structure:

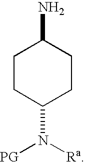

In some embodiments PG is H. In other embodiments PG is an amine protecting group, such as tert-butylcarboxy.

In certain embodiments, the present invention provides a method for the preparation of a compound of Formula D, or a salt thereof: 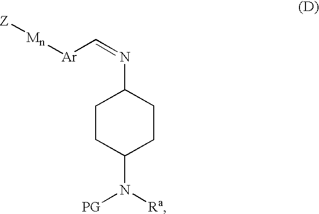
comprising treating a compound of Formula C, or a salt thereof: 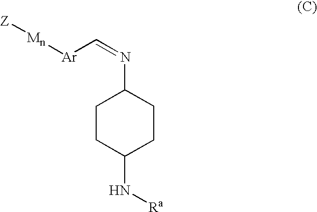
with an amine protecting group source under amine protection conditions, wherein, as valence and stability permit,

Ar is a substituted or unsubstituted aryl or heteroaryl, such as a substituted or unsubstituted phenyl;

M, independently for each occurrence, represents a substituted or unsubstituted methylene group (e.g., substituted with lower alkyl, oxo, hydroxyl, etc.), NR⁴ (wherein R⁴ is H, substituted or unsubstituted alkyl, such as substituted or unsubstituted lower alkyl, or an amine protecting group), O, S, S(O), or S(O)₂, preferably selected such that no two heteroatoms are adjacent to each other;

n is an integer from 0-4, such as 0, 1, or 2, particularly 0;

Z is H, a substituted or unsubstituted aryl, carbocyclyl, heterocyclyl, or heteroaryl, or a nitro, cyano, or halogen;

R^{a} is H, substituted or unsubstituted alkyl, such as a substituted or unsubstituted lower alkyl, for example, methyl, or an amine protecting group; and

PG is an amine protecting group, such as tert-butylcarboxy. Suitable amine-protecting groups are moieties that, when attached to the amine, mask, reduce, or block the reactivity of the amine. The protecting group may then be removed as desired, e.g., selectively, during the course of a synthesis. Representative amine protecting groups include, but are not limited to, trifluoroacetyl, benzyl, benzyloxycarbonyl (“CBZ”), tert-butoxycarbonyl (“Boc”), trimethylsilyl (“TMS”), 2-trimethylsilyl-ethanesulfonyl (“TES”), trityl and substituted trityl groups, allyloxycarbonyl, 9-fluorenylmethyloxycarbonyl (“FMOC”), nitro-veratryloxycarbonyl (“NVOC”) and the like.

In certain embodiments, the amine protecting group source is a carbonate. In some embodiments, the amine protecting group source is a tert-butylcarboxy source, such as di-tert-butyl dicarbonate.

In some embodiments, the amine protecting group source and the compound of Formula C are combined in about a 1:0.9 to 1 ratio, respectively.

In certain embodiments, the amine protection conditions comprise maintaining a temperature between about −10 and about 35° C., such as between about 0 and about 27° C., particularly between about 10 and about 25° C.

In some embodiments, the amine protection conditions comprise one or more organic or aqueous solvent systems or combinations thereof. For example, in certain embodiments, the amine protection conditions comprise and alkyl acetate, such as isopropyl acetate, wherein the alkyl acetate may represent part of the solvent system, for example, about 10, 20, 30, 50, 70, or 90% of the solvent system, or substantially all of the solvent system, for example about 95% or greater of the solvent system.

In certain embodiments, n is 0.

In certain embodiments, Z is H. In other embodiments, Z is a substituted or unsubstituted aryl, carbocyclyl, heterocyclyl, or heteroaryl. For example, in some embodiments Z is a substituted or unsubstituted thiophene, thianthrene, furan, pyran, isobenzofuran, chromene, xanthene, phenoxathiin, pyrrole, imidazole, pyrazole, isothiazole, isoxazole, pyridine, pyrazine, pyrimidine, pyridazine, indolizine, isoindole, indole, indazole, purine, quinolizine, isoquinoline, quinoline, phthalazine, naphthyridine, quinoxaline, quinazoline, cinnoline, pteridine, carbazole, carboline, phenanthridine, acridine, phenanthroline, phenazine, phenarsazine, phenothiazine, furazan, phenoxazine, pyrrolidine, oxolane, thiolane, oxazole, piperidine, piperazine, morpholine, lactone, lactam, azetidinone, pyrrolidinone, sultam, or sultone. In particular embodiments, Z is a substituted or unsubstituted aryl or heteroaryl, such as a substituted or unsubstituted phenyl. In other embodiments, Z is a substituted or unsubstituted carbocyclyl or heterocyclyl.

In some embodiments, Z is a substituted or unsubstituted aryl ring, such as a phenyl ring.

In other embodiments, Z is a substituted or unsubstituted heteroaryl ring, such as a substituted or unsubstituted pyridine, pyrimidine, pyrazine, pyridazine, triazine, tetrazine, pyrrole, pyrazole, or imidazole ring or N-oxide thereof. In specific embodiments, Z is a substituted or unsubstituted pyridine, or pyrazine ring or N-oxide thereof, particularly a substituted or unsubstituted pyridine ring or N-oxide thereof.

In some examples, Z is a substituted or unsubstituted pyridine ring or N-oxide thereof where connection of the pyridine ring to the phenyl ring bearing R¹ and R² can occur at any location on the pyridine ring, for example, at a 2-, 3-, or 4-position relative to the nitrogen of the pyridine ring, i.e., at an ortho-, meta-, or para-position relative to the nitrogen of the pyridine ring. In certain embodiments, Z is a substituted or unsubstituted pyridine N-oxide.

In other embodiments, Z is a substituted or unsubstituted pyrimidine ring or N-oxide thereof where connection of the pyrimidine ring to the phenyl ring bearing R¹ and R² can occur at any location on the pyrimidine ring, for example, at a 2-, 3-, 4-, 5-, or 6-position relative to N1 of the pyrimidine ring.

In some embodiments, Z is unsubstituted. In other embodiments, Z is substituted with one or more groups selected from halogen, lower alkyl, lower alkenyl, —CN, azido, —NR^{x}R^{x}, —CO₂OR^{x}, —C(O)—NR^{x}R^{x}, —C(O)—R^{x}, —NR^{x}—C(O)—R^{x}, NR^{x}SO₂R^{x}, SR^{x}, S(O)R^{x}, SO₂R^{x}, —SO₂NR^{x}R^{x}, —(C(R^{x})₂)ₙ—OR^{x}, —(C(R^{x})₂)ₙ—NR^{x}R^{x}, and —(C(R^{x})₂)ₙ—SO₂R^{x}; wherein R^{x} are, independently for each occurrence, H or lower alkyl; and n is, independently for each occurrence, an integer from 0 to 2. For example, in some embodiments, Z is substituted with halogen, such as fluoro.

In certain embodiments, Z is substituted with one or more electron withdrawing groups. For example, sometimes Z is substituted with one or more groups selected from halogen, —CN, azido, —CO₂OR^{x}, —C(O)—NR^{x}R^{x}, and —C(O)—R^{x}.

Combinations of the various values for Z are contemplated. In some instances, suitable compounds include those where Z is a pyridine ring or N-oxide thereof and Z is substituted by fluoro at any carbon position on the pyridine ring, such as a 2-, 3-, or 4-position relative to the nitrogen of the pyridine ring.

In certain embodiments, the compound of Formula C has the structure: 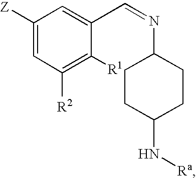
wherein R¹ and R² are, independently, H, halogen, hydroxyl, lower alkyl, or lower alkoxy, provided that at least one of R¹ and R² is not H.

In certain embodiments, R¹ and R² are, independently, H, fluorine, hydroxyl, lower alkyl, methoxy, or ethoxy, provided that at least one of R¹ and R² is not H.

In particular embodiments, R¹ is hydroxyl, ethyl, propyl, methoxy, or ethoxy; and R² is H, fluorine, hydroxyl, lower alkyl, methoxy, or ethoxy. In certain such embodiments, R² is H.

In certain embodiments, at least one of R¹ or R² is methoxy. For example, in some embodiments, R¹ is methoxy and R² is H, while in others, R² is methoxy and R¹ is H. In some instances, both R¹ and R² are methoxy.

In certain embodiments, at least one of R¹ or R² is lower alkyl. For example, sometimes R¹ is lower alkyl, such as methyl, ethyl, or propyl.

In certain embodiments, at least one of R¹ or R² is hydroxyl. For example, sometimes R¹ is hydroxyl.

In certain embodiments, Z is a substituted or unsubstituted aryl or heteroaryl ring; R^{a} is methyl; R¹ is halogen, such as fluoro or chloro, methoxy, or ethoxy; and R² is H. In some embodiments, R¹ is methoxy. In other embodiments, R¹ is fluoro. In some instances, R¹ is ethoxy. In some instances, Z is not a substituted or unsubstituted pyridine N-oxide ring or a pyridine ring substituted with one or more halogens. In certain embodiments, Z is substituted with one or more electron withdrawing groups.

In certain embodiments, Z is a substituted or unsubstituted aryl or heteroaryl ring; R^{a} is H or methyl; R¹ is H; and R² is halogen, such as fluoro or chloro, hydroxyl, methyl, ethyl, methoxy, or ethoxy. In some embodiments, R² is methoxy. In certain embodiments, R^{a} is methyl. In certain embodiments, Z is substituted with one or more electron withdrawing groups.

In certain embodiments, Z is a substituted or unsubstituted aryl or heteroaryl ring; R^{a} is H or methyl; R¹ is hydroxyl, methyl, or ethyl; and R² is H, halogen, such as fluoro or chloro, hydroxyl, methyl, ethyl, methoxy, or ethoxy. In certain embodiments, R^{a} is methyl. In certain embodiments, R² is H. In certain embodiments, R¹ is hydroxyl. In certain embodiments, R¹ is methyl. In certain embodiments, R¹ is ethyl. In some embodiments, one or both of R¹ or R² is hydroxyl; for example, sometimes R¹ is hydroxyl. In certain embodiments, Z is substituted with one or more electron withdrawing groups.

In certain embodiments, Z is a substituted or unsubstituted pyridine N-oxide ring; R^{a} is H or methyl; and R¹ and R² are, independently, H, halogen, such as fluoro or chloro, hydroxyl, methyl, ethyl, methoxy, or ethoxy, provided that at least one of R¹ and R² is not H. In certain embodiments, one or both of R¹ or R² is methoxy; for example, sometimes R¹ is methoxy, sometimes R² is methoxy, and sometimes both R¹ and R² are methoxy. In certain embodiments, one or both of R¹ or R² is fluoro; for example, sometimes R¹ is fluoro, sometimes R² is fluoro, and sometimes both R¹ and R² are fluoro. In certain embodiments, R² is H. In certain embodiments, R¹ is hydroxyl. In certain embodiments, R¹ is methyl. In certain embodiments, R¹ is ethyl. In some embodiments, one or both of R¹ or R² is hydroxyl; for example, sometimes R¹ is hydroxyl. In certain embodiments, Z is substituted with one or more electron withdrawing groups.

In certain embodiments, Z is a pyridine ring substituted with one or more halogens, such as fluoro and/or chloro, and optionally further substituted; R^{a} is H or methyl; and R¹ and R² are, independently, H, halogen, such as fluoro or chloro, hydroxyl, methyl, ethyl, methoxy, or ethoxy, provided that at least one of R¹ and R² is not H. In certain embodiments, one or both of R¹ or R² is methoxy; for example, sometimes R¹ is methoxy, sometimes R² is methoxy, and sometimes both R¹ and R² are methoxy. In certain embodiments, one or both of R¹ or R² is fluoro; for example, sometimes R¹ is fluoro, sometimes R² is fluoro, and sometimes both R¹ and R² are fluoro. In certain embodiments, one or both of R¹ or R² is fluoro; for example, sometimes R¹ is fluoro, sometimes R² is fluoro, and sometimes both R¹ and R² are fluoro. In certain embodiments, R² is H. In certain embodiments, R¹ is hydroxyl. In certain embodiments, R¹ is methyl. In certain embodiments, R¹ is ethyl. In some embodiments, one or both of R¹ or R² is hydroxyl; for example, sometimes R¹ is hydroxyl. In certain embodiments, Z is substituted with one or more electron withdrawing groups.

In certain embodiments, Z is a substituted or unsubstituted aryl or heteroaryl ring; R^{a} is H; and R¹ and R² are, independently, H, halogen, such as fluoro or chloro, hydroxyl, methyl, ethyl, methoxy, or ethoxy, provided that at least one of R¹ and R² is not H. In certain embodiments, one or both of R¹ or R² is methoxy; for example, sometimes R¹ is methoxy, sometimes R² is methoxy, and sometimes both R¹ and R² are methoxy. In certain embodiments, R² is H. In certain embodiments, R¹ is hydroxyl. In certain embodiments, R¹ is methyl. In certain embodiments, R¹ is ethyl. In some embodiments, one or both of R¹ or R² is hydroxyl; for example, sometimes R¹ is hydroxyl. In certain embodiments, Z is substituted with one or more electron withdrawing groups.

In some embodiments, the compound of Formula C has the structure:

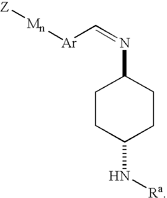

In certain embodiments, the present invention provides a method for the preparation of a compound of Formula E, or a salt thereof: 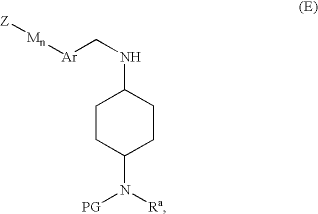
comprising treating a compound of Formula D, or a salt thereof: 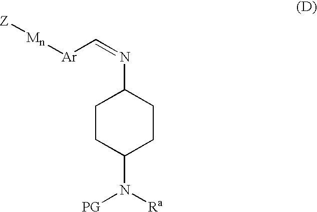
with a reducing agent under imine reduction conditions, wherein, as valence and stability permit,

Ar is a substituted or unsubstituted aryl or heteroaryl, such as a substituted or unsubstituted phenyl;

M, independently for each occurrence, represents a substituted or unsubstituted methylene group (e.g., substituted with lower alkyl, oxo, hydroxyl, etc.), NR⁴ (wherein R⁴ is H, substituted or unsubstituted alkyl, such as substituted or unsubstituted lower alkyl, or an amine protecting group), O, S, S(O), or S(O)₂, preferably selected such that no two heteroatoms are adjacent to each other;

n is an integer from 0-4, such as 0, 1, or 2, particularly 0;

Z is H, a substituted or unsubstituted aryl, carbocyclyl, heterocyclyl, or heteroaryl, or a nitro, cyano, or halogen;

R^{a} is H, substituted or unsubstituted alkyl, such as a substituted or unsubstituted lower alkyl, for example, methyl, or an amine protecting group; and

PG is an amine protecting group, such as tert-butylcarboxy.

In certain embodiments, the reducing agent is a borohydride, such as a group I metal borohydride. For example, in certain embodiments, the reducing agent is sodium borohydride, sodium cyanoborohydride, or sodium triacetoxyborohydride, particularly sodium triacetoxyborohydride.

In some embodiments, the reducing agent and the compound of Formula D are combined in about a 4:1 to 3 ratio, respectively.

In certain embodiments, the imine reduction conditions comprise maintaining a temperature below room temperature, for example, about 2, 5, 7, 10, 15, 20, 30, or 40° C. below room temperature. In certain embodiments, the imine reduction conditions comprise maintaining a temperature between about −10 and about 20° C., such as between about −3 and about 10° C., particularly between about 2 and about 5° C.

In some embodiments, the imine reduction conditions comprise one or more organic or aqueous solvent systems or combinations thereof. For example, in certain embodiments, the imine reduction conditions comprise an alkyl alcohol, such as methanol, wherein the alkyl alcohol may represent part of the solvent system, for example, about 10, 20, 30, 50, 70, or 90% of the solvent system, or substantially all of the solvent system, for example about 95% or greater of the solvent system.

In certain embodiments, n is 0.

In certain embodiments, Z is H. In other embodiments, Z is a substituted or unsubstituted aryl, carbocyclyl, heterocyclyl, or heteroaryl. For example, in some embodiments Z is a substituted or unsubstituted thiophene, thianthrene, furan, pyran, isobenzofuran, chromene, xanthene, phenoxathiin, pyrrole, imidazole, pyrazole, isothiazole, isoxazole, pyridine, pyrazine, pyrimidine, pyridazine, indolizine, isoindole, indole, indazole, purine, quinolizine, isoquinoline, quinoline, phthalazine, naphthyridine, quinoxaline, quinazoline, cinnoline, pteridine, carbazole, carboline, phenanthridine, acridine, phenanthroline, phenazine, phenarsazine, phenothiazine, furazan, phenoxazine, pyrrolidine, oxolane, thiolane, oxazole, piperidine, piperazine, morpholine, lactone, lactam, azetidinone, pyrrolidinone, sultam, or sultone. In particular embodiments, Z is a substituted or unsubstituted aryl or heteroaryl, such as a substituted or unsubstituted phenyl. In other embodiments, Z is a substituted or unsubstituted carbocyclyl or heterocyclyl.

In some embodiments, Z is a substituted or unsubstituted aryl ring, such as a phenyl ring.

In other embodiments, Z is a substituted or unsubstituted heteroaryl ring, such as a substituted or unsubstituted pyridine, pyrimidine, pyrazine, pyridazine, triazine, tetrazine, pyrrole, pyrazole, or imidazole ring or N-oxide thereof. In specific embodiments, Z is a substituted or unsubstituted pyridine, or pyrazine ring or N-oxide thereof, particularly a substituted or unsubstituted pyridine ring or N-oxide thereof.

In some examples, Z is a substituted or unsubstituted pyridine ring or N-oxide thereof where connection of the pyridine ring to the phenyl ring bearing R¹ and R² can occur at any location on the pyridine ring, for example, at a 2-, 3-, or 4-position relative to the nitrogen of the pyridine ring, i.e., at an ortho-, meta-, or para-position relative to the nitrogen of the pyridine ring. In certain embodiments, Z is a substituted or unsubstituted pyridine N-oxide.

In other embodiments, Z is a substituted or unsubstituted pyrimidine ring or N-oxide thereof where connection of the pyrimidine ring to the phenyl ring bearing R¹ and R² can occur at any location on the pyrimidine ring, for example, at a 2-, 3-, 4-, 5-, or 6-position relative to N1 of the pyrimidine ring.

In some embodiments, Z is unsubstituted. In other embodiments, Z is substituted with one or more groups selected from halogen, lower alkyl, lower alkenyl, —CN, azido, —NR^{x}R^{x}, —CO₂OR^{x}, —C(O)—NR^{x}R^{x}, —C(O)—R^{x}, —NR^{x}—C(O)—R^{x}, —NR^{x}SO₂R^{x}, —SR^{x}, —S(O)R^{x}, —SO₂R^{x}, —SO₂NR^{x}R^{x}, —(C(R^{x})₂)ₙ—OR^{x}, —(C(R^{x})₂)ₙ—NR^{x}R^{x}, and —(C(R^{x})₂)ₙ—SO₂R^{x}; wherein R^{x} are, independently for each occurrence, H or lower alkyl; and n is, independently for each occurrence, an integer from 0 to 2. For example, in some embodiments, Z is substituted with halogen, such as fluoro.

In certain embodiments, Z is substituted with one or more electron withdrawing groups. For example, sometimes Z is substituted with one or more groups selected from halogen, —CN, azido, —CO₂OR^{x}, —C(O)—NR^{x}R^{x}, and —C(O)—R^{x}.

Combinations of the various values for Z are contemplated. In some instances, suitable compounds include those where Z is a pyridine ring or N-oxide thereof and Z is substituted by fluoro at any carbon position on the pyridine ring, such as a 2-, 3-, or 4-position relative to the nitrogen of the pyridine ring.

In some embodiments, the compound of Formula D has the structure: 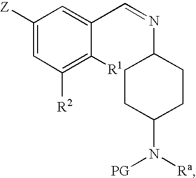
wherein R¹ and R² are, independently, H, halogen, hydroxyl, lower alkyl, or lower alkoxy, provided that at least one of R¹ and R² is not H.

In certain embodiments, R¹ and R² are, independently, H, fluorine, hydroxyl, lower alkyl, methoxy, or ethoxy, provided that at least one of R¹ and R² is not H.

In particular embodiments, R¹ is hydroxyl, ethyl, propyl, methoxy, or ethoxy; and R² is H, fluorine, hydroxyl, lower alkyl, methoxy, or ethoxy. In certain such embodiments, R² is H.

In certain embodiments, at least one of R¹ or R² is methoxy. For example, in some embodiments, R¹ is methoxy and R² is H, while in others, R² is methoxy and R¹ is H. In some instances, both R¹ and R² are methoxy.

In certain embodiments, at least one of R¹ or R² is lower alkyl. For example, sometimes R¹ is lower alkyl, such as methyl, ethyl, or propyl.

In certain embodiments, at least one of R¹ or R² is hydroxyl. For example, sometimes R¹ is hydroxyl.

In certain embodiments, Z is a substituted or unsubstituted aryl or heteroaryl ring; R^{a} is methyl; R¹ is halogen, such as fluoro or chloro, methoxy, or ethoxy; and R² is H. In some embodiments, R¹ is methoxy. In other embodiments, R¹ is fluoro. In some instances, R¹ is ethoxy. In some instances, Z is not a substituted or unsubstituted pyridine N-oxide ring or a pyridine ring substituted with one or more halogens. In certain embodiments, Z is substituted with one or more electron withdrawing groups.

In certain embodiments, Z is a substituted or unsubstituted aryl or heteroaryl ring; R^{a} is H or methyl; R¹ is H; and R² is halogen, such as fluoro or chloro, hydroxyl, methyl, ethyl, methoxy, or ethoxy. In some embodiments, R² is methoxy. In certain embodiments, R^{a} is methyl. In certain embodiments, Z is substituted with one or more electron withdrawing groups.

In certain embodiments, Z is a substituted or unsubstituted aryl or heteroaryl ring; R^{a} is H or methyl; R¹ is hydroxyl, methyl, or ethyl; and R² is H, halogen, such as fluoro or chloro, hydroxyl, methyl, ethyl, methoxy, or ethoxy. In certain embodiments, R^{a} is methyl. In certain embodiments, R² is H. In certain embodiments, R¹ is hydroxyl. In certain embodiments, R¹ is methyl. In certain embodiments, R¹ is ethyl. In some embodiments, one or both of R¹ or R² is hydroxyl; for example, sometimes R¹ is hydroxyl. In certain embodiments, Z is substituted with one or more electron withdrawing groups.

In certain embodiments, Z is a substituted or unsubstituted pyridine N-oxide ring; R^{a} is H or methyl; and R¹ and R² are, independently, H, halogen, such as fluoro or chloro, hydroxyl, methyl, ethyl, methoxy, or ethoxy, provided that at least one of R¹ and R² is not H. In certain embodiments, one or both of R¹ or R² is methoxy; for example, sometimes R¹ is methoxy, sometimes R² is methoxy, and sometimes both R¹ and R² are methoxy. In certain embodiments, one or both of R¹ or R² is fluoro; for example, sometimes R¹ is fluoro, sometimes R² is fluoro, and sometimes both R¹ and R² are fluoro. In certain embodiments, R² is H. In certain embodiments, R¹ is hydroxyl. In certain embodiments, R¹ is methyl. In certain embodiments, R¹ is ethyl. In some embodiments, one or both of R¹ or R² is hydroxyl; for example, sometimes R¹ is hydroxyl. In certain embodiments, Z is substituted with one or more electron withdrawing groups.

In certain embodiments, Z is a pyridine ring substituted with one or more halogens, such as fluoro and/or chloro, and optionally further substituted; R^{a} is H or methyl; and R¹ and R² are, independently, H, halogen, such as fluoro or chloro, hydroxyl, methyl, ethyl, methoxy, or ethoxy, provided that at least one of R¹ and R² is not H. In certain embodiments, one or both of R¹ or R² is methoxy; for example, sometimes R¹ is methoxy, sometimes R² is methoxy, and sometimes both R¹ and R² are methoxy. In certain embodiments, one or both of R¹ or R² is fluoro; for example, sometimes R¹ is fluoro, sometimes R² is fluoro, and sometimes both R¹ and R² are fluoro. In certain embodiments, one or both of R¹ or R² is fluoro; for example, sometimes R¹ is fluoro, sometimes R² is fluoro, and sometimes both R¹ and R² are fluoro. In certain embodiments, R² is H. In certain embodiments, R¹ is hydroxyl. In certain embodiments, R¹ is methyl. In certain embodiments, R¹ is ethyl. In some embodiments, one or both of R¹ or R² is hydroxyl; for example, sometimes R¹ is hydroxyl. In certain embodiments, Z is substituted with one or more electron withdrawing groups.

In certain embodiments, Z is a substituted or unsubstituted aryl or heteroaryl ring; R^{a} is H; and R¹ and R² are, independently, H, halogen, such as fluoro or chloro, hydroxyl, methyl, ethyl, methoxy, or ethoxy, provided that at least one of R¹ and R² is not H. In certain embodiments, one or both of R¹ or R² is methoxy; for example, sometimes R¹ is methoxy, sometimes R² is methoxy, and sometimes both R¹ and R² are methoxy. In certain embodiments, R² is H. In certain embodiments, R¹ is hydroxyl. In certain embodiments, R¹ is methyl. In certain embodiments, R¹ is ethyl. In some embodiments, one or both of R¹ or R² is hydroxyl; for example, sometimes R¹ is hydroxyl. In certain embodiments, Z is substituted with one or more electron withdrawing groups.

In certain embodiments, the compound of Formula D has the structure:

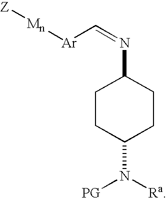

In certain embodiments, the present invention provides a method for the preparation of a compound of Formula G, or a salt thereof: 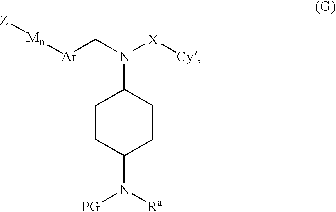
comprising treating a compound of Formula E, or a salt thereof, with a compound of Formula F, or a salt thereof: 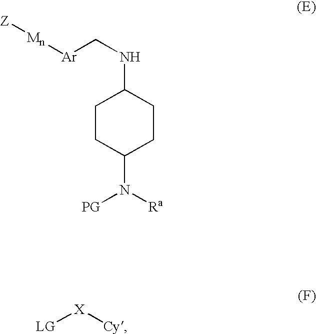
under acylation conditions, wherein, as valence and stability permit,

Ar is a substituted or unsubstituted aryl or heteroaryl, such as a substituted or unsubstituted phenyl;

M, independently for each occurrence, represents a substituted or unsubstituted methylene group (e.g., substituted with lower alkyl, oxo, hydroxyl, etc.), NR⁴ (wherein R⁴ is H, substituted or unsubstituted alkyl, such as substituted or unsubstituted lower alkyl, or an amine protecting group), O, S, S(O), or S(O)₂, preferably selected such that no two heteroatoms are adjacent to each other;

n is an integer from 0-4, such as 0, 1, or 2, particularly 0;

Z is H, a substituted or unsubstituted aryl, carbocyclyl, heterocyclyl, or heteroaryl, or a nitro, cyano, or halogen,

R^{a} is H, substituted or unsubstituted alkyl, such as a substituted or unsubstituted lower alkyl, for example, methyl, or an amine protecting group;

PG is an amine protecting group, such as tert-butylcarboxy;

LG is a leaving group, such as halogen, for example chloro;

X is —C(═O)—, —C(═S)—, —S(O₂)—, —S(O)—, —C(═NCN)—, or —P(═O)(OR)—, particularly —C(═O)-—C(═S)—, or —S(O₂)—; and

Cy′ is a substituted or unsubstituted benzothiophene, such as a substituted or unsubstituted 2-benzothiophene.

In certain embodiments, the acylation conditions comprise an amine base, such as a trialkyl amine base, for example, diisopropylethylamine.

In some embodiments, the amine base and the compound of Formula E are combined in about a 6:1 to 3 ratio, respectively.

In certain embodiments, the compound of Formula F and the compound of Formula E are combined in about a 1.5 to 1:1 ratio, respectively.

In some embodiments, the acylation conditions comprise maintaining a temperature below room temperature, for example, about 2, 5, 7, 10, 15, 20, 30, or 40° C. below room temperature. In certain embodiments, the acylation conditions comprise maintaining a temperature between about −20 and about 20° C., such as between about −10 and about 10° C., particularly between about −6 and about 6° C.

In some embodiments, the acylation conditions comprise one or more organic or aqueous solvent systems or combinations thereof. For example, in certain embodiments, the acylation conditions comprise an alkyl acetate, for example isopropyl acetate, wherein the alkyl acetate may represent part of the solvent system, for example, about 10, 20, 30, 50, 70, or 90% of the solvent system, or substantially all of the solvent system, for example about 95% or greater of the solvent system.

In certain embodiments the product of this reaction is purified by trituration with an alkyl acetate, such as ethyl acetate (e.g. without a need for chromatography or other purification steps).

In certain embodiments, n is 0.

In certain embodiments, Z is H. In other embodiments, Z is a substituted or unsubstituted aryl, carbocyclyl, heterocyclyl, or heteroaryl. For example, in some embodiments Z is a substituted or unsubstituted thiophene, thianthrene, furan, pyran, isobenzofuran, chromene, xanthene, phenoxathiin, pyrrole, imidazole, pyrazole, isothiazole, isoxazole, pyridine, pyrazine, pyrimidine, pyridazine, indolizine, isoindole, indole, indazole, purine, quinolizine, isoquinoline, quinoline, phthalazine, naphthyridine, quinoxaline, quinazoline, cinnoline, pteridine, carbazole, carboline, phenanthridine, acridine, phenanthroline, phenazine, phenarsazine, phenothiazine, furazan, phenoxazine, pyrrolidine, oxolane, thiolane, oxazole, piperidine, piperazine, morpholine, lactone, lactam, azetidinone, pyrrolidinone, sultam, or sultone. In particular embodiments, Z is a substituted or unsubstituted aryl or heteroaryl, such as a substituted or unsubstituted phenyl. In other embodiments, Z is a substituted or unsubstituted carbocyclyl or heterocyclyl.

In some embodiments, Z is a substituted or unsubstituted aryl ring, such as a phenyl ring.

In other embodiments, Z is a substituted or unsubstituted heteroaryl ring, such as a substituted or unsubstituted pyridine, pyrimidine, pyrazine, pyridazine, triazine, tetrazine, pyrrole, pyrazole, or imidazole ring or N-oxide thereof. In specific embodiments, Z is a substituted or unsubstituted pyridine, or pyrazine ring or N-oxide thereof, particularly a substituted or unsubstituted pyridine ring or N-oxide thereof.

In some examples, Z is a substituted or unsubstituted pyridine ring or N-oxide thereof where connection of the pyridine ring to the phenyl ring bearing R¹ and R² can occur at any location on the pyridine ring, for example, at a 2-, 3-, or 4-position relative to the nitrogen of the pyridine ring, i.e., at an ortho-, meta-, or para-position relative to the nitrogen of the pyridine ring. In certain embodiments, Z is a substituted or unsubstituted pyridine N-oxide.

In other embodiments, Z is a substituted or unsubstituted pyrimidine ring or N-oxide thereof where connection of the pyrimidine ring to the phenyl ring bearing R¹ and R² can occur at any location on the pyrimidine ring, for example, at a 2-, 3-, 4-, 5-, or 6-position relative to N1 of the pyrimidine ring.

In some embodiments, Z is unsubstituted. In other embodiments, Z is substituted with one or more groups selected from halogen, lower alkyl, lower alkenyl, —CN, azido, —NR^{x}R^{x}, —CO₂OR^{x}, —C(O)—NR^{x}R^{x}, —C(O)—R^{x}, —NR^{x}—C(O)—R^{x}, —NR^{x}SO₂R^{x}, —SR^{x}, —S(O)R^{x}, —SO₂R^{x}, —SO₂NR^{x}R^{x}, —(C(R^{x})₂)ₙ—OR^{x}, (C(R^{x})₂)ₙ—NR^{x}R^{x}, and —(C(R^{x})₂)ₙ—SO₂R^{x}; wherein R^{x} are, independently for each occurrence, H or lower alkyl; and n is, independently for each occurrence, an integer from 0 to 2. For example, in some embodiments, Z is substituted with halogen, such as fluoro.

In certain embodiments, Z is substituted with one or more electron withdrawing groups. For example, sometimes Z is substituted with one or more groups selected from halogen, —CN, azido, —CO₂OR^{x}, —C(O)—NR^{x}R^{x}, and —C(O)—R^{x}.

Combinations of the various values for Z are contemplated. In some instances, suitable compounds include those where Z is a pyridine ring or N-oxide thereof and Z is substituted by fluoro at any carbon position on the pyridine ring, such as a 2-, 3-, or 4-position relative to the nitrogen of the pyridine ring.

In some embodiments, the compound of Formula E has the structure: 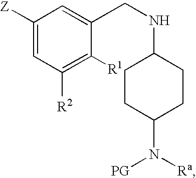
wherein R¹ and R² are, independently, H, halogen, hydroxyl, lower alkyl, or lower alkoxy, provided that at least one of R¹ and R² is not H.

In certain embodiments, R¹ and R² are, independently, H, fluorine, hydroxyl, lower alkyl, methoxy, or ethoxy, provided that at least one of R¹ and R² is not H.

In particular embodiments, R¹ is hydroxyl, ethyl, propyl, methoxy, or ethoxy; and R² is H, fluorine, hydroxyl, lower alkyl, methoxy, or ethoxy. In certain such embodiments, R² is H.

In certain embodiments, at least one of R¹ or R² is methoxy. For example, in some embodiments, R¹ is methoxy and R² is H, while in others, R² is methoxy and R¹ is H. In some instances, both R¹ and R² are methoxy.

In certain embodiments, at least one of R¹ or R² is lower alkyl. For example, sometimes R¹ is lower alkyl, such as methyl, ethyl, or propyl.

In certain embodiments, at least one of R¹ or R² is hydroxyl. For example, sometimes R¹ is hydroxyl.

In certain embodiments, Z is a substituted or unsubstituted aryl or heteroaryl ring; R^{a} is methyl; R¹ is halogen, such as fluoro or chloro, methoxy, or ethoxy; and R² is H. In some embodiments, R¹ is methoxy. In other embodiments, R¹ is fluoro. In some instances, R¹ is ethoxy. In some instances, Z is not a substituted or unsubstituted pyridine N-oxide ring or a pyridine ring substituted with one or more halogens. In certain embodiments, Z is substituted with one or more electron withdrawing groups.

In certain embodiments, Z is a substituted or unsubstituted aryl or heteroaryl ring; R^{a} is H or methyl; R¹ is H; and R² is halogen, such as fluoro or chloro, hydroxyl, methyl, ethyl, methoxy, or ethoxy. In some embodiments, R² is methoxy. In certain embodiments, R^{a} is methyl. In certain embodiments, Z is substituted with one or more electron withdrawing groups.

In certain embodiments, Z is a substituted or unsubstituted aryl or heteroaryl ring; R^{a} is H or methyl; R¹ is hydroxyl, methyl, or ethyl; and R² is H, halogen, such as fluoro or chloro, hydroxyl, methyl, ethyl, methoxy, or ethoxy. In certain embodiments, R^{a} is methyl. In certain embodiments, R² is H. In certain embodiments, R¹ is hydroxyl. In certain embodiments, R¹ is methyl. In certain embodiments, R¹ is ethyl. In some embodiments, one or both of R¹ or R² is hydroxyl; for example, sometimes R¹ is hydroxyl. In certain embodiments, Z is substituted with one or more electron withdrawing groups.

In certain embodiments, Z is a substituted or unsubstituted pyridine N-oxide ring; R^{a} is H or methyl; and R¹ and R² are, independently, H, halogen, such as fluoro or chloro, hydroxyl, methyl, ethyl, methoxy, or ethoxy, provided that at least one of R¹ and R² is not H. In certain embodiments, one or both of R¹ or R² is methoxy; for example, sometimes R¹ is methoxy, sometimes R² is methoxy, and sometimes both R¹ and R² are methoxy. In certain embodiments, one or both of R¹ or R² is fluoro; for example, sometimes R¹ is fluoro, sometimes R² is fluoro, and sometimes both R¹ and R² are fluoro. In certain embodiments, R² is H. In certain embodiments, R¹ is hydroxyl. In certain embodiments, R¹ is methyl. In certain embodiments, R¹ is ethyl. In some embodiments, one or both of R¹ or R² is hydroxyl; for example, sometimes R¹ is hydroxyl. In certain embodiments, Z is substituted with one or more electron withdrawing groups.

In certain embodiments, Z is a pyridine ring substituted with one or more halogens, such as fluoro and/or chloro, and optionally further substituted; R^{a} is H or methyl; and R¹ and R² are, independently, H, halogen, such as fluoro or chloro, hydroxyl, methyl, ethyl, methoxy, or ethoxy, provided that at least one of R¹ and R² is not H. In certain embodiments, one or both of R¹ or R² is methoxy; for example, sometimes R¹ is methoxy, sometimes R² is methoxy, and sometimes both R¹ and R² are methoxy. In certain embodiments, one or both of R¹ or R² is fluoro; for example, sometimes R¹ is fluoro, sometimes R² is fluoro, and sometimes both R¹ and R² are fluoro. In certain embodiments, one or both of R¹ or R² is fluoro; for example, sometimes R¹ is fluoro, sometimes R² is fluoro, and sometimes both R¹ and R² are fluoro. In certain embodiments, R² is H. In certain embodiments, R¹ is hydroxyl. In certain embodiments, R¹ is methyl. In certain embodiments, R¹ is ethyl. In some embodiments, one or both of R¹ or R² is hydroxyl; for example, sometimes R¹ is hydroxyl. In certain embodiments, Z is substituted with one or more electron withdrawing groups.

In certain embodiments, Z is a substituted or unsubstituted aryl or heteroaryl ring; R^{a} is H; and R¹ and R² are, independently, H, halogen, such as fluoro or chloro, hydroxyl, methyl, ethyl, methoxy, or ethoxy, provided that at least one of R¹ and R² is not H. In certain embodiments, one or both of R¹ or R² is methoxy; for example, sometimes R¹ is methoxy, sometimes R² is methoxy, and sometimes both R¹ and R² are methoxy. In certain embodiments, R² is H. In certain embodiments, R¹ is hydroxyl. In certain embodiments, R¹ is methyl. In certain embodiments, R¹ is ethyl. In some embodiments, one or both of R¹ or R² is hydroxyl; for example, sometimes R¹ is hydroxyl. In certain embodiments, Z is substituted with one or more electron withdrawing groups.

In certain embodiments, the compound of Formula E has the structure:

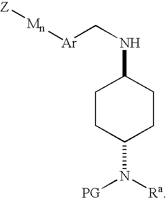

In some embodiments, Cy′ is substituted with one or more halogen and/or alkyl groups. For example, in some instances Cy′ is substituted with one or more chloro and/or fluoro groups.

In particular embodiments, Cy′ is a 3-halo-benzo[b]thien-2-yl, for example a 3-chloro-benzo[b]thien-2-yl or a 3-fluoro-benzo[b]thien-2-yl, or a 3-methyl-benzo[b]thien-2-yl. The benzo ring of Cy′ may be substituted with from 1-4 substituents such as halogen, nitro, cyano, methyl (e.g., including halomethyl, such as CHCl₂ and CF₃), and ethyl (e.g., including haloethyl, such as CH₂CCl₃, C₂F₅, etc.), preferably with halogen and methyl (e.g., including halomethyl, such as CHCl₂ and CF₃). In certain such embodiments, Cy′ represents a 3-chloro-benzo[b]thien-2-yl, 3-fluoro-benzo[b]thien-2-yl, or 3-methyl-benzo[b]thien-2-yl, wherein the benzo ring is substituted with fluoro at the 4-position (peri to the 3-substituent on the thienyl ring) and, optionally, at the 7-position (‘peri’ to the S of the thienyl ring). Alternatively, the benzo ring may be unsubstituted. In certain embodiments, the benzo ring is selected from: 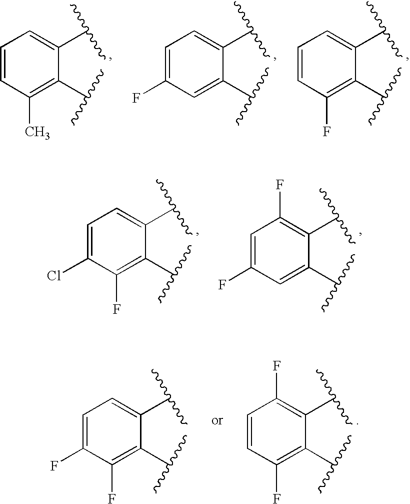
In yet further embodiments, Cy′ is selected from:

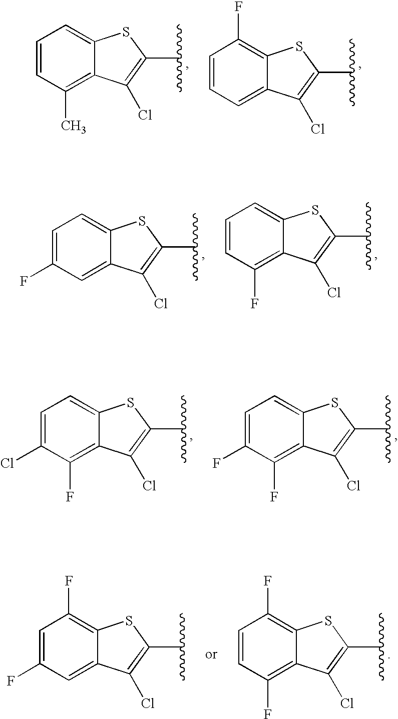

In certain embodiments, the present invention provides a method for the preparation of a compound of Formula I, or a pharmaceutically acceptable salt thereof: 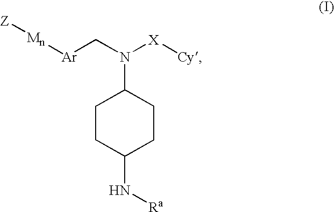
comprising subjecting a compound of Formula G, or a salt thereof: 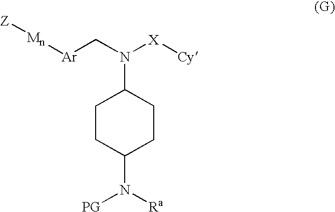
to amine deprotection conditions, wherein, as valence and stability permit,

Ar is a substituted or unsubstituted aryl or heteroaryl, such as a substituted or unsubstituted phenyl;

M, independently for each occurrence, represents a substituted or unsubstituted methylene group (e.g., substituted with lower alkyl, oxo, hydroxyl, etc.), NR⁴ (wherein R⁴ is H, substituted or unsubstituted alkyl, such as substituted or unsubstituted lower alkyl, or an amine protecting group), O, S, S(O), or S(O)₂, preferably selected such that no two heteroatoms are adjacent to each other;

n is an integer from 0-4, such as 0, 1, or 2, particularly 0;

Z is H, a substituted or unsubstituted aryl, carbocyclyl, heterocyclyl, or heteroaryl, or a nitro, cyano, or halogen,

R^{a} is H, a substituted or unsubstituted alkyl, such as a substituted or unsubstituted lower alkyl, for example, methyl, or an amine protecting group;

PG is an amine protecting group, such as tert-butylcarboxy;

X is —C(═O)—, —C(═S)—, —S(O₂)—, —S(O)—, —C(═NCN)—, or —P(═O)(OR)—, particularly —C(═O)-—C(═S)—, or —S(O₂)—; and

Cy′ is a substituted or unsubstituted benzothiophene, such as a substituted or unsubstituted 2-benzothiophene.

In certain embodiments, PG is a carbamate amine protecting group and the amine deprotection conditions comprise an amine protecting group removing reagent that removes carbamate amine protecting groups.

In some embodiments, the amine deprotection conditions comprise an acid, such as HCl. In certain embodiments, the acid and the compound of Formula G are combined in about a 4 to 2.5:1 ratio, respectively.

In certain embodiments, the amine deprotection conditions comprise maintaining a temperature above room temperature, for example, about 5, 10, 20, 40, 60, or 80° C. above room temperature. In certain embodiments, the amine deprotection conditions comprise maintaining a temperature between about 45 and about 95° C., such as between about 55 and about 85° C., particularly between about 65 and about 75° C.

In some embodiments, the amine deprotection conditions comprise one or more organic or aqueous solvent systems or combinations thereof. For example, in certain embodiments, the amine deprotection conditions comprise an alkyl alcohol, such as ethanol, wherein the alkyl alcohol may represent part of the solvent system, for example, about 10, 20, 30, 50, 70, or 90% of the solvent system, or substantially all of the solvent system, for example about 95% or greater of the solvent system.

In some embodiments, the present methods provide a pharmaceutically acceptable salt of the compound of Formula I. In certain such embodiments, the salt is formed by further treating the compound of Formula I with an acid, for example HCl.

In certain embodiments, n is 0.

In certain embodiments, Z is H. In other embodiments, Z is a substituted or unsubstituted aryl, carbocyclyl, heterocyclyl, or heteroaryl. For example, in some embodiments Z is a substituted or unsubstituted thiophene, thianthrene, furan, pyran, isobenzofuran, chromene, xanthene, phenoxathiin, pyrrole, imidazole, pyrazole, isothiazole, isoxazole, pyridine, pyrazine, pyrimidine, pyridazine, indolizine, isoindole, indole, indazole, purine, quinolizine, isoquinoline, quinoline, phthalazine, naphthyridine, quinoxaline, quinazoline, cinnoline, pteridine, carbazole, carboline, phenanthridine, acridine, phenanthroline, phenazine, phenarsazine, phenothiazine, furazan, phenoxazine, pyrrolidine, oxolane, thiolane, oxazole, piperidine, piperazine, morpholine, lactone, lactam, azetidinone, pyrrolidinone, sultam, or sultone. In particular embodiments, Z is a substituted or unsubstituted aryl or heteroaryl, such as a substituted or unsubstituted phenyl. In other embodiments, Z is a substituted or unsubstituted carbocyclyl or heterocyclyl.

In some embodiments, Z is a substituted or unsubstituted aryl ring, such as a phenyl ring.

In other embodiments, Z is a substituted or unsubstituted heteroaryl ring, such as a substituted or unsubstituted pyridine, pyrimidine, pyrazine, pyridazine, triazine, tetrazine, pyrrole, pyrazole, or imidazole ring or N-oxide thereof. In specific embodiments, Z is a substituted or unsubstituted pyridine, or pyrazine ring or N-oxide thereof, particularly a substituted or unsubstituted pyridine ring or N-oxide thereof.

In some examples, Z is a substituted or unsubstituted pyridine ring or N-oxide thereof where connection of the pyridine ring to the phenyl ring bearing R¹ and R² can occur at any location on the pyridine ring, for example, at a 2-, 3-, or 4-position relative to the nitrogen of the pyridine ring, i.e., at an ortho-, meta-, or para-position relative to the nitrogen of the pyridine ring. In certain embodiments, Z is a substituted or unsubstituted pyridine N-oxide.

In other embodiments, Z is a substituted or unsubstituted pyrimidine ring or N-oxide thereof where connection of the pyrimidine ring to the phenyl ring bearing R¹ and R² can occur at any location on the pyrimidine ring, for example, at a 2-, 3-, 4-, 5-, or 6-position relative to N1 of the pyrimidine ring.

In some embodiments, Z is unsubstituted. In other embodiments, Z is substituted with one or more groups selected from halogen, lower alkyl, lower alkenyl, —CN, azido, —NR^{x}R^{x}, —CO₂OR^{x}, —C(O)—NR^{x}R^{x}, —C(O)—R^{x}, —NR^{x}—C(O)—R^{x}, NR^{x}SO₂R^{x}, SR^{x}, —S(O)R^{x}, —SO₂R^{x}, —SO₂NR^{x}R^{x}, —(C(R^{x})₂)ₙ—OR^{x}, (C(R^{x})₂)ₙ—NR^{x}R^{x}, and —(C(R^{x})₂)ₙ—SO₂R^{x}; wherein R^{x} are, independently for each occurrence, H or lower alkyl; and n is, independently for each occurrence, an integer from 0 to 2. For example, in some embodiments, Z is substituted with halogen, such as fluoro.

In certain embodiments, Z is substituted with one or more electron withdrawing groups. For example, sometimes Z is substituted with one or more groups selected from halogen, —CN, azido, —CO₂OR^{x}, —C(O)—NR^{x}R^{x}, and —C(O)—R^{x}.

Combinations of the various values for Z are contemplated. In some instances, suitable compounds include those where Z is a pyridine ring or N-oxide thereof and Z is substituted by fluoro at any carbon position on the pyridine ring, such as a 2-, 3-, or 4-position relative to the nitrogen of the pyridine ring.

In certain embodiments, the compound of Formula G has the structure: 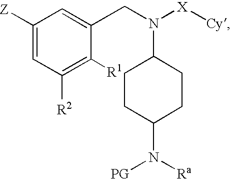
wherein R¹ and R² are, independently, H, halogen, hydroxyl, lower alkyl, or lower alkoxy, provided that at least one of R¹ and R² is not H.

In certain embodiments, R¹ and R² are, independently, H, fluorine, hydroxyl, lower alkyl, methoxy, or ethoxy, provided that at least one of R¹ and R² is not H.

In particular embodiments, R¹ is hydroxyl, ethyl, propyl, methoxy, or ethoxy; and R² is H, fluorine, hydroxyl, lower alkyl, methoxy, or ethoxy. In certain such embodiments, R² is H.

In certain embodiments, at least one of R¹ or R² is methoxy. For example, in some embodiments, R¹ is methoxy and R² is H, while in others, R² is methoxy and R¹ is H. In some instances, both R¹ and R² are methoxy.

In certain embodiments, at least one of R¹ or R² is lower alkyl. For example, sometimes R¹ is lower alkyl, such as methyl, ethyl, or propyl.

In certain embodiments, at least one of R¹ or R² is hydroxyl. For example, sometimes R¹ is hydroxyl.

In certain embodiments, Z is a substituted or unsubstituted aryl or heteroaryl ring; R^{a} is methyl; R¹ is halogen, such as fluoro or chloro, methoxy, or ethoxy; and R² is H. In some embodiments, R¹ is methoxy. In other embodiments, R¹ is fluoro. In some instances, R¹ is ethoxy. In some instances, Z is not a substituted or unsubstituted pyridine N-oxide ring or a pyridine ring substituted with one or more halogens. In certain embodiments, Z is substituted with one or more electron withdrawing groups.

In certain embodiments, Z is a substituted or unsubstituted aryl or heteroaryl ring; R^{a} is H or methyl; R¹ is H; and R² is halogen, such as fluoro or chloro, hydroxyl, methyl, ethyl, methoxy, or ethoxy. In some embodiments, R² is methoxy. In certain embodiments, R^{a} is methyl. In certain embodiments, Z is substituted with one or more electron withdrawing groups.

In certain embodiments, Z is a substituted or unsubstituted aryl or heteroaryl ring; R^{a} is H or methyl; R¹ is hydroxyl, methyl, or ethyl; and R² is H, halogen, such as fluoro or chloro, hydroxyl, methyl, ethyl, methoxy, or ethoxy. In certain embodiments, R^{a} is methyl. In certain embodiments, R² is H. In certain embodiments, R¹ is hydroxyl. In certain embodiments, R¹ is methyl. In certain embodiments, R¹ is ethyl. In some embodiments, one or both of R¹ or R² is hydroxyl; for example, sometimes R¹ is hydroxyl. In certain embodiments, Z is substituted with one or more electron withdrawing groups.

In certain embodiments, Z is a substituted or unsubstituted pyridine N-oxide ring; R^{a} is H or methyl; and R¹ and R² are, independently, H, halogen, such as fluoro or chloro, hydroxyl, methyl, ethyl, methoxy, or ethoxy, provided that at least one of R¹ and R² is not H. In certain embodiments, one or both of R¹ or R² is methoxy; for example, sometimes R¹ is methoxy, sometimes R² is methoxy, and sometimes both R¹ and R² are methoxy. In certain embodiments, one or both of R¹ or R² is fluoro; for example, sometimes R¹ is fluoro, sometimes R² is fluoro, and sometimes both R¹ and R² are fluoro. In certain embodiments, R² is H. In certain embodiments, R¹ is hydroxyl. In certain embodiments, R¹ is methyl. In certain embodiments, R¹ is ethyl. In some embodiments, one or both of R¹ or R² is hydroxyl; for example, sometimes R¹ is hydroxyl. In certain embodiments, Z is substituted with one or more electron withdrawing groups.

In certain embodiments, Z is a pyridine ring substituted with one or more halogens, such as fluoro and/or chloro, and optionally further substituted; R^{a} is H or methyl; and R¹ and R² are, independently, H, halogen, such as fluoro or chloro, hydroxyl, methyl, ethyl, methoxy, or ethoxy, provided that at least one of R¹ and R² is not H. In certain embodiments, one or both of R¹ or R² is methoxy; for example, sometimes R¹ is methoxy, sometimes R² is methoxy, and sometimes both R¹ and R² are methoxy. In certain embodiments, one or both of R¹ or R² is fluoro; for example, sometimes R¹ is fluoro, sometimes R² is fluoro, and sometimes both R¹ and R² are fluoro. In certain embodiments, one or both of R¹ or R² is fluoro; for example, sometimes R¹ is fluoro, sometimes R² is fluoro, and sometimes both R¹ and R² are fluoro. In certain embodiments, R² is H. In certain embodiments, R¹ is hydroxyl. In certain embodiments, R¹ is methyl. In certain embodiments, R¹ is ethyl. In some embodiments, one or both of R¹ or R² is hydroxyl; for example, sometimes R¹ is hydroxyl. In certain embodiments, Z is substituted with one or more electron withdrawing groups.

In certain embodiments, Z is a substituted or unsubstituted aryl or heteroaryl ring; R^{a} is H; and R¹ and R² are, independently, H, halogen, such as fluoro or chloro, hydroxyl, methyl, ethyl, methoxy, or ethoxy, provided that at least one of R¹ and R² is not H. In certain embodiments, one or both of R¹ or R² is methoxy; for example, sometimes R¹ is methoxy, sometimes R² is methoxy, and sometimes both R¹ and R² are methoxy. In certain embodiments, R² is H. In certain embodiments, R¹ is hydroxyl. In certain embodiments, R¹ is methyl. In certain embodiments, R¹ is ethyl. In some embodiments, one or both of R¹ or R² is hydroxyl; for example, sometimes R¹ is hydroxyl. In certain embodiments, Z is substituted with one or more electron withdrawing groups.

In some embodiments, the compound of Formula G has the structure:

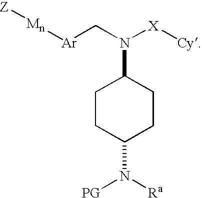

In some embodiments, Cy′ is substituted with one or more halogen and/or alkyl groups. For example, in some instances Cy′ is substituted with one or more chloro and/or fluoro groups.

In particular embodiments, Cy′ is a 3-halo-benzo[b]thien-2-yl, for example a 3-chloro-benzo[b]thien-2-yl or a 3-fluoro-benzo[b]thien-2-yl, or a 3-methyl-benzo[b]thien-2-yl. The benzo ring of Cy′ may be substituted with from 1-4 substituents such as halogen, nitro, cyano, methyl (e.g., including halomethyl, such as CHCl₂ and CF₃), and ethyl (e.g., including haloethyl, such as CH₂CCl₃, C₂F₅, etc.), preferably with halogen and methyl (e.g., including halomethyl, such as CHCl₂ and CF₃). In certain such embodiments, Cy′ represents a 3-chloro-benzo[b]thien-2-yl, 3-fluoro-benzo[b]thien-2-yl, or 3-methyl-benzo[b]thien-2-yl, wherein the benzo ring is substituted with fluoro at the 4-position (peri to the 3-substituent on the thienyl ring) and, optionally, at the 7-position (‘peri’ to the S of the thienyl ring). Alternatively, the benzo ring may be unsubstituted. In certain embodiments, the benzo ring is selected from: 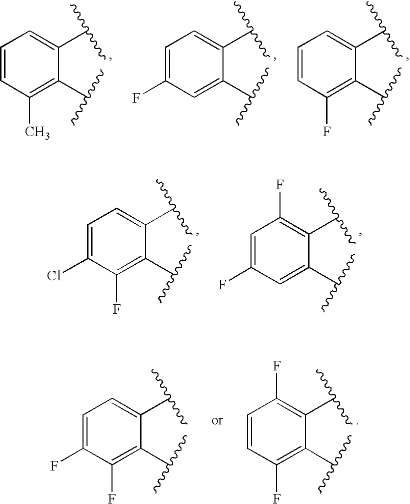
In yet further embodiments, Cy′ is selected from:

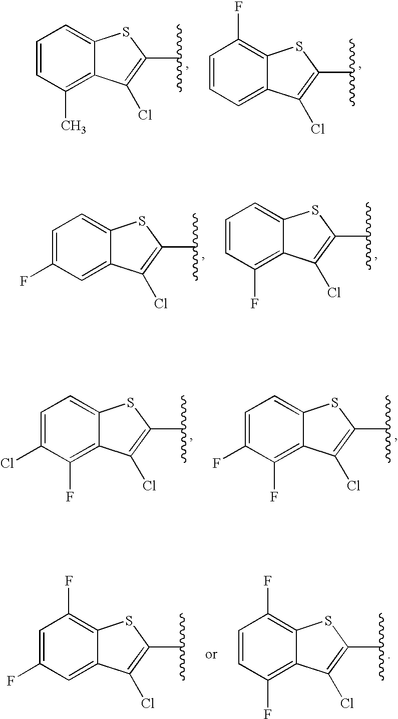

In certain embodiments, the present invention provides a method for the preparation of a compound of Formula B wherein n is 0, or a salt thereof: 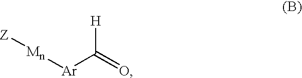
comprising treating a compound of Formula B1, or a salt thereof, with a compound of Formula B2, or a salt thereof: 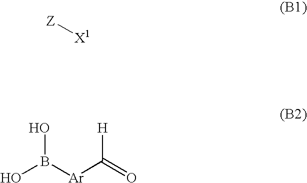
under carbon-carbon bond-forming conditions, wherein, as valence and stability permit,

Ar is a substituted or unsubstituted aryl or heteroaryl, such as a substituted or unsubstituted phenyl;

Z is a substituted or unsubstituted aryl or heteroaryl;

X¹ is a suitable leaving group, such as halogen, triflate or mesylate; and

the carbon-carbon bond-forming conditions comprise a catalytic amount of a palladium source, such as Pd(OAc)₂ or (Ph₃P)₂PdCl₂, a metal carbonate, ethanol or dimethoxyethane solvent, and water.

In certain embodiments, X¹ is a halogen, such as chloro or bromo.

In some embodiments, the carbon-carbon bond-forming conditions comprise N-acetylcysteine.

In particular embodiments, the carbon-carbon bond-forming conditions comprise Pd(OAc)₂ in an amount about 10 to about 0.1 mole percent, for example, about 4 to about 1 mole percent, relative to the compound of Formula B2. In other embodiments, the carbon-carbon bond-forming conditions comprise (Ph₃P)₂PdCl₂ in an amount about 1 to about 0.01 mole percent, for example, about 0.15 to about 0.05 mole percent, relative to the compound of Formula B2.

In certain embodiments, the compound of Formula B1 and the compound of Formula B2 are combined in about a 9 to 11:9 to 11 ratio, respectively. In other embodiments, the compound of Formula B1 and the compound of Formula B2 are combined in about a 1.8 to 1.2:1 ratio, respectively.

In some embodiments, the carbon-carbon bond-forming conditions comprise maintaining a temperature between about 27 and about 120° C., such as between about 50 and about 100° C., such as between about 70 and about 90° C., particularly between 55 and about 75° C.

In certain embodiments, Z is a substituted or unsubstituted aryl, such as a substituted or unsubstituted phenyl. In alternate embodiments, Z is a substituted or unsubstituted heteroaryl, such as a substituted or unsubstituted thiophene, furan, pyran, isobenzofuran, chromene, pyrrole, imidazole, pyrazole, isothiazole, isoxazole, pyridine, pyrazine, pyrimidine, pyridazine, indolizine, isoindole, indole, indazole, purine, isoquinoline, quinoline, or oxazole.

In some embodiments, Z is a substituted or unsubstituted aryl ring, such as a phenyl ring.

In other embodiments, Z is a substituted or unsubstituted heteroaryl ring, such as a substituted or unsubstituted pyridine, pyrimidine, pyrazine, pyridazine, triazine, tetrazine, pyrrole, pyrazole, or imidazole ring or N-oxide thereof. In specific embodiments, Z is a substituted or unsubstituted pyridine, or pyrazine ring or N-oxide thereof, particularly a substituted or unsubstituted pyridine ring or N-oxide thereof.

In some examples, Z is a substituted or unsubstituted pyridine ring or N-oxide thereof where connection of the pyridine ring to the phenyl ring bearing R¹ and R² can occur at any location on the pyridine ring, for example, at a 2-, 3-, or 4-position relative to the nitrogen of the pyridine ring, i.e., at an ortho-, meta-, or para-position relative to the nitrogen of the pyridine ring. In certain embodiments, Z is a substituted or unsubstituted pyridine N-oxide.

In other embodiments, Z is a substituted or unsubstituted pyrimidine ring or N-oxide thereof where connection of the pyrimidine ring to the phenyl ring bearing R¹ and R² can occur at any location on the pyrimidine ring, for example, at a 2-, 3-, 4-, 5-, or 6-position relative to N1 of the pyrimidine ring.

In some embodiments, Z is unsubstituted. In other embodiments, Z is substituted with one or more groups selected from halogen, lower alkyl, lower alkenyl, —CN, azido, —NR^{x}R^{x}, —CO₂OR^{x}, —C(O)—NR^{x}R^{x}, —C(O)—R^{x}, —NR^{x}—C(O)—R^{x}, —NR^{x}SO₂R^{x}, —SR^{x}, —S(O)R^{x}, —SO₂R^{x}, —SO₂NR^{x}R^{x}, —(C(R^{x})₂)ₙ—OR^{x}, —(C(R^{x})₂)ₙ—NR^{x}R^{x}, and —(C(R^{x})₂)ₙ—SO₂R^{x}; wherein R^{x} are, independently for each occurrence, H or lower alkyl; and n is, independently for each occurrence, an integer from 0 to 2. For example, in some embodiments, Z is substituted with halogen, such as fluoro.

In certain embodiments, Z is substituted with one or more electron withdrawing groups. For example, sometimes Z is substituted with one or more groups selected from halogen, —CN, azido, —CO₂OR^{x}, —C(O)—NR^{x}R^{x}, and —C(O)—R^{x}.

Combinations of the various values for Z are contemplated. In some instances, suitable compounds include those where Z is a pyridine ring or N-oxide thereof and Z is substituted by fluoro at any carbon position on the pyridine ring, such as a 2-, 3-, or 4-position relative to the nitrogen of the pyridine ring.

In certain embodiments, the compound of Formula B2 has the structure: 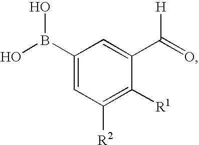
wherein R¹ and R² are, independently, H, halogen, hydroxyl, lower alkyl, or lower alkoxy, provided that at least one of R¹ and R² is not H.

In certain embodiments, R¹ and R² are, independently, H, fluorine, hydroxyl, lower alkyl, methoxy, or ethoxy, provided that at least one of R¹ and R² is not H.

In particular embodiments, R¹ is hydroxyl, ethyl, propyl, methoxy, or ethoxy; and R² is H, fluorine, hydroxyl, lower alkyl, methoxy, or ethoxy. In certain such embodiments, R² is H.

In certain embodiments, at least one of R¹ or R² is methoxy. For example, in some embodiments, R¹ is methoxy and R² is H, while in others, R² is methoxy and R¹ is H. In some instances, both R¹ and R² are methoxy.

In certain embodiments, at least one of R¹ or R² is lower alkyl. For example, sometimes R¹ is lower alkyl, such as methyl, ethyl, or propyl.

In certain embodiments, at least one of R¹ or R² is hydroxyl. For example, sometimes R¹ is hydroxyl.

In certain embodiments, Z is a substituted or unsubstituted aryl or heteroaryl ring; R^{a} is methyl; R¹ is halogen, such as fluoro or chloro, methoxy, or ethoxy; and R² is H. In some embodiments, R¹ is methoxy. In other embodiments, R¹ is fluoro. In some instances, R¹ is ethoxy. In some instances, Z is not a substituted or unsubstituted pyridine N-oxide ring or a pyridine ring substituted with one or more halogens. In certain embodiments, Z is substituted with one or more electron withdrawing groups.

In certain embodiments, Z is a substituted or unsubstituted aryl or heteroaryl ring; R^{a} is H or methyl; R¹ is H; and R² is halogen, such as fluoro or chloro, hydroxyl, methyl, ethyl, methoxy, or ethoxy. In some embodiments, R² is methoxy. In certain embodiments, R^{a} is methyl. In certain embodiments, Z is substituted with one or more electron withdrawing groups.

In certain embodiments, Z is a substituted or unsubstituted aryl or heteroaryl ring; R^{a} is H or methyl; R¹ is hydroxyl, methyl, or ethyl; and R² is H, halogen, such as fluoro or chloro, hydroxyl, methyl, ethyl, methoxy, or ethoxy. In certain embodiments, R^{a} is methyl. In certain embodiments, R² is H. In certain embodiments, R¹ is hydroxyl. In certain embodiments, R¹ is methyl. In certain embodiments, R¹ is ethyl. In some embodiments, one or both of R¹ or R² is hydroxyl; for example, sometimes R¹ is hydroxyl. In certain embodiments, Z is substituted with one or more electron withdrawing groups.

In certain embodiments, Z is a substituted or unsubstituted pyridine N-oxide ring; R^{a} is H or methyl; and R¹ and R² are, independently, H, halogen, such as fluoro or chloro, hydroxyl, methyl, ethyl, methoxy, or ethoxy, provided that at least one of R¹ and R² is not H. In certain embodiments, one or both of R¹ or R² is methoxy; for example, sometimes R¹ is methoxy, sometimes R² is methoxy, and sometimes both R¹ and R² are methoxy. In certain embodiments, one or both of R¹ or R² is fluoro; for example, sometimes R¹ is fluoro, sometimes R² is fluoro, and sometimes both R¹ and R² are fluoro. In certain embodiments, R² is H. In certain embodiments, R¹ is hydroxyl. In certain embodiments, R¹ is methyl. In certain embodiments, R¹ is ethyl. In some embodiments, one or both of R¹ or R² is hydroxyl; for example, sometimes R¹ is hydroxyl. In certain embodiments, Z is substituted with one or more electron withdrawing groups.

In certain embodiments, Z is a pyridine ring substituted with one or more halogens, such as fluoro and/or chloro, and optionally further substituted; R^{a} is H or methyl; and R¹ and R² are, independently, H, halogen, such as fluoro or chloro, hydroxyl, methyl, ethyl, methoxy, or ethoxy, provided that at least one of R¹ and R² is not H. In certain embodiments, one or both of R¹ or R² is methoxy; for example, sometimes R¹ is methoxy, sometimes R² is methoxy, and sometimes both R¹ and R² are methoxy. In certain embodiments, one or both of R¹ or R² is fluoro; for example, sometimes R¹ is fluoro, sometimes R² is fluoro, and sometimes both R¹ and R² are fluoro. In certain embodiments, one or both of R¹ or R² is fluoro; for example, sometimes R¹ is fluoro, sometimes R² is fluoro, and sometimes both R¹ and R² are fluoro. In certain embodiments, R² is H. In certain embodiments, R¹ is hydroxyl. In certain embodiments, R¹ is methyl. In certain embodiments, R¹ is ethyl. In some embodiments, one or both of R¹ or R² is hydroxyl; for example, sometimes R¹ is hydroxyl. In certain embodiments, Z is substituted with one or more electron withdrawing groups.

In certain embodiments, Z is a substituted or unsubstituted aryl or heteroaryl ring; R^{a} is H; and R¹ and R² are, independently, H, halogen, such as fluoro or chloro, hydroxyl, methyl, ethyl, methoxy, or ethoxy, provided that at least one of R¹ and R² is not H. In certain embodiments, one or both of R¹ or R² is methoxy; for example, sometimes R¹ is methoxy, sometimes R² is methoxy, and sometimes both R¹ and R² are methoxy. In certain embodiments, R² is H. In certain embodiments, R¹ is hydroxyl. In certain embodiments, R¹ is methyl. In certain embodiments, R¹ is ethyl. In some embodiments, one or both of R¹ or R² is hydroxyl; for example, sometimes R¹ is hydroxyl. In certain embodiments, Z is substituted with one or more electron withdrawing groups.

Variations of compounds of Formula B in which n is from 1-4 may be prepared in any of a number of ways well known to those of skill in the art. For example, compounds such as those of Formula B: 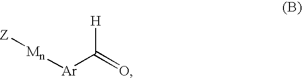
wherein n is 1 and M represents an oxo-substituted methylene (e.g., a carbonyl), may be prepared using reactions well known in the chemical arts, such as by using a Friedel-Crafts acylation reaction. In additional examples, compounds such as those of Formula B wherein n is 1-4 and M represents a methylene, may be prepared using reactions well known in the chemical arts, such as by using a Friedel-Crafts alkylation reaction. In additional examples, compounds such as those of Formula B wherein n is 2-4 and M represents a methylene, may be prepared using reactions well known in the chemical arts, such as by using an olefin metathesis reaction followed by a reduction of the resulting double bond. In further examples, compounds such as those of Formula B wherein one occurrence of M represents an oxo-substituted methylene (e.g., a carbonyl) and another occurrence of M is an oxygen, may be prepared using reactions well known in the chemical arts, such as by using an esterification reaction of an alcohol and a carboxylic acid. In other examples, compounds such as those of Formula B wherein one occurrence of M represents an oxo-substituted methylene (e.g., a carbonyl) and another occurrence of M is —NR^{a}—, may be prepared using reactions well known in the chemical arts, such as by using an amide synthesis through the reaction of an amine and a carbonyl derivative such as an acid halide, a carboxylic acid or an ester. In additional examples, compounds such as those of Formula B wherein M represents an ether, may be prepared using reactions well known in the chemical arts, such as by using an etherification reaction of an alcohol, such as phenol or benzyl alcohol, with an alkyl halide, such as ZX′ wherein X′ is a halogen.

In certain embodiments, the present invention provides a method for the preparation of a compound according to Formula A3, or a salt thereof: 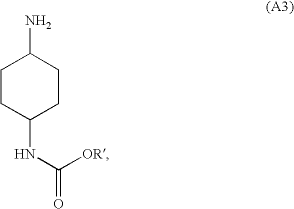
comprising treating a compound of Formula A1, or a salt thereof, with a compound of Formula A2: 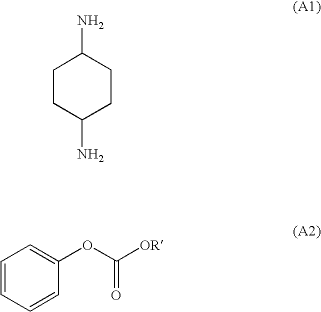
under carbamate forming conditions, wherein, as valence and stability permit,

R′ is a substituted or unsubstituted alkyl, such as a substituted or unsubstituted lower alkyl, for example, tert-butyl.

In some embodiments, the compound of Formula A1 and the compound of Formula A2 are combined in about a 9 to 11:9 to 11 ratio. respectively.

In certain embodiments, the carbamate forming conditions comprise maintaining a temperature between about 40 and about 120° C., such as between about 60 and about 100° C., particularly between about 80 and about 90° C.

In some embodiments, the carbamate forming conditions comprise one or more organic or aqueous solvent systems or combinations thereof. For example, in certain embodiments, the carbamate forming conditions comprise an alkyl alcohol, such as ethanol, wherein the alkyl alcohol may represent part of the solvent system, for example, about 10, 20, 30, 50, 70, or 90% of the solvent system, or substantially all of the solvent system, for example about 95% or greater of the solvent system.

In some embodiments, the compound of Formula A1 has the structure:

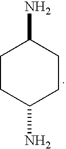

In certain embodiments, the present invention provides a method for the preparation of a compound of Formula A4, or a salt thereof: 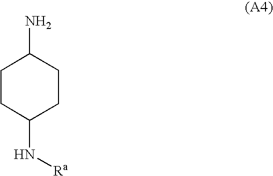
comprising treating a compound of Formula A3, or a salt thereof: 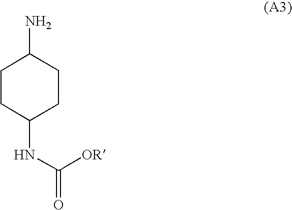
with a reducing agent under reducing conditions, wherein, as valence and stability permit,

R^{a} is substituted or unsubstituted alkyl, such as a substituted or unsubstituted lower alkyl, for example, methyl;

R′ is a substituted or unsubstituted alkyl, such as a substituted or unsubstituted lower alkyl, for example, tert-butyl; and

the reducing conditions comprise ethereal solvent, such as diethyl ether, tetrahydrofuran (THF), or dioxane.

In certain embodiments, the reducing agent is a metal hydride, such as an aluminum hydride, for example a lithium aluminum hydride or an organo aluminum hydride, such as diisobutyl aluminum hydride. In some embodiments, the reducing agent is a metal borohydride, such as a sodium borohydride or a lithium borohydride. Additives can be added to potentiate the metal hydrides, such ast trimethylsilyl chloride (TMS-Cl), for example, combinations of sodium or lithium borohydride with TMS-Cl are contemplated. In some embodiments, the reducing agent is a borane compound, such as diborane or 9-borabicyclo[3.3.1]nonane (9-BBN), or other boranes known in the art. The reducing agent may be in solution, for example an ethereal solution such as diethyl ether, THF, or dioxane.

In certain embodiments, the reducing agent and the compound of Formula A3 are in about a 5 to 1:1 ratio, respectively, for example about 3:1.

In certain instances, the reducing conditions comprise maintaining a temperature between 30° C. and about 110° C., for example, between about 40° C. and about 90° C., between about 50° C. and about 80° C., and/or between about 60° C. and about 70° C. In some embodiments, combination of the reducing agent and the compound of Formula A3 occurs at a temperature between about −10° C. and about 10° C., between about 10° C. and about 30° C., or between about 30 and about 40° C., after which the temperature is adjusted to the reflux temperature of the solvent, for example to about 35° C., 65° C., or 101° C.+/−5° C.

In some embodiments, the compound of Formula A3 has the structure:

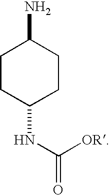

In certain embodiments, the present invention provides a method for the preparation of a compound of Formula A, or a salt thereof: 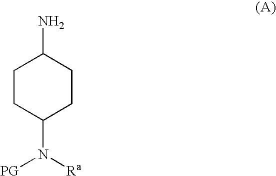
comprising treating a compound of Formula A4, or a salt thereof: 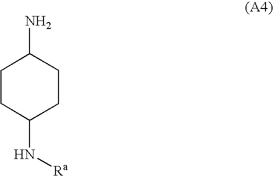
with an aldehyde under imine forming conditions followed by treating with an amine protecting group source under amine protecting conditions followed by treating with imine cleavage conditions, wherein, as valence and stability permit,

R^{a} is substituted or unsubstituted alkyl, such as a substituted or unsubstituted lower alkyl, for example, methyl, or an amine protecting group;

PG is an amine protecting group; and

the imine formation conditions comprise an alkyl alcohol solvent, such as methanol.

In certain embodiments, the amine protecting group source is a carbonate. In some embodiments, the amine protecting group source is a tert-butylcarboxy source, such as di-tert-butyl dicarbonate.

In some embodiments, the aldehyde is a substituted or unsubstituted aryl or heteroaryl aldehyde, such as benzaldehyde.

In certain embodiments, the aldehyde and the compound of Formula A4 are in about a 9 to 11:9 to 11 ratio, respectively.

In some embodiments, the amine protecting group source and the compound of Formula A4 are combined in about a 9 to 11:9 to 11 ratio, respectively.

In certain instances, the imine formation conditions comprise maintaining a temperature below about 60° C., for example, below about 50, 40, or 30° C. In certain instances, the imine formation conditions comprise maintaining a temperature between about 20 and about 45° C., such as between about 25 and about 35° C., particularly at about room temperature.

In some embodiments, the compound of Formula A4 has the structure:

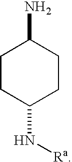

In certain embodiments, the present invention provides a method for the preparation of a compound according to Formula C, or a salt thereof: 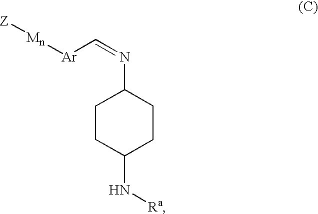
comprising treating a compound of Formula A4, or a salt thereof, with a compound of Formula B, or a salt thereof: 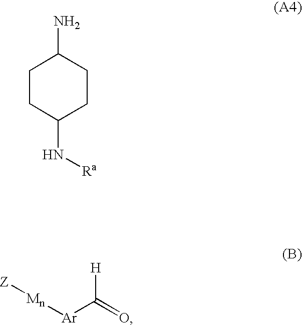
under imine formation conditions, wherein, as valence and stability permit,

Ar is a substituted or unsubstituted aryl or heteroaryl, such as a substituted or unsubstituted phenyl;

M, independently for each occurrence, represents a substituted or unsubstituted methylene group (e.g., substituted with lower alkyl, oxo, hydroxyl, etc.), NR⁴ (wherein R⁴ is H, substituted or unsubstituted alkyl, such as substituted or unsubstituted lower alkyl, or an amine protecting group), O, S, S(O), or S(O)₂, preferably selected such that no two heteroatoms are adjacent to each other;

n is an integer from 0-4, such as 0, 1, or 2, particularly 0;

Z is H, a substituted or unsubstituted aryl, carbocyclyl, heterocyclyl, or heteroaryl, or a nitro, cyano, or halogen; and

R^{a} is substituted or unsubstituted alkyl, such as a substituted or unsubstituted lower alkyl, for example, methyl, or an amine protecting group.

In some embodiments, the compound of Formula A4 and the compound of Formula B are combined in about a 1.4 to 1:1 ratio, respectively.

In certain embodiments, the imine formation conditions comprise maintaining a temperature between about 15 and about 45° C., such as between about 20 and about 30° C., particularly at about room temperature.

In certain embodiments the reaction mixture is substantially free of orthoformates, such as trimethyl orthoformate, wherein substantially free means that the substance in question makes up less than 10%, or less than 5%, or less than 4%, or less than 3%, or less than 2%, or less than 1% of the reaction mixture.

In some embodiments, the imine formation conditions comprise one or more organic or aqueous solvent systems or combinations thereof. For example, in certain embodiments, the imine formation conditions comprise an alkyl acetate, such as isopropyl acetate, wherein the alkyl acetate may represent part of the solvent system, for example, about 10, 20, 30, 50, 70, or 90% of the solvent system, or substantially all of the solvent system, for example about 95% or greater of the solvent system.

In certain embodiments, n is 0.

In certain embodiments, Z is H. In other embodiments, Z is a substituted or unsubstituted aryl, carbocyclyl, heterocyclyl, or heteroaryl. For example, in some embodiments Z is a substituted or unsubstituted thiophene, thianthrene, furan, pyran, isobenzofuran, chromene, xanthene, phenoxathiin, pyrrole, imidazole, pyrazole, isothiazole, isoxazole, pyridine, pyrazine, pyrimidine, pyridazine, indolizine, isoindole, indole, indazole, purine, quinolizine, isoquinoline, quinoline, phthalazine, naphthyridine, quinoxaline, quinazoline, cinnoline, pteridine, carbazole, carboline, phenanthridine, acridine, phenanthroline, phenazine, phenarsazine, phenothiazine, furazan, phenoxazine, pyrrolidine, oxolane, thiolane, oxazole, piperidine, piperazine, morpholine, lactone, lactam, azetidinone, pyrrolidinone, sultam, or sultone. In particular embodiments, Z is a substituted or unsubstituted aryl or heteroaryl, such as a substituted or unsubstituted phenyl. In other embodiments, Z is a substituted or unsubstituted carbocyclyl or heterocyclyl.

In some embodiments, Z is a substituted or unsubstituted aryl ring, such as a phenyl ring.

In other embodiments, Z is a substituted or unsubstituted heteroaryl ring, such as a substituted or unsubstituted pyridine, pyrimidine, pyrazine, pyridazine, triazine, tetrazine, pyrrole, pyrazole, or imidazole ring or N-oxide thereof. In specific embodiments, Z is a substituted or unsubstituted pyridine, or pyrazine ring or N-oxide thereof, particularly a substituted or unsubstituted pyridine ring or N-oxide thereof.

In some examples, Z is a substituted or unsubstituted pyridine ring or N-oxide thereof where connection of the pyridine ring to the phenyl ring bearing R¹ and R² can occur at any location on the pyridine ring, for example, at a 2-, 3-, or 4-position relative to the nitrogen of the pyridine ring, i.e., at an ortho-, meta-, or para-position relative to the nitrogen of the pyridine ring. In certain embodiments, Z is a substituted or unsubstituted pyridine N-oxide.

In other embodiments, Z is a substituted or unsubstituted pyrimidine ring or N-oxide thereof where connection of the pyrimidine ring to the phenyl ring bearing R¹ and R² can occur at any location on the pyrimidine ring, for example, at a 2-, 3-, 4-, 5-, or 6-position relative to N1 of the pyrimidine ring.

In some embodiments, Z is unsubstituted. In other embodiments, Z is substituted with one or more groups selected from halogen, lower alkyl, lower alkenyl, —CN, azido, —NR^{x}R^{x}, —CO₂OR^{x}, —C(O)—NR^{x}R^{x}, —C(O)—R^{x}, —NR^{x}—C(O)—R^{x}, NR^{x}SO₂R^{x}, SR^{x}, —S(O)R^{x}, SO₂R^{x}, —SO₂NR^{x}R^{x}, —(C(R^{x})₂)ₙ—OR^{x}, —(C(R^{x})₂)ₙ—NR^{x}R^{x}, and —(C(R^{x})₂)ₙ—SO₂R^{x}; wherein R^{x} are, independently for each occurrence, H or lower alkyl; and n is, independently for each occurrence, an integer from 0 to 2. For example, in some embodiments, Z is substituted with halogen, such as fluoro.

In certain embodiments, Z is substituted with one or more electron withdrawing groups. For example, sometimes Z is substituted with one or more groups selected from halogen, —CN, azido, —CO₂OR^{x}, —C(O)—NR^{x}R^{x}, and —C(O)—R^{x}.

Combinations of the various values for Z are contemplated. In some instances, suitable compounds include those where Z is a pyridine ring or N-oxide thereof and Z is substituted by fluoro at any carbon position on the pyridine ring, such as a 2-, 3-, or 4-position relative to the nitrogen of the pyridine ring.

In certain embodiments, the compound of Formula B has the structure: 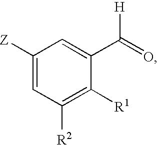
wherein R¹ and R² are, independently, H, halogen, hydroxyl, lower alkyl, or lower alkoxy, provided that at least one of R¹ and R² is not H.

In certain embodiments, R¹ and R² are, independently, H, fluorine, hydroxyl, lower alkyl, methoxy, or ethoxy, provided that at least one of R¹ and R² is not H.

In particular embodiments, R¹ is hydroxyl, ethyl, propyl, methoxy, or ethoxy; and R² is H, fluorine, hydroxyl, lower alkyl, methoxy, or ethoxy. In certain such embodiments, R² is H.

In certain embodiments, at least one of R¹ or R² is methoxy. For example, in some embodiments, R¹ is methoxy and R² is H, while in others, R² is methoxy and R¹ is H. In some instances, both R¹ and R² are methoxy.

In certain embodiments, at least one of R¹ or R² is lower alkyl. For example, sometimes R¹ is lower alkyl, such as methyl, ethyl, or propyl.

In certain embodiments, at least one of R¹ or R² is hydroxyl. For example, sometimes R¹ is hydroxyl.

In certain embodiments, Z is a substituted or unsubstituted aryl or heteroaryl ring; R^{a} is methyl; R¹ is halogen, such as fluoro or chloro, methoxy, or ethoxy; and R² is H. In some embodiments, R¹ is methoxy. In other embodiments, R¹ is fluoro. In some instances, R¹ is ethoxy. In some instances, Z is not a substituted or unsubstituted pyridine N-oxide ring or a pyridine ring substituted with one or more halogens. In certain embodiments, Z is substituted with one or more electron withdrawing groups.

In certain embodiments, Z is a substituted or unsubstituted aryl or heteroaryl ring; R^{a} is H or methyl; R¹ is H; and R² is halogen, such as fluoro or chloro, hydroxyl, methyl, ethyl, methoxy, or ethoxy. In some embodiments, R² is methoxy. In certain embodiments, R^{a} is methyl. In certain embodiments, Z is substituted with one or more electron withdrawing groups.

In certain embodiments, Z is a substituted or unsubstituted aryl or heteroaryl ring; R^{a} is H or methyl; R¹ is hydroxyl, methyl, or ethyl; and R² is H, halogen, such as fluoro or chloro, hydroxyl, methyl, ethyl, methoxy, or ethoxy. In certain embodiments, R^{a} is methyl. In certain embodiments, R² is H. In certain embodiments, R¹ is hydroxyl. In certain embodiments, R¹ is methyl. In certain embodiments, R¹ is ethyl. In some embodiments, one or both of R¹ or R² is hydroxyl; for example, sometimes R¹ is hydroxyl. In certain embodiments, Z is substituted with one or more electron withdrawing groups.

In certain embodiments, Z is a substituted or unsubstituted pyridine N-oxide ring; R^{a} is H or methyl; and R¹ and R² are, independently, H, halogen, such as fluoro or chloro, hydroxyl, methyl, ethyl, methoxy, or ethoxy, provided that at least one of R¹ and R² is not H. In certain embodiments, one or both of R¹ or R² is methoxy; for example, sometimes R¹ is methoxy, sometimes R² is methoxy, and sometimes both R¹ and R² are methoxy. In certain embodiments, one or both of R¹ or R² is fluoro; for example, sometimes R¹ is fluoro, sometimes R² is fluoro, and sometimes both R¹ and R² are fluoro. In certain embodiments, R² is H. In certain embodiments, R¹ is hydroxyl. In certain embodiments, R¹ is methyl. In certain embodiments, R¹ is ethyl. In some embodiments, one or both of R¹ or R² is hydroxyl; for example, sometimes R¹ is hydroxyl. In certain embodiments, Z is substituted with one or more electron withdrawing groups.

In certain embodiments, Z is a pyridine ring substituted with one or more halogens, such as fluoro and/or chloro, and optionally further substituted; R^{a} is H or methyl; and R¹ and R² are, independently, H, halogen, such as fluoro or chloro, hydroxyl, methyl, ethyl, methoxy, or ethoxy, provided that at least one of R¹ and R² is not H. In certain embodiments, one or both of R¹ or R² is methoxy; for example, sometimes R¹ is methoxy, sometimes R² is methoxy, and sometimes both R¹ and R² are methoxy. In certain embodiments, one or both of R¹ or R² is fluoro; for example, sometimes R¹ is fluoro, sometimes R² is fluoro, and sometimes both R¹ and R² are fluoro. In certain embodiments, one or both of R¹ or R² is fluoro; for example, sometimes R¹ is fluoro, sometimes R² is fluoro, and sometimes both R¹ and R² are fluoro. In certain embodiments, R² is H. In certain embodiments, R¹ is hydroxyl. In certain embodiments, R¹ is methyl. In certain embodiments, R¹ is ethyl. In some embodiments, one or both of R¹ or R² is hydroxyl; for example, sometimes R¹ is hydroxyl. In certain embodiments, Z is substituted with one or more electron withdrawing groups.

In certain embodiments, Z is a substituted or unsubstituted aryl or heteroaryl ring; R^{a} is H; and R¹ and R² are, independently, H, halogen, such as fluoro or chloro, hydroxyl, methyl, ethyl, methoxy, or ethoxy, provided that at least one of R¹ and R² is not H. In certain embodiments, one or both of R¹ or R² is methoxy; for example, sometimes R¹ is methoxy, sometimes R² is methoxy, and sometimes both R¹ and R² are methoxy. In certain embodiments, R² is H. In certain embodiments, R¹ is hydroxyl. In certain embodiments, R¹ is methyl. In certain embodiments, R¹ is ethyl. In some embodiments, one or both of R¹ or R² is hydroxyl; for example, sometimes R¹ is hydroxyl. In certain embodiments, Z is substituted with one or more electron withdrawing groups.

In some embodiments, the compound of Formula A4 has the structure:

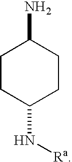

In certain embodiments, the present invention provides a method for the preparation of a compound according to Formula E, or a salt thereof: 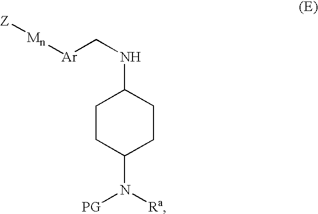
comprising treating a compound of Formula A, or a salt thereof, with a compound of Formula B, or a salt thereof: 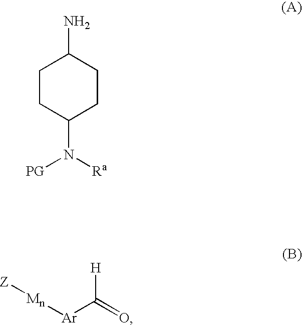
under imine formation conditions followed by treating with a reducing agent under imine reduction conditions, wherein, as valence and stability permit,

Ar is a substituted or unsubstituted aryl or heteroaryl, such as a substituted or unsubstituted phenyl;

M, independently for each occurrence, represents a substituted or unsubstituted methylene group (e.g., substituted with lower alkyl, oxo, hydroxyl, etc.), NR⁴ (wherein R⁴ is H, substituted or unsubstituted alkyl, such as substituted or unsubstituted lower alkyl, or an amine protecting group), O, S, S(O), or S(O)₂, preferably selected such that no two heteroatoms are adjacent to each other;

n is an integer from 0-4, such as 0, 1, or 2, particularly 0;

Z is H, a substituted or unsubstituted aryl, carbocyclyl, heterocyclyl, or heteroaryl, or a nitro, cyano, or halogen;

R^{a} is H, a substituted or unsubstituted alkyl, such as a substituted or unsubstituted lower alkyl, for example, methyl, or an amine protecting group;

PG is an amine protecting group, such as tert-butylcarboxy; and

the imine formation conditions comprise an alkyl alcohol solvent, such as methanol.

In certain embodiments, the reducing agent is a borohydride, such as a group I metal borohydride. For example, in certain embodiments, the reducing agent is sodium borohydride, sodium cyanoborohydride, or sodium triacetoxyborohydride, particularly sodium triacetoxyborohydride.

In some embodiments, the compound of Formula A and the compound of Formula B are combined in about a 1.4 to 1:1 ratio, respectively.

In certain embodiments, the reducing agent and the compound of Formula B are combined in about a 3.5 to 2:1 ratio, respectively.

In certain embodiments, the imine formation conditions comprise maintaining a temperature between about 15 and about 45° C., such as between about 25 and about 35° C., particularly at about room temperature.

In some embodiments, the imine reduction conditions comprise maintaining a temperature below room temperature, for example, about 2, 5, 10, 15, 20, or 30° C. below room temperature. In some embodiments, the imine reduction conditions comprise maintaining a temperature between about −20 and about 35° C., such as between about −20 and about 27° C., particularly between about −10 and about 10° C. In some embodiments, the imine reduction conditions comprise maintaining a temperature in two or more different ranges sequentially. For example, in certain embodiments, the imine reduction conditions comprise maintaining a temperature initially between about −20 and about 27° C., such as between about −10 and about 10° C., and subsequently between about −15 and about 35° C., such as between about 25 and about 30° C.

In certain embodiments the reaction mixture is substantially free of orthoformates, such as trimethyl orthoformate, wherein substantially free means that the substance in question makes up less than 10%, or less than 5%, or less than 4%, or less than 3%, or less than 2%, or less than 1% of the reaction mixture.

In certain embodiments, n is 0.

In certain embodiments, Z is H. In other embodiments, Z is a substituted or unsubstituted aryl, carbocyclyl, heterocyclyl, or heteroaryl. For example, in some embodiments Z is a substituted or unsubstituted thiophene, thianthrene, furan, pyran, isobenzofuran, chromene, xanthene, phenoxathiin, pyrrole, imidazole, pyrazole, isothiazole, isoxazole, pyridine, pyrazine, pyrimidine, pyridazine, indolizine, isoindole, indole, indazole, purine, quinolizine, isoquinoline, quinoline, phthalazine, naphthyridine, quinoxaline, quinazoline, cinnoline, pteridine, carbazole, carboline, phenanthridine, acridine, phenanthroline, phenazine, phenarsazine, phenothiazine, furazan, phenoxazine, pyrrolidine, oxolane, thiolane, oxazole, piperidine, piperazine, morpholine, lactone, lactam, azetidinone, pyrrolidinone, sultam, or sultone. In particular embodiments, Z is a substituted or unsubstituted aryl or heteroaryl, such as a substituted or unsubstituted phenyl. In other embodiments, Z is a substituted or unsubstituted carbocyclyl or heterocyclyl.

In some embodiments, Z is a substituted or unsubstituted aryl ring, such as a phenyl ring.

In other embodiments, Z is a substituted or unsubstituted heteroaryl ring, such as a substituted or unsubstituted pyridine, pyrimidine, pyrazine, pyridazine, triazine, tetrazine, pyrrole, pyrazole, or imidazole ring or N-oxide thereof. In specific embodiments, Z is a substituted or unsubstituted pyridine, or pyrazine ring or N-oxide thereof, particularly a substituted or unsubstituted pyridine ring or N-oxide thereof.

In some examples, Z is a substituted or unsubstituted pyridine ring or N-oxide thereof where connection of the pyridine ring to the phenyl ring bearing R¹ and R² can occur at any location on the pyridine ring, for example, at a 2-, 3-, or 4-position relative to the nitrogen of the pyridine ring, i.e., at an ortho-, meta-, or para-position relative to the nitrogen of the pyridine ring. In certain embodiments, Z is a substituted or unsubstituted pyridine N-oxide.

In other embodiments, Z is a substituted or unsubstituted pyrimidine ring or N-oxide thereof where connection of the pyrimidine ring to the phenyl ring bearing R¹ and R² can occur at any location on the pyrimidine ring, for example, at a 2-, 3-, 4-, 5-, or 6-position relative to N1 of the pyrimidine ring.

In some embodiments, Z is unsubstituted. In other embodiments, Z is substituted with one or more groups selected from halogen, lower alkyl, lower alkenyl, —CN, azido, —NR^{x}R^{x}, —CO₂OR^{x}, —C(O)—NR^{x}R^{x}, —C(O)—R^{x}, —NR^{x}—C(O)—R^{x}, —NR^{x}SO₂R^{x}, —SR^{x}, S(O)R^{x}, —SO₂R^{x}, —SO₂NR^{x}R^{x}, —(C(R^{x})₂)ₙ—OR^{x}, —(C(R^{x})₂)ₙ—NR^{x}R^{x}, and —(C(R^{x})₂)ₙ—SO₂R^{x}; wherein R^{x} are, independently for each occurrence, H or lower alkyl; and n is, independently for each occurrence, an integer from 0 to 2. For example, in some embodiments, Z is substituted with halogen, such as fluoro.

In certain embodiments, Z is substituted with one or more electron withdrawing groups. For example, sometimes Z is substituted with one or more groups selected from halogen, —CN, azido, —CO₂OR^{x}, —C(O)—NR^{x}R^{x}, and —C(O)—R^{x}.

Combinations of the various values for Z are contemplated. In some instances, suitable compounds include those where Z is a pyridine ring or N-oxide thereof and Z is substituted by fluoro at any carbon position on the pyridine ring, such as a 2-, 3-, or 4-position relative to the nitrogen of the pyridine ring.

In certain embodiments, the compound of Formula B has the structure: 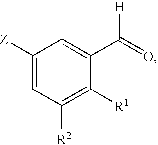
wherein R¹ and R² are, independently, H, halogen, hydroxyl, lower alkyl, or lower alkoxy, provided that at least one of R¹ and R² is not H.

In certain embodiments, R¹ and R² are, independently, H, fluorine, hydroxyl, lower alkyl, methoxy, or ethoxy, provided that at least one of R¹ and R² is not H.

In particular embodiments, R¹ is hydroxyl, ethyl, propyl, methoxy, or ethoxy; and R² is H, fluorine, hydroxyl, lower alkyl, methoxy, or ethoxy. In certain such embodiments, R² is H.

In certain embodiments, at least one of R¹ or R² is methoxy. For example, in some embodiments, R¹ is methoxy and R² is H, while in others, R² is methoxy and R¹ is H. In some instances, both R¹ and R² are methoxy.

In certain embodiments, at least one of R¹ or R² is lower alkyl. For example, sometimes R¹ is lower alkyl, such as methyl, ethyl, or propyl.

In certain embodiments, at least one of R¹ or R² is hydroxyl. For example, sometimes R¹ is hydroxyl.

In certain embodiments, Z is a substituted or unsubstituted aryl or heteroaryl ring; R^{a} is methyl; R¹ is halogen, such as fluoro or chloro, methoxy, or ethoxy; and R² is H. In some embodiments, R¹ is methoxy. In other embodiments, R¹ is fluoro. In some instances, R¹ is ethoxy. In some instances, Z is not a substituted or unsubstituted pyridine N-oxide ring or a pyridine ring substituted with one or more halogens. In certain embodiments, Z is substituted with one or more electron withdrawing groups.

In certain embodiments, Z is a substituted or unsubstituted aryl or heteroaryl ring; R^{a} is H or methyl; R¹ is H; and R² is halogen, such as fluoro or chloro, hydroxyl, methyl, ethyl, methoxy, or ethoxy. In some embodiments, R² is methoxy. In certain embodiments, R^{a} is methyl. In certain embodiments, Z is substituted with one or more electron withdrawing groups.

In certain embodiments, Z is a substituted or unsubstituted aryl or heteroaryl ring; R^{a} is H or methyl; R¹ is hydroxyl, methyl, or ethyl; and R² is H, halogen, such as fluoro or chloro, hydroxyl, methyl, ethyl, methoxy, or ethoxy. In certain embodiments, R^{a} is methyl. In certain embodiments, R² is H. In certain embodiments, R¹ is hydroxyl. In certain embodiments, R¹ is methyl. In certain embodiments, R¹ is ethyl. In some embodiments, one or both of R¹ or R² is hydroxyl; for example, sometimes R¹ is hydroxyl. In certain embodiments, Z is substituted with one or more electron withdrawing groups.

In certain embodiments, Z is a substituted or unsubstituted pyridine N-oxide ring; R^{a} is H or methyl; and R¹ and R² are, independently, H, halogen, such as fluoro or chloro, hydroxyl, methyl, ethyl, methoxy, or ethoxy, provided that at least one of R¹ and R² is not H. In certain embodiments, one or both of R¹ or R² is methoxy; for example, sometimes R¹ is methoxy, sometimes R² is methoxy, and sometimes both R¹ and R² are methoxy. In certain embodiments, one or both of R¹ or R² is fluoro; for example, sometimes R¹ is fluoro, sometimes R² is fluoro, and sometimes both R¹ and R² are fluoro. In certain embodiments, R² is H. In certain embodiments, R¹ is hydroxyl. In certain embodiments, R¹ is methyl. In certain embodiments, R¹ is ethyl. In some embodiments, one or both of R¹ or R² is hydroxyl; for example, sometimes R¹ is hydroxyl. In certain embodiments, Z is substituted with one or more electron withdrawing groups.

In certain embodiments, Z is a pyridine ring substituted with one or more halogens, such as fluoro and/or chloro, and optionally further substituted; R^{a} is H or methyl; and R¹ and R² are, independently, H, halogen, such as fluoro or chloro, hydroxyl, methyl, ethyl, methoxy, or ethoxy, provided that at least one of R¹ and R² is not H. In certain embodiments, one or both of R¹ or R² is methoxy; for example, sometimes R¹ is methoxy, sometimes R² is methoxy, and sometimes both R¹ and R² are methoxy. In certain embodiments, one or both of R¹ or R² is fluoro; for example, sometimes R¹ is fluoro, sometimes R² is fluoro, and sometimes both R¹ and R² are fluoro. In certain embodiments, one or both of R¹ or R² is fluoro; for example, sometimes R¹ is fluoro, sometimes R² is fluoro, and sometimes both R¹ and R² are fluoro. In certain embodiments, R² is H. In certain embodiments, R¹ is hydroxyl. In certain embodiments, R¹ is methyl. In certain embodiments, R¹ is ethyl. In some embodiments, one or both of R¹ or R² is hydroxyl; for example, sometimes R¹ is hydroxyl. In certain embodiments, Z is substituted with one or more electron withdrawing groups.

In certain embodiments, Z is a substituted or unsubstituted aryl or heteroaryl ring; R^{a} is H; and R¹ and R² are, independently, H, halogen, such as fluoro or chloro, hydroxyl, methyl, ethyl, methoxy, or ethoxy, provided that at least one of R¹ and R² is not H. In certain embodiments, one or both of R¹ or R² is methoxy; for example, sometimes R¹ is methoxy, sometimes R² is methoxy, and sometimes both R¹ and R² are methoxy. In certain embodiments, R² is H. In certain embodiments, R¹ is hydroxyl. In certain embodiments, R¹ is methyl. In certain embodiments, R¹ is ethyl. In some embodiments, one or both of R¹ or R² is hydroxyl; for example, sometimes R¹ is hydroxyl. In certain embodiments, Z is substituted with one or more electron withdrawing groups.

In some embodiments, the compound of Formula A has the structure:

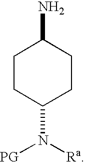

In certain embodiments, the present invention provides a method for the preparation of a compound according to Formula I, or a pharmaceutically acceptable salt thereof: 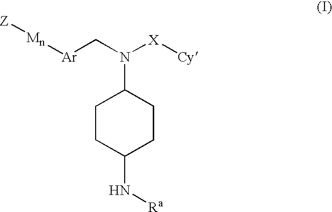
comprising:

a) treating a compound of Formula A4, or a salt thereof, with a compound of Formula B, or a salt thereof: 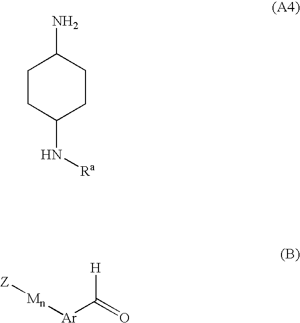
under imine formation conditions to generate a compound of Formula C, or a salt thereof:

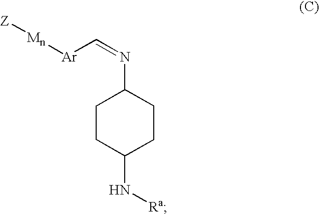

b) treating the compound of Formula C, or a salt thereof, with an amine protecting group source under amine protecting conditions to generate a compound of Formula D, or a salt thereof:

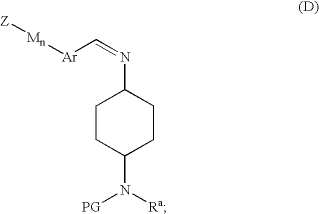

c) treating the compound of Formula D, or a salt thereof, with a reducing agent under imine reduction conditions to generate a compound of Formula E, or a salt thereof:

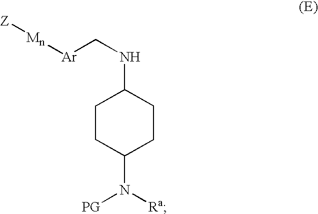

d) treating the compound of Formula E, or a salt thereof, with a compound of Formula F, or a salt thereof: 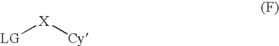
under acylation conditions to generate a compound of Formula G, or a salt thereof:

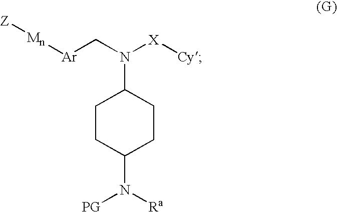

e) treating the compound of Formula G, or a salt thereof, under conditions that remove PG to prepare a compound of Formula I, or a pharmaceutically acceptable salt thereof, wherein, as valence and stability permit,

Ar is a substituted or unsubstituted aryl or heteroaryl, such as a substituted or unsubstituted phenyl;

M, independently for each occurrence, represents a substituted or unsubstituted methylene group (e.g., substituted with lower alkyl, oxo, hydroxyl, etc.), NR⁴ (wherein R⁴ is H, substituted or unsubstituted alkyl, such as substituted or unsubstituted lower alkyl, or an amine protecting group), O, S, S(O), or S(O)₂, preferably selected such that no two heteroatoms are adjacent to each other;

n is an integer from 0-4, such as 0, 1, or 2, particularly 0;

Z is H, a substituted or unsubstituted aryl, carbocyclyl, heterocyclyl, or heteroaryl, or a nitro, cyano, or halogen;

X is —C(═O)—, —C(═S)—, —S(O₂)—, —S(O)—, —C(═NCN)—, or —P(═O)(OR)—, particularly —C(═O)-—C(═S)—, or —S(O₂)—;

R^{a} is H or substituted or unsubstituted alkyl, such as substituted or unsubstituted lower alkyl, for example methyl, or an amine protecting group;

PG is an amine protecting group, such as tert-butylcarboxy;

Cy′ is a substituted or unsubstituted benzothiophene, such as a substituted or unsubstituted 2-benzothiophene; and

LG is a leaving group, such as halogen, for example chloro.

In some embodiments, the present methods further comprise forming a pharmaceutically acceptable salt of the compound of Formula I.

In certain instances, the compound of Formula C is isolated and optionally purified. In other embodiments, steps a) and b) are performed in succession without isolating or purifying the compound of Formula C.

In certain instances, the compound of Formula E is isolated and optionally purified. In other embodiments steps c) and d) are performed in succession without isolating or purifying the compound of Formula E.

In certain embodiments, n is 0.

In certain embodiments, Z is H. In other embodiments, Z is a substituted or unsubstituted aryl, carbocyclyl, heterocyclyl, or heteroaryl. For example, in some embodiments Z is a substituted or unsubstituted thiophene, thianthrene, furan, pyran, isobenzofuran, chromene, xanthene, phenoxathiin, pyrrole, imidazole, pyrazole, isothiazole, isoxazole, pyridine, pyrazine, pyrimidine, pyridazine, indolizine, isoindole, indole, indazole, purine, quinolizine, isoquinoline, quinoline, phthalazine, naphthyridine, quinoxaline, quinazoline, cinnoline, pteridine, carbazole, carboline, phenanthridine, acridine, phenanthroline, phenazine, phenarsazine, phenothiazine, furazan, phenoxazine, pyrrolidine, oxolane, thiolane, oxazole, piperidine, piperazine, morpholine, lactone, lactam, azetidinone, pyrrolidinone, sultam, or sultone. In particular embodiments, Z is a substituted or unsubstituted aryl or heteroaryl, such as a substituted or unsubstituted phenyl. In other embodiments, Z is a substituted or unsubstituted carbocyclyl or heterocyclyl.

In some embodiments, Z is a substituted or unsubstituted aryl ring, such as a phenyl ring.

In other embodiments, Z is a substituted or unsubstituted heteroaryl ring, such as a substituted or unsubstituted pyridine, pyrimidine, pyrazine, pyridazine, triazine, tetrazine, pyrrole, pyrazole, or imidazole ring or N-oxide thereof. In specific embodiments, Z is a substituted or unsubstituted pyridine, or pyrazine ring or N-oxide thereof, particularly a substituted or unsubstituted pyridine ring or N-oxide thereof.

In some examples, Z is a substituted or unsubstituted pyridine ring or N-oxide thereof where connection of the pyridine ring to the phenyl ring bearing R¹ and R² can occur at any location on the pyridine ring, for example, at a 2-, 3-, or 4-position relative to the nitrogen of the pyridine ring, i.e., at an ortho-, meta-, or para-position relative to the nitrogen of the pyridine ring. In certain embodiments, Z is a substituted or unsubstituted pyridine N-oxide.

In other embodiments, Z is a substituted or unsubstituted pyrimidine ring or N-oxide thereof where connection of the pyrimidine ring to the phenyl ring bearing R¹ and R² can occur at any location on the pyrimidine ring, for example, at a 2-, 3-, 4-, 5-, or 6-position relative to N1 of the pyrimidine ring.

In some embodiments, Z is unsubstituted. In other embodiments, Z is substituted with one or more groups selected from halogen, lower alkyl, lower alkenyl, —CN, azido, —NR^{x}R^{x}, —CO₂OR^{x}, —C(O)—NR^{x}R^{x}, —C(O)—R^{x}, —NR^{x}—C(O)—R^{x}, —NR^{x}SO₂R^{x}, —SR^{x}, —S(O)R^{x}, —SO₂R^{x}, —SO₂NR^{x}R^{x}, —(C(R^{x})₂)ₙ—OR^{x}, —(C(R^{x})₂)ₙ—NR^{x}R^{x}, and —(C(R^{x})₂)ₙ—SO₂R^{x}; wherein R^{x} are, independently for each occurrence, H or lower alkyl; and n is, independently for each occurrence, an integer from 0 to 2. For example, in some embodiments, Z is substituted with halogen, such as fluoro.

In certain embodiments, Z is substituted with one or more electron withdrawing groups. For example, sometimes Z is substituted with one or more groups selected from halogen, —CN, azido, —CO₂OR^{x}, —C(O)—NR^{x}R^{x}, and —C(O)—R^{x}.

Combinations of the various values for Z are contemplated. In some instances, suitable compounds include those where Z is a pyridine ring or N-oxide thereof and Z is substituted by fluoro at any carbon position on the pyridine ring, such as a 2-, 3-, or 4-position relative to the nitrogen of the pyridine ring.

In certain embodiments, Ar has the structure: 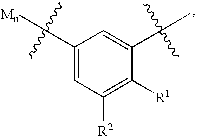
wherein R¹ and R² are, independently, H, halogen, hydroxyl, lower alkyl, or lower alkoxy, provided that at least one of R¹ and R² is not H.

In certain embodiments, R¹ and R² are, independently, H, fluorine, hydroxyl, lower alkyl, methoxy, or ethoxy, provided that at least one of R¹ and R² is not H.

In particular embodiments, R¹ is hydroxyl, ethyl, propyl, methoxy, or ethoxy; and R² is H, fluorine, hydroxyl, lower alkyl, methoxy, or ethoxy. In certain such embodiments, R² is H.

In certain embodiments, at least one of R¹ or R² is methoxy. For example, in some embodiments, R¹ is methoxy and R² is H, while in others, R² is methoxy and R¹ is H. In some instances, both R¹ and R² are methoxy.

In certain embodiments, at least one of R¹ or R² is lower alkyl. For example, sometimes R¹ is lower alkyl, such as methyl, ethyl, or propyl.

In certain embodiments, at least one of R¹ or R² is hydroxyl. For example, sometimes R¹ is hydroxyl.

In certain embodiments, Z is a substituted or unsubstituted aryl or heteroaryl ring; R^{a} is methyl; R¹ is halogen, such as fluoro or chloro, methoxy, or ethoxy; and R² is H. In some embodiments, R¹ is methoxy. In other embodiments, R¹ is fluoro. In some instances, R¹ is ethoxy. In some instances, Z is not a substituted or unsubstituted pyridine N-oxide ring or a pyridine ring substituted with one or more halogens. In certain embodiments, Z is substituted with one or more electron withdrawing groups.

In certain embodiments, Z is a substituted or unsubstituted aryl or heteroaryl ring; R^{a} is H or methyl; R¹ is H; and R² is halogen, such as fluoro or chloro, hydroxyl, methyl, ethyl, methoxy, or ethoxy. In some embodiments, R² is methoxy. In certain embodiments, R^{a} is methyl. In certain embodiments, Z is substituted with one or more electron withdrawing groups.

In certain embodiments, Z is a substituted or unsubstituted aryl or heteroaryl ring; R^{a} is H or methyl; R¹ is hydroxyl, methyl, or ethyl; and R² is H, halogen, such as fluoro or chloro, hydroxyl, methyl, ethyl, methoxy, or ethoxy. In certain embodiments, R^{a} is methyl. In certain embodiments, R² is H. In certain embodiments, R¹ is hydroxyl. In certain embodiments, R¹ is methyl. In certain embodiments, R¹ is ethyl. In some embodiments, one or both of R¹ or R² is hydroxyl; for example, sometimes R¹ is hydroxyl. In certain embodiments, Z is substituted with one or more electron withdrawing groups.

In certain embodiments, Z is a substituted or unsubstituted pyridine N-oxide ring; R^{a} is H or methyl; and R¹ and R² are, independently, H, halogen, such as fluoro or chloro, hydroxyl, methyl, ethyl, methoxy, or ethoxy, provided that at least one of R¹ and R² is not H. In certain embodiments, one or both of R¹ or R² is methoxy; for example, sometimes R¹ is methoxy, sometimes R² is methoxy, and sometimes both R¹ and R² are methoxy. In certain embodiments, one or both of R¹ or R² is fluoro; for example, sometimes R¹ is fluoro, sometimes R² is fluoro, and sometimes both R¹ and R² are fluoro. In certain embodiments, R² is H. In certain embodiments, R¹ is hydroxyl. In certain embodiments, R¹ is methyl. In certain embodiments, R¹ is ethyl. In some embodiments, one or both of R¹ or R² is hydroxyl; for example, sometimes R¹ is hydroxyl. In certain embodiments, Z is substituted with one or more electron withdrawing groups.

In certain embodiments, Z is a pyridine ring substituted with one or more halogens, such as fluoro and/or chloro, and optionally further substituted; R^{a} is H or methyl; and R¹ and R² are, independently, H, halogen, such as fluoro or chloro, hydroxyl, methyl, ethyl, methoxy, or ethoxy, provided that at least one of R¹ and R² is not H. In certain embodiments, one or both of R¹ or R² is methoxy; for example, sometimes R¹ is methoxy, sometimes R² is methoxy, and sometimes both R¹ and R² are methoxy. In certain embodiments, one or both of R¹ or R² is fluoro; for example, sometimes R¹ is fluoro, sometimes R² is fluoro, and sometimes both R¹ and R² are fluoro. In certain embodiments, one or both of R¹ or R² is fluoro; for example, sometimes R¹ is fluoro, sometimes R² is fluoro, and sometimes both R¹ and R² are fluoro. In certain embodiments, R² is H. In certain embodiments, R¹ is hydroxyl. In certain embodiments, R¹ is methyl. In certain embodiments, R¹ is ethyl. In some embodiments, one or both of R¹ or R² is hydroxyl; for example, sometimes R¹ is hydroxyl. In certain embodiments, Z is substituted with one or more electron withdrawing groups.

In certain embodiments, Z is a substituted or unsubstituted aryl or heteroaryl ring; R^{a} is H; and R¹ and R² are, independently, H, halogen, such as fluoro or chloro, hydroxyl, methyl, ethyl, methoxy, or ethoxy, provided that at least one of R¹ and R² is not H. In certain embodiments, one or both of R¹ or R² is methoxy; for example, sometimes R¹ is methoxy, sometimes R² is methoxy, and sometimes both R¹ and R² are methoxy. In certain embodiments, R² is H. In certain embodiments, R¹ is hydroxyl. In certain embodiments, R¹ is methyl. In certain embodiments, R¹ is ethyl. In some embodiments, one or both of R¹ or R² is hydroxyl; for example, sometimes R¹ is hydroxyl. In certain embodiments, Z is substituted with one or more electron withdrawing groups.

In certain embodiments, the compound of Formula A4 has the structure:

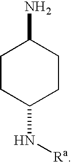

In some embodiments, Cy′ is substituted with one or more halogen and/or alkyl groups. For example, in some instances Cy′ is substituted with one or more chloro and/or fluoro groups.

In particular embodiments, Cy′ is a 3-halo-benzo[b]thien-2-yl, for example a 3-chloro-benzo[b]thien-2-yl or a 3-fluoro-benzo[b]thien-2-yl, or a 3-methyl-benzo[b]thien-2-yl. The benzo ring of Cy′ may be substituted with from 1-4 substituents such as halogen, nitro, cyano, methyl (e.g., including halomethyl, such as CHCl₂ and CF₃), and ethyl (e.g., including haloethyl, such as CH₂CCl₃, C₂F₅, etc.), preferably with halogen and methyl (e.g., including halomethyl, such as CHCl₂ and CF₃). In certain such embodiments, Cy′ represents a 3-chloro-benzo[b]thien-2-yl, 3-fluoro-benzo[b]thien-2-yl, or 3-methyl-benzo[b]thien-2-yl, wherein the benzo ring is substituted with fluoro at the 4-position (peri to the 3-substituent on the thienyl ring) and, optionally, at the 7-position (‘peri’ to the S of the thienyl ring). Alternatively, the benzo ring may be unsubstituted. In certain embodiments, the benzo ring is selected from: 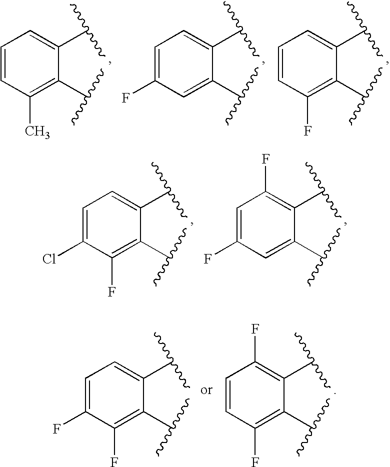
In yet further embodiments, Cy′ is selected from:

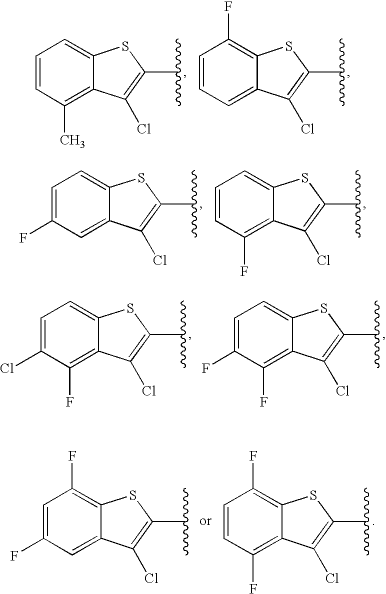

In certain embodiments, the present invention provides a method for the preparation of a compound according to Formula I, or a pharmaceutically acceptable salt thereof: 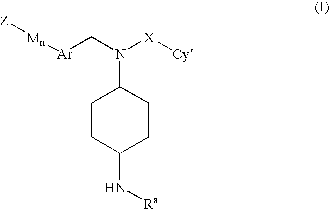
comprising:

a) treating a compound of Formula A, or a salt thereof, with a compound of Formula B, or a salt thereof: 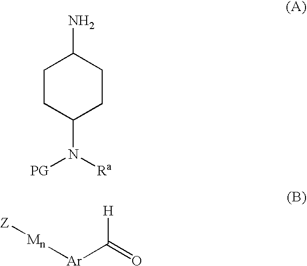
under imine formation conditions to generate a compound of Formula D, or a salt thereof:

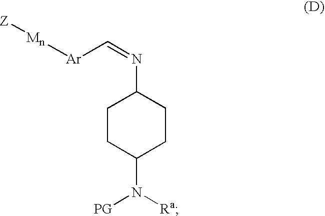

b) treating the compound of Formula D, or a salt thereof, with a reducing agent under imine reduction conditions to generate a compound of Formula E, or a salt thereof:

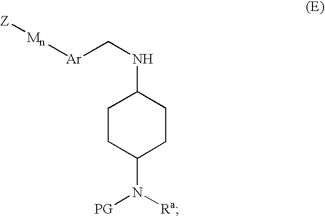

c) treating the compound of Formula E, or a salt thereof, with a compound of Formula F, or a salt thereof: 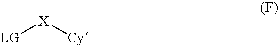
under acylation conditions to generate a compound of Formula G, or a salt thereof:

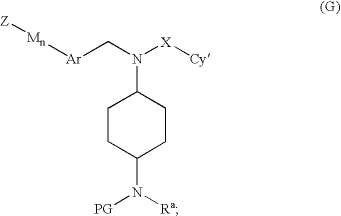

d) treating the compound of Formula G, or a salt thereof, under conditions that remove PG to prepare a compound of Formula I, or a pharmaceutically acceptable salt thereof, wherein, as valence and stability permit,

Ar is a substituted or unsubstituted aryl or heteroaryl, such as a substituted or unsubstituted phenyl;

M, independently for each occurrence, represents a substituted or unsubstituted methylene group (e.g., substituted with lower alkyl, oxo, hydroxyl, etc.), NR⁴ (wherein R⁴ is H, substituted or unsubstituted alkyl, such as substituted or unsubstituted lower alkyl, or an amine protecting group), O, S, S(O), or S(O)₂, preferably selected such that no two heteroatoms are adjacent to each other;

n is an integer from 0-4, such as 0, 1, or 2, particularly 0;

Z is H, a substituted or unsubstituted aryl, carbocyclyl, heterocyclyl, or heteroaryl, or a nitro, cyano, or halogen;

X is —C(═O)—, —C(═S)—, —S(O₂)—, —S(O)—, —C(═NCN)—, or —P(═O)(OR)—, particularly —C(═O)-—C(═S)—, or —S(O₂)—;

R^{a} is H or substituted or unsubstituted alkyl, such as substituted or unsubstituted lower alkyl, for example methyl, or an amine protecting group;

PG is an amine protecting group, such as tert-butylcarboxy;

Cy′ is a substituted or unsubstituted benzothiophene, such as a substituted or unsubstituted 2-benzothiophene; and

LG is a leaving group, such as halogen, for example chloro.

In some embodiments, the present methods further comprise forming a pharmaceutically acceptable salt of the compound of Formula I.

In certain instances, the compound of Formula D is isolated and optionally purified. In other embodiments, steps a) and b) are performed in succession without isolating or purifying the compound of Formula D.

In certain instances, the compound of Formula E is isolated and optionally purified. In other embodiments steps b) and c) are performed in succession without isolating or purifying the compound of Formula E.

In certain embodiments, n is 0.

In certain embodiments, Z is H. In other embodiments, Z is a substituted or unsubstituted aryl, carbocyclyl, heterocyclyl, or heteroaryl. For example, in some embodiments Z is a substituted or unsubstituted thiophene, thianthrene, furan, pyran, isobenzofuran, chromene, xanthene, phenoxathiin, pyrrole, imidazole, pyrazole, isothiazole, isoxazole, pyridine, pyrazine, pyrimidine, pyridazine, indolizine, isoindole, indole, indazole, purine, quinolizine, isoquinoline, quinoline, phthalazine, naphthyridine, quinoxaline, quinazoline, cinnoline, pteridine, carbazole, carboline, phenanthridine, acridine, phenanthroline, phenazine, phenarsazine, phenothiazine, furazan, phenoxazine, pyrrolidine, oxolane, thiolane, oxazole, piperidine, piperazine, morpholine, lactone, lactam, azetidinone, pyrrolidinone, sultam, or sultone. In particular embodiments, Z is a substituted or unsubstituted aryl or heteroaryl, such as a substituted or unsubstituted phenyl. In other embodiments, Z is a substituted or unsubstituted carbocyclyl or heterocyclyl.

In some embodiments, Z is a substituted or unsubstituted aryl ring, such as a phenyl ring.

In other embodiments, Z is a substituted or unsubstituted heteroaryl ring, such as a substituted or unsubstituted pyridine, pyrimidine, pyrazine, pyridazine, triazine, tetrazine, pyrrole, pyrazole, or imidazole ring or N-oxide thereof. In specific embodiments, Z is a substituted or unsubstituted pyridine, or pyrazine ring or N-oxide thereof, particularly a substituted or unsubstituted pyridine ring or N-oxide thereof.

In some examples, Z is a substituted or unsubstituted pyridine ring or N-oxide thereof where connection of the pyridine ring to the phenyl ring bearing R¹ and R² can occur at any location on the pyridine ring, for example, at a 2-, 3-, or 4-position relative to the nitrogen of the pyridine ring, i.e., at an ortho-, meta-, or para-position relative to the nitrogen of the pyridine ring. In certain embodiments, Z is a substituted or unsubstituted pyridine N-oxide.

In other embodiments, Z is a substituted or unsubstituted pyrimidine ring or N-oxide thereof where connection of the pyrimidine ring to the phenyl ring bearing R¹ and R² can occur at any location on the pyrimidine ring, for example, at a 2-, 3-, 4-, 5-, or 6-position relative to N1 of the pyrimidine ring.

In some embodiments, Z is unsubstituted. In other embodiments, Z is substituted with one or more groups selected from halogen, lower alkyl, lower alkenyl, —CN, azido, —NR^{x}R^{x}, —CO₂OR^{x}, —C(O)—NR^{x}R^{x}, C(O)—R^{x}, —NR^{x}—C(O)—R^{x}, NR^{x}SO₂R^{x}, —SR^{x}, —S(O)R^{x}, —SO₂R^{x}, —SO₂NR^{x}R^{x}, —(C(R^{x})₂)ₙ—OR^{x}, —(C(R^{x})₂)ₙ—NR^{x}R^{x}, and —(C(R^{x})₂)ₙ—SO₂R^{x}; wherein R^{x} are, independently for each occurrence, H or lower alkyl; and n is, independently for each occurrence, an integer from 0 to 2. For example, in some embodiments, Z is substituted with halogen, such as fluoro.

In certain embodiments, Z is substituted with one or more electron withdrawing groups. For example, sometimes Z is substituted with one or more groups selected from halogen, —CN, azido, —CO₂OR^{x}, —C(O)—NR^{x}R^{x}, and —C(O)—R^{x}.

Combinations of the various values for Z are contemplated. In some instances, suitable compounds include those where Z is a pyridine ring or N-oxide thereof and Z is substituted by fluoro at any carbon position on the pyridine ring, such as a 2-, 3-, or 4-position relative to the nitrogen of the pyridine ring.

In certain embodiments, Ar has the structure: 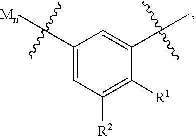
wherein R¹ and R² are, independently, H, halogen, hydroxyl, lower alkyl, or lower alkoxy, provided that at least one of R¹ and R² is not H.

In certain embodiments, R¹ and R² are, independently, H, fluorine, hydroxyl, lower alkyl, methoxy, or ethoxy, provided that at least one of R¹ and R² is not H.

In particular embodiments, R¹ is hydroxyl, ethyl, propyl, methoxy, or ethoxy; and R² is H, fluorine, hydroxyl, lower alkyl, methoxy, or ethoxy. In certain such embodiments, R² is H.

In certain embodiments, at least one of R¹ or R² is methoxy. For example, in some embodiments, R¹ is methoxy and R² is H, while in others, R² is methoxy and R¹ is H. In some instances, both R¹ and R² are methoxy.

In certain embodiments, at least one of R¹ or R² is lower alkyl. For example, sometimes R¹ is lower alkyl, such as methyl, ethyl, or propyl.

In certain embodiments, at least one of R¹ or R² is hydroxyl. For example, sometimes R¹ is hydroxyl.

In certain embodiments, Z is a substituted or unsubstituted aryl or heteroaryl ring; R^{a} is methyl; R¹ is halogen, such as fluoro or chloro, methoxy, or ethoxy; and R² is H. In some embodiments, R¹ is methoxy. In other embodiments, R¹ is fluoro. In some instances, R¹ is ethoxy. In some instances, Z is not a substituted or unsubstituted pyridine N-oxide ring or a pyridine ring substituted with one or more halogens. In certain embodiments, Z is substituted with one or more electron withdrawing groups.

In certain embodiments, Z is a substituted or unsubstituted aryl or heteroaryl ring; R^{a} is H or methyl; R¹ is H; and R² is halogen, such as fluoro or chloro, hydroxyl, methyl, ethyl, methoxy, or ethoxy. In some embodiments, R² is methoxy. In certain embodiments, R^{a} is methyl. In certain embodiments, Z is substituted with one or more electron withdrawing groups.

In certain embodiments, Z is a substituted or unsubstituted aryl or heteroaryl ring; R^{a} is H or methyl; R¹ is hydroxyl, methyl, or ethyl; and R² is H, halogen, such as fluoro or chloro, hydroxyl, methyl, ethyl, methoxy, or ethoxy. In certain embodiments, R^{a} is methyl. In certain embodiments, R² is H. In certain embodiments, R¹ is hydroxyl. In certain embodiments, R¹ is methyl. In certain embodiments, R¹ is ethyl. In some embodiments, one or both of R¹ or R² is hydroxyl; for example, sometimes R¹ is hydroxyl. In certain embodiments, Z is substituted with one or more electron withdrawing groups.

In certain embodiments, Z is a substituted or unsubstituted pyridine N-oxide ring; R^{a} is H or methyl; and R¹ and R² are, independently, H, halogen, such as fluoro or chloro, hydroxyl, methyl, ethyl, methoxy, or ethoxy, provided that at least one of R¹ and R² is not H. In certain embodiments, one or both of R¹ or R² is methoxy; for example, sometimes R¹ is methoxy, sometimes R² is methoxy, and sometimes both R¹ and R² are methoxy. In certain embodiments, one or both of R¹ or R² is fluoro; for example, sometimes R¹ is fluoro, sometimes R² is fluoro, and sometimes both R¹ and R² are fluoro. In certain embodiments, R² is H. In certain embodiments, R¹ is hydroxyl. In certain embodiments, R¹ is methyl. In certain embodiments, R¹ is ethyl. In some embodiments, one or both of R¹ or R² is hydroxyl; for example, sometimes R¹ is hydroxyl. In certain embodiments, Z is substituted with one or more electron withdrawing groups.

In certain embodiments, Z is a pyridine ring substituted with one or more halogens, such as fluoro and/or chloro, and optionally further substituted; R^{a} is H or methyl; and R¹ and R² are, independently, H, halogen, such as fluoro or chloro, hydroxyl, methyl, ethyl, methoxy, or ethoxy, provided that at least one of R¹ and R² is not H. In certain embodiments, one or both of R¹ or R² is methoxy; for example, sometimes R¹ is methoxy, sometimes R² is methoxy, and sometimes both R¹ and R² are methoxy. In certain embodiments, one or both of R¹ or R² is fluoro; for example, sometimes R¹ is fluoro, sometimes R² is fluoro, and sometimes both R¹ and R² are fluoro. In certain embodiments, one or both of R¹ or R² is fluoro; for example, sometimes R¹ is fluoro, sometimes R² is fluoro, and sometimes both R¹ and R² are fluoro. In certain embodiments, R² is H. In certain embodiments, R¹ is hydroxyl. In certain embodiments, R¹ is methyl. In certain embodiments, R¹ is ethyl. In some embodiments, one or both of R¹ or R² is hydroxyl; for example, sometimes R¹ is hydroxyl. In certain embodiments, Z is substituted with one or more electron withdrawing groups.

In certain embodiments, Z is a substituted or unsubstituted aryl or heteroaryl ring; R^{a} is H; and R¹ and R² are, independently, H, halogen, such as fluoro or chloro, hydroxyl, methyl, ethyl, methoxy, or ethoxy, provided that at least one of R¹ and R² is not H. In certain embodiments, one or both of R¹ or R² is methoxy; for example, sometimes R¹ is methoxy, sometimes R² is methoxy, and sometimes both R¹ and R² are methoxy. In certain embodiments, R² is H. In certain embodiments, R¹ is hydroxyl. In certain embodiments, R¹ is methyl. In certain embodiments, R¹ is ethyl. In some embodiments, one or both of R¹ or R² is hydroxyl; for example, sometimes R¹ is hydroxyl. In certain embodiments, Z is substituted with one or more electron withdrawing groups.

In certain embodiments, the compound of Formula A has the structure:

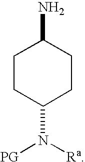

In some embodiments, Cy′ is substituted with one or more halogen and/or alkyl groups. For example, in some instances Cy′ is substituted with one or more chloro and/or fluoro groups.

In particular embodiments, Cy′ is a 3-halo-benzo[b]thien-2-yl, for example a 3-chloro-benzo[b]thien-2-yl or a 3-fluoro-benzo[b]thien-2-yl, or a 3-methyl-benzo[b]thien-2-yl. The benzo ring of Cy′ may be substituted with from 1-4 substituents such as halogen, nitro, cyano, methyl (e.g., including halomethyl, such as CHCl₂ and CF₃), and ethyl (e.g., including haloethyl, such as CH₂CCl₃, C₂F₅, etc.), preferably with halogen and methyl (e.g., including halomethyl, such as CHCl₂ and CF₃). In certain such embodiments, Cy′ represents a 3-chloro-benzo[b]thien-2-yl, 3-fluoro-benzo[b]thien-2-yl, or 3-methyl-benzo[b]thien-2-yl, wherein the benzo ring is substituted with fluoro at the 4-position (peri to the 3-substituent on the thienyl ring) and, optionally, at the 7-position (‘peri’ to the S of the thienyl ring). Alternatively, the benzo ring may be unsubstituted. In certain embodiments, the benzo ring is selected from: 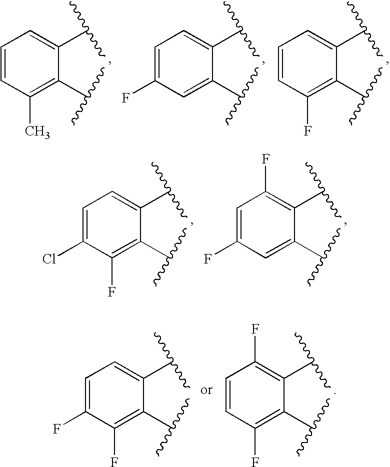
In yet further embodiments, Cy′ is selected from:

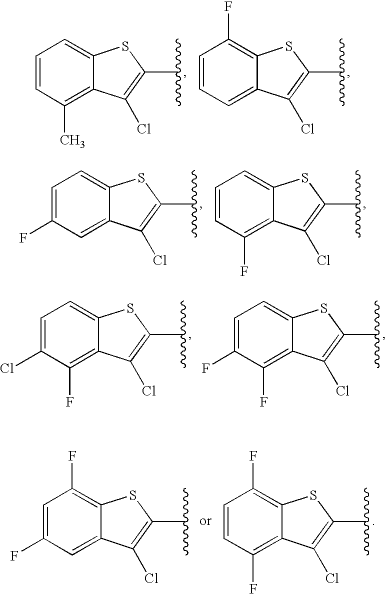

In certain embodiments, compounds according to Formula I of the present can be represented by Formula V:

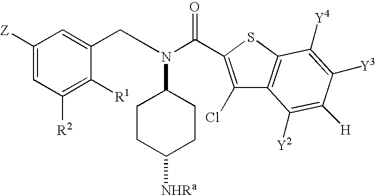

### Formula V

wherein, as valence and stability permit,
Z is a substituted or unsubstituted aryl or heteroaryl ring;
R^{a} is H or methyl;
R¹ and R² are, independently, H, halogen, hydroxyl, lower alkyl, methoxy, or ethoxy, provided that at least one of R¹ and R² is not H;
Y² and Y⁴ are, independently, H or fluorine; and
Y³ is H or fluorine.

In certain embodiments, Z is a substituted or unsubstituted aryl or heteroaryl ring; R^{a} is methyl; R¹ is halogen, such as fluoro or chloro, methoxy, or ethoxy; R² is H; Y² and Y⁴ are, independently, H or fluoro; and Y³ is H or fluoro. In some embodiments, R¹ is methoxy. In other embodiments, R¹ is fluoro. In some instances, R¹ is ethoxy. In some instances, Z is not a substituted or unsubstituted pyridine N-oxide ring or a pyridine ring substituted with one or more halogens. In certain embodiments, Z is substituted with one or more electron withdrawing groups.

In certain embodiments, Z is a substituted or unsubstituted aryl or heteroaryl ring; R^{a} is H or methyl; R¹ is H; R² is halogen, such as fluoro or chloro, hydroxyl, methyl, ethyl, methoxy, or ethoxy; Y² and Y⁴ are, independently, H or fluoro; and Y³ is H or fluoro. In some embodiments, R² is methoxy. In certain embodiments, R^{a} is methyl. In certain embodiments, Z is substituted with one or more electron withdrawing groups.

In certain embodiments, Z is a substituted or unsubstituted aryl or heteroaryl ring; R^{a} is H or methyl; R¹ is hydroxyl, methyl, or ethyl; R² is H, halogen, such as fluoro or chloro, hydroxyl, methyl, ethyl, methoxy, or ethoxy; Y² and Y⁴ are, independently, H or fluoro; and Y³ is H or fluoro. In certain embodiments, R^{a} is methyl. In certain embodiments, R² is H. In certain embodiments, R¹ is hydroxyl. In certain embodiments, R¹ is methyl. In certain embodiments, R¹ is ethyl. In some embodiments, one or both of R¹ or R² is hydroxyl; for example, sometimes R¹ is hydroxyl. In certain embodiments, Z is substituted with one or more electron withdrawing groups.

In certain embodiments, Z is a substituted or unsubstituted pyridine N-oxide ring; R^{a} is H or methyl; R¹ and R² are, independently, H, halogen, such as fluoro or chloro, hydroxyl, methyl, ethyl, methoxy, or ethoxy, provided that at least one of R¹ and R² is not H; Y² and Y⁴ are, independently, H or fluoro; and Y³ is H or fluoro. In certain embodiments, one or both of R¹ or R² is methoxy; for example, sometimes R¹ is methoxy, sometimes R² is methoxy, and sometimes both R¹ and R² are methoxy. In certain embodiments, one or both of R¹ or R² is fluoro; for example, sometimes R¹ is fluoro, sometimes R² is fluoro, and sometimes both R¹ and R² are fluoro. In certain embodiments, R² is H. In certain embodiments, R¹ is hydroxyl. In certain embodiments, R¹ is methyl. In certain embodiments, R¹ is ethyl. In some embodiments, one or both of R¹ or R² is hydroxyl; for example, sometimes R¹ is hydroxyl. In certain embodiments, Z is substituted with one or more electron withdrawing groups.

In certain embodiments, Z is a pyridine ring substituted with one or more halogens, such as fluoro and/or chloro, and optionally further substituted; R^{a} is H or methyl; R¹ and R² are, independently, H, halogen, such as fluoro or chloro, hydroxyl, methyl, ethyl, methoxy, or ethoxy, provided that at least one of R¹ and R² is not H; Y² and Y⁴ are, independently, H or fluoro; and Y³ is H or fluoro. In certain embodiments, one or both of R¹ or R² is methoxy; for example, sometimes R¹ is methoxy, sometimes R² is methoxy, and sometimes both R¹ and R² are methoxy. In certain embodiments, one or both of R¹ or R² is fluoro; for example, sometimes R¹ is fluoro, sometimes R² is fluoro, and sometimes both R¹ and R² are fluoro. In certain embodiments, one or both of R¹ or R² is fluoro; for example, sometimes R¹ is fluoro, sometimes R² is fluoro, and sometimes both R¹ and R² are fluoro. In certain embodiments, R² is H. In certain embodiments, R¹ is hydroxyl. In certain embodiments, R¹ is methyl. In certain embodiments, R¹ is ethyl. In some embodiments, one or both of R¹ or R² is hydroxyl; for example, sometimes R¹ is hydroxyl. In certain embodiments, Z is substituted with one or more electron withdrawing groups.

In certain embodiments, Z is a substituted or unsubstituted aryl or heteroaryl ring; R^{a} is H; R¹ and R² are, independently, H, halogen, such as fluoro or chloro, hydroxyl, methyl, ethyl, methoxy, or ethoxy, provided that at least one of R¹ and R² is not H; Y² and Y⁴ are, independently, H or fluoro; and Y³ is H or fluoro. In certain embodiments, one or both of R¹ or R² is methoxy; for example, sometimes R¹ is methoxy, sometimes R² is methoxy, and sometimes both R¹ and R² are methoxy. In certain embodiments, R² is H. In certain embodiments, R¹ is hydroxyl. In certain embodiments, R¹ is methyl. In certain embodiments, R¹ is ethyl. In some embodiments, one or both of R¹ or R² is hydroxyl; for example, sometimes R¹ is hydroxyl. In certain embodiments, Z is substituted with one or more electron withdrawing groups.

Variations, modifications, and other implementations of what is described herein will occur to those of ordinary skill without departing from the spirit and the scope of the invention. Accordingly, the invention is not to be limited only to the preceding illustrative description. For additional illustrative features that may be used with the invention, including the embodiments described here, refer to the documents listed herein above and incorporated by reference in their entirety. All operative combinations between the above described illustrative embodiments and those features described in WO 01/74344, U.S. Ser. No. 03/027234, U.S. Ser. No. 03/0139457, and U.S. 60/763,154 are considered to be potentially patentable embodiments of the invention.

Moreover, all various combinations of the above definitions for variables Ar, R′ R¹, R², R⁴, R^{a}, R^{x}, M, X, Y², Y³, Y⁴, Z, Cy′, PG, LG, n, etc. recited herein are contemplated. Thus, although one or more combinations of the above variables may not be explicitly recited herein as a discrete combination, the present invention includes such combinations.

Similarly, various individual chemical reactions as disclosed herein may be combined and used in a variety of sequences and combinations, as would be understood by one of skill in the art. Accordingly, the present invention contemplates all sequences and combinations of chemical reactions disclosed herein.

### 3. Compounds

In certain embodiments, the present invention provides methods for the preparation of one or more of the following compounds:

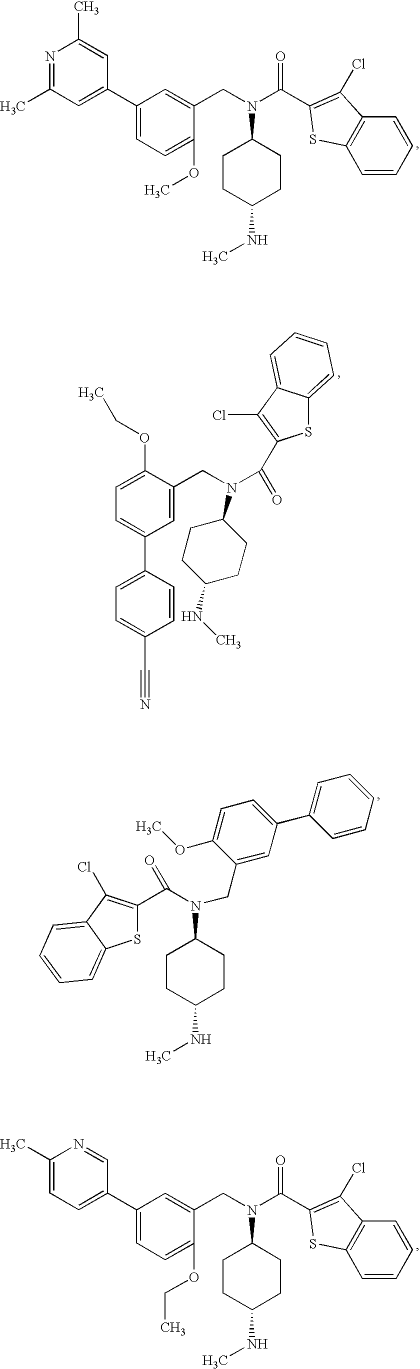

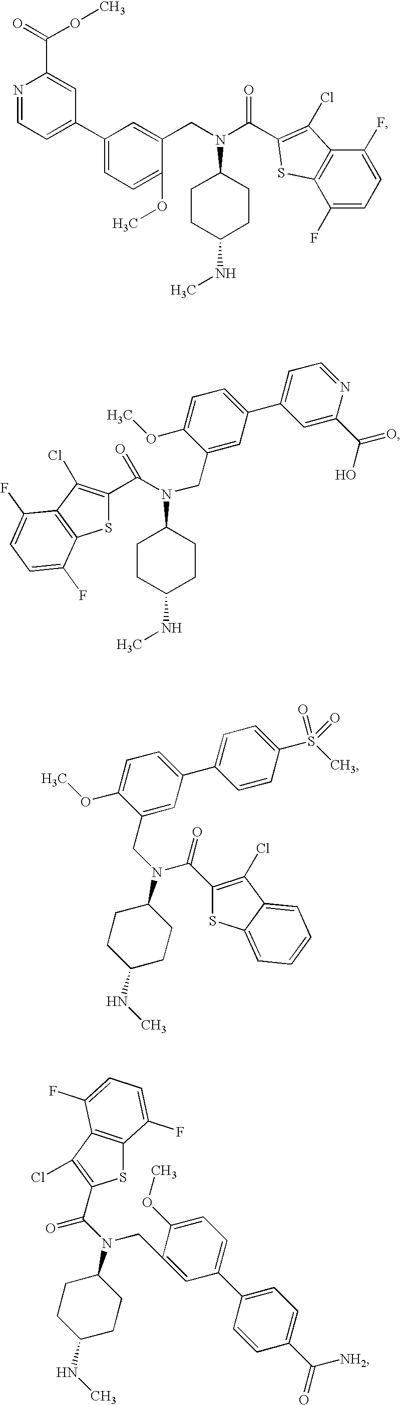

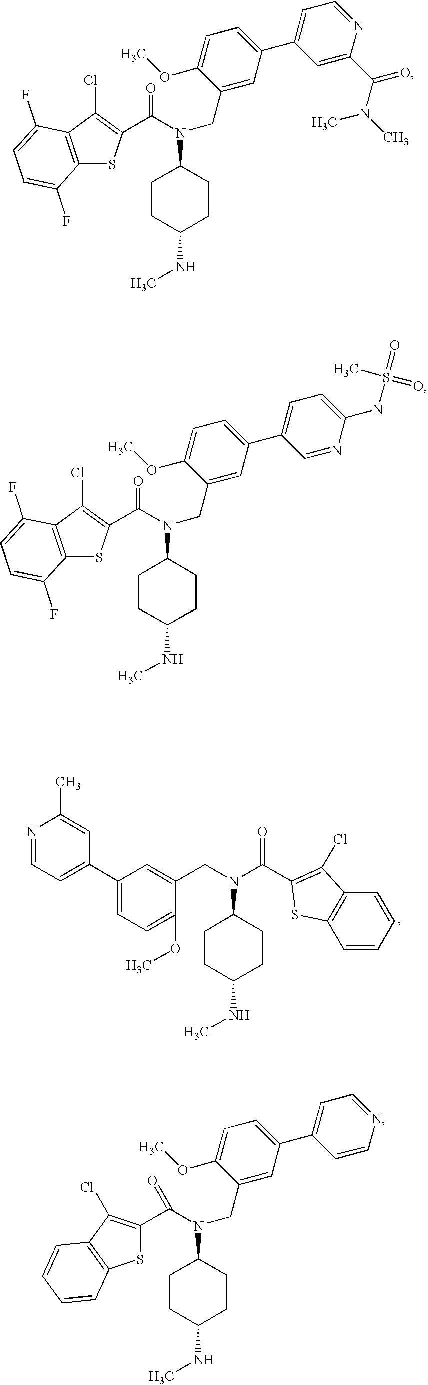

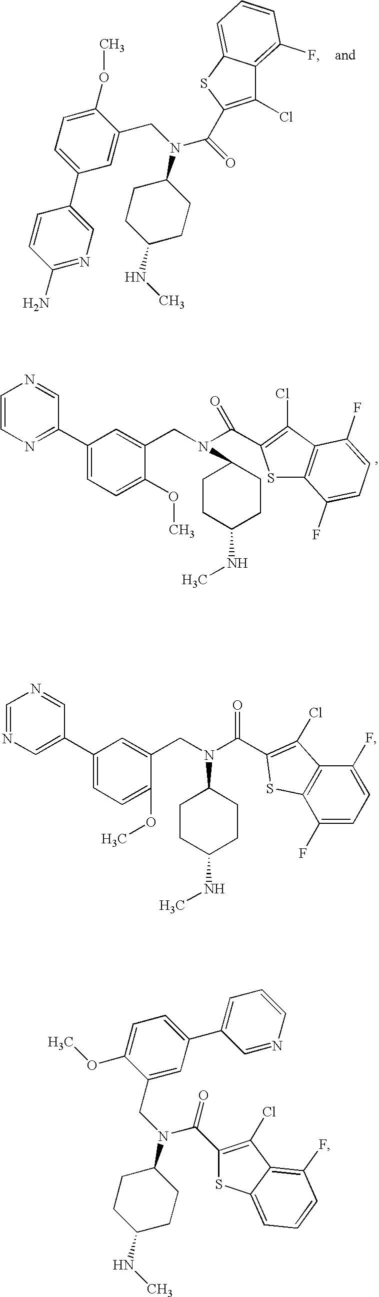

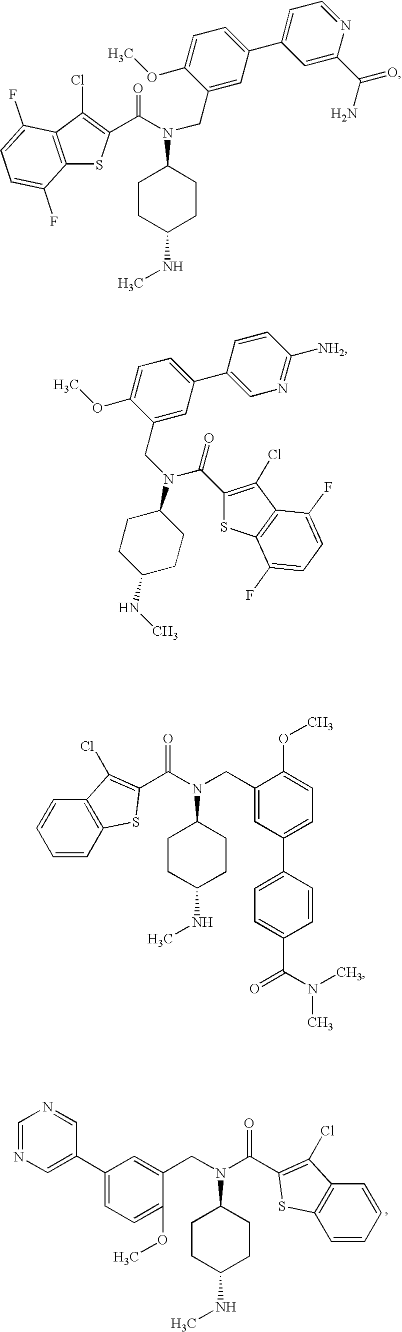

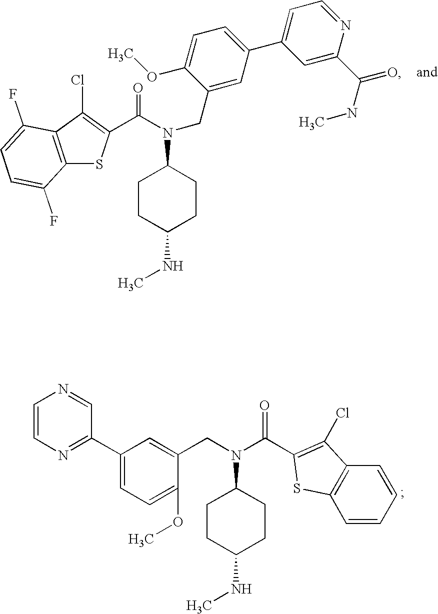

or one or more of the following compounds:

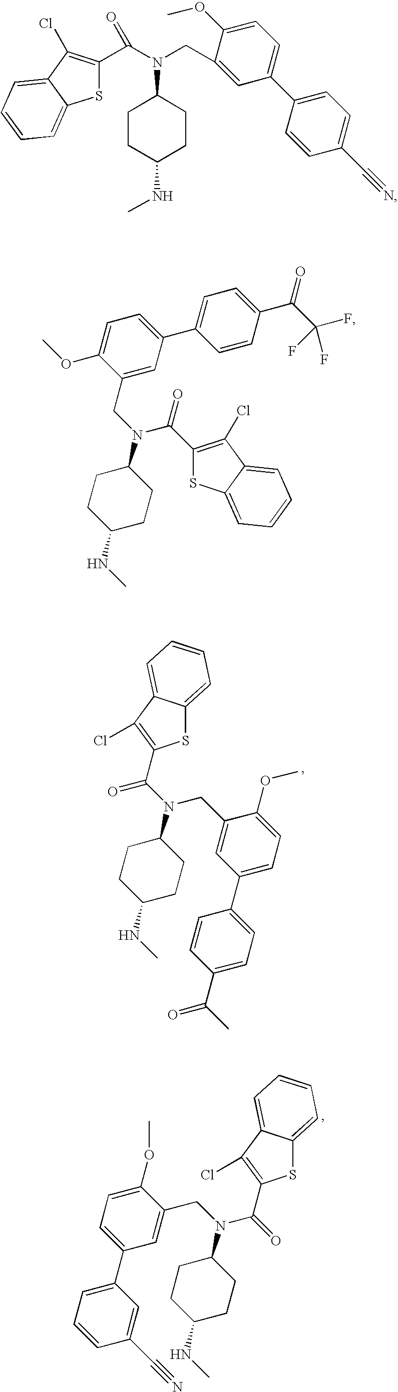

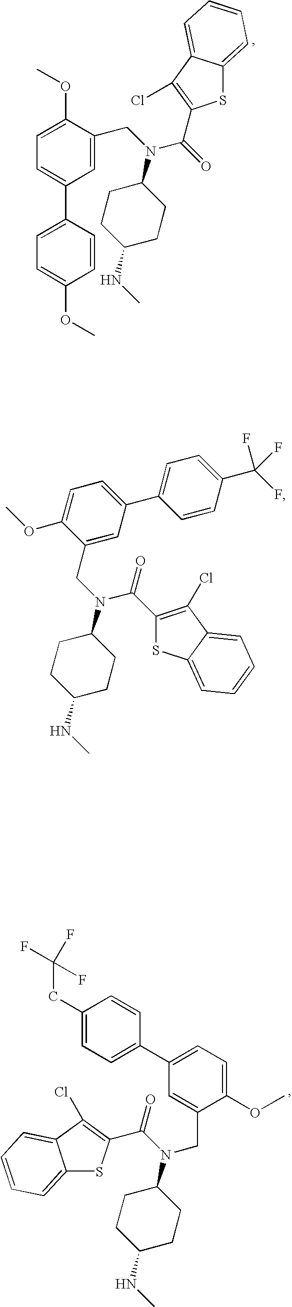

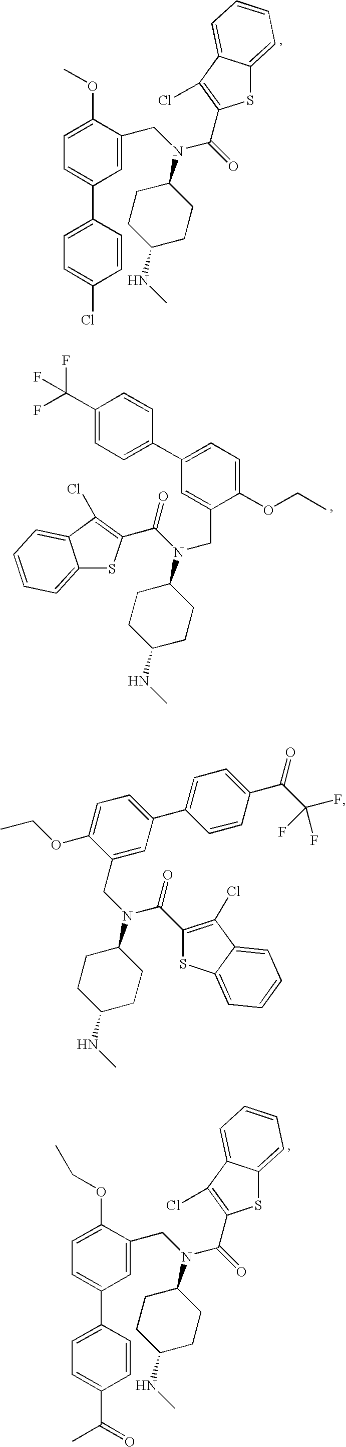

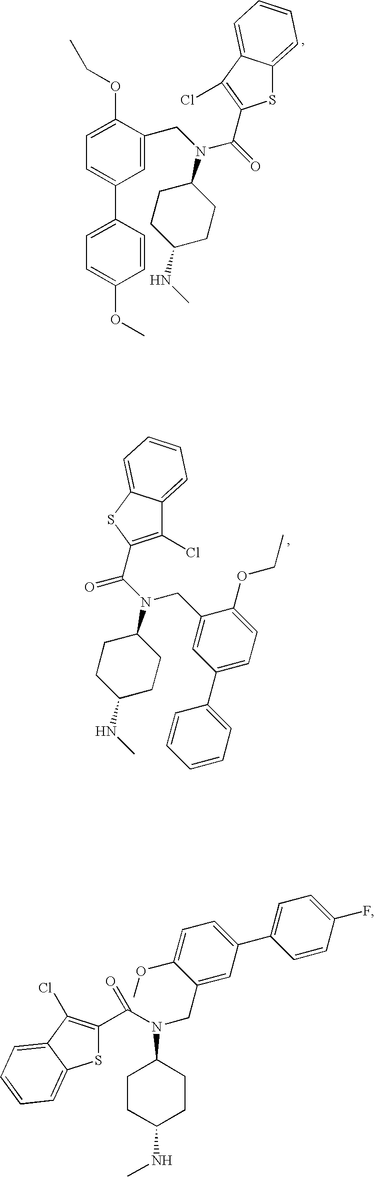

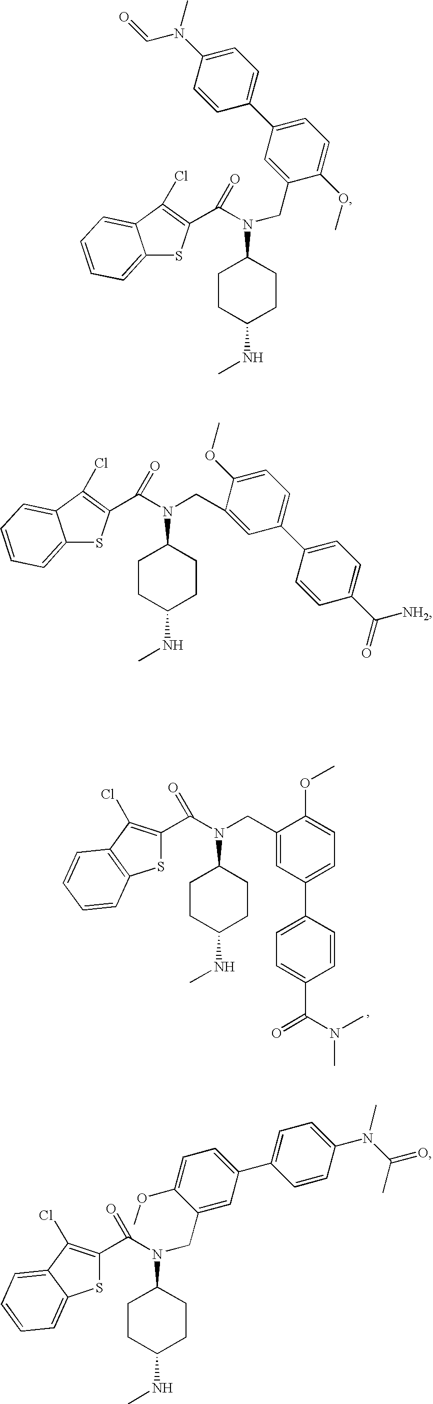

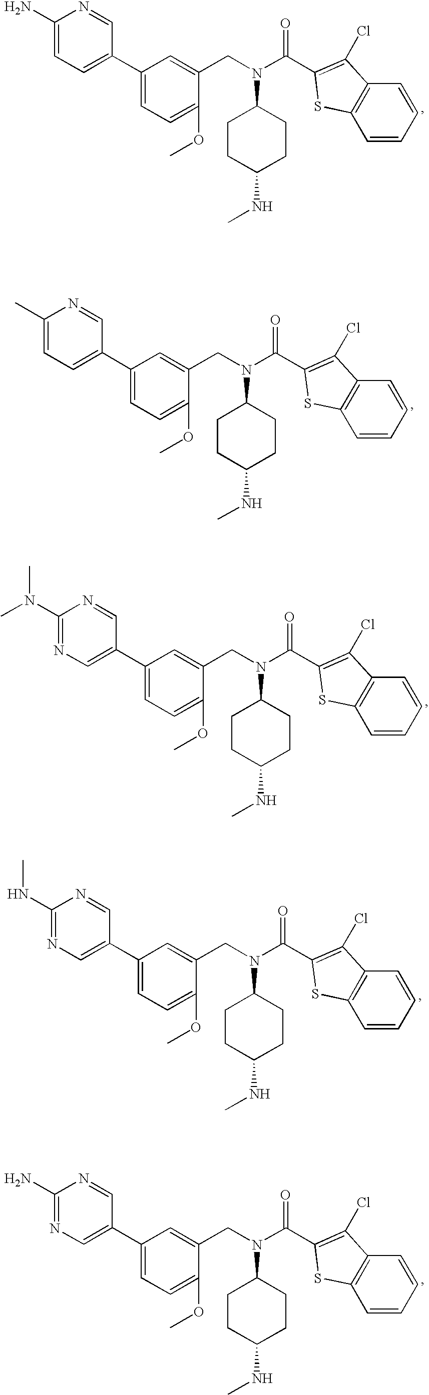

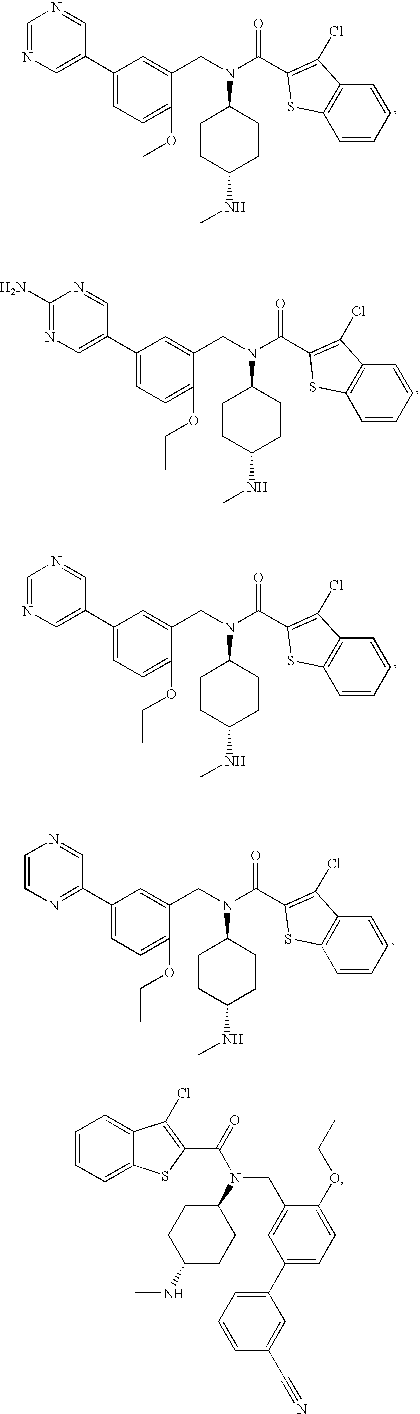

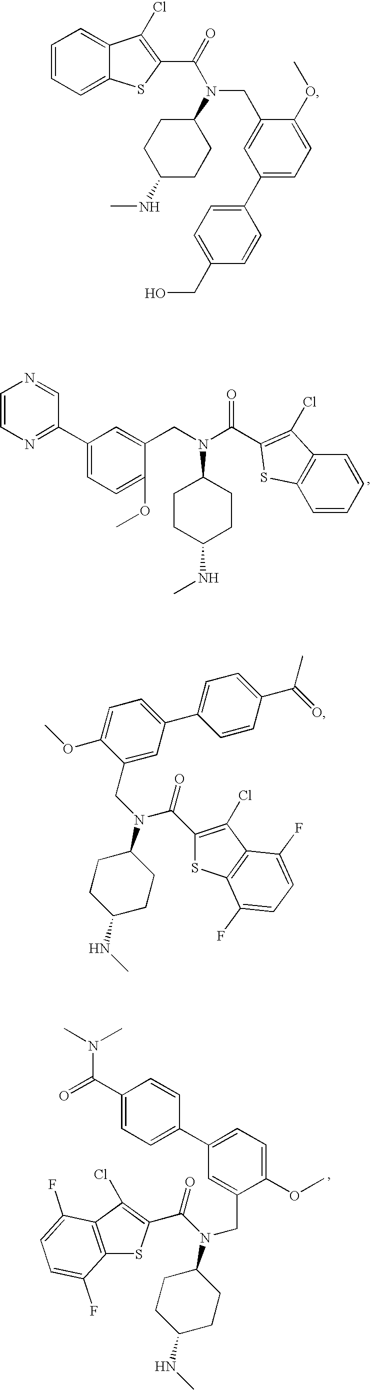

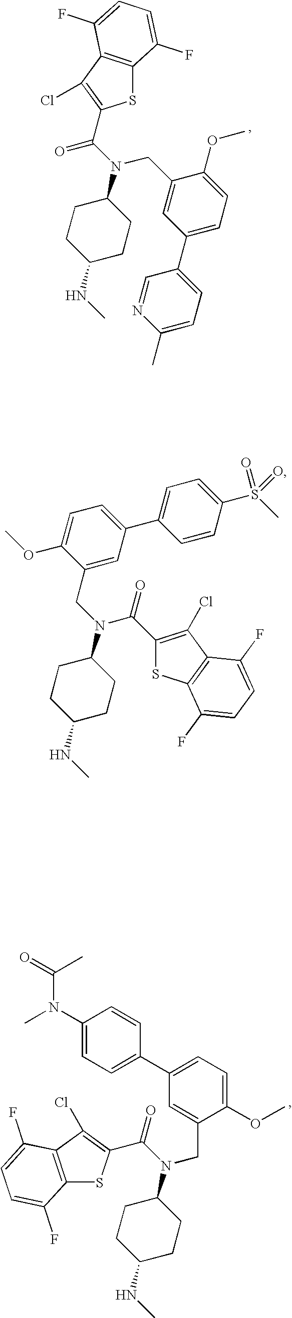

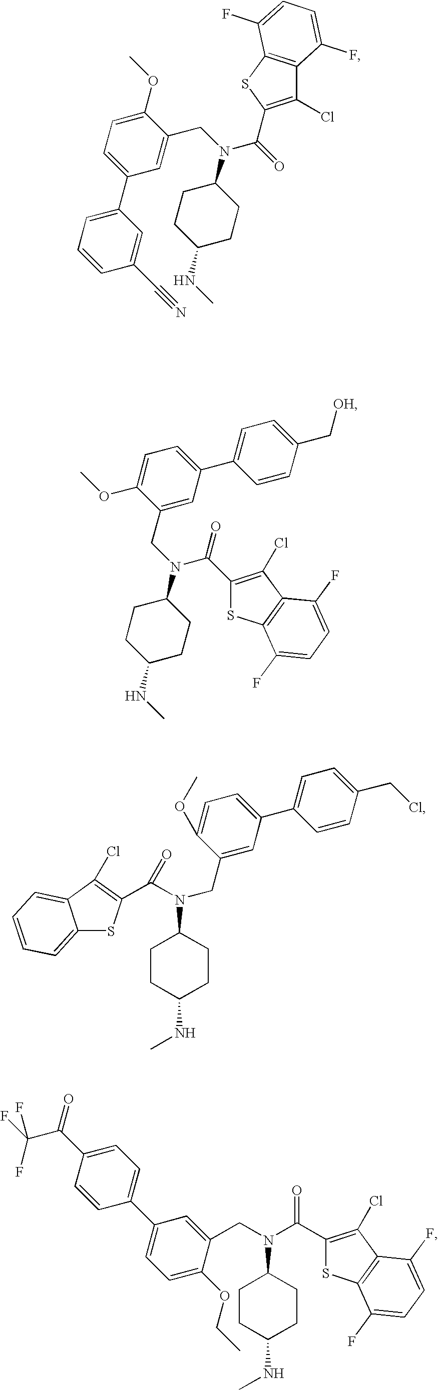

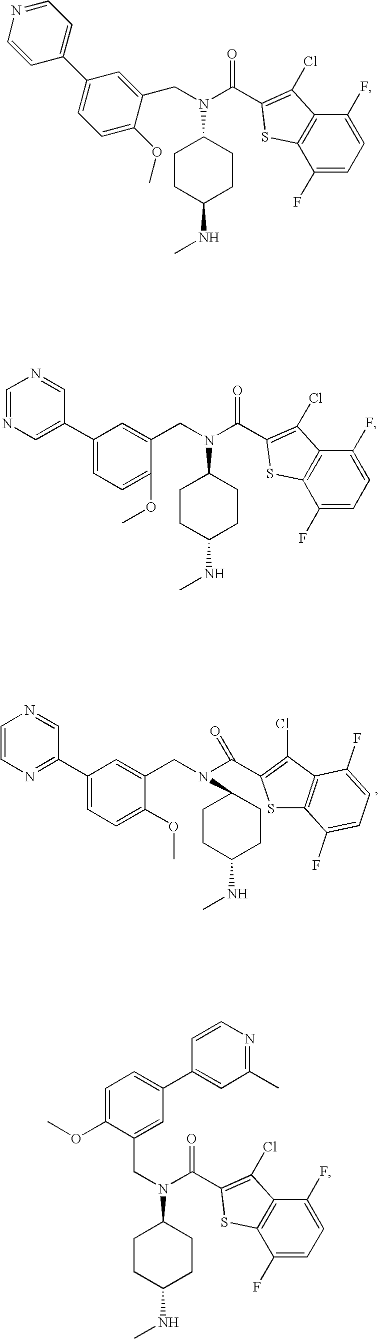

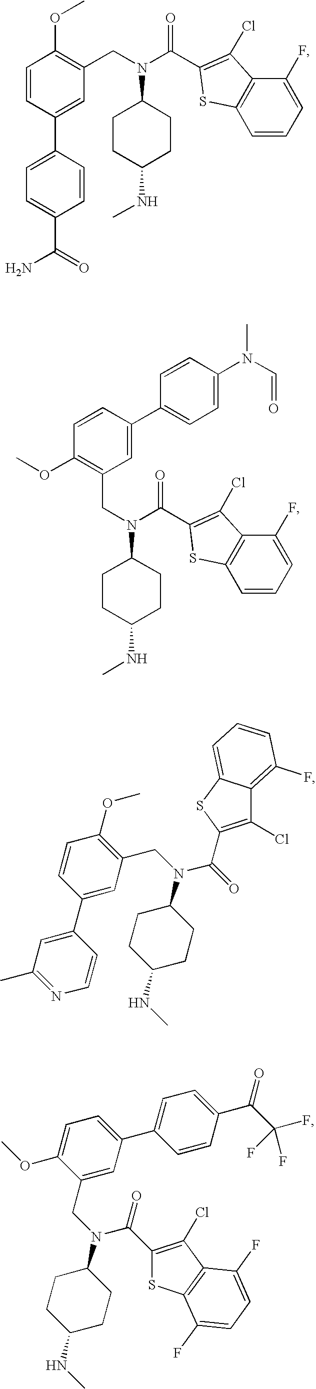

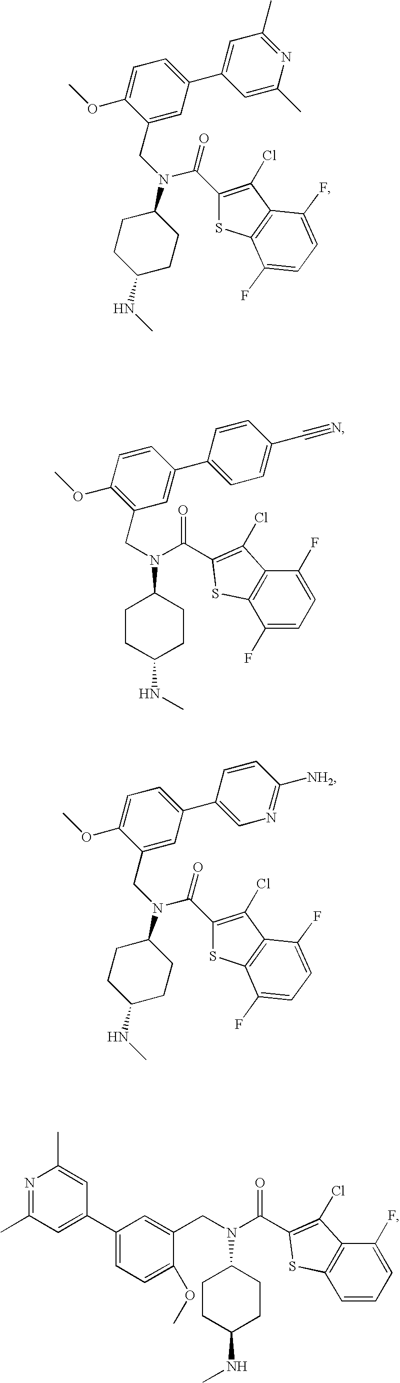

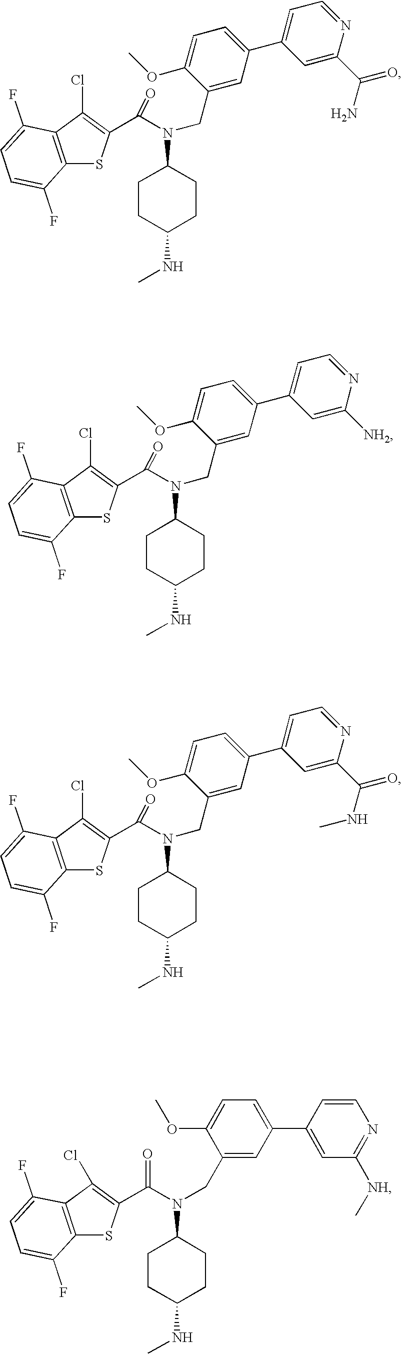

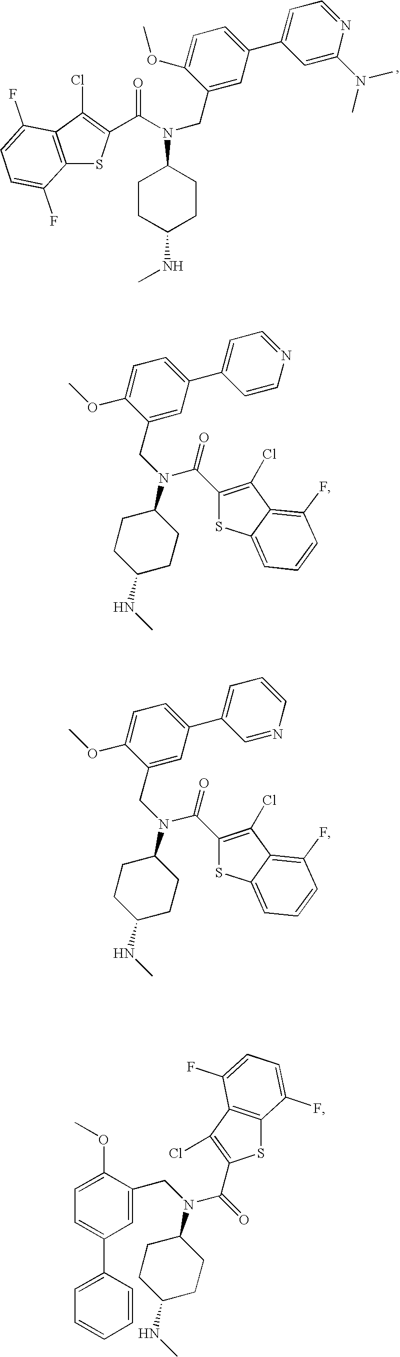

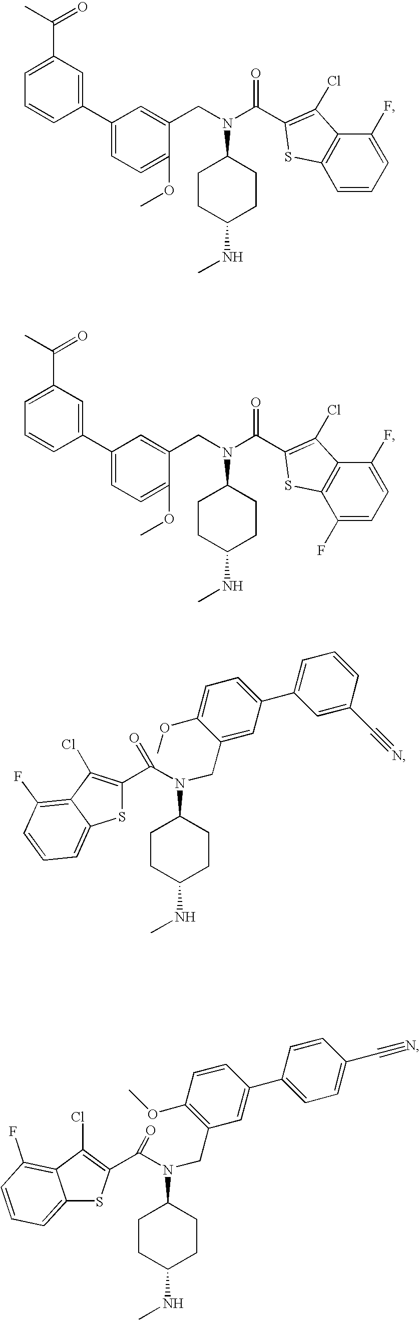

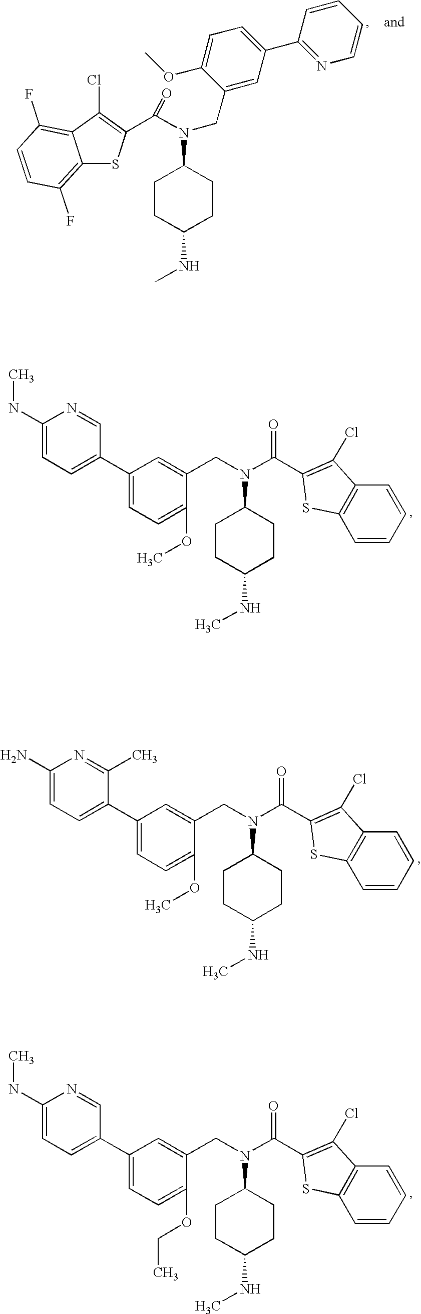

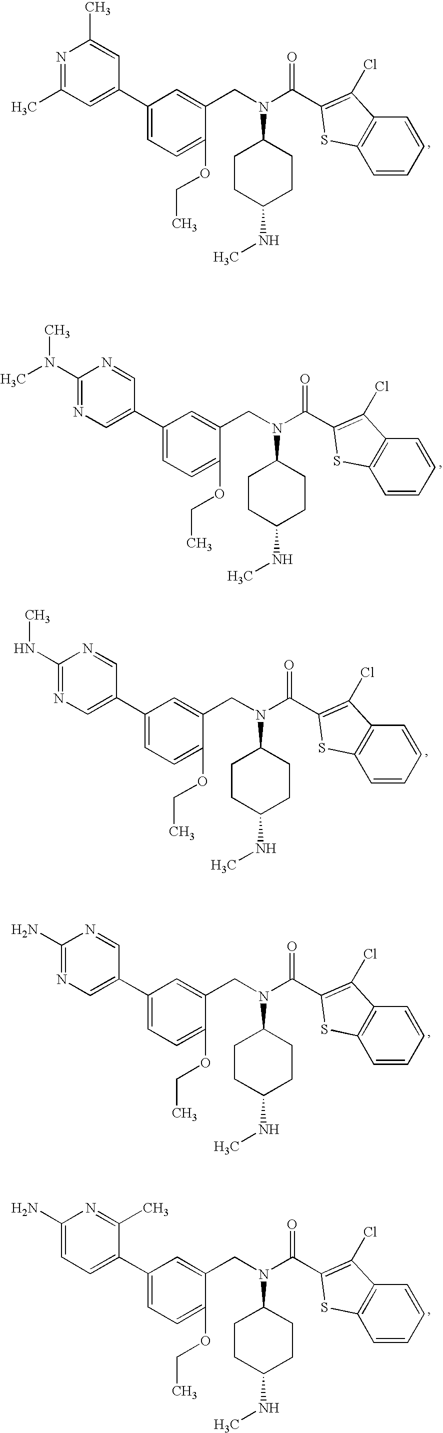

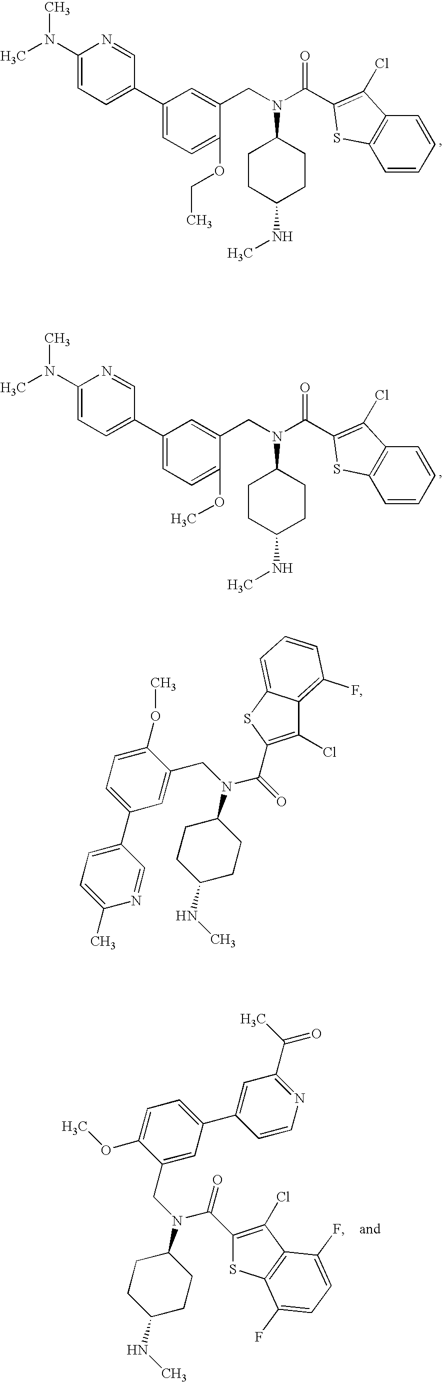

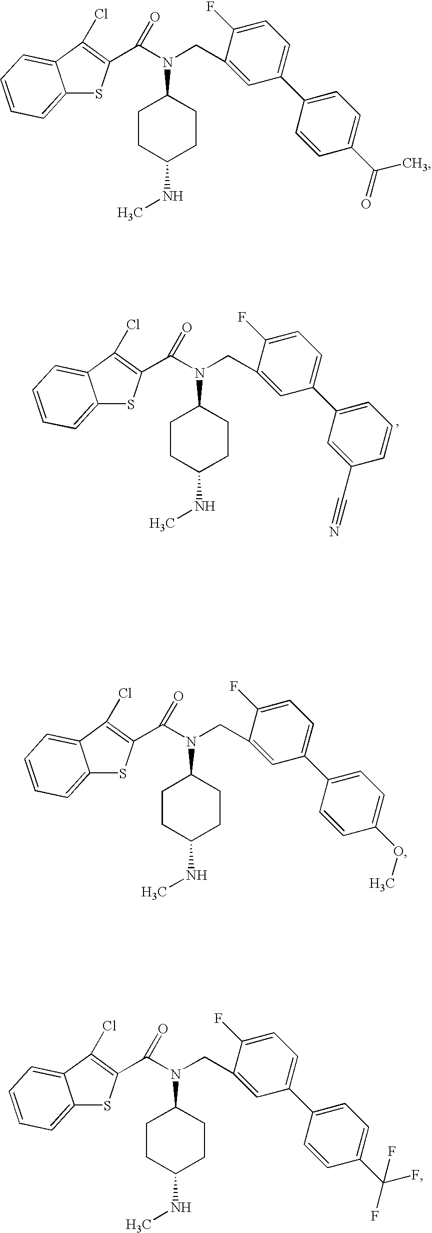

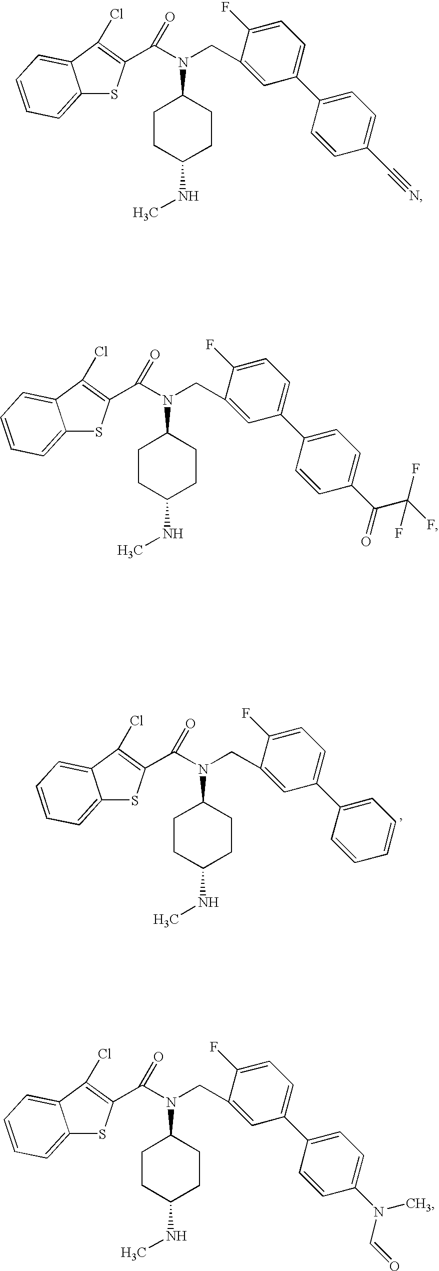

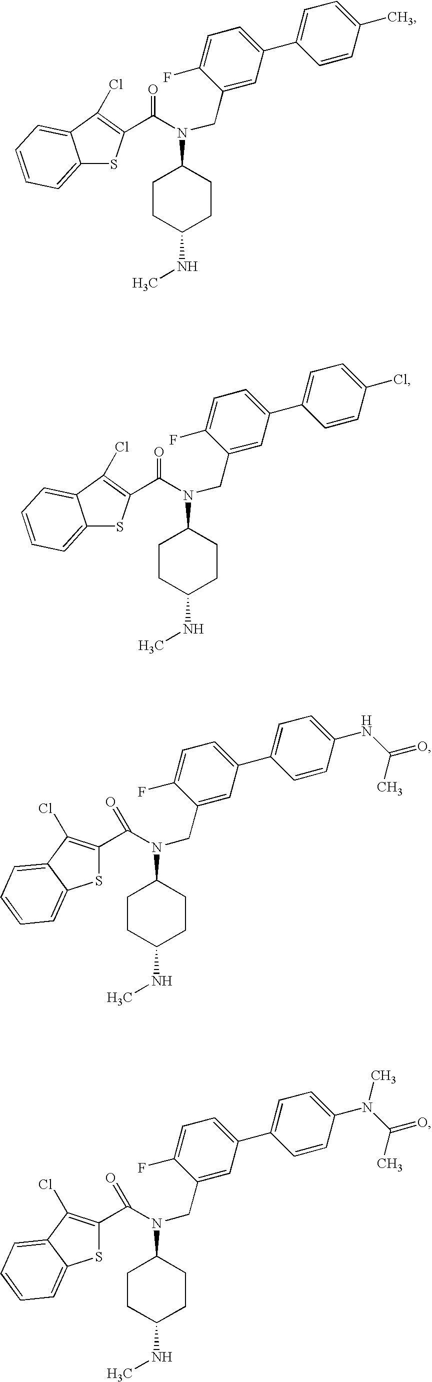

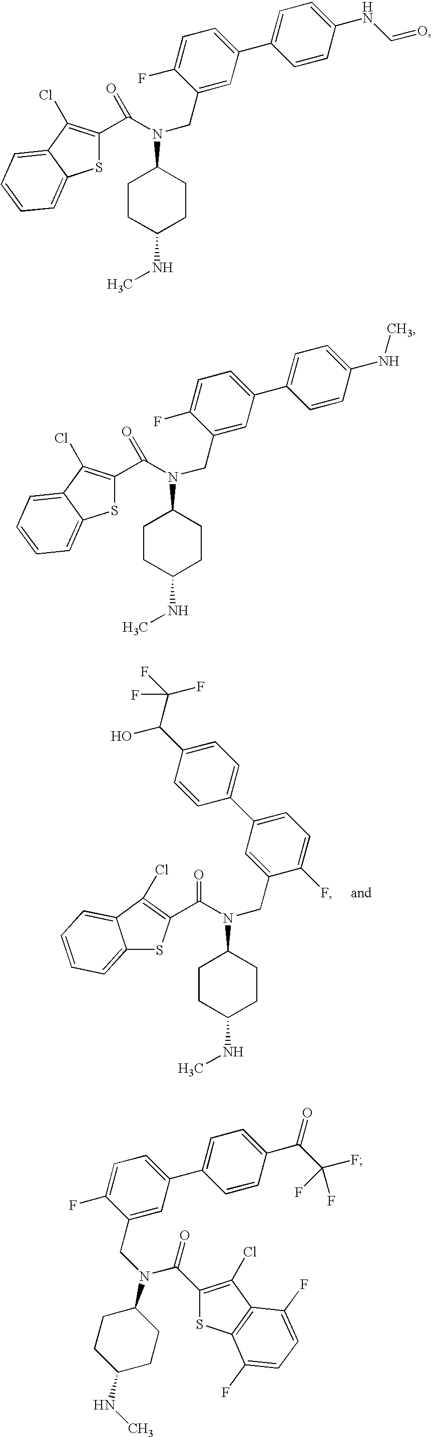

or one or more of the following compounds:

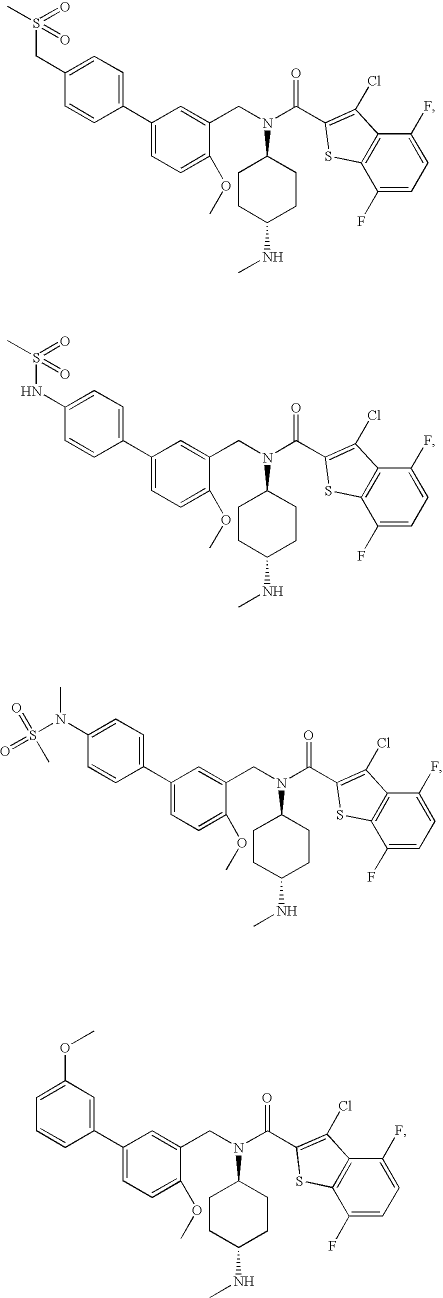

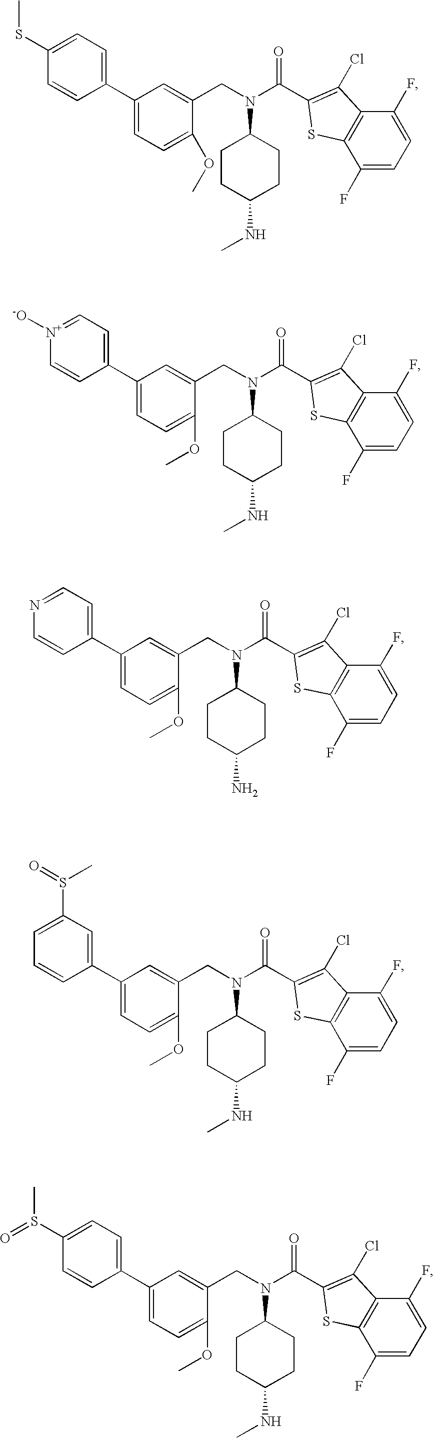

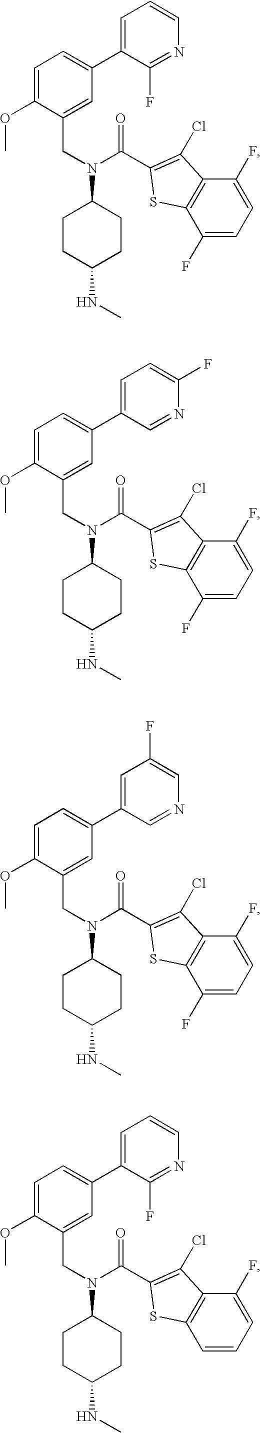

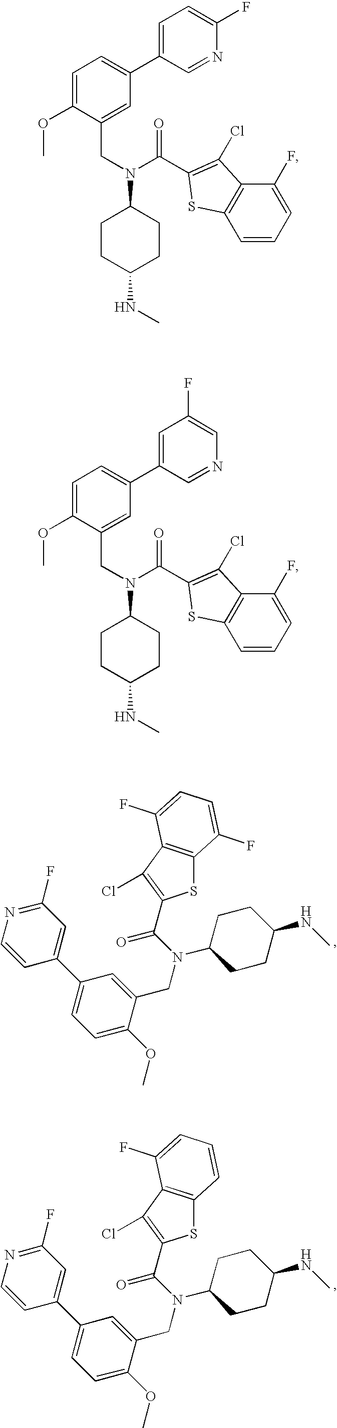

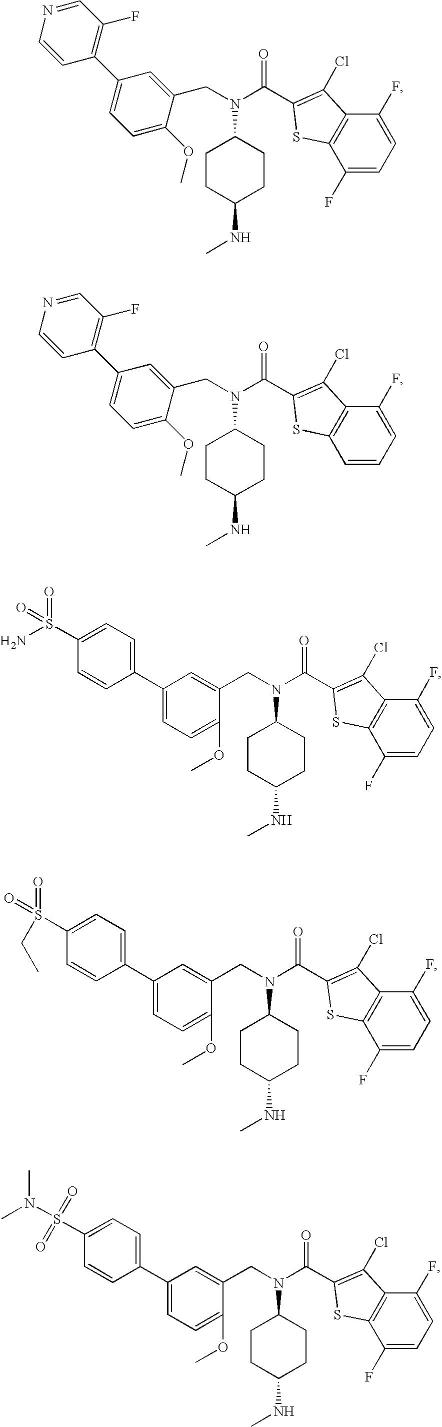

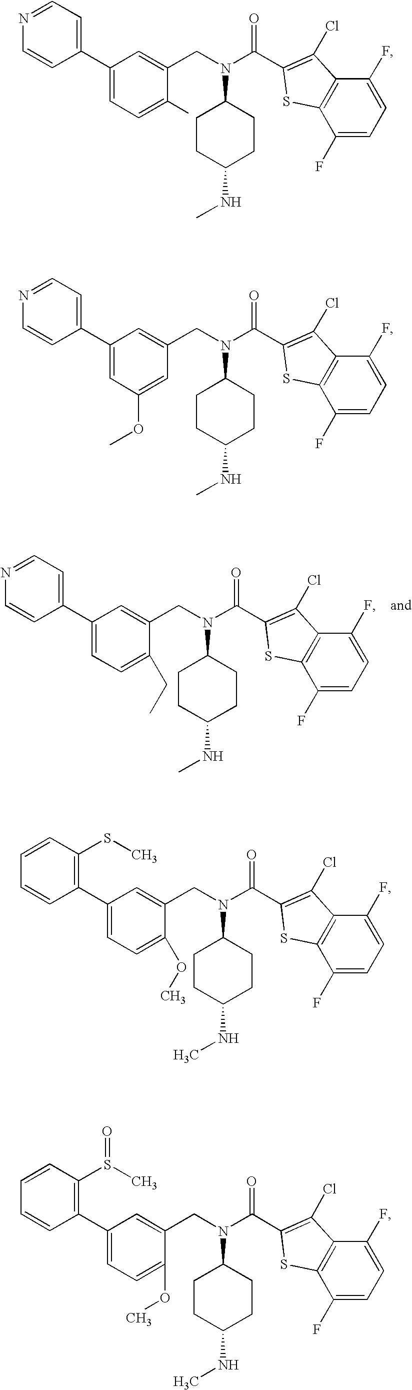

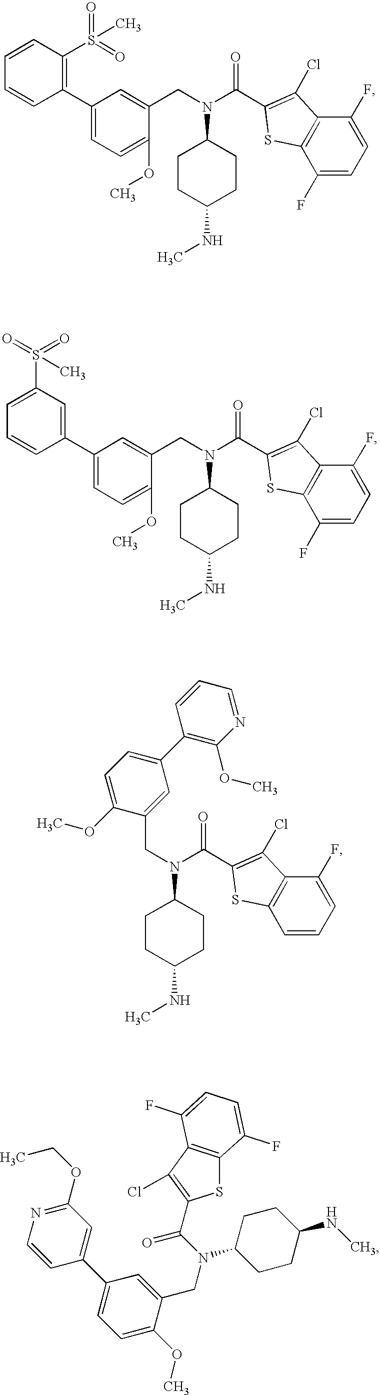

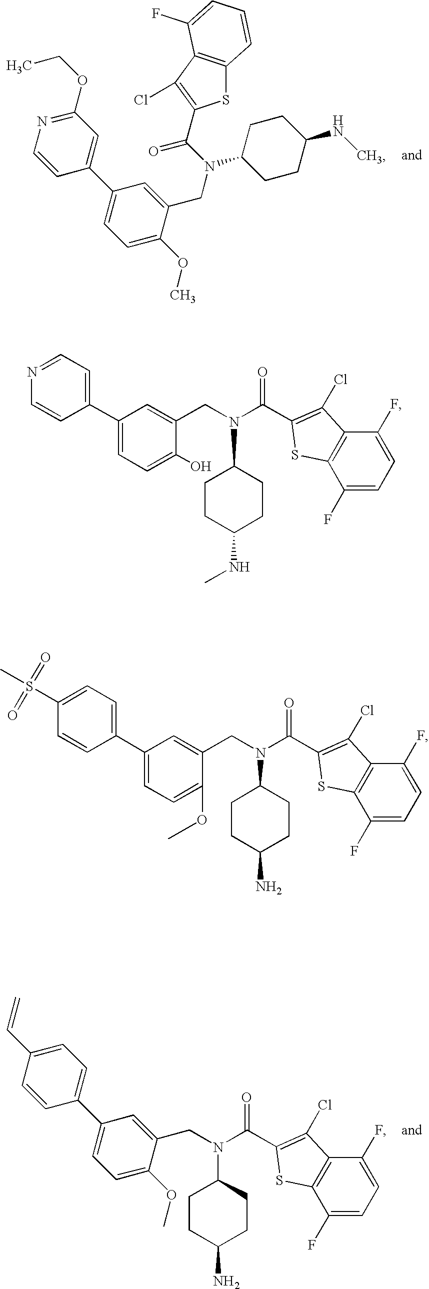

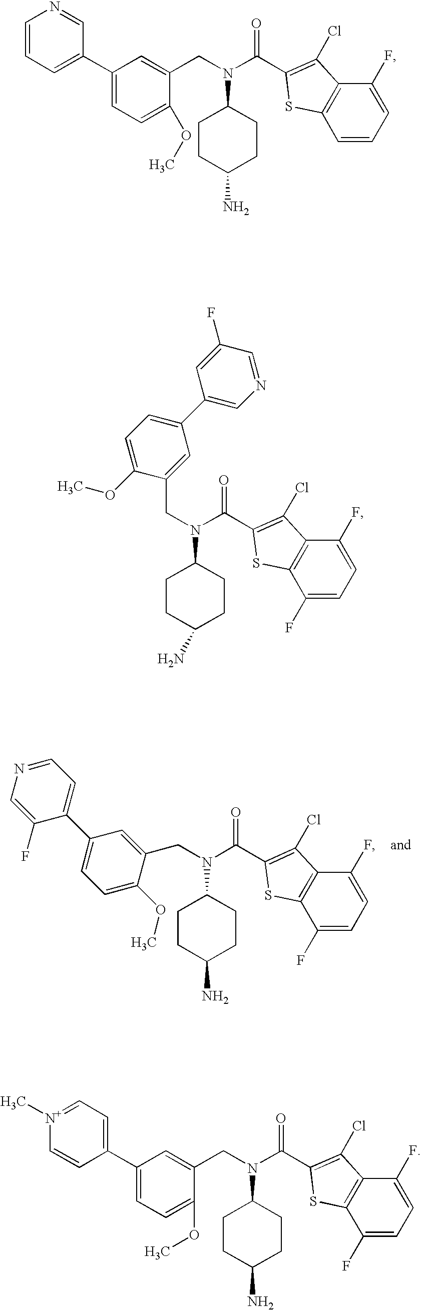

For the purpose of clarity, despite any appearances to the contrary, all of the compounds whose structures are depicted above in section 3 have a cyclohexyl ring with two substituents disposed trans on the ring.

### EXEMPLIFICATION

The invention now being generally described, it will be more readily understood by reference to the following examples which are included merely for purposes of illustration of certain aspects and embodiments of the present invention, and are not intended to limit the invention.

### Compound Synthesis

Compounds of the present invention have been synthesized according to the following general methods.

### ABBREVIATIONS

AcOH glacial acetic acid

BOC tert-butoxycarbonyl

br. broad

n-BuLi n-butyllithium

conc. concentrated

d doublet

DCM dichloromethane

DIPEA N,N-diisopropylethylamine

DMF N,N-dimethylformamide

DMSO dimethyl sulfoxide

equiv. equivalent

EtOAc ethyl acetate

EtOH ethanol

h hours

HPLC high performance liquid chromatography

LC liquid chromatography

MeOH methanol

min minutes

MS mass spectroscopy

NMR nuclear magnetic resonance

obsc. obscured

PhMe toluene

ppm parts per million

RT ambient (room) temperature

s singlet

TBME tert-butyl methyl ether

TFA trifluoroacetic acid

THF tetrahydrofuran

vol volume (1 vol=1 mL: 1 g)

General Methods

Method A—Suzuki Coupling (Thermal Conditions)

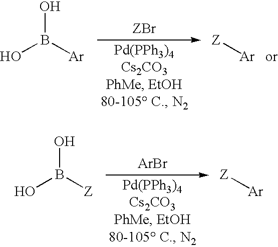

A stirred suspension of the boronic acid (1 equiv.), aryl halide/triflate (1-1.2 equiv.), cesium carbonate (2-2.2 equiv.) and tetrakis(triphenyl-phosphine)palladium(0) (0.05-0.1 equiv.) in toluene (40 vol) and EtOH (10 vol) at RT was degassed with nitrogen for 15 minutes. The mixture was then warmed to 80-105° C. (external temperature). The reaction was monitored by LC/MS and, if incomplete after 3-4 h, more tetrakis(triphenyl-phosphine)palladium(0) (0.05-0.1 equiv.) was added and the reaction heated further (1-2 h). On completion, the reaction mixture was allowed to cool to RT then filtered through celite, washing the solid residues with DCM (100 vol). The filtrate was then reduced in vacuo and the residue purified by chromatography (EtOAc in heptane plus 0.5% triethylamine) to afford the desired biaryl, Z-Ar.

Method B—Suzuki Coupling (Microwave Conditions)

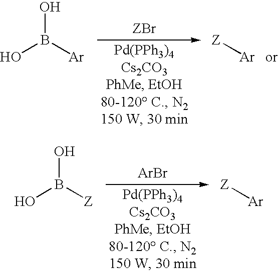

A suspension of the boronic acid (1 equiv.), aryl halide/triflate (1-1.2 equiv.), cesium carbonate (2-2.2 equiv.) and tetrakis(triphenylphosphine)palladium(0) (0.05-0.1 equiv.) in toluene (8 vol) and EtOH (2 vol) at RT was degassed with nitrogen for 15 minutes. The mixture was then irradiated (150 W, Discover® System microwave reactor by CEM Corporation, Matthews, N.C., USA) at 80° C. for 30 min. On cooling, the reaction was analysed by LC/MS and, if incomplete, irradiated again (150 W) at 120° C. for 30 min. If the reaction was still incomplete at this juncture, more tetrakis(triphenylphosphine) palladium(0) (0.05-0.1 equiv.) was added and the reaction irradiated again (150 W) at 120° C. for 30 min. On completion, the reaction mixture was cooled to RT then filtered through celite, washing the solid residues with DCM (100 vol). The filtrate was then reduced in vacuo and the residue purified by chromatography (EtOAc in heptane plus 0.5% triethylamine) to afford the desired biaryl, Z-Ar.

Method C—Reductive Amination

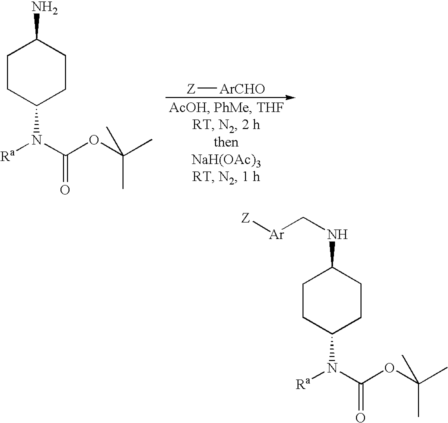

A stirred suspension of amine (1-1.2 equiv.) and aldehyde (1 equiv.) in a 1:1 mixture of THF (15 vol) and toluene (15 vol) was treated with AcOH (1.2 equiv.) at RT. After stirring a minimum of 2 h, the reaction mixture was treated with sodium triacetoxyborohydride (1.4 equiv.) and stirred a minimum of 1 h. Reaction progress was monitored by LC/MS. On completion, the reaction mixture was quenched with aqueous NaHCO₃ (30 vol) and extracted into EtOAc (3×30 vol). The combined organic phases were then dried over Na₂SO₄ and the solvents removed in vacuo to afford the desired crude amine.

Method D—Amide Formation

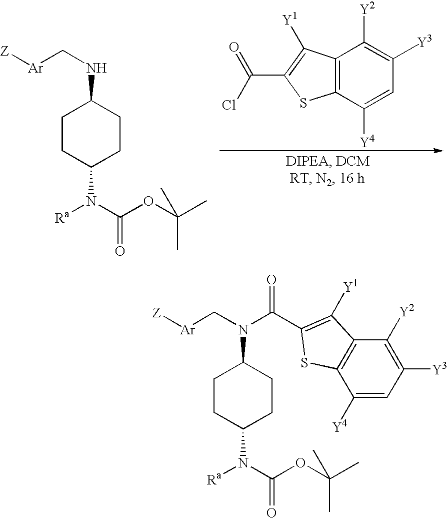

A stirred solution of the amine (1 equiv.) and DIPEA (2.2 equiv.) in DCM (30 vol) was treated with the benzo[b]thiophene acid chloride (1.2-1.5 equiv.) in one portion at 0° C. The solution was then allowed to warm to RT and the reaction progress monitored by LC/MS (typical duration 16 h). On completion the DCM was removed in vacuo and the residue purified by chromatography (EtOAc in heptane plus 0.5% triethylamine) to afford the desired amide.

Method E—BOC Deprotection (HCl in 1,4-dioxane)

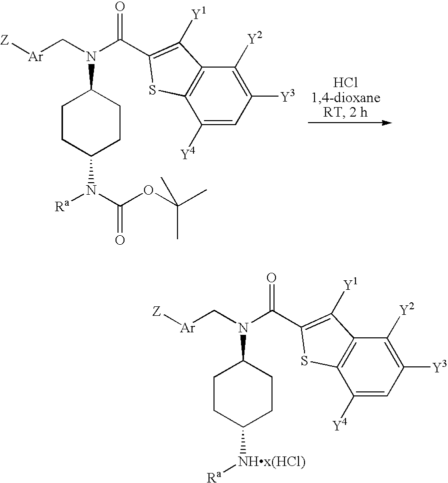

The tert-butyl carbamate was dissolved in a 4 M solution of HCl in 1,4-dioxane (40 vol) and stirred at RT. LC/MS was used to monitor the reaction (typical duration 2 h). On completion, the solvents were removed in vacuo to afford the amine as the HCl salt.

Method F—BOC Deprotection (HCl in EtOH)

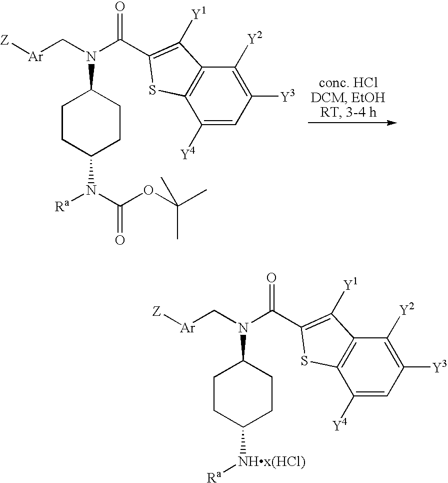

A solution of the tert-butyl carbamate in DCM (15 vol) at RT was diluted with EtOH (50 vol) then conc. HCl (15 vol) was added in one portion. LC/MS was then used to monitor the reaction (typical duration 3-4 h). On completion, the solvents were removed in vacuo to afford the amine as the HCl salt.

Method G—BOC Deprotection (TFA in DCM)

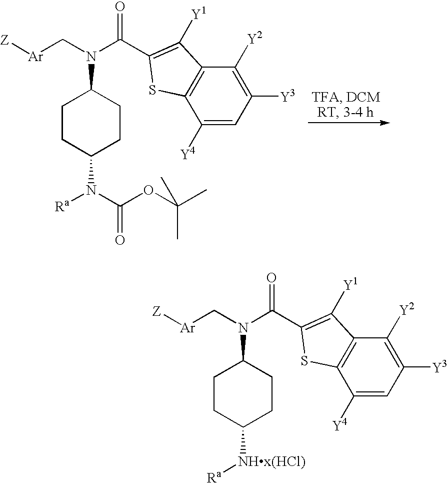

A stirred solution of the tert-butyl carbamate in DCM (120 vol) was treated with TFA (30 vol) at RT. LC/MS was then used to monitor the reaction (typical duration 2-3 h). On completion, the solvents were removed in vacuo to afford the amine as the TFA salt.

Method H—HCl salt formation

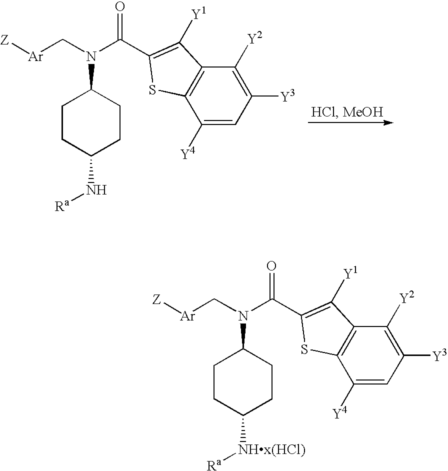

A stirred solution of the amine in MeOH (100 vol) was treated with conc. HCl (25 vol) at RT. The solvents were then removed in vacuo to afford the amine as the HCl salt.

Method J—TFA Salt Formation

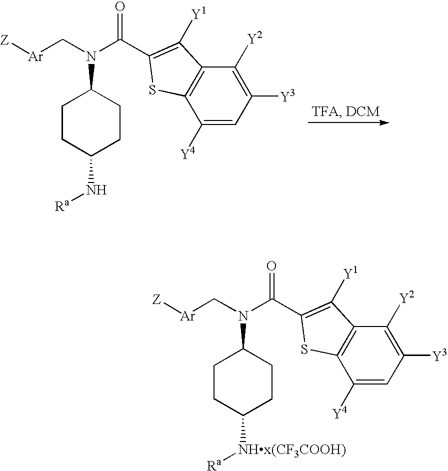

A stirred solution of the amine in DCM (100 vol) was treated with TFA (25 vol) at RT. The solvents were then removed in vacuo to afford the amine as the TFA salt.

Method L1—Synthetic Procedures Used for a Biaryl Library

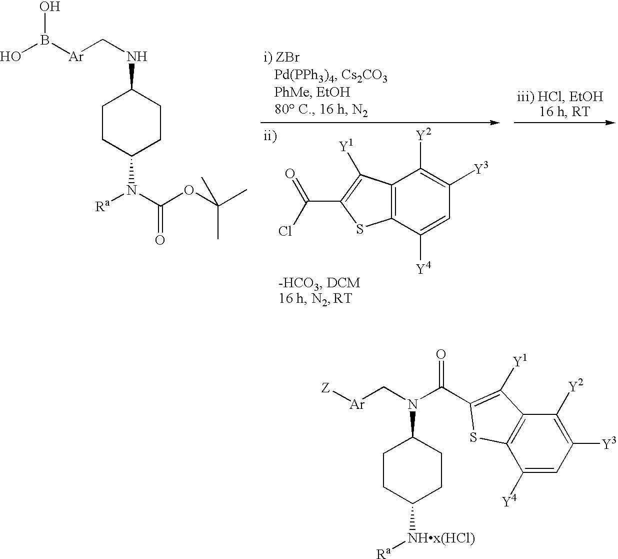

The boronic acid (1.2 equiv.), aryl halide (1.0 equiv.), cesium carbonate (2.2 equiv.) and tetrakis(triphenylphosphine)palladium(0) (0.01 equiv.) were combined and suspended in a mixture of toluene (4 mL) and EtOH (1 mL). The reaction mixtures were warmed to 80° C. (external temperature), degassed with nitrogen for 5 minutes then agitated for 16 h at 80° C. The reactions were allowed to cool to RT then the solvents removed in vacuo. The crude residues were purified by chromatography using firstly 20% EtOAc in heptane to remove triphenylphosphine oxide, then 20% MeOH in EtOAc to isolate the desired amines. On removal of the solvents in vacuo, the amines were dissolved in DCM (4 mL) and treated with bicarbonate resin (2 equiv.) followed by 3-chlorobenzo[b]thiophene-2-carbonyl chloride (1.2 equiv.) at RT. After stirring 16 h, the reaction mixtures were filtered then directly purified by chromatography using firstly 20% EtOAc in heptane to remove reaction by-products, then 50% EtOAc in heptane to isolate the desired amides. On removal of the solvents in vacuo, the amides were dissolved in EtOH (0.66 mL) and treated with conc. HCl (0.33 mL) at RT. After stirring 16 h, the solvents were removed in vacuo, the residues taken up in acetonitrile and analysed by LC/MS. If the purity of the final compound was >50% no further purification was attempted. Thus the acetonitrile was simply removed in vacuo to yield the title compound. If the purity of the final compound was <50%, the title compound was obtained after preparative HPLC.

Method L2—Synthetic Procedures Used for a Biaryl Library

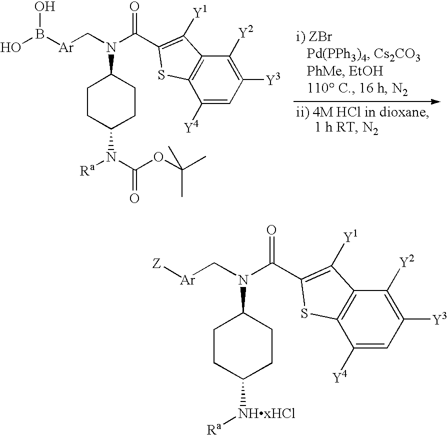

Cesium carbonate (2.2 equiv.) and tetrakis(triphenylphosphine)palladium(0) (0.01 equiv.) were combined and degassed with nitrogen for 5 minutes. A solution of the boronic acid (1.2 equiv.) and aryl halide (1.0 equiv.) in toluene (3 mL) and EtOH (1 mL) was then added, the reaction mixtures degassed with nitrogen for a further 5 minutes, then warmed for 16 h at 110° C. (external temperature). The reactions were allowed to cool to RT then the solvents removed in vacuo. The crude residues were purified by chromatography (EtOAc in heptane) to give the intermediate Suzuki products. These were treated with a 4 M solution of HCl in 1,4-dioxane (0.5 mL) for 1 h at RT, after which the solvents were removed in vacuo. The reaction residues were then dissolved in MeOH and analysed by LC/MS. If the purity of the final compound was >50% no further purification was attempted. Thus the MeOH was simply removed in vacuo to yield the title compound. If contamination by triphenylphosphine oxide was extensive, the MeOH was removed in vacuo and the residue taken up in water (1 mL). This aqueous solution was washed with TBME (3×1 mL) then reduced in vacuo to afford the title compound.

Compounds of the present invention have also been synthesized by the methods detailed in the following scheme:

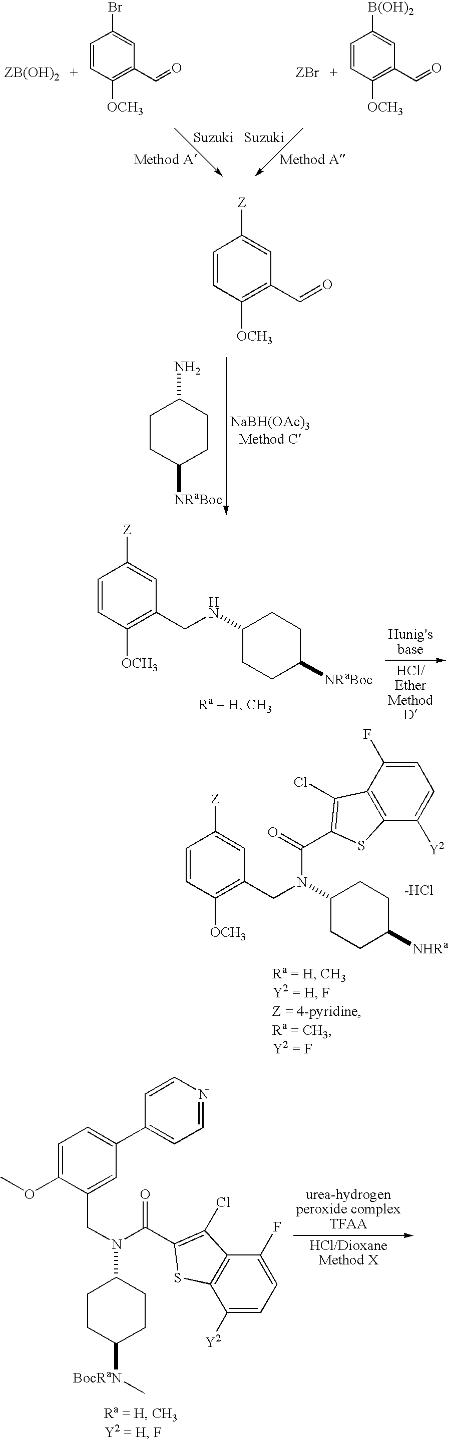

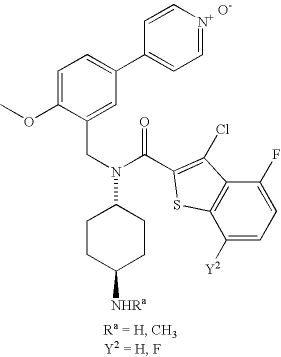

Method A′:

A suspension of the 5-bromo-2-methoxybenzaldehyde (1.0 equiv.), aryl boronic acid (1.0 equiv.), and cesium carbonate (2.2 equiv.) in ethanol (0.4 mL) and toluene (1.6 mL) was degassed with Argon for 30 minutes. Then tetrakis(triphenylphosphine) palladium(0) (0.05 equiv.) was added. The reaction mixture was filtered through a pad of celite and the solids were washed with dichloromethane. The dark filtrate was reduced in vacuo and the crude residue was purified by column chromatography (20-40% ethyl acetate in heptanes) to give the desired biaryl aldehyde as a solid. Product was analyzed by HPLC, MS and Hnmr.

Method A″:

A suspension of the 3-formyl-4-methoxyphenylboronic acid (1.0 equiv.), aryl bromide (1.0 equiv.), and cesium carbonate (2.2 equiv.) in ethanol (0.4 mL) and toluene (1.6 mL) was degassed with Argon for 30 minutes. Then tetrakis(triphenylphosphine) palladium(0) (0.05 equiv.) was used. The reaction mixture was then filtered through a pad of celite and the solids obtained washed with dichloromethane. The dark filtrate was then reduced in vacuo and the crude residue obtained purified by column chromatography (20-40% ethyl acetate in heptanes) to give the desired biaryl aldehyde as a solid. Product was analyzed by HPLC, MS and Hnmr.

Method C′:

To a stirred solution of the amine (1.2 equiv.) in anhydrous methanol (5 mL) with 4 Å molecular sieves (4-5) was added biaryl aldehyde (1.0 equiv.) in one portion. Additional methanol was added to dissolve all the reactants. The solution was then stirred for 2 hours at ambient temperature, and then cooled to 0° C. Sodium triacetoxyborohydride (2.5 equiv.) was added in several portions over the course of 45 minutes. The reaction mixture was then left to stir at ambient temperature for 16 hours. The suspension was then filtered over a celite bed and methanol was removed in vacuo. The crude residue was purified by column chromatography (gradient elution—10% methanol in dichloromethane with 0.5% ammonium hydroxide) to give the desired secondary amine as a solid. Product was analyzed by HPLC, MS and Hnmr.

Method D′:

To a stirred solution of the biaryl secondary amine (1.0 equiv.) in dichloromethane (5.5 mL) at 0° C. was added di-isopropylethylamine (2.2 equiv.) followed by the acid chloride (1.5 equiv.) in one portion. The reaction mixture was then stirred for 16 hours, over which a noticeable darkening occurred. The dichloromethane was then removed in vacuo and the crude, brown residue obtained purified by column chromatography (gradient elution—10% methanol in dichloromethane with 0.5% ammonium hydroxide) to give the desired amide as a solid. The isolated solid was then taken in dioxane and treated with 4N HCl/dioxane (2.00 equiv.) to give the hydrochloride salt of the desired amide product. Product was analyzed by HPLC, MS and Hnmr.

Method X:

Trifluoroacetic anhydride (1.0 mmol) was added dropwise to a stirred suspension of ura-hydrogen peroxide complex (1.1 mmol) in methylene chloride at 0° C. The mixture was stirred for 5 minutes and then a solution of the Boc-protected biaryl was added dropwise. The reaction was warmed to room temperature and stirring continued for 3 hours. Upon reaction is complete as indicated by HPLC, methylene chloride was added and the reaction mixture was washed with aqueous sodium bicarbonate and brine. The dichloromethane was then removed in vacuo and the crude, brown residue obtained purified by column chromatography (gradient elution—10% methanol in dichloromethane with 0.5% ammonium hydroxide) to give the desired amide as a solid. The isolated solid was then taken in dioxane and treated with 4N HCl/dioxane (2.00 equiv.) to give the hydrochloride salt of the desired amide product. Product was analyzed by HPLC, MS and Hnmr.

Common Intermediates

### Synthesis of trans-4-(BOC-methylamino)cyclohexylamine (3)

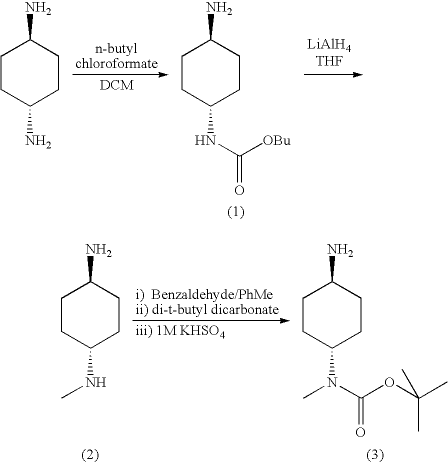

### n-Butyl trans-4-(aminocyclohexyl)-carbamate (1)

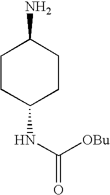

A solution of n-butylchloroformate (102 mL, 0.79 mol) in DCM (1.8 L) was added to a solution of 1,4-trans-dicyclohexane (180 g, 1.58 mol) in DCM (1.8 L) while maintaining the temperature between 0-5° C. (ice/salt bath). The resulting suspension was stirred 60-70 minutes then allowed to warm to 5-10° C. A solution of Na₂CO₃ (92.0 g, 0.87 mol) in water (720 mL) was added and the reaction stirred a further 5 minutes whilst warming to RT. The mixture was then transferred to a separating funnel and allowed to stand for approximately 5 minutes. The phases were separated and the aqueous phase washed with DCM (720 mL). The DCM phases were combined, dried over Na₂SO₄ (360 g), filtered and concentrated in vacuo to give a crude mixture of diamine, bis- and mono-carbamate. The crude material (164 g) was suspended in water (410 mL) and stirred vigorously for 10-15 minutes at RT. The solid bis-carbamate was then removed by filtration, the filter cake washed with water (164 mL) and the aqueous filtrate extracted with TBME (3×3.28 L). The TBME phases were combined, washed with water (246 mL), dried over Na₂SO₄ (492 g), filtered and concentrated in vacuo to afford the title product.

Yield: 87.5 g (52%).

### trans-4-(Methylamino)cyclohexylamine (2)

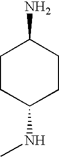

A solution of n-butyl carbamate 1 (102 g, 0.48 mol) in THF (1 L) was added over 30 minutes to a suspension of LiAlH₄ (90.4 g, 2.38 mol) in THF (2 L) at 0° C. Once the addition was complete, the reaction was heated to reflux 2 h then left to cool to RT over 16 h. The reaction was then cooled to 0-5° C. and quenched by careful addition of water (90 mL), 15% NaOH (90 mL) then more water (270 mL) over a period of 70 minutes. The resultant suspension was stirred at RT 1 h then filtered, washing the filter cake with TBME (2×2 L) and DCM (2×2 L). The solvents were removed in vacuo and residual water/n-butanol removed by azeotroping with toluene (2 L) to afford the title compound.

Yield: 55.4 g (91%).

### trans-4-(BOC-methylamino)cyclohexylamine (3)

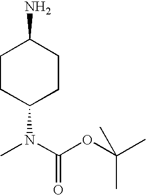

A solution of amine 2 (55.4 g, 0.43 mol) and benzaldehyde (46 mL, 0.45 mol) in toluene (554 mL) was heated at reflux using a Dean-Stark apparatus for 6 h, during which time the calculated volume of water (7.5 mL) was removed. The solution was then cooled to RT, at which point di-tert-butyl dicarbonate (103.7 g, 0.43 mol) was added portionwise over 15 minutes. The reaction was stirred 3 h and monitored by ¹H-NMR. On reaching completion, the toluene was removed in vacuo and the residue suspended in 1 M KHSO₄ (1.47 L). After stirring 4 h at RT, the reaction mixture was extracted with TBME (600 mL then 3×300 mL). The aqueous phase was then basified to pH 13 with 6 M NaOH (75 mL) and extracted with DCM (2×735 mL). The combined DCM phases were washed with brine (2×500 mL), dried (Na₂SO₄), filtered and reduced in vacuo to afford the title compound.

Yield: 67.8 g (69%).

### 3-{[4-(BOC-methyl-amino)-cyclohexylamino]-methyl}-4-methoxy-benzene boronic acid (4)

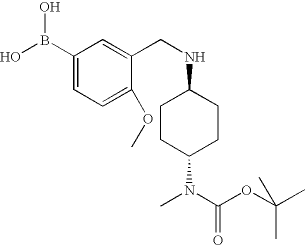

A stirred suspension of amine 3 (7.84 g, 34.3 mmol) and 3-formyl-4-methoxyphenyl boronic acid (6.18 g, 34.3 mmol) in THF (43 mL) and toluene (43 mL) was treated with AcOH (2.40 mL, 41.2 mmol) at RT. After stirring 1 h, further aliquots of THF and toluene were added (24 mL) to improve the solubility of the reagents. The addition of MeOH (30 mL) finally produced a homogeneous solution. After stirring 3 h, the resultant thick yellow suspension was treated with sodium triacetoxyborohydride (10.2 g, 48 mmol) and stirred a further hour. Analysis by LC/MS at this juncture confirmed the reaction was complete. The solvents were removed in vacuo and the viscous residue diluted with aqueous NaHCO₃ (150 mL) and extracted into EtOAc (3×150 mL). The combined organic phases were dried (MgSO₄) and the solvent removed in vacuo to afford the title compound.

Yield: 10.31 g (77%) @ 73% purity by LC trace. This material contained approximately 20% residual 3-formyl-4-methoxyphenyl boronic acid.

LC/MS tᵣ 1.12 min.

MS (ES+) m/z 393 (M+H).

### 3-{[[4-(BOC-methyl-amino)-cyclohexyl]-(3-chlorobenzo[b]thiophene-2-carbonyl)-amino]-methyl}-4-methoxy-benzene boronic acid (5)

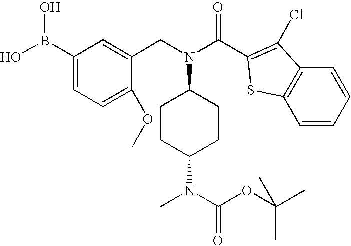

A solution of boronic acid 4 (1.95 g, 4.99 mmol) in DCM (20 mL) was treated with DIPEA (1.04 mL, 5.98 mmol), followed by 3-chlorobenzo[b]thiophene-2-carbonyl chloride (1.38 g, 5.98 mmol) in one portion. The resulting solution was stirred 16 h at RT. LC/MS at this juncture showed the reaction had reached completion. The reaction mixture was washed with water (3×10 mL) and brine (1×10 mL). The organic layer was dried over MgSO₄ and the solvent removed in vacuo to afford the title compound.

Yield: 2.50 g (86%).

LC/MS tᵣ 1.76 min.

MS (ES+) m/z 589, 587 (M+H), 533, 531 (M−C(CH₃)₃+H).

### 3-Chloro-4-fluorobenzo[b]thiophene-2-carbonyl chloride (6)

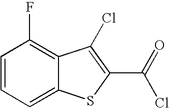

A stirred suspension of 2-fluorocinnamic acid (5.0 g, 30 mmol) in thionyl chloride (7.70 mL, 0.105 mol) was treated carefully with pyridine (0.82 mL, 7.5 mmol) at RT then heated 2 h at 140° C. (external temperature). The refluxing reaction mixture was then treated with heptane (5 mL), heated a further 5 minutes and the resultant supernatant decanted off and cooled to 0° C. The forthcoming precipitate was isolated by filtration, washed with heptane (2×2.5 mL) and dried to afford the title compound.

Yield: 2.69 g (36%).

### 3-{[[4-(BOC-methyl-amino)-cyclohexyl]-(3-chloro-4,fluoro-benzo[b]thiophene-2-carbonyl)-amino]-methyl}-4-methoxy-benzene boronic acid (7)

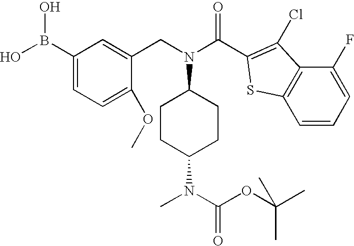

A solution of boronic acid 4 (300 mg, 0.77 mmol) in DCM (7.5 mL) was treated with triethylamine (160 μL, 1.45 mmol), followed by acid chloride 6 (223 mg, 1.50 mmol) in one portion. The resulting solution was stirred 16 h at RT. LC/MS at this juncture showed the reaction had reached completion. The reaction mixture was washed with water (3×10 mL) and brine (1×10 mL). The organic layer was dried over MgSO₄ and the solvent removed in vacuo. Purification by column chromatography (gradient elution—70% EtOAc in heptane increasing to 100% EtOAc, then 50% MeOH in EtOAc) gave the title compound.

Yield: 385 mg (83%).

LC/MS tᵣ 1.71 min.

MS (ES+) m/z 551, 549 (M−C(CH₃)₃+H).

### 3-Chloro-4,7-difluorobenzo[b]thiophene-2-carbonyl chloride (8)

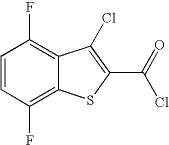

A stirred suspension of 2,5-difluorocinnamic acid (26 g, 0.14 mol) in thionyl chloride (36 mL, 0.49 mol) was treated carefully with pyridine (2.85 mL, 35 mmol) at RT then heated 16 h at 140° C. (external temperature). The hot reaction mixture was then decanted into refluxing heptane (260 mL) and heated at reflux 10 minutes before cooling to 0° C. The resultant precipitate was isolated by filtration, washed with heptane (2×25 mL) and dried to afford the title compound.

Yield: 17.4 g (46%).

### 3-{[[4-(BOC-methyl-amino)-cyclohexyl]-(3-chloro-4,7-difluoro-benzo[b]thiophene-2-carbonyl)-amino]-methyl}-4-methoxy-benzene boronic acid (9)

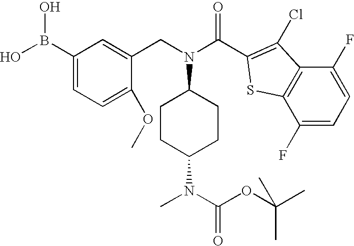

A solution of boronic acid 4 (7.60 g, 19.4 mmol) in DCM (300 mL) at 0° C. was treated with DIPEA (7.40 mL, 42.7 mmol), followed by acid chloride 8 (6.21 g, 23.3 mmol) in one portion. After warming to RT, the solution was stirred 3 h. Analysis by LC/MS at this juncture confirmed the reaction was complete. Thus the solvent was removed in vacuo and the residue purified by chromatography (gradient elution—60% EtOAc in heptane with 0.5% triethylamine increasing to neat EtOAc with 0.5% triethylamine, then 10-20% MeOH in EtOAc with 0.5% triethylamine) to afford the title compound.

Yield: 5.70 g (47%).

LC/MS tᵣ 1.69 min.

MS (ES+) m/z 625, 623 (M+H).

### 4-Ethoxy-3-formyl-benzene boronic acid (10)

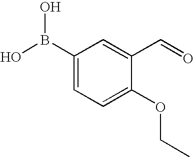

To a solution of 5-bromo-2-ethoxybenzaldehyde (1.0 g, 4.37 mmol) in EtOH (10 mL) was added triethylorthoformate (1.09 mL, 6.55 mmol) and ammonium chloride (12 mg, 0.22 mmol). The resulting solution was stirred 1 h at 45° C. to afford the protected aldehyde. TLC (1:1 EtOAc:heptane) at this juncture confirmed consumption of the starting material. The solvent was removed in vacuo to give the protected aldehyde as a yellow oil (1.5 g). This was dissolved in diethyl ether (10 mL) and cooled to −78° C. under N₂. n-BuLi (3.0 mL, 4.80 mmol, 1.6 M in hexanes) was added dropwise via syringe over 10 minutes and the reaction mixture stirred 30 minutes at −78° C. Triethylborate (2.95 mL, 10.9 mmol) was added as a solution in diethyl ether (5 mL) dropwise via syringe over 10 minutes, then stirring at −78° C. was continued for 4 h. 6 M HCl (1.3 mL) was added, the reaction warmed to RT, stirred 16 h then heated to reflux 1.5 h. The reaction was then cooled (0° C.) and basified with 4 M NaOH to pH 14. The layers were separated and the organic layer washed with 2 M NaOH (2×20 mL); the aqueous phases were then combined and washed with TBME (2×40 mL). The aqueous layer was cooled to 0° C. and acidified with 2 M HCl to pH 1. The resultant precipitate was isolated by filtration to give the title compound.

Yield: 306 mg (36%).

LC/MS tᵣ 1.05 min.

MS (ES+) nm/z 195 (M+H).

### 3-{[4-(BOC-methyl-amino)-cyclohexylamino]-methyl}-4-ethoxy-benzene boronic acid (11)

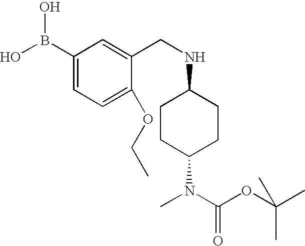

A stirred suspension of amine 3 (1.18 g, 5.16 mmol) and aldehyde 10 (835 mg, 4.30 mmol) in THF (12 mL) and toluene (12 mL) was treated with AcOH (310 μL, 5.16 mmol) at RT. After stirring 2 h the reaction mixture was treated with sodium triacetoxyborohydride (1.28 g, 5.16 mmol) and stirred a further 16 h. Analysis by LC/MS at this juncture confirmed the reaction was complete. The reaction was quenched with aqueous NaHCO₃ (50 mL) and extracted into EtOAc (2×50 mL). The combined organic phases were dried over Na₂SO₄ and the solvents removed in vacuo to give the title compound.

Yield: 1.69 g (96%).

LC/MS tᵣ 1.26 min.

MS (ES+) m/z 407 (M+H).

### 3-{[[4-(BOC-methyl-amino)-cyclohexyl]-(3-chloro-benzo[b]thiophene-2-carbonyl)-amino]-methyl}-4-ethoxy-benzene boronic acid (12)

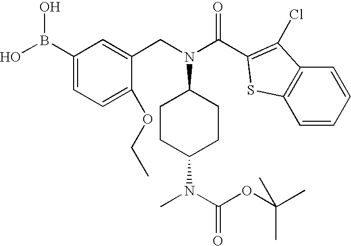

A solution of boronic acid 11 (700 mg, 1.72 mmol) in DCM (15 mL) at 0° C. was treated with triethylamine (0.48 mL, 3.45 mmol), followed by 3-chlorobenzo-[b]thiophene-2-carbonyl chloride (439 mg, 1.90 mmol) in one portion. After warming to RT, the solution was stirred 16 h. Analysis by LC/MS at this juncture confirmed the reaction was complete. Thus the reaction was diluted with aqueous NaHCO₃ (20 mL) and extracted into EtOAc (3×25 mL). The combined EtOAc phases were dried (Na₂SO₄) and reduced in vacuo to afford the title compound.

Yield: 978 mg (94%).

LC/MS tᵣ 1.86 min.

MS (ES+) m/z 547, 545 (M−C(CH₃)₃+H).

### Exemplary Compounds

### Synthesis of Compound 301

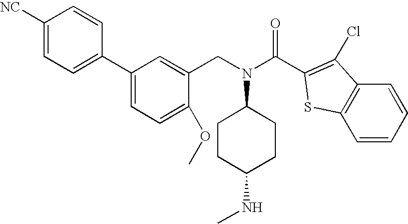

### tert-Butyl {4-[(4′-cyano-4-methoxy-biphenyl-3-ylmethyl)-amino]-cyclohexyl}-methyl-carbamate (13)

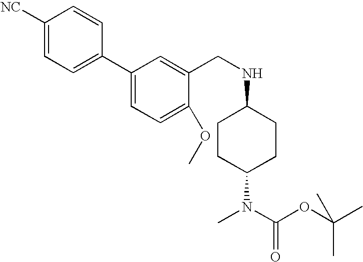

Boronic acid 4 (300 mg, 0.76 mmol) was coupled to 4-bromobenzonitrile (167 mg, 0.92 mmol) using Method B to give the title compound.

Yield: 367 mg (quant.). Contains ca. 36% triphenylphosphine oxide.

LC/MS tᵣ 1.42 min.

MS (ES+) m/z 450 (M+H), 394 (M−C(CH₃)₃+H).

### tert-Butyl {4-[(3-chloro-benzo[b]thiophene-2-carbonyl)-(4′-cyano-4-methoxy-biphenyl-3-ylmethyl)-amino]-cyclohexyl}-methyl-carbamate (14)

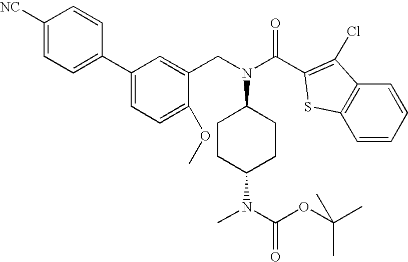

Biaryl amine 13 (367 mg, 0.82 mmol) was treated with 3-chlorobenzo-[b]thiophene-2-carbonyl chloride (226 mg, 0.98 mmol) using Method D to give the title compound.

Yield: 110 mg (21%).

LC/MS tᵣ 1.97 min.

MS (ES+) m/z 590, 588 (M−C(CH₃)₃+H).

### 3-Chloro-benzo[b]thiophene-2-carboxylic acid (4′-cyano-4-methoxy-biphenyl-3-ylmethyl)-(4-methylamino-cyclohexyl)-amide hydrochloride (15)

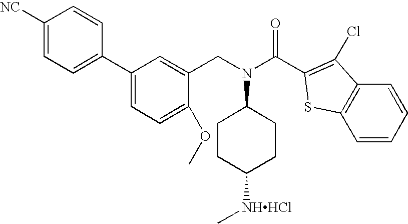

tert-Butyl carbamate 14 (110 mg, 0.17 mmol) was deprotected using Method F to give the title compound.

Yield: 100 mg (quant.).

LC/MS tᵣ 1.47 min.

MS (ES+) m/z 546, 544 (M+H), 515, 513 (M−31+H).

### Synthesis of Compound 302

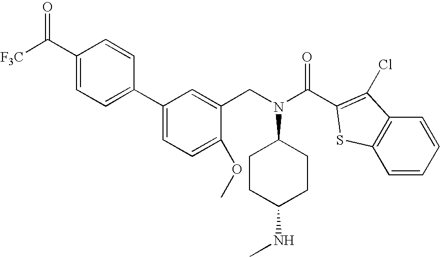

### tert-Butyl (4-{[4-methoxy-4′-(2,2,2-trifluoro-acetyl)-biphenyl-3-ylmethyl]-amino}-cyclohexyl)-methyl-carbamate (16)

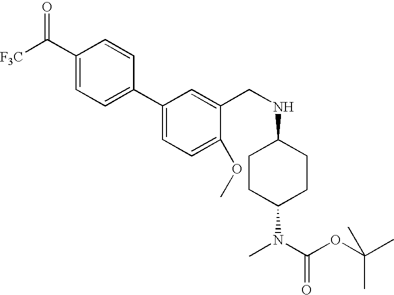

Boronic acid 4 (620 mg, 1.60 mmol) was coupled to 4′-bromo-2,2,2-trifluoroacetophenone (400 mg, 1.60 mmol) using Method B to give the title compound.

Yield: 467 mg (57%).

LC/MS tᵣ 1.34 min.

MS (ES+) m/z 521 (M+H) 465 (M−C(CH₃)₃+H).

### tert-Butyl (4-{(3-chloro-benzo[b]thiophene-2-carbonyl)-[4-methoxy-4′-(2,2,2-trifluoro-acetyl)-biphenyl-3-ylmethyl]-amino}-cyclohexyl)-methyl-carbamate (17)

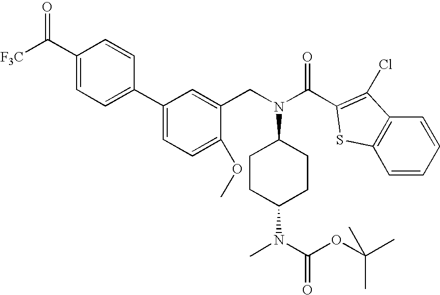

Biaryl amine 16 (467 mg, 0.90 mmol) was treated with 3-chlorobenzo-[b]thiophene-2-carbonyl chloride (290 mg, 1.26 mmol) using Method D to give the title compound.

Yield: 539 mg (88%).

LC/MS tᵣ 1.94 min.

MS (ES+) m/z 735, 733 (M+H₂O+H) 679, 677 (M−C(CH₃)₃+H₂O+H).

### 3-Chloro-benzo[b]thiophene-2-carboxylic acid [4-methoxy-4′-(2,2,2-trifluoro-acetyl)biphenyl-3-ylmethyl]-(4-methylamino-cyclohexyl)-amide trifluoroacetate (18)

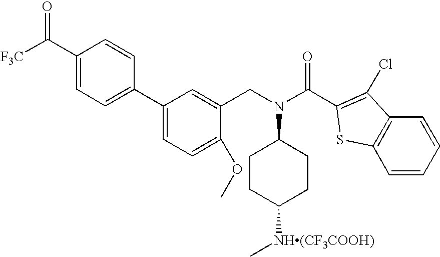

tert-Butyl carbamate 17 (539 mg, 0.75 mmol) was deprotected using Method E. Preparative HPLC then gave the title compound as the TFA salt.

Yield: 410 mg (88%).

LC/MS tᵣ 1.63 min.

MS (ES+) m/z 635, 633 (M+H₂O+H), 604, 602 (M−31+H₂O+H).

### Synthesis of Compound 303

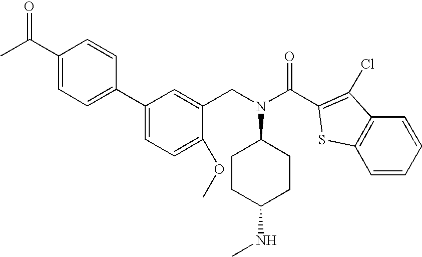

### tert-Butyl {4-[(4′-acetyl-4-methoxy-biphenyl-3-ylmethyl)-amino]-cyclohexyl}-methyl-carbamate (19)

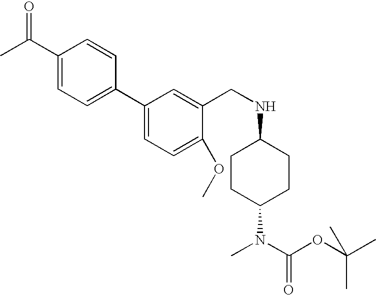

Boronic acid 4 (329 mg, 0.83 mmol) was coupled to 4′-bromoacetophenone (200 mg, 1.00 mmol) using Method B to give the title compound.

Yield: 400 mg (quant.). Contains ca. 15% triphenylphosphine oxide.

LC/MS tᵣ 1.42 min.

MS (ES+) m/z 467 (M+H), 411 (M−C(CH₃)₃+H).

### tert-Butyl {4-[(4′-acetyl-4-methoxy-biphenyl-3-ylmethyl)-(3-chloro-benzo[b]thiophene-2-carbonyl)-amino]-cyclohexyl}-methyl-carbamate (20)

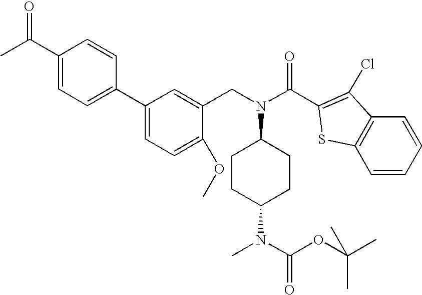

Biaryl amine 19 (400 mg, 0.86 mmol) was treated with 3-chloro-benzo[b]thiophene-2-carbonyl chloride (237 mg, 1.03 mmol) using Method D to give the title compound.

Yield: 134 mg (24%).

LC/MS tᵣ 2.06 nm.

MS (ES+) m/z 607, 605 (M−C(CH₃)₃+H).

### 3-Chloro-benzo[b]thiophene-2-carboxylic acid (4′-acetyl-4-methoxy-biphenyl-3-ylmethyl)-(4-methylamino-cyclohexyl)-amide hydrochloride (21)

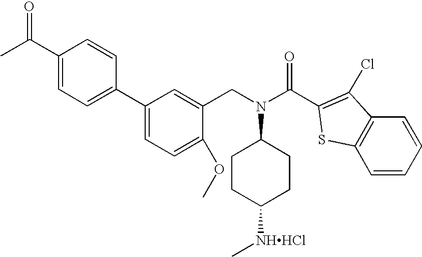

tert-Butyl carbamate 20 (134 mg, 0.20 mmol) was deprotected using Method E to give the title compound.

Yield: 94 mg (83%).

LC/MS tᵣ 1.48 min.

MS (ES+) m/z 563, 561 (M+H), 532, 530 (M−31+H).

### Synthesis of Compound 304

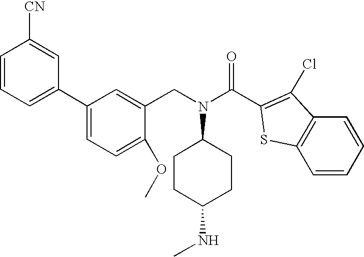

### tert-Butyl {4-[(3′-cyano-4-methoxy-biphenyl-3-ylmethyl)-amino]-cyclohexyl}-methyl-carbamate (22)

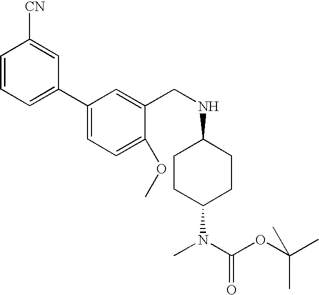

Boronic acid 4 (359 mg, 0.92 mmol) was coupled to 3-bromobenzonitrile (200 mg, 1.10 mmol) using Method B to give the title compound.

Yield: 386 mg (93%).

LC/MS tᵣ 1.46 min.

MS (ES+) m/z 450 (M+H), 394 (M−C(CH₃)₃+H).

### tert-Butyl {4-[(3-chloro-benzo[b]thiophene-2-carbonyl)-(3′-cyano-4-methoxy-biphenyl-3-ylmethyl)-amino]-cyclohexyl}-methyl-carbamate (23)

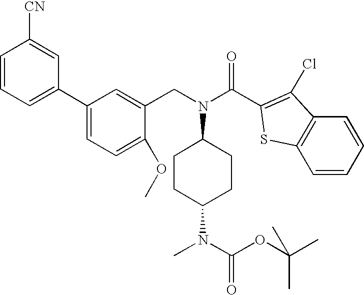

Biaryl amine 22 (386 mg, 0.86 mmol) was treated with 3-chloro-benzo[b]thiophene-2-carbonyl chloride (238 mg, 1.03 mmol) using Method D to give the title compound.

Yield: 60 mg (11%).

LC/MS tᵣ 1.94 min.

MS (ES+) m/z 590, 588 (M−C(CH₃)₃+H).

### 3-Chloro-benzo[b]thiophene-2-carboxylic acid (3′-cyano-4-methoxy-biphenyl-3-ylmethyl)-(4-methylamino-cyclohexyl)-amide hydrochloride (24)

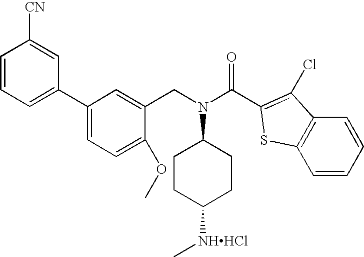

tert-Butyl carbamate 23 (60 mg, 0.11 mmol) was deprotected using Method E to give the title compound.

Yield: 51 mg (quant.).

LC/MS tᵣ 1.51 min.

MS (ES+) m/z 546, 544 (M+H), 515, 513 (M−31+H).

### Synthesis of Compound 305

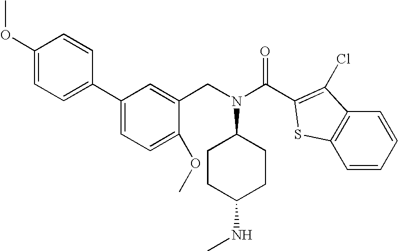

### tert-Butyl {4-[(4,4′-dimethoxy-biphenyl-3-ylmethyl)-amino]-cyclohexyl}-methyl-carbamate (25)

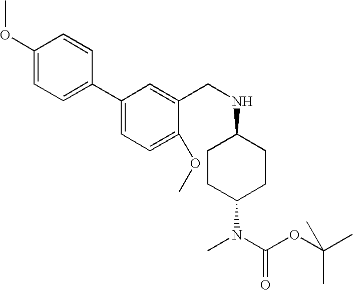

Boronic acid 4 (329 mg, 1.00 mmol) was coupled to 4-bromoanisole (125 μL, 1.00 mmol) using Method B to give the title compound.

Yield: 212 mg (47%).

LC/MS tᵣ 1.59 min.

MS (ES+) m/z 455 (M+H).

### tert-Butyl {4-[(3-chloro-benzo[b]thiophene-2-carbonyl)-(4,4′-dimethoxy-biphenyl-3-ylmethyl)-amino]-cyclohexyl}-methyl-carbamate (26)

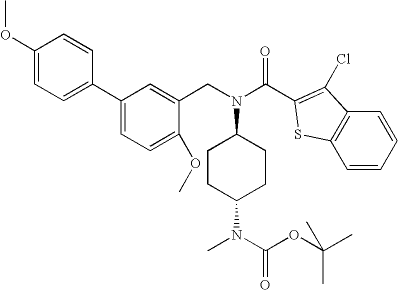

Biaryl amine 25 (212 mg, 0.46 mmol) was treated with 3-chloro-benzo[b]thiophene-2-carbonyl chloride (108 mg, 0.46 mmol) using Method D to give the title compound.

Yield: 213 mg (76%).

LC/MS tᵣ 2.18 min.

MS (ES+) m/z 595, 593 (M−C(CH₃)₃+H).

### 3-Chloro-benzo[b]thiophene-2-carboxylic acid (4,4′-dimethoxy-biphenyl-3-ylmethyl)-(4-methylamino-cyclohexyl)-amide hydrochloride (27)

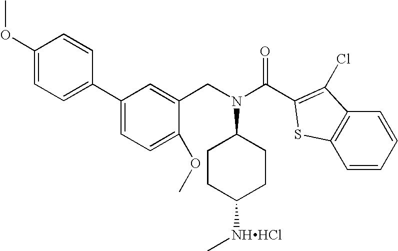

tert-Butyl carbamate 26 (159 mg, 0.23 mmol) was deprotected using Method E to give the title compound.

Yield: 130 mg (quant.).

LC/MS tᵣ 1.57 min.

MS (ES+) m/z 551, 549 (M+H).

### Synthesis of Compound 306

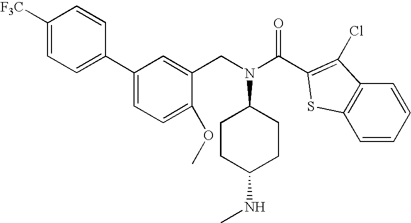

### tert-Butyl {4-[(4-methoxy-4′-trifluoromethyl-biphenyl-3-ylmethyl)-amino]-cyclohexyl}-methyl-carbamate (28)

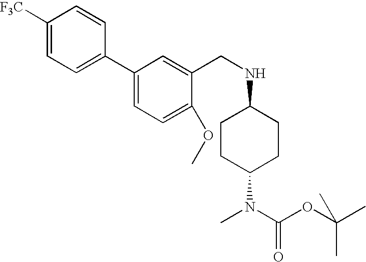

Boronic acid 4 (349 mg, 0.90 mmol) was coupled to 1-bromo-4-trifluoro-methylbenzene (200 mg, 0.90 mmol) using Method B to give the title compound. Yield: 465 mg (quant.). Contains ca. 22% triphenylphosphine oxide.

LC/MS tᵣ 1.61 min.

MS (ES+) m/z 493 (M+H), 437 (M−C(CH₃)₃+H).

### tert-Butyl 4-[(3-chloro-benzo[b]thiophene-2-carbonyl)-(4-methoxy-4′-trifluoromethyl-biphenyl-3-ylmethyl)-amino]-cyclohexyl}-methyl-carbamate (29)

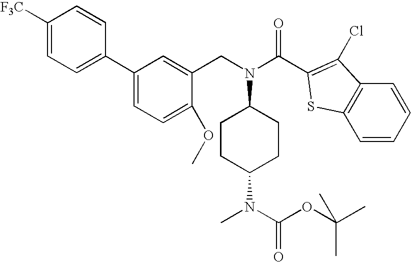

Biaryl amine 28 (465 mg, 0.94 mmol) was treated with 3-chloro-benzo[b]thiophene-2-carbonyl chloride (306 mg, 1.32 mmol) using Method D to give the title compound.

Yield: 266 mg (41%).

LC/MS tᵣ 2.23 min.

MS (ES+) m/z 689, 687 (M+H), 633, 631 (M−C(CH₃)₃+H).

### 3-Chloro-benzo[b]thiophene-2-carboxylic acid (4-methoxy-4′-trifluoromethyl-biphenyl-3-ylmethyl)-(4-methylamino-cyclohexyl)-amide trifluoroacetate (30)

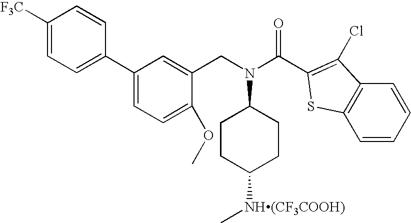

tert-Butyl carbamate 29 (366 mg, 0.53 mmol) was deprotected using Method E. Preparative HPLC then gave the title compound as the TFA salt.

Yield: 95 mg (26%).

LC/MS tᵣ 1.86 min.

MS (ES+) m/z 589, 587 (M+H), 558, 556 (M−31+H).

### Synthesis of Compound 307

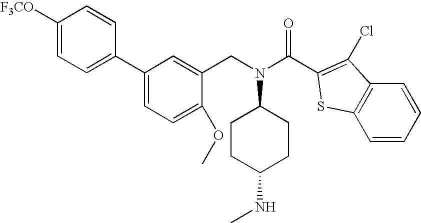

### tert-Butyl {4-[(4-methoxy-4′-trifluoromethoxy-biphenyl-3-ylmethyl)-amino]-cyclohexyl}-methyl-carbamate (31)

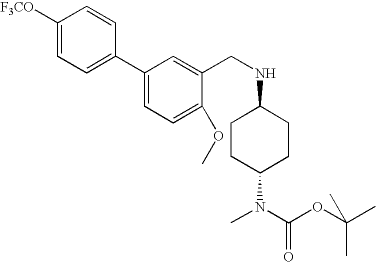

Boronic acid 4 (271 mg, 0.69 mmol) was coupled to 1-bromo-4-(trifluoromethoxy)benzene (200 mg, 0.83 mmol) using Method B to give the title compound.

Yield: 220 mg (62%).

### tert-Butyl {4-[(3-chloro-benzo[b]thiophene-2-carbonyl)-(4-methoxy-4′-trifluoromethoxy-biphenyl-3-ylmethyl)-amino]-cyclohexyl}-methyl-carbamate (32)

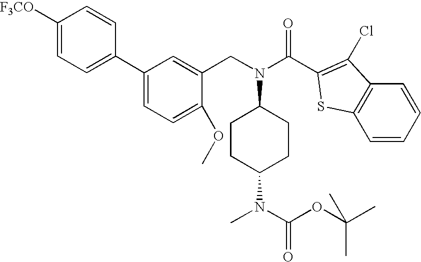

Biaryl amine 31 (220 mg, 0.43 mmol) was treated with 3-chloro-benzo[b]thiophene-2-carbonyl chloride (120 mg, 0.52 mmol) using Method D to give the title compound.

Yield: 150 mg (53%).

### 3-Chloro-benzo[b]thiophene-2-carboxylic acid (4-methoxy-4′-trifluoromethoxy-biphenyl-3-ylmethyl)-(4-methylamino-cyclohexyl)-amide hydrochloride (33)

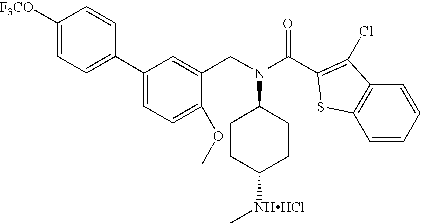

tert-Butyl carbamate 32 (150 mg, 0.21 mmol) was deprotected using Method F to give the title compound.

Yield: 110 mg (86%).

LC/MS tᵣ 1.90 min.

MS (ES+) m/z 605, 603 (M+H).

### Synthesis of Compound 308

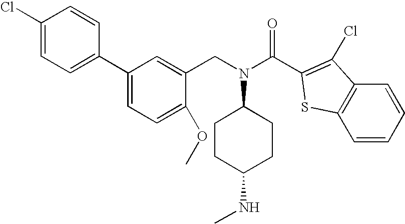

### tert-Butyl {4-[(4′-chloro-4-methoxy-biphenyl-3-ylmethyl)-amino]-cyclohexyl}-methyl-carbamate (34)

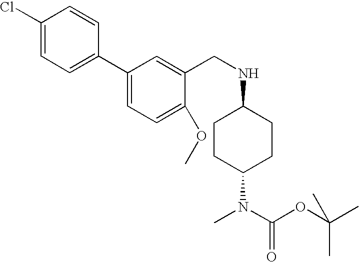

Boronic acid 4 (342 mg, 0.87 mmol) was coupled to 1-bromo-4-chlorobenzene (200 mg, 1.05 mmol) using Method B to give the title compound.

Yield: 348 mg (87%).

### tert-Butyl {4-[(3-chloro-benzo[b]thiophene-2-carbonyl)-(4′-chloro-4-methoxy-biphenyl-3-ylmethyl)-amino]-cyclohexyl}-methyl-carbamate (35)

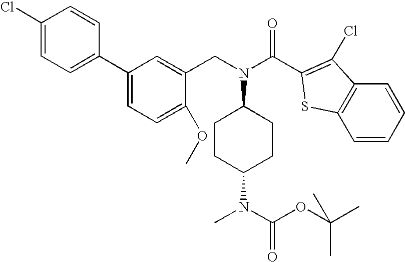

Biaryl amine 34 (348 mg, 0.76 mmol) was treated with 3-chloro-benzo[b]thiophene-2-carbonyl chloride (210 mg, 0.91 mmol) using Method D to give the title compound.

Yield: 162 mg (33%).

### 3-Chloro-benzo[b]thiophene-2-carboxylic acid (4′-chloro-4-methoxy-biphenyl-3-ylmethyl)-(4-methylamino-cyclohexyl)-amide hydrochloride (36)

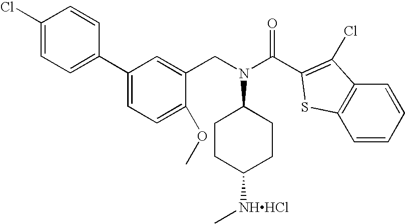

tert-Butyl carbamate 35 (162 mg, 0.25 mmol) was deprotected using Method F to give the title compound.

Yield: 100 mg (73%).

LC/MS tᵣ 1.66 min.

MS (ES+) m/z 555, 553 (M+H), 524, 522 (M−31+H).

### Synthesis of Compound 309

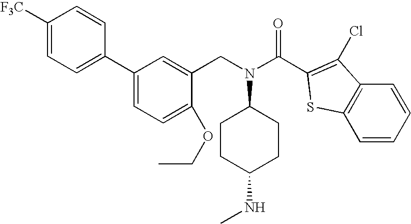

### 3-Chloro-benzo[b]thiophene-2-carboxylic acid (4-ethoxy-4′-trifluoromethyl-biphenyl-3-ylmethyl)-(4-methylamino-cyclohexyl)-amide hydrochloride (37)

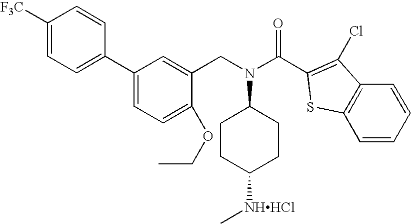

The title compound was prepared from boronic acid 11 (20 mg, 49 μmol) and 1-bromo-4-trifluoromethylbenzene (9.2 mg, 41 μmol) in accordance with Method L1.

Yield: 8.4 mg (32%).

LC/MS tᵣ 1.61 min.

MS (ES+) m/z 644, 642 (M+CH₃CN+H), 603, 601 (M+H).

### Synthesis of Compound R2

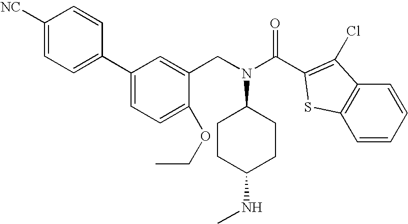

### 4′-Ethoxy-3′-formyl-biphenyl-4-carbonitrile (38)

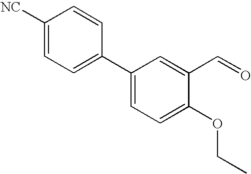

4-cyanophenylboronic acid (500 mg, 3.40 mmol) was coupled to 5-bromo-2-ethoxybenzaldehyde (780 mg, 3.40 mmol) using Method A to give the title compound.

Yield: 625 mg (73%).

LC/MS tᵣ 1.58 min.

MS (ES+) m/z 252 (M+H).

### tert-Butyl {4-[(4′-cyano-4-ethoxy-biphenyl-3-ylmethyl)-amino]-cyclohexyl}-methyl-carbamate (39)

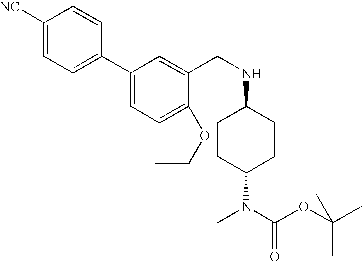

Amine 3 (300 mg, 1.19 mmol) was treated with aldehyde 38 (272 mg, 1.19 mmol) in accordance with Method C to give the crude title compound.

Yield: 613 mg.

LC/MS tᵣ 1.39 min.

MS (ES+) m/z 464 (M+H).

### tert-Butyl {4-[(3-chloro-benzo[b]thiophene-2-carbonyl)-(4′-cyano-4-ethoxy-biphenyl-3-ylmethyl)-amino]-cyclohexyl}-methyl-carbamate (40)

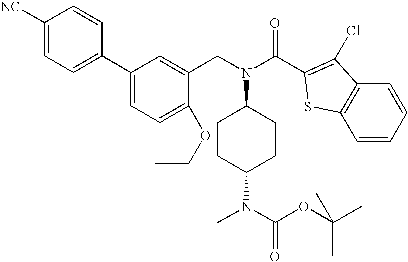

Crude biaryl amine 39 (613 mg, 1.32 mmol) was treated with 3-chloro-benzo[b]thiophene-2-carbonyl chloride (366 mg, 1.58 mmol) using Method D to give the title compound.

Yield: 550 mg (70% over two steps).

LC/MS tᵣ 2.03 min.

MS (ES+) m/z 660, 658 (M+H), 604, 602 (M−C(CH₃)₃+H).

### 3-Chloro-benzo[b]thiophene-2-carboxylic acid (4′-cyano-4-ethoxy-biphenyl-3-ylmethyl)-(4-methylamino-cyclohexyl)-amide hydrochloride (41)

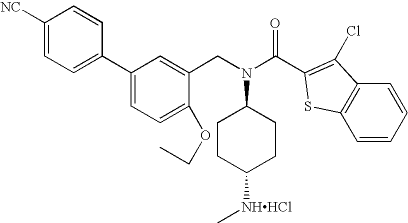

tert-Butyl carbamate 40 (550 mg, 0.25 mmol) was deprotected using Method F. Purification by column chromatography (5% MeOH in DCM with 0.5% triethylamine) then acidification of the free base using Method H gave the title compound.

Yield: 226 mg (27%).

LC/MS tᵣ 1.53 min.

MS (ES+) m/z 560, 558 (M+H).

### Synthesis of Compound 311

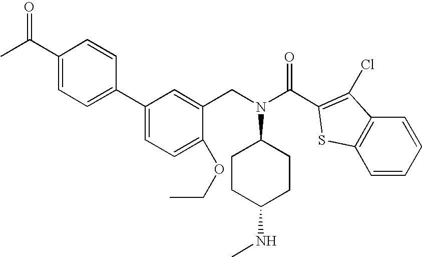

### tert-Butyl {4-[(4′-acetyl-4-ethoxy-biphenyl-3-ylmethyl)-(3-chloro-benzo[b]thiophene-2-carbonyl)-amino]-cyclohexyl}-methyl-carbamate (42)

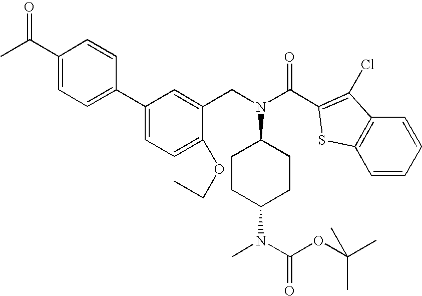

Boronic acid 12 (200 mg, 0.33 mmol) was coupled to 4′-bromoacetophenone (79 mg, 0.39 mmol) using Method B to give the title compound.

Yield: 64 mg (24%).

LC/MS tᵣ 2.02 min.

MS (ES+) m/z 677, 675 (M+H), 621, 619 (M−C(CH₃)₃+H).

### 3-Chloro-benzo[b]thiophene-2-carboxylic acid (4′-acetyl-4-ethoxy-biphenyl-3-ylmethyl)-(4-methylamino-cyclohexyl)-amide hydrochloride (43)

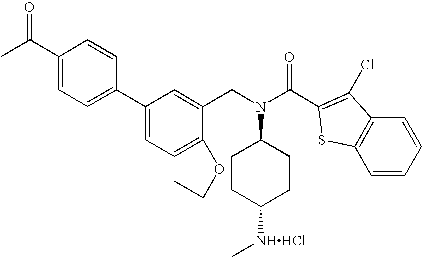

tert-Butyl carbamate 42 (64 mg, 0.51 mmol) was deprotected using Method E to give the title compound.

Yield: 50 mg (92%).

LC/MS tᵣ 1.69 min.

MS (ES+) m/z 577, 575 (M+H), 546, 544 (M−31+H).

### Synthesis of Compound 312

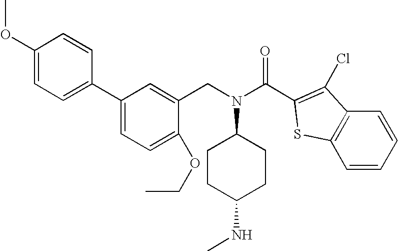

### 3-Chloro-benzo[b]thiophene-2-carboxylic acid (4-ethoxy-4′-methoxy-biphenyl-3-ylmethyl)-(4-methylamino-cyclohexyl)-amide hydrochloride (44)

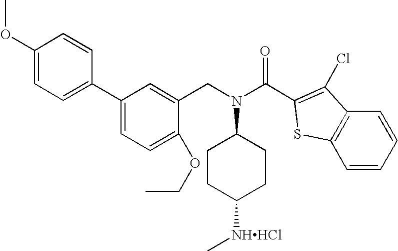

The title compound was prepared from boronic acid 11 (20 mg, 49 μmol) and 4-bromoanisole (7.7 mg, 41 μmol) in accordance with Method L1.

Yield: 36.6 mg (148%).

LC/MS tᵣ 1.48 min.

MS (ES+) m/z 606, 604 (M+CH₃CN+H), 565, 563 (M+H).

### Synthesis of Compound 313

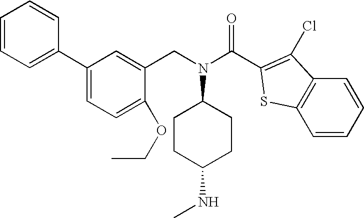

### 3-Chloro-benzo[b]thiophene-2-carboxylic acid (4-ethoxy-biphenyl-3-ylmethyl)-(4-methylamino-cyclohexyl)-amide hydrochloride (45)

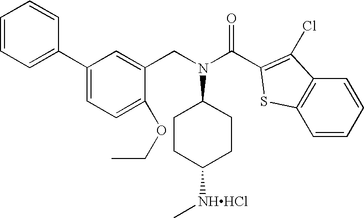

The title compound was prepared from boronic acid 11 (20 mg, 49 μmol) and bromobenzene (6.4 mg, 41 μmol) in accordance with Method L1.

Yield: 7.7 mg (33%).

LC/MS tᵣ 1.50 min.

MS (ES+) m/z 576, 574 (M+CH₃CN+H), 535, 533 (M+H).

### Synthesis of Compound R3

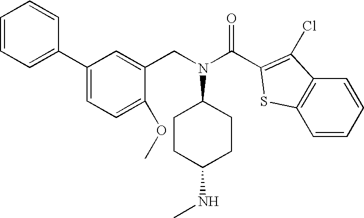

### tert-Butyl {4-[(4-methoxy-biphenyl-3-ylmethyl)-amino]-cyclohexyl}-methyl-carbamate (46)

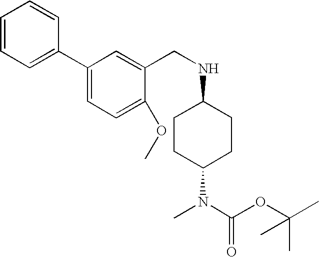

Boronic acid 4 (1.0 g, 2.55 mmol) was coupled to bromobenzene (0.32 mL, 3.0 mmol) using Method A to give the title compound.

Yield: 437 mg (40%).

LC/MS tᵣ 1.51 min.

MS (ES+) m/z 425 (M+H).

### tert-Butyl {4-[(3-chloro-benzo[b]thiophene-2-carbonyl)-(4-methoxy-biphenyl-3-ylmethyl)-amino]-cyclohexyl}-methyl-carbamate (47)

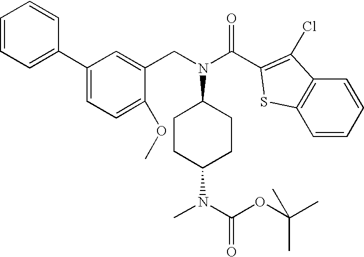

Biaryl amine 46 (423 mg, 1.00 mmol) was treated with 3-chloro-benzo[b]thiophene-2-carbonyl chloride (507 mg, 2.20 mmol) using Method D to give the title compound.

Yield: 320 mg (52%).

LC/MS tᵣ 2.06 min.

MS (ES+) m/z 621, 619 (M+H), 565, 563 (M−C(CH₃)₃+H).

### 3-Chloro-benzo[b]thiophene-2-carboxylic acid (4-methoxy-biphenyl-3-ylmethyl)-(4-methylamino-cyclohexyl)-amide hydrochloride (48)

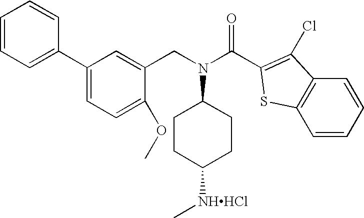

tert-Butyl carbamate 47 (320 mg, 0.52 mmol) was deprotected using Method F to give the title compound.

Yield: 184 mg (64%).

LC/MS tᵣ 1.42 min.

MS (ES+) m/z 521, 519 (M+H).

### Synthesis of Compound 314

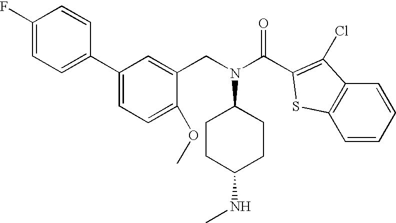

### 3-Chloro-benzo[b]thiophene-2-carboxylic acid (4′-fluoro-4-methoxy-biphenyl-3-ylmethyl)-(4-methylamino-cyclohexyl)-amide hydrochloride (49)

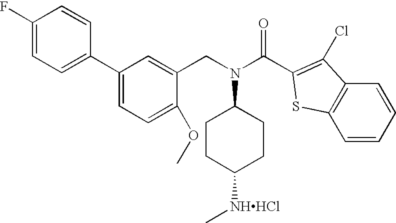

The title compound was prepared from boronic acid 4 (20 mg, 51 μmol) and 1-bromo-4-fluorobenzene (7.4 mg, 42 μmol) in accordance with Method L1.

Yield: 5.8 mg (24%).

LC/MS tᵣ 1.53 min.

MS (ES+) m/z 539, 537 (M+H), 508, 506 (M−31+H).

### Synthesis of Compound 315

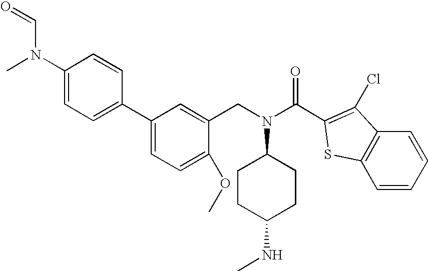

### N-(4-Bromophenyl)-formamide (50)

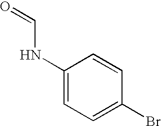

A stirred solution of formic acid (2.5 mL, 88% in water) was cooled to 0° C., treated with acetic anhydride (0.82 mL, 9.0 mmol) then warmed to RT over 30 minutes. The solution was then cooled to 0° C. and 4-bromoaniline (500 mg, 3.0 mmol) added dropwise over 5 minutes. After stirring a further 16 h at RT, the reaction mixture was diluted with water (20 mL) and extracted into DCM (3×20 mL). The combined DCM phases were washed with 1 M HCl (20 mL) and aqueous NaHCO₃ (20 mL), dried over Na₂SO₄ and reduced in vacuo. Purification by column chromatography (20% EtOAc in heptane) afforded the title compound.

Yield: 468 mg (78%).

### N-(4-Bromophenyl)-N-methyl-formamide (51)

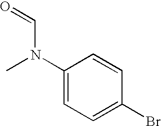

A solution of N-(4-bromophenyl)-formamide 50 (250 mg, 1.25 mmol) in DMF (3 mL) was added dropwise to a suspension of sodium hydride (65 mg, 1.63 mmol, 60% dispersion in mineral oil) in DMF (3 mL) at 0° C. After stirring 1 h, iodomethane (113 μg, 1.63 mmol) was added, the reaction mixture warmed to RT and stirred 16 h. The reaction was then quenched with water (10 mL) and extracted into EtOAc (3×10 mL). The combined organic phases were dried over Na₂SO₄ and reduced in vacuo to give the title compound.

Yield: 223 mg (83%).

LC/MS tᵣ 1.19 min.

MS (ES+) m/z 216, 214 (M+H).

### tert-Butyl (4-{[4′-(formyl-methyl-amino)-4-methoxy-biphenyl-3-ylmethyl]-amino}-cyclohexyl)-methyl-carbamate (52)

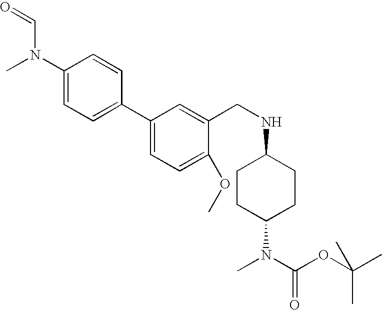

Boronic acid 4 (260 mg, 0.66 mmol) was coupled to aryl bromide 51 (170 mg, 0.79 mmol) using Method B to give the title compound.

Yield: 300 mg (94%).

LC/MS tᵣ 1.30 mm.

MS (ES+) m/z 426 (M−C(CH₃)₃+H).

### tert-Butyl (4-{(3-chloro-benzo[b]thiophene-2-carbonyl)-[4′-(formyl-methyl-amino)-4-methoxy-biphenyl-3-ylmethyl]-amino}-cyclohexyl)-methyl-carbamate (53)

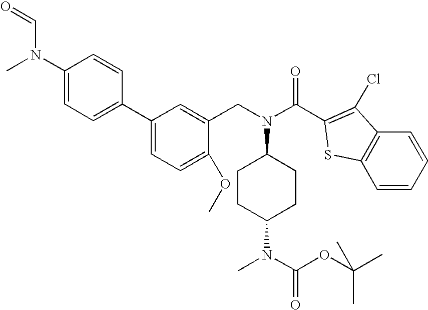

Biaryl amine 52 (325 mg, 0.64 mmol) was treated with 3-chloro-benzo[b]thiophene-2-carbonyl chloride (171 mg, 0.74 mmol) using Method D to give the title compound.

Yield: 160 mg (35%).

LC/MS tᵣ 2.09 min.

MS (ES+) m/z 578, 576 (MCO₂C(CH₃)₃+H).

### 3-Chloro-benzo[b]thiophene-2-carboxylic acid [4′-(formyl-methyl-amino)-4-methoxy-biphenyl-3-ylmethyl]-(4-methylamino-cyclohexyl)-amide trifluoroacetate (54)

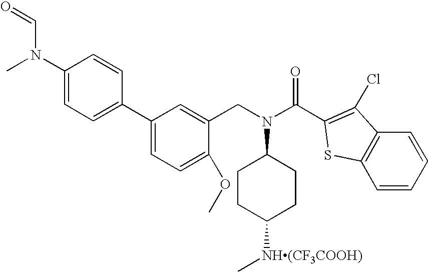

tert-Butyl carbamate 53 (190 mg, 0.28 mmol) was deprotected using Method G to give the title compound.

Yield: 170 mg (quant.).

LC/MS tᵣ 1.58 min.

MS (ES+) m/z 578, 576 (M+H).

### Synthesis of Compound 316

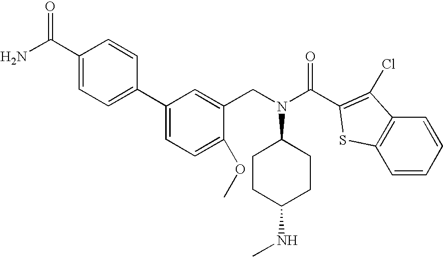

### tert-Butyl {4-[(4′-carbamoyl-4-methoxy-biphenyl-3-ylmethyl)-amino]-cyclohexyl}-methyl-carbamate (55)

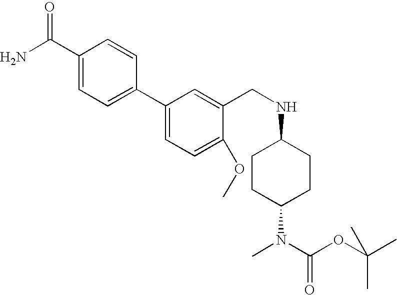

Boronic acid 4 (326 mg, 0.83 mmol) was coupled to 4-bromobenzamide (200 mg, 0.99 mmol) using Method B to give the title compound.

Yield: 267 mg (69%).

LC/MS tᵣ 1.29 min.

MS (ES+) m/z 468 (M+H), 412 (M−C(CH₃)₃+H).

### tert-Butyl {4-[(4′-carbamoyl-4-methoxy-biphenyl-3-ylmethyl)-(3-chloro-benzo[b]thiophene-2-carbonyl)-amino]-cyclohexyl}-methyl-carbamate (56)

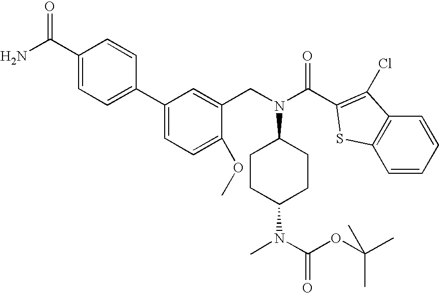

Biaryl amine 55 (267 mg, 0.57 mmol) was treated with 3-chloro-benzo[b]thiophene-2-carbonyl chloride (158 mg, 0.69 mmol) using Method D to give the title compound.

Yield: 266 mg (70%).

LC/MS tᵣ 1.85 min.

MS (ES+) m/z 608, 606 (M−C(CH₃)₃+H).

### 3-Chloro-benzo[b]thiophene-2-carboxylic acid (4′-carbamoyl-4-methoxy-biphenyl-3-ylmethyl)-(4-methylamino-cyclohexyl)-amide hydrochloride (57)

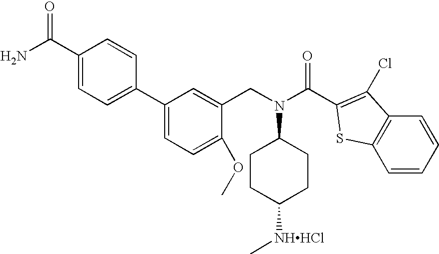

tert-Butyl carbamate 56 (266 mg, 0.39 mmol) was deprotected using Method E to give the title compound.

Yield: 220 mg (97%).

LC/MS tᵣ 1.29 min.

MS (ES+) m/z 564, 562 (M+H), 533, 531 (M−31+H).

### Synthesis of Compound 317

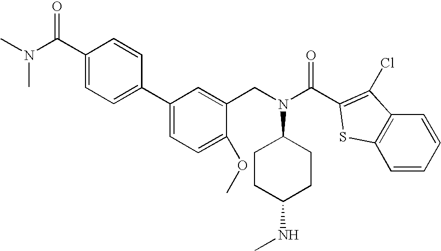

### 4-Bromo-N,N′-dimethylbenzamide (58)

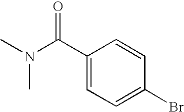

A suspension of hexane-washed sodium hydride (600 mg, 15 mmol, 60% dispersion in mineral oil) in DMF (12 mL) was treated portionwise with 4-bromobenzamide (1.0 g, 5.0 mmol) at 0° C. over 1-2 minutes. After warming to RT and stirring 1 h, iodomethane (7.50 mL, 15 mmol, 2.0 M solution in TBME) was added via syringe and the reaction stirred a further 16 h. The reaction mixture was then diluted with water (50 mL) and extracted into TBME (3×25 mL). The TBME phases were combined, washed with water (2×25 mL) and brine (25 mL), dried (MgSO₄) and reduced in vacuo to a clear oil. Column chromatography (gradient elution—50-70% EtOAc in heptane) afforded the title compound.

Yield: 863 mg (76%).

LC/MS tᵣ 1.12 min.

MS (ES+) m/z 230, 228 (M+H).

### tert-Butyl 4-[(4′-dimethylcarbamoyl-4-methoxy-biphenyl-3-ylmethyl)-amino]-cyclohexyl}-methyl-carbamate (59)

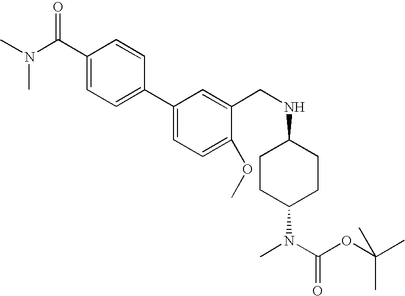

Boronic acid 4 (354 mg, 0.84 mmol) was coupled to aryl bromide 58 (230 mg, 1.01 mmol) using Method B to give the title compound.

Yield: 110 mg (29%).

LC/MS tᵣ 1.36 min.

MS (ES+) m/z 496 (M+H).

### tert-Butyl {4-[(3-chloro-benzo[b]thiophene-2-carbonyl)-(4′-dimethylcarbamoyl-5-methoxy-biphenyl-3-ylmethyl)-amino]-cyclohexyl}-methyl-carbamate (60)

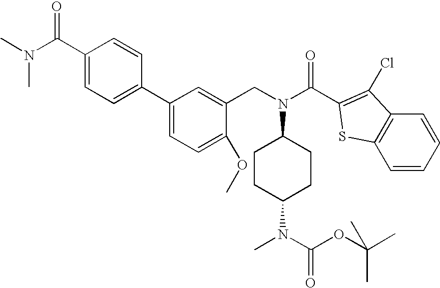

Biaryl amine 59 (110 mg, 0.22 mmol) was treated with 3-chloro-benzo[b]thiophene-2-carbonyl chloride (56 mg, 0.24 mmol) using Method D to give the title compound.

Yield: 100 mg (65%).

LC/MS tᵣ 1.93 min.

MS (ES+) m/z 592, 590 (M−CO₂C(CH₃)₃+H).

### 3-Chloro-benzo[b]thiophene-2-carboxylic acid (4′-dimethylcarbamoyl-4-methoxy-biphenyl-3-ylmethyl)-(4-methylamino-cyclohexyl)-amide hydrochloride (61)

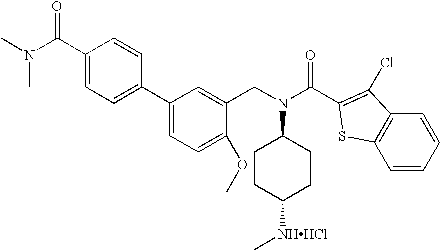

tert-Butyl carbamate 60 (120 mg, 0.18 mmol) was deprotected using Method E to give the title compound.

Yield: 120 mg (quant.).

LC/MS tᵣ 1.39 min.

MS (ES+) m/z 592, 590 (M+H).

### Synthesis of Compound 318

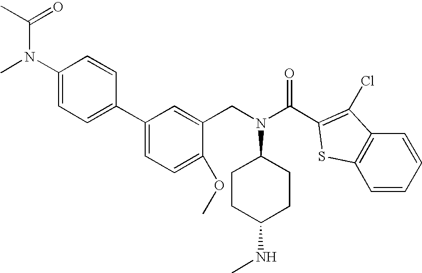

### N-(4-Bromophenyl)-N-methyl-acetamide (62)

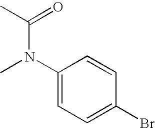

4-bromoacetanilide (1.0 g, 4.67 mmol) in DMF (5 mL) was added dropwise to a suspension of sodium hydride (224 mg, 5.61 mmol, 60% dispersion in mineral oil) in DMF (5 mL) at 0° C. After stirring 1 h at 0° C., iodomethane (349 μl, 5.61 mmol) was added and the reaction mixture warmed to RT and stirred 16 h. The reaction was quenched with water (15 mL) and extracted into EtOAc (3×15 mL); the combined organic phases were then dried over Na₂SO₄ and reduced in vacuo. Purification by column chromatography (40% EtOAc in heptane) gave the title compound.

Yield: 780 mg (74%).

LC/MS tᵣ 1.17 min.

MS (ES+) m/z 230, 228 (M+H).

### tert-Butyl {4-[[4′-(acetyl-methyl-amino)-4-methoxy-biphenyl-3-ylmethyl]-(3-chloro-benzo[b]thiophene-2-carbonyl)-amino]-cyclohexyl}-methyl-carbamate (63)

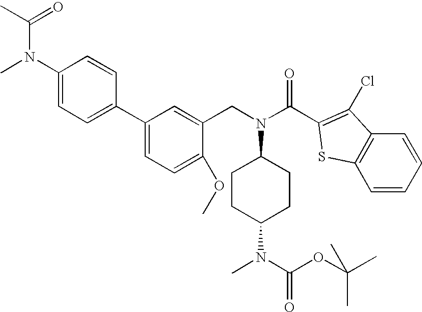

Boronic acid 5 (200 mg, 0.34 mmol) was coupled to aryl bromide 62 (93 mg, 0.41 mmol) using Method B to give the title compound.

Yield: 170 mg (72%).

LC/MS t_{f} 1.96 min.

MS (ES+) m/z 636, 634 (M−C(CH₃)₃+H), 592, 590 (M−CO₂C(CH₃)₃+H).

### 3-Chloro-benzo[b]thiophene-2-carboxylic acid [4′-(acetyl-methyl-amino)-4-methoxy-biphenyl-3-ylmethyl]-(4-methylamino-cyclohexyl)-amide hydrochloride (64)

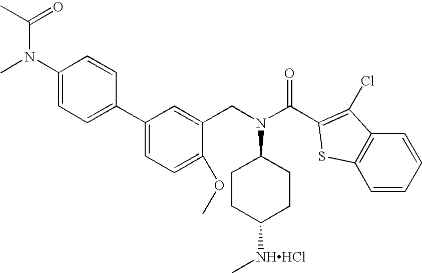

tert-Butyl carbamate 63 (170 mg, 0.25 mmol) was deprotected using Method E. On removal of the solvent in vacuo, the residue was dissolved in MeOH (100 μL) and water (3 mL) and this aqueous phase washed with TBME (3×3 mL). The water was then removed in vacuo to afford the title compound.

Yield: 120 mg (83%).

LC/MS tᵣ 1.39 min.

MS (ES+) m/z 592, 590 (M+H), 561, 559 (M−31+H).

### Synthesis of Compound 319

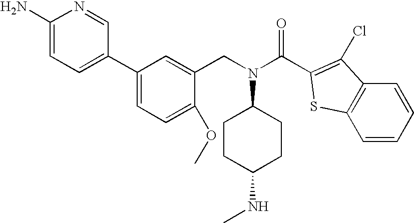

### tert-Butyl {4-[[5-(6-amino-pyridin-3-yl)-2-methoxy-benzyl]-(3-chloro-benzo[b]thiophene-2-carbonyl)-amino]-cyclohexyl}-methyl-carbamate (65)

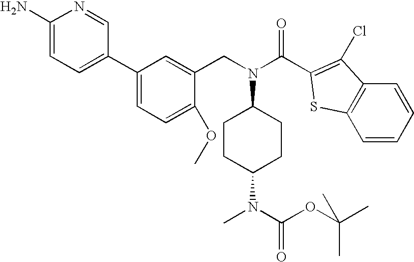

Boronic acid 5 (175 mg, 0.29 mmol) was coupled to 2-amino-5-bromopyridine (62 mg, 0.36 mmol) using Method B to give the title compound.

Yield: 66 mg (35%).

LC/MS tᵣ 1.67 min.

MS (ES+) m/z 637, 635 (M+H), 581, 579 (M−C(CH₃)₃+H).

### 3-Chloro-benzo[b]thiophene-2-carboxylic acid [5-(6-amino-pyridin-3-yl)-2-methoxy-benzyl]-(4-methylamino-cyclohexyl)-amide dihydrochloride (66)

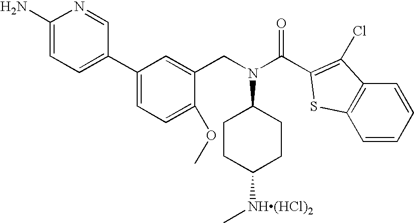

tert-Butyl carbamate 65 (66 mg, 0.10 mmol) was deprotected using Method E to give the title compound.

Yield: 62 mg (98%).

LC/MS tᵣ 1.17 min.

MS (ES+) m/z 537, 535 (M+H).

### Synthesis of Compound 320

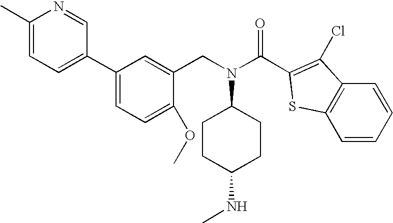

### 6-Methylpyridin-3-yl trifluoromethanesulfonate (67)

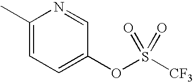

A solution of 5-hydroxy-2-methylpyridine (1.0 g, 9.17 mmol) and trifluoro-methanesulfonic anhydride (1.85 mL, 11.0 mmol) in DCM (20 mL) was treated with triethylamine (1.53 mL, 11.0 mmol) dropwise via syringe at 0° C. After 4 h stirring at RT, the reaction mixture was reduced in vacuo to afford the crude title compound.

Yield: 2.10 g (95%).

### tert-Butyl {4-[2-methoxy-5-(6-methyl-pyridin-3-yl)-benzylamino]-cyclohexyl}-methyl-carbamate (68)

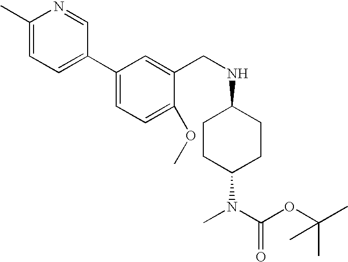

Boronic acid 4 (1.6 g, 4.08 mmol) was coupled to pyridyl triflate 67 (982 mg, 4.08 mmol) using Method A to give the title compound.

Yield: 813 mg (45%) but the compound is contaminated by the following co-eluting dimer:

LC/MS tᵣ 1.09 min.

MS (ES+) m/z 440 (M+H), 384 (M−C(CH₃)₃+H).

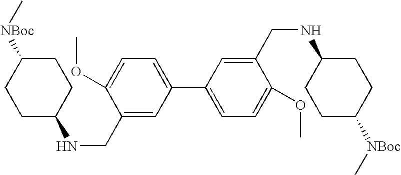

### tert-Butyl (4-{(3-chloro-benzo[b]thiophene-2-carbonyl)-[2-methoxy-5-(6-methyl-pyridin-3-yl)-benzyl]-amino}-cyclohexyl)-methyl-carbamate (69)

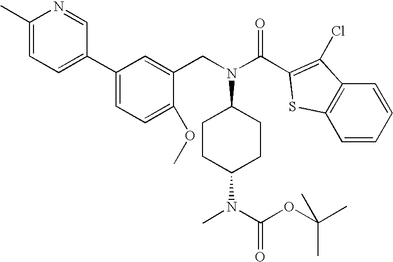

Biaryl amine 68 (175 mg, 0.40 mmol) was treated with 3-chlorobenzo-[b]thiophene-2-carbonyl chloride (110 mg, 0.48 mmol) using Method D to give the title compound.

Yield: 81 mg (32%).

LC/MS tᵣ 1.61 min.

MS (ES+) m/z 636, 634 (M+H).

### 3-Chloro-benzo[b]thiophene-2-carboxylic acid [2-methoxy-5-(6-methyl-pyridin-3-yl)-benzyl]-(4-methylamino-cyclohexyl)-amide dihydrochloride (70)

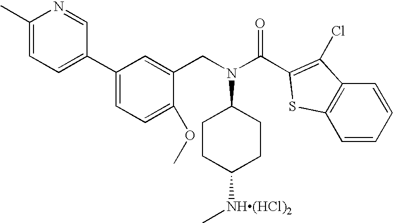

tert-Butyl carbamate 69 (81 mg, 0.13 mmol) was deprotected using Method F to give the title compound.

Yield: 74 mg (95%).

LC/MS tᵣ 1.56 min.

MS (ES+) m/z 650, 648 (M+CF₃COOH+H), 536, 534 (M+H).

### Synthesis of Compound 321

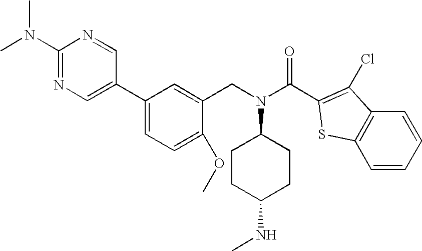

### 3-Chloro-benzo[b]thiophene-2-carboxylic acid [5-(2-dimethylamino-pyrimidin-5-yl)-2-methoxy-benzyl]-(4-methylamino-cyclohexyl)-amide dihydrochloride (71)

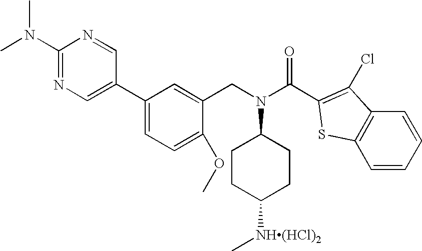

The title compound was prepared from boronic acid 5 (25 mg, 43 μmol) and 5-bromo-2-(dimethylamino)pyrimidine (7.2 mg, 36 μmol) in accordance with Method L2.

Yield: 12.0 mg (52%).

LC/MS tᵣ 1.25 min.

MS (ES+) m/z 566, 564 (M+H).

### Synthesis of Compound 322

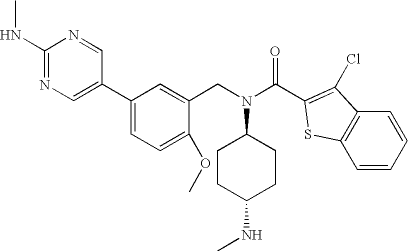

### 3-Chloro-benzo[b]thiophene-2-carboxylic acid [2-methoxy-5-(2-methylamino-pyrimidin-5-yl)-benzyl]-(4-methylamino-cyclohexyl)-amide dihydrochloride (72)

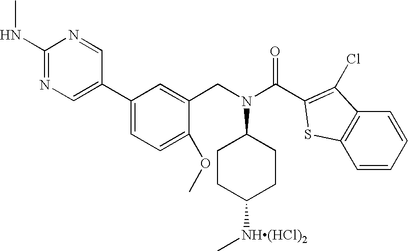

The title compound was prepared from boronic acid 5 (25 mg, 43 μmol) and 5-bromo-2-(methylamino)pyrimidine (6.7 mg, 36 μmol) in accordance with Method L2.

Yield: 5.0 mg (22%).

LC/MS tᵣ 1.17 min.

MS (ES+) m/z 552, 550 (M+H).

### Synthesis of Compound 323

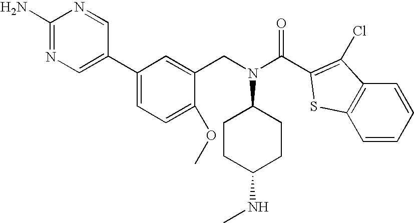

### 3-Chloro-benzo[b]thiophene-2-carboxylic acid [5-(2-amino-pyrimidin-5-yl)-2-methoxy-benzyl]-(4-methylamino-cyclohexyl)-amide dihydrochloride (73)

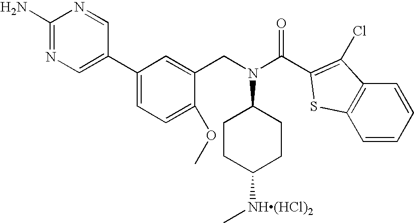

The title compound was prepared from boronic acid 5 (25 mg, 43 μmol) and 2-amino-5-bromopyrimidine (6.2 mg, 36 μmol) in accordance with Method L2.

Yield: 5.7 mg (26%).

LC/MS tᵣ 1.13 min.

MS (ES+) m/z 538, 536 (M+H).

### Synthesis of Compound 324

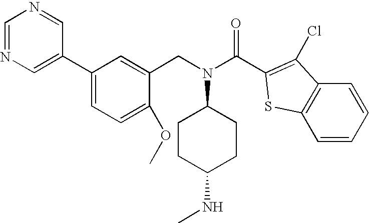

### 3-Chloro-benzo[b]thiophene-2-carboxylic acid (2-methoxy-5-pyrimidin-5-yl-benzyl)-(4-methylamino-cyclohexyl)-amide dihydrochloride (74)

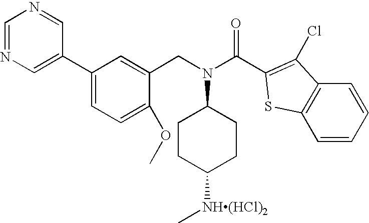

The title compound was prepared from boronic acid 5 (25 mg, 43 mmol) and 5-bromopyrimidine (5.6 mg, 36 μmol) in accordance with Method L2.

Yield: 4.1 mg (18%).

LC/MS tᵣ 1.24 min.

MS (ES+) m/z 523, 521 (M+H), 492, 490 (M−31+H).

### Synthesis of Compound R4

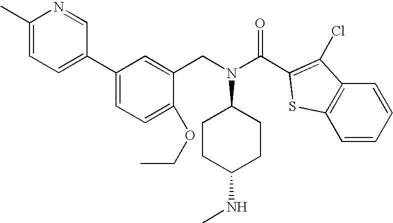

### tert-Butyl {4-[2-ethoxy-5-(6-methyl-pyridin-3-yl)-benzylamo]-cyclohexyl}-methyl-carbamate (75)

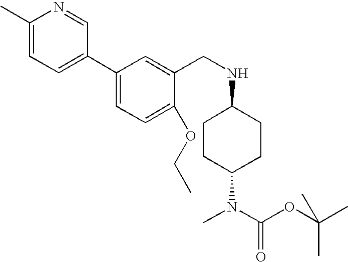

Boronic acid 11 (812 mg, 2.0 mmol) was coupled to 5-bromo-2-methylpyridine (344 mg, 2.0 mmol) using Method A to give the title compound.

Yield: 373 mg (41%).

LC/MS tᵣ 1.12 min.

MS (ES+) m/z 907 (2M+H), 454 (M+H).

### tert-Butyl (4-{(3-chloro-benzo[b]thiophene-2-carbonyl)-[2-ethoxy-5-(6-methyl-pyridin-3-yl)-benzyl]-amino}-cyclohexyl)-methyl-carbamate (76)

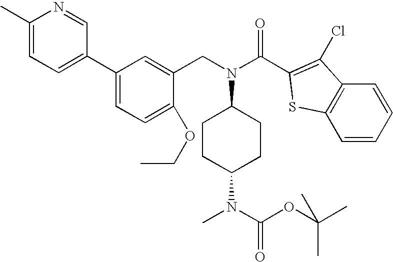

Biaryl amine 75 (373 mg, 0.82 mmol) was treated with 3-chlorobenzo-[b]thiophene-2-carbonyl chloride (227 mg, 1.80 mmol) using Method D to give the title compound.

Yield: 224 mg (42%).

LC/MS tᵣ 1.59 min.

MS (ES+) m/z 650, 648 (M+H).

### 3-Chloro-benzo[b]thiophene-2-carboxylic acid [2-ethoxy-5-(6-methyl-pyridin-3-yl)-benzyl]-(4-methylamino-cyclohexyl)-amide dihydrochloride (77)

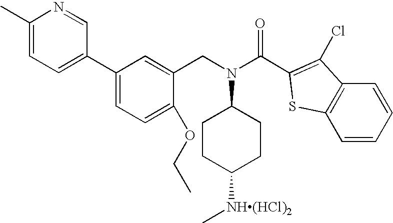

tert-Butyl carbamate 76 (200 mg, 0.31 mmol) was deprotected using Method F to give the title compound.

Yield: 192 mg (quant.).

LC/MS tᵣ 1.73 min.

MS (ES+) m/z 550, 548 (M+H).

### Synthesis of Compound 325

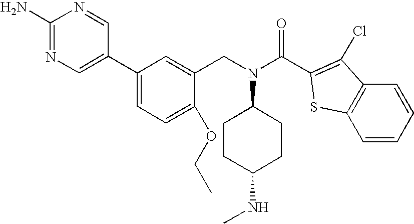

### 3-Chloro-benzo[b]thiophene-2-carboxylic acid [5-(2-amino-pyrimidin-5-yl)-2-ethoxy-benzyl]-(4-methylamino-cyclohexyl)-amide dihydrochloride (78)

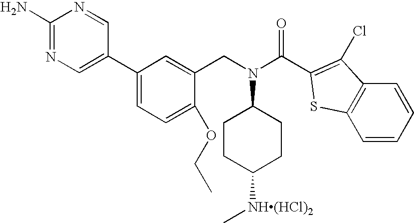

The title compound was prepared from boronic acid 12 (25 mg, 42 μmol) and 2-amino-5-bromopyrimidine (6.0 mg, 35 μmol) in accordance with Method L2.

Yield: 8.1 mg (37%).

LC/MS tᵣ 1.1-8 min.

MS (ES+) m/z 552, 550 (M+H).

### Synthesis of Compound 326

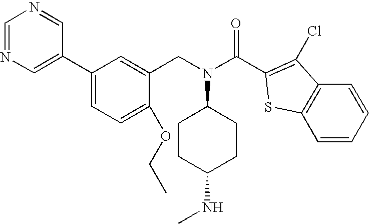

### 3-Chloro-benzo[b]thiophene-2-carboxylic acid (2-ethoxy-5-pyrimidin-5-yl-benzyl)-(4-methylamino-cyclohexyl)-amide dihydrochloride (79)

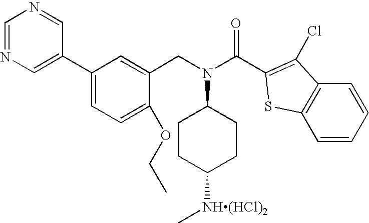

The title compound was prepared from boronic acid 12 (25 mg, 42 μmol) and 5-bromopyrimidine (5.5 mg, 35 μmol) in accordance with Method L2.

Yield: 6.3 mg (30%).

LC/MS tᵣ 1.26 min.

MS (ES+) m/z 537, 535 (M+H).

### Synthesis of Compound 327

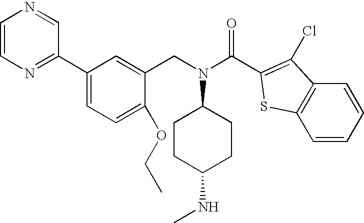

### 3-Chloro-benzo[b]thiophene-2-carboxylic acid (2-ethoxy-5-pyrazin-2-yl-benzyl)-(4-methylamino-cyclohexyl)-amide dihydrochloride (80)

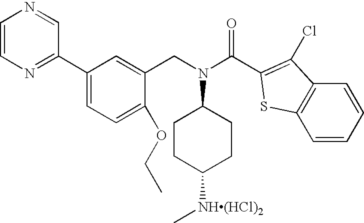

The title compound was prepared from boronic acid 12 (25 mg, 42 μmol) and 2-chloropyrazine (4.0 mg, 35 μmol) in accordance with Method L2.

Yield: 13.6 mg (64%).

LC/MS tᵣ 1.76 min.

MS (ES+) m/z 537, 535 (M+H).

### Synthesis of Compound 328

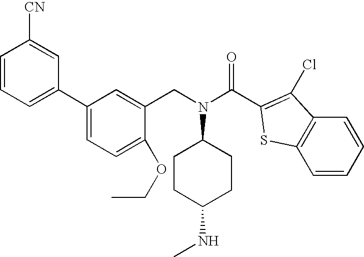

### tert-Butyl {4-[(3′-cyano-4-ethoxy-biphenyl-3-ylmethyl)-amino]-cyclohexyl}-methyl-carbamate (81)

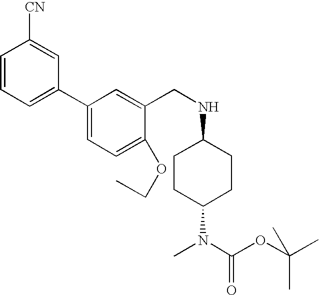

Boronic acid 11 (374 mg, 0.92 mmol) was coupled to 3-bromobenzonitrile (200 mg, 1.10 mmol) using Method B to give the title compound.

Yield: 390 mg (91%).

LC/MS tᵣ 1.56 min.

MS (ES+) m/z 464 (M+H), 408 (M−C(CH₃)₃+H).

### tert-Butyl {4-[(3-chloro-benzo[b]thiophene-2-carbonyl)-(3′-cyano-4-ethoxy-biphenyl-3-ylmethyl)-amino]-cyclohexyl}-methyl-carbamate (82)

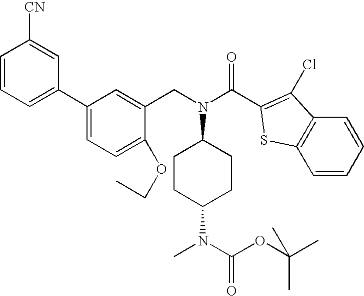

Biaryl amine 81 (390 mg, 0.84 mmol) was treated with 3-chlorobenzo-[b]thiophene-2-carbonyl chloride (233 mg, 1.01 mmol) using Method D to give the title compound.

Yield: 329 mg (59%).

LC/MS tᵣ 2.16 min.

MS (ES+) m/z 604, 602 (M−C(CH₃)₃+H).

### 3-Chloro-benzo[b]thiophene-2-carboxylic acid (3′-cyano-4-ethoxy-biphenyl-3-ylmethyl)-(4-methylamino-cyclohexyl)-amide hydrochloride (83)

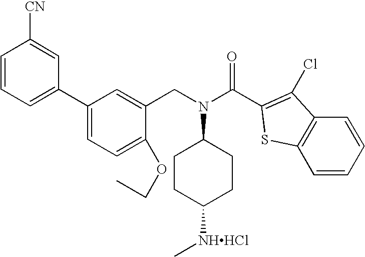

tert-Butyl carbamate 82 (329 mg, 0.50 mmol) was deprotected using Method E. The solvents were then removed in vacuo and the residue purified by column chromatography (gradient elution—100% EtOAc increasing to 50% MeOH in EtOAc with 5% triethylamine). Treatment of the isolated free base in accordance with Method H gave the title compound.

Yield: 100 mg (36%).

LC/MS tᵣ 1.60 min.

MS (ES+) m/z 560, 558 (M+H), 529, 527 (M−31+H).

### Synthesis of Compound 329

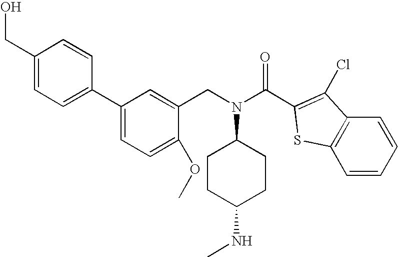

### tert-Butyl {4-[(3-chloro-benzo[b]thiophene-2-carbonyl)-(4′-hydroxymethyl-4-methoxy-biphenyl-3-ylmethyl)-amino]-cyclohexyl}-methyl-carbamate (84)

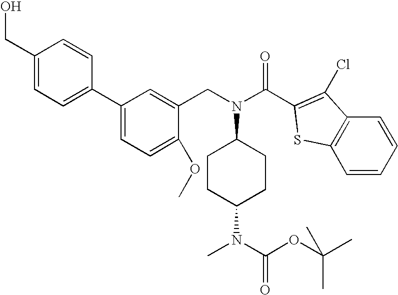

Boronic acid 5 (210 mg, 0.36 mmol) was coupled to (4-bromophenyl)-methanol (80 mg, 0.43 mmol) using Method B to give the title compound.

Yield: 150 mg (64%).

LC/MS tᵣ 1.99 min.

MS (ES+) m/z 595, 593 (M−C(CH₃)₃+H).

### 3-Chloro-benzo[b]thiophene-2-carboxylic acid (4′-hydroxymethyl-4-methoxy-biphenyl-3-ylmethyl)-(4-methylamino-cyclohexyl)-amide hydrochloride (85)

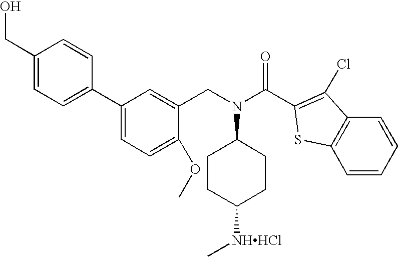

tert-Butyl carbamate 84 (70 mg, 0.11 mmol) was deprotected using Method E to give the title compound.

Yield: 64 mg (quant.).

LC/MS tᵣ 1.50 min.

MS (ES+) m/z 551, 549 (M+H), 520, 518 (M−31+H).

### Synthesis of Compound 330

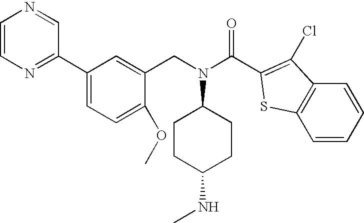

### 3-Chloro-benzo[b]thiophene-2-carboxylic acid (2-methoxy-5-pyrazin-2-yl-benzyl)-(4-methylamino-cyclohexyl)-amide bis(trifluoroacetate) (86)

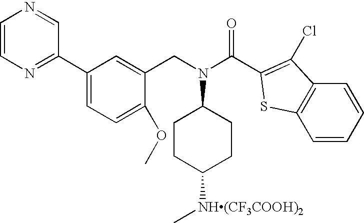

Boronic acid 5 (25 mg, 43 μmol) and 2-chloropyrazine (4.1 mg, 36 μmol) were coupled in accordance with Method L1. The title compound was obtained after preparative HPLC.

Yield: 3.2 mg (10%).

LC/MS tᵣ 1.34 min.

MS (ES+) m/z 523, 521 (M+H), 492, 490 (M−31+H).

### Synthesis of Compound 331

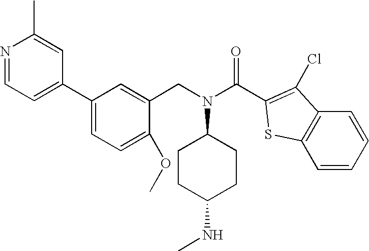

### 2-Methoxy-5-(2-methyl-pyridin-4-yl)-benzaldehyde (87)

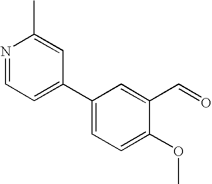

A stirred suspension of 3-formyl-4-methoxybenzeneboronic acid (700 mg, 3.89 mmol), 4-chloro-2-picoline (494 mg, 3.89 mmol), potassium carbonate (1.45 g, 10.5 mmol) and tetrakis(triphenylphosphine)palladium(0) (225 mg, 0.19 mmol) in 1,2-dimethoxyethane (14 mL) and water (5 mL) was degassed at RT with N₂ for 15 minutes then warmed for 16 h at 85° C. The reaction mixture was then cooled to RT and 2 M HCl (15 mL) added. The aqueous layer was washed with TBME (15 mL) then basified to pH 10 with aqueous K₂CO₃ and extracted into EtOAc (3×50 mL). The combined organic phases were dried over Na₂SO₄ and the solvents removed in vacuo to give the title compound.

Yield: 820 mg (93%).

LC/MS tᵣ 0.91 min.

MS (ES+) m/z 228 (M+H).

### tert-Butyl {4-[2-methoxy-5-(2-methyl-pyridin-4-yl)-benzylamino]-cyclohexyl}-methyl-carbamate (88)

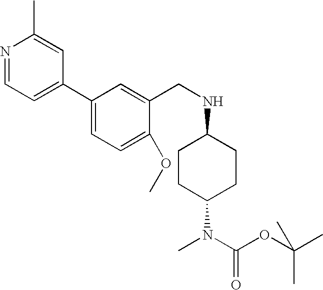

Amine 3 (823 mg, 3.61 mmol) was treated with aldehyde 87 (820 mg, 3.61 mmol) in accordance with Method C. The reaction mixture was then diluted with TBME (10 mL) and 0.5 M HCl (10 mL) and the layers separated. The aqueous phase was basified to pH 10 with aqueous K₂CO₃ and extracted into EtOAc (3×50 mL). The combined organic phases were dried over Na₂SO₄ and the solvents removed in vacuo to give the title compound.

Yield: 720 mg (45%).

LC/MS tᵣ 1.08 min.

MS (ES+) m/z 440 (M+H), 384 (M−C(CH₃)₃+H).

### 3-Chloro-benzo[b]thiophene-2-carboxylic acid [2-methoxy-5-(2-methyl-pyridin-4-yl)-benzyl]-(4-methylamino-cyclohexyl)-amide dihydrochloride (89)

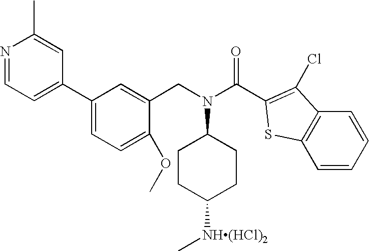

Biaryl amine 88 (250 mg, 0.57 mmol) was treated with 3-chlorobenzo-[b]thiophene-2-carbonyl chloride (157 mg, 0.68 mmol) using Method D. The resultant amide (175 mg, 0.28 mmol) isolated after chromatography was then directly deprotected using Method F to afford the title compound.

Yield: 138 mg (40% over two steps).

LC/MS tᵣ 1.08 min.

MS (ES+) m/z 536, 534 (M+H).

### Synthesis of Compound R1

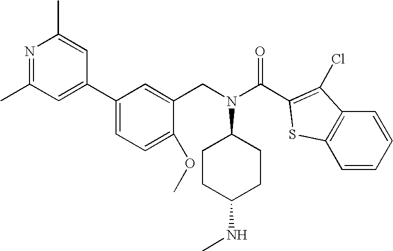

### 2,6-Dimethyl-pyridin-4-ol (90)

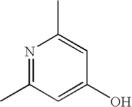

Five separate batches of dehydroacetic acid (1.5 g, 8.92 mmol) were each suspended in conc. ammonia (4 mL) and irradiated at 120° C. for 20 minutes (150 W, Discover® System microwave reactor by CEM Corporation, Matthews, N.C., USA) in the microwave. Once cooled, the solutions were combined and evaporated to dryness to afford the title compound.

Yield: 5.91 g (108%) @ 73% purity by LC/MS. The remaining mass balance is unreacted dehydroacetic acid.

LC/MS tᵣ 0.67 min.

MS (ES+) m/z 168 (M+CH₃CN+H).

### 2,6-Dimethyl-pyridin-4-yl trifluoromethanesulfonate (91)

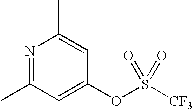

A suspension of pyridone 90 (5.0 g, 40.6 mmol) in DCM (100 mL) at 0° C. was treated with triethylamine (8.50 mL, 60.9 mmol) followed by trifluoromethane-sulfonic anhydride (10.2 mL, 60.9 mmol), added dropwise via syringe over 5 minutes. After warming to RT and stirring 2 h, the reaction mixture was washed with aqueous NaHCO₃ (3×100 mL) and reduced in vacuo to afford the title compound.

Yield: 9.25 g (quant.).

LC/MS tᵣ 0.96 min.

MS (ES+) m/z 256 (M+H).

### tert-Butyl {4-[5-(2,6-dimethyl-pyridin-4-yl)-2-methoxy-benzylamino]-cyclohexyl}-methyl-carbamate (92)

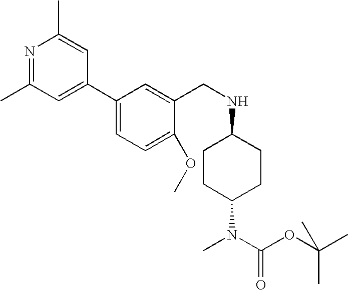

Boronic acid 4 (400 mg, 1.02 mmol) was coupled to pyridyl triflate 91 (260 mg, 1.02 mmol) using Method B to give the title compound.

Yield: 268 mg (58%).

LC/MS tᵣ 1.07 min.

MS (ES+) m/z 454 (M+H).

### tert-Butyl (4-{(3-chloro-benzo[b]thiophene-2-carbonyl)-[5-(2,6-dimethyl-pyridin-4-yl)-2-methoxy-benzyl]-amino}-cyclohexyl)-methyl-carbamate (93)

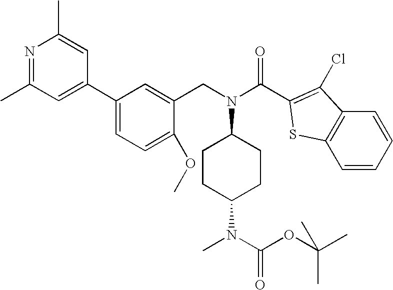

Biaryl amine 92 (100 mg, 0.22 mmol) was treated with 3-chlorobenzo-[b]thiophene-2-carbonyl chloride (61 mg, 0.26 mmol) using Method D to give the title compound.

Yield: 91 mg (64%).

LC/MS tᵣ 1.63 min.

MS (ES+) m/z 650, 648 (M+H), 594, 592 (M−C(CH₃)₃+H).

### 3-Chloro-benzo[b]thiophene-2-carboxylic acid [5-(2,6-dimethyl-pyridin-4-yl)-2-methoxy-benzyl]-(4-methylamino-cyclohexyl)-amide dihydrochloride (94)

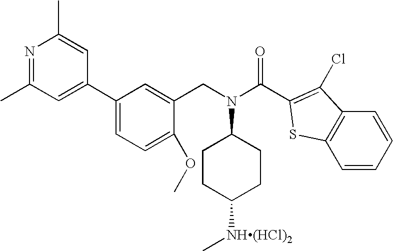

tert-Butyl carbamate 93 (91 mg, 0.14 mmol) was deprotected using Method F to give the title compound.

Yield: 73 mg (96%).

LC/MS tᵣ 1.71 min.

MS (ES+) m/z 550, 548 (M+H).

### Synthesis of Compound 332

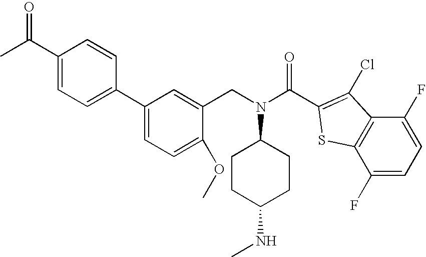

### tert-Butyl {4-[(4′-acetyl-4-methoxy-biphenyl-3-ylmethyl)-(3-chloro-4,7-difluoro-benzo[b]thiophene-2-carbonyl)-amino]-cyclohexyl}-methyl-carbamate (95)

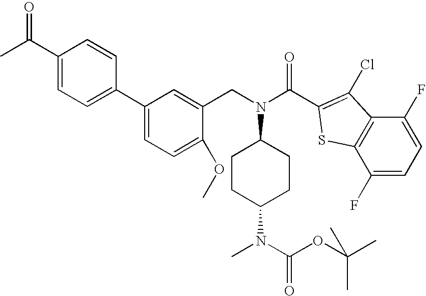

Boronic acid 9 (700 mg, 1.12 mmol) was coupled to 4′-bromoacetophenone (268 mg, 1.34 mmol) using Method A to give the title compound.

Yield: 396 mg (51%).

LC/MS tᵣ 1.96 min.

MS (ES+) m/z 699, 697 (M+H), 643, 641 (M−C(CH₃)₃+H).

### 3-Chloro-4,7-difluoro-benzo[b]thiophene-2-carboxylic acid (4′-acetyl-4-methoxy-biphenyl-3-ylmethyl)-(4-methylamino-cyclohexyl)-amide hydrochloride (96)

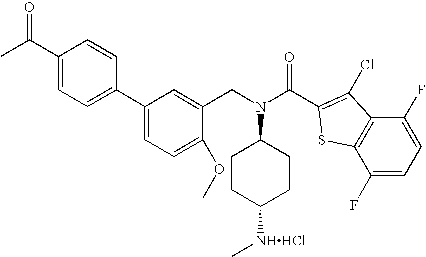

tert-Butyl carbamate 95 (396 mg, 0.57 mmol) was deprotected using Method F to give the title compound.

Yield: 354 mg (98%).

LC/MS tᵣ 1.49 min.

MS (ES+) m/z 599, 597 (M+H).

¹H NMR δ_{H} ppm (400 MHz, D₆-DMSO, 95° C.): 8.59 (2H, br. s), 7.85 (2H, d), 7.54 (2H, d), 7.48 (1H, dd), 7.43 (1H, s), 7.25-7.13 (2H, m), 6.95 (1H, d), 4.54 (2H, s), 3.75 (1H, br. s), 3.71 (3H, s), 2.79-2.70 (1H, obsc. m), 2.44 (3H, s), 2.31 (3H, s), 2.00-1.91 (2H, m), 1.78-1.61 (4H, m), 1.32-1.19 (2H, m).

### Synthesis of Compound 333

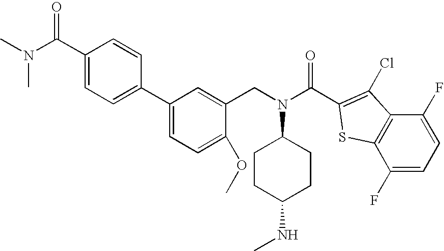

### tert-Butyl {4-[(3-chloro-4,7-difluoro-benzo[b]thiophene-2-carbonyl)-(4′-dimethylcarbamoyl-4-methoxy-biphenyl-3-ylmethyl)-amino]-cyclohexyl}-methyl-carbamate (97)

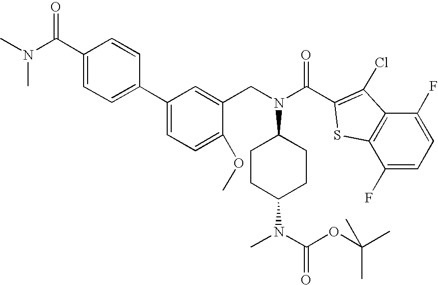

Boronic acid 9 (700 mg, 1.12 mmol) was coupled to aryl bromide 58 (306 mg, 1.34 mmol) using Method A to give the title compound.

Yield: 250 mg (32%) containing triphenylphosphine oxide (ca. 27%) plus 317 mg (40%) cruder material.

LC/MS tᵣ 1.84 min.

MS (ES+) m/z 728, 726 (M+H), 672, 670 (M−C(CH₃)₃+H).

### 3-Chloro-4,7-difluoro-benzo[b]thiophene-2-carboxylic acid (4′-dimethylcarbamoyl-4-methoxy-biphenyl-3-ylmethyl)-(4-methylamino-cyclohexyl)-amide hydrochloride (98)

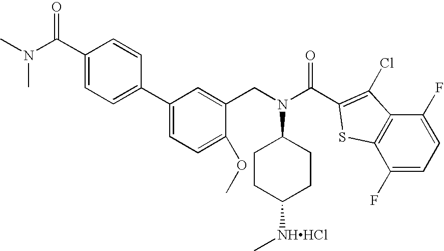

tert-Butyl carbamate 97 (250 mg, 0.34 mmol) was deprotected using Method F. The isolated salt was then dissolved in the minimum amount of DCM and added dropwise to cold (0° C.) TBME (25 mL). The resultant white precipitate was isolated by filtration and dried to afford the title compound.

Yield: 169 mg (75%).

LC/MS tᵣ 1.38 min.

MS (ES+) m/z 628, 626 (M+H).

¹H NMR δ_{H} ppm (400 MHz, D₆-DMSO, 95° C.): 8.46 (2H, br. s), 7.41 (2H, d), 7.38 (1H, obsc. dd), 7.35 (1H, s), 7.27 (2H, d), 7.21-7.08 (2H, m), 6.88 (1H, d), 4.49 (2H, s), 3.70 (1H, br. s), 3.66 (3H, s), 2.80 (6H, s), 2.74-2.64 (1H, obsc. m), 2.27 (3H, s), 1.95-1.85 (2H, m), 1.74-1.56 (4H, m), 1.27-1.13 (2H, m).

### Synthesis of Compound 334

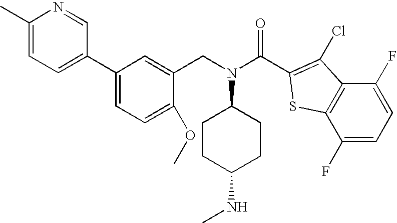

### tert-Butyl (4-{(3-chloro-4,7-difluoro-benzo[b]thiophene-2-carbonyl)-[2-methoxy-5-(6-methyl-pyridin-3-yl)-benzyl]-amino}-cyclohexyl)-methyl-carbamate (99)

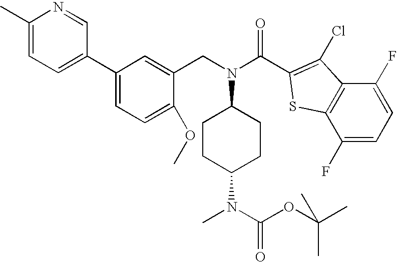

Biaryl amine 68 (813 mg, 1.85 mmol) was treated with acid chloride 8 (542 mg, 2.03 mmol) using Method D to give the title compound.

Yield: 133 mg (11%).

LC/MS tᵣ 1.67 min.

MS (ES+) m/z 672, 670 (M+H), 616, 614 (M−C(CH₃)₃+H).

### 3-Chloro-4,7-difluoro-benzo[b]thiophene-2-carboxylic acid [2-methoxy-5-(6-methyl-pyridin-3-yl)-benzyl]-(4-methylamino-cyclohexyl)-amide bis(trifluoroacetate) (100)

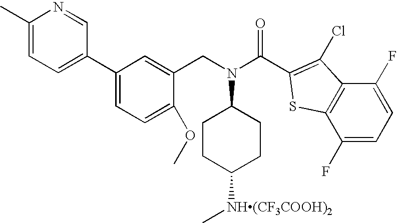

tert-Butyl carbamate 99 (133 mg, 0.20 mmol) was deprotected using Method G to afford the title compound.

Yield: 158 mg (quant.).

LC/MS tᵣ 1.72 min.

MS (ES+) m/z 572, 570 (M+H).

### Synthesis of Compound R7

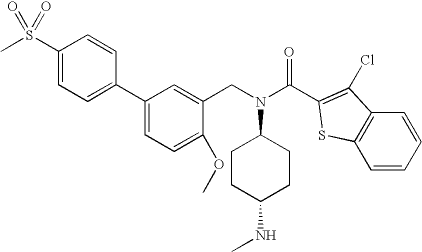

### tert-Butyl {4-[(3-chloro-benzo[b]thiophene-2-carbonyl)-(4 methanesulfonyl-4-methoxy-biphenyl-3-ylmethyl)-amino]-cyclohexyl}-methyl-carbamate (101)

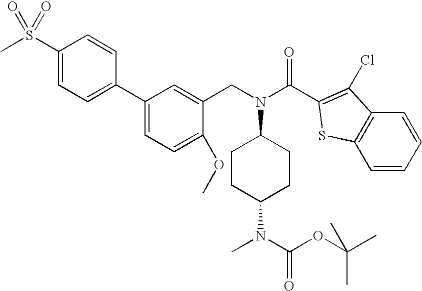

Boronic acid 5 (330 mg, 0.84 mmol) was coupled to 4′-bromophenyl methyl sulfone (237 mg, 1.01 mmol) using Method A to give the title compound.

Yield: 176 mg (30%).

### 3-Chloro-benzo[b]thiophene-2-carboxylic acid (4′-methanesulfonyl-4-methoxy-biphenyl-3-ylmethyl)-(4-methylamino-cyclohexyl)-amide hydrochloride (102)

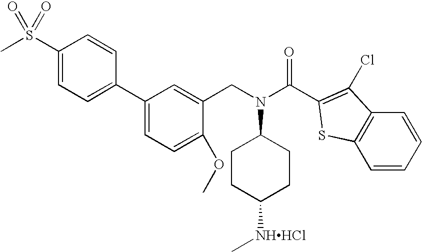

tert-Butyl carbamate 101 (173 mg, 0.25 mmol) was deprotected using Method F to afford the title compound.

Yield: 176 mg (quant.).

LC/MS tᵣ 1.41 min.

MS (ES+) m/z 599, 597 (M+H), 568, 566 (M−31+H).

### Synthesis of Compound 335

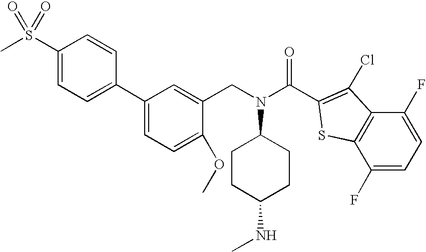

### tert-Butyl {4-[(4′-methanesulfonyl-4-methoxy-biphenyl-3-ylmethyl)-amino]-cyclohexyl}-methyl-carbamate (103)

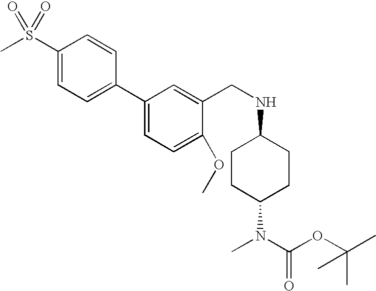

Boronic acid 4 (3.0 g, 7.64 mmol) was coupled to 4′-bromophenyl methyl sulfone (1.80 g, 7.64 mmol) using Method A to give the title compound.

Yield: 1.84 g (48%).

LC/MS tᵣ 1.28 min.

MS (ES+) m/z 503 (M+H).

### tert-Butyl {4-[(3-Chloro-4,7-difluoro-benzo[b]thiophene-2-carbonyl)-(4′-methanesulfonyl-4-methoxy-biphenyl-3-ylmethyl)-amino]-cyclohexyl}-methyl-carbamate (104)

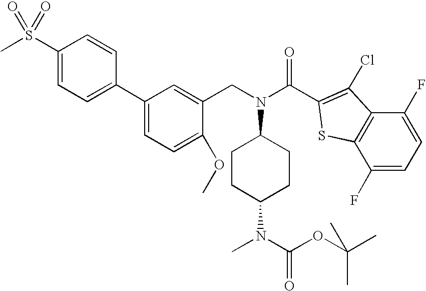

Biaryl amine 103 (1.84 g, 3.66 mmol) was treated with acid chloride 8 (1.17 g, 4.38 mmol) using Method D to give the title compound.

Yield: 2.10 g (78%).

LC/MS tᵣ 1.84 min.

MS (ES+) m/z 735, 733 (M+H), 679, 677 (M−C(CH₃)₃+H).

### 3-Chloro-4,7-difluoro-benzo[b]thiophene-2-carboxylic acid (4′-methanesulfonyl-4-methoxy-biphenyl-3-ylmethyl)-(4-methylamino-cyclohexyl)-amide hydrochloride (105)

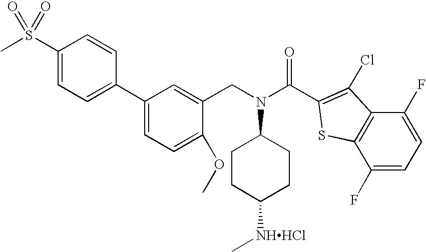

tert-Butyl carbamate 104 (2.10 g, 2.86 mmol) was deprotected using Method F then purified by column chromatography (gradient elution—1-5% MeOH in DCM with 0.5% triethylamine). The free base was then converted to the title compound using Method H.

Yield: 1.64 g (85%).

LC/MS tᵣ 1.94 min.

MS (ES+) m/z 635, 633 (M+H).

¹H NMR δ_{H} ppm (400 MHz, D₆-DMSO, 95° C.): 9.13 (2H, br. s), 7.98 (2H, d), 7.82 (2H, d), 7.65 (1H, dd), 7.60 (1H, s), 7.41-7.26 (2H, m), 7.12 (1H, d), 4.71 (2H, s), 3.92 (1H, br. s), 3.88 (3H, s), 3.22 (3H, s), 2.95-2.83 (1H, m), 2.46 (3H, s), 2.20-2.08 (2H, m), 1.94-1.76 (4H, m), 1.54-1.39 (2H, m).

### Synthesis of Compound 336

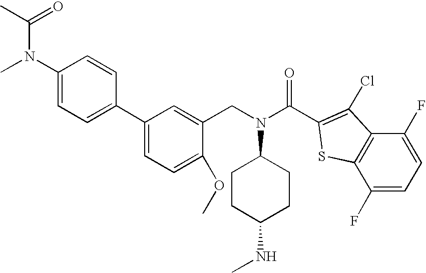

### tert-Butyl (4-{[4′-(acetyl-methyl-amino)-4-methoxy-biphenyl-3-ylmethyl]-amino}-cyclohexyl)-methyl-carbamate (106)

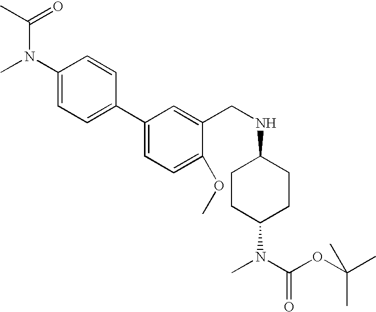

Boronic acid 4 (650 mg, 1.04 mmol) was coupled to aryl bromide 62 (238 mg, 1.04 mmol) using Method A to give the title compound.

Yield: 420 mg (51%).

LC/MS tᵣ 1.27 min.

MS (ES+) m/z 496 (M+H).

### tert-Butyl {4-[[4′-(acetyl-methyl-amino)-4-methoxy-biphenyl-3-ylmethyl]-(3-chloro-4,7-difluoro-benzo[b]thiophene-2-carbonyl)-amino]-cyclohexyl}-methyl-carbamate (107)

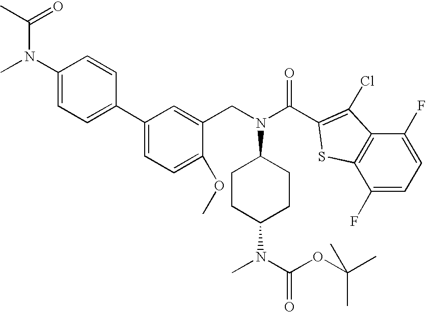

Biaryl amine 106 (420 mg, 0.84 mmol) was treated with acid chloride 8 (249 mg, 0.93 mmol) using Method D to give the title compound.

Yield: 280 mg (46%).

LC/MS tᵣ 1.85 min.

MS (ES+) m/z 672, 670 (M−C(CH₃)₃+H), 628, 626 (M−CO₂C(CH₃)₃+H).

### 3-Chloro-4,7-difluoro-benzo[b]thiophene-2-carboxylic acid [4′-(acetyl-methyl-amino)-4-methoxy-biphenyl-3-ylmethyl]-(4-methylamino-cyclohexyl)-amide hydrochloride (108)

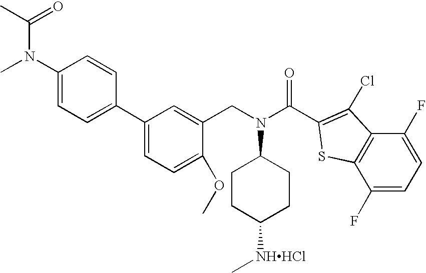

tert-Butyl carbamate 107 (280 mg, 0.38 mmol) was deprotected using Method F to give the title compound.

Yield: 185 mg (77%).

LC/MS tᵣ 1.33 min.

MS (ES+) m/z 628, 626 (M+H).

### Synthesis of Compound 337

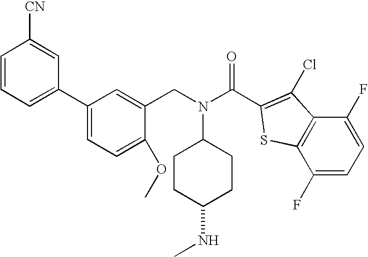

### tert-Butyl {4-[(3-chloro-4,7-difluoro-benzo[b]thiophene-2-carbonyl)-(3′-cyano-4-methoxy-biphenyl-3-ylmethyl)-amino]-cyclohexyl}-methyl-carbamate (109)

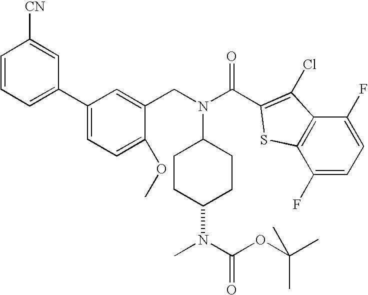

Biaryl amine 22 (475 mg, 1.06 mmol) was treated with acid chloride 8 (338 mg, 1.27 mmol) using Method D to give the title compound.

Yield: 395 mg (55%).

LC/MS tᵣ 1.97 nm.

MS (ES+) m/z 626, 624 (M−C(CH₃)₃+H).

### 3-Chloro-4,7-difluoro-benzo[b]thiophene-2-carboxylic acid (3′-cyano-4-methoxy-biphenyl-3-ylmethyl)-(4-methylamino-cyclohexyl)-amide hydrochloride (110)

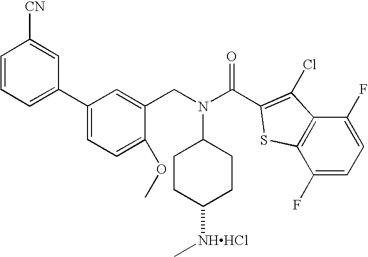

tert-Butyl carbamate 109 (395 mg, 0.58 mmol) was deprotected using Method F. On removal of the solvents in vacuo, the residue was dissolved in water (15 mL) and washed with TBME (3×10 mL). Reduction of the aqueous phase in vacuo afforded the title product.

Yield: 340 mg (95%).

LC/MS tᵣ 1.46 min.

MS (ES+) m/z 582, 580 (M+H).

### Synthesis of Compound R8

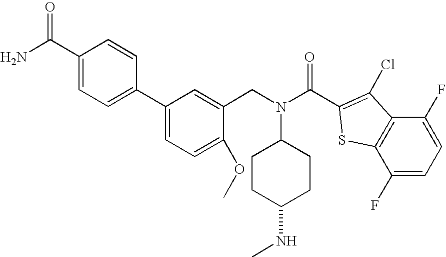

### tert-Butyl {4-[(4′-carbamoyl-4-methoxy-biphenyl-3-ylmethyl)-(3-chloro-4,7-difluoro-benzo[b]thiophene-2-carbonyl)-amino]-cyclohexyl}-methyl-carbamate (111)

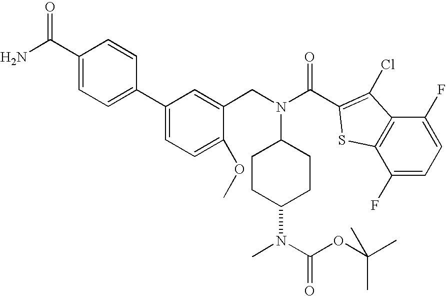

Biaryl amine 55 (80 mg, 0.17 mmol) was treated with acid chloride 8 (55 mg, 0.21 mmol) using Method D to give the title compound.

Yield: 50 mg (46%).

LC/MS tᵣ 1.88 min.

MS (ES+) m/z 644, 642 (M−C(CH₃)₃+H).

### 3-Chloro-4,7-difluoro-benzo[b]thiophene-2-carboxylic acid (4′-carbamoyl-4-methoxy-biphenyl-3-ylmethyl)-(4-methylamino-cyclohexyl)-amide hydrochloride (112)

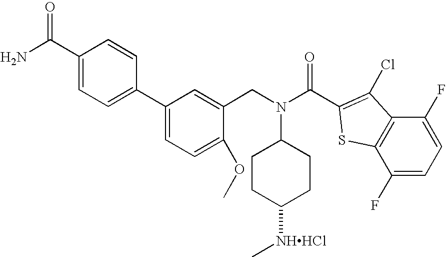

tert-Butyl carbamate 111 (50 mg, 72 μmol) was deprotected using Method F to afford the title compound.

Yield: 40 mg (88%).

LC/MS tᵣ 1.26 min.

MS (ES+) m/z 600, 598 (M+H), 569, 567 (M−31+H).

### Synthesis of Compound 338

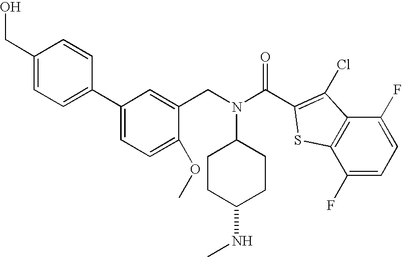

### tert-Butyl {4-[(3-chloro-4,7-difluoro-benzo[b]thiophene-2-carbonyl)-(4′-formyl-4-methoxy-biphenyl-3-ylmethyl)-amino]-cyclohexyl}-methyl-carbamate (113)

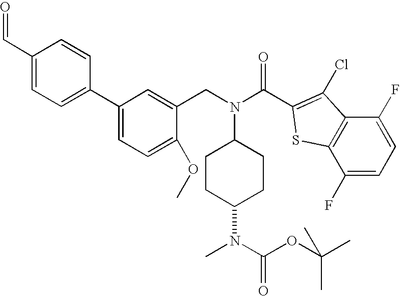

Boronic acid 9 (600 mg, 0.96 mmol) was coupled to 4-bromobenzaldehyde (213 mg, 1.15 mmol) using Method A to give the title compound.

Yield: 338 mg (52%).

LC/MS tᵣ 1.95 min.

MS (ES+) m/z 685, 683 (M+H), 629, 627 (M−C(CH₃)₃+H).

### 3-Chloro-4,7-difluoro-benzo[b]thiophene-2-carboxylic acid (4′-formyl-4-methoxy-biphenyl-3-ylmethyl)-(4-methylamino-cyclohexyl)-amide (114)

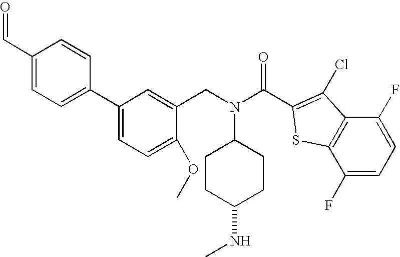

tert-Butyl carbamate 113 (338 mg, 0.49 mmol) was deprotected using Method F. However, the insolubility of the starting material made it difficult to achieve full removal of the BOC-group. After stirring 4 h at RT, further portions of conc. HCl (3 mL), EtOH (18 mL) and DCM (4 mL) were added and the reaction stirred 1 h. 1,4-Dioxane (20 mL) was then added and the reaction stirred another hour. The reaction mixture was then reduced in vacuo and aqueous NaHCO₃ (50 mL) added. The aqueous phase was extracted with DCM (3×50 mL) and the combined DCM phases dried (Na₂SO₄) and reduced in vacuo to give the title compound.

Yield: 315 mg (110%), containing ca. 38% unreacted starting material.

LC/MS tᵣ 1.44 min.

MS (ES+) m/z 585, 583 (M+H).

### 3-Chloro-4,7-difluoro-benzo[b]thiophene-2-carboxylic acid (4′-hydroxymethyl-4-methoxy-biphenyl-3-ylmethyl)-(4-methylamino-cyclohexyl)-amide trifluoroacetate (115)

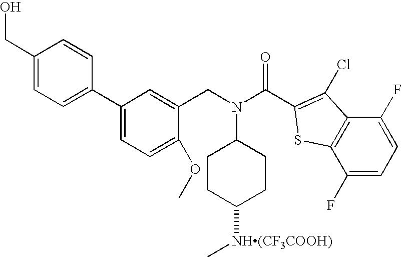

A stirred solution of crude aldehyde 114 (315 mg, ca. 0.49 mmol) in MeOH (10 mL) was treated with sodium borohydride (23 mg, 0.59 mmol) in one portion at 0° C. The reaction mixture was then warmed to RT and stirred 1 h. On reduction in vacuo, the white residue was suspended in water (50 mL) and extracted into DCM (3×25 mL). The combined DCM phases were then dried (Na₂SO₄) and reduced in vacuo. Purification was then attempted by column chromatography (gradient elution—5-10% MeOH in DCM with 0.5% triethylamine).

The purest fractions from this column were combined, converted to the TFA salt using Method J then dissolved in the minimum amount of DCM and added dropwise to cold (0° C.) TBME (25 mL). The resultant precipitate was isolated by filtration and dried to afford the title compound (46 mg, 16%).

The cruder fractions from the column were combined and further purified by preparative HPLC to give more of the title compound (43 mg, 15%).

LC/MS tᵣ 1.43 min.

MS (ES+) m/z 587, 585 (M+H).

¹H NMR δ_{H} ppm (400 MHz, D₆-DMSO, 95° C.): 8.60 (2H, br. s), 7.70-7.61 (4H, m), 7.54-7.40 (2H, m), 7.53 (2H, d), 7.19 (1H, d), 5.41 (1H, s), 4.82 (2H, s), 4.70 (2H, s), 4.08-3.93 (1H, obsc. m), 3.98 (3H, s), 3.06-2.95 (1H, obsc. m), 2.63 (3H, obsc. s), 2.27-2.18 (2H, m), 2.06-1.89 (4H, m), 1.59-1.46 (2H, m).

### Synthesis of Compound 339

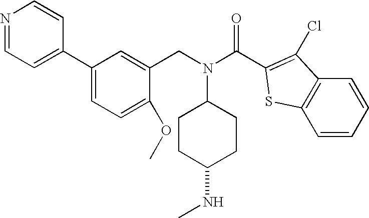

### tert-Butyl {4-[(3-Chloro-benzo[b]thiophene-2-carbonyl)-(2-methoxy-5-pyridin-4-yl-benzyl)-amino]-cyclohexyl}-methyl-carbamate (116)

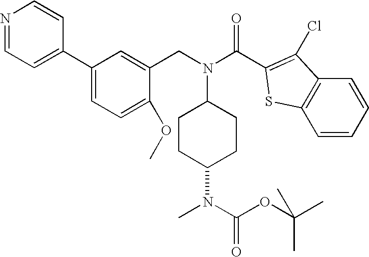

Boronic acid 5 (350 mg, 0.59 mmol) was coupled to 4-bromopyridine hydrochloride (115 mg, 0.59 mmol) using Method B to give the title compound.

Yield: 75 mg (20%).

LC/MS tᵣ 1.61 min.

MS (ES+) m/z 622, 620 (M+H), 566, 564 (M−C(CH₃)₃+H).

### 3-Chloro-benzo[b]thiophene-2-carboxylic acid (2-methoxy-5-pyridin-4-yl-benzyl)-(4-methylamino-cyclohexyl)-amide dihydrochloride (117)

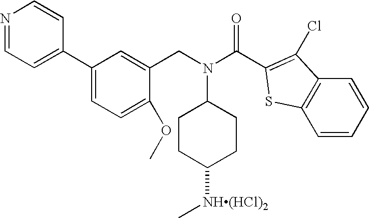

tert-Butyl carbamate 116 (75 mg, 0.12 mmol) was deprotected using Method E. On removal of the solvent in vacuo, the residue obtained was dissolved in water (5 mL) and washed with TBME (3×5 mL). Reduction of the aqueous phase in vacuo afforded the title product.

Yield: 55 mg (87%).

LC/MS tᵣ 1.07 min.

MS (ES+) m/z 522, 520 (M+H).

### Synthesis of Compound 340

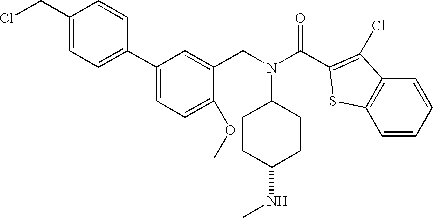

### 3-Chloro-benzo[b]thiophene-2-carboxylic acid (4′-chloromethyl-4-methoxy-biphenyl-3-ylmethyl)-(4-methylamino-cyclohexyl)-amide hydrochloride (118)

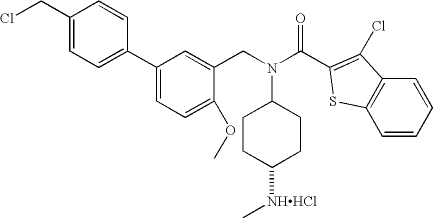

tert-Butyl carbamate 84 (10 mg, 15 μmol) was treated with HCl using Method F to afford the title compound.

Yield: 5 mg (55%).

LC/MS tᵣ 1.50 min.

MS (ES+) m/z 571, 569, 567 (M+H).

### Synthesis of Compound 341

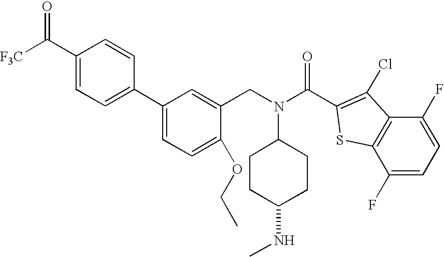

### tert-Butyl (4-{[4-ethoxy-4′-(2,2,2-trifluoro-1,1-dihydroxy-ethyl)-biphenyl-3-ylmethyl]-amino}-cyclohexyl)-methyl-carbamate (119)

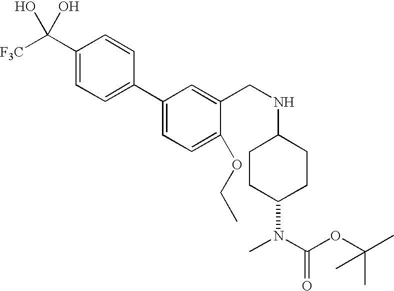

Boronic acid 11 (609 mg, 1.50 mmol) was coupled to 1-(4-bromophenyl)-2,2,2-trifluoroethanone (379 mg, 1.50 mmol) using Method B in two equal batches to give the title compound.

Yield: 487 mg (59%).

### tert-Butyl (4-{(3-chloro-4,7-difluoro-benzo[b]thiophene-2-carbonyl)-[4-ethoxy-4′-(2,2,2-trifluoro-acetyl)-biphenyl-3-ylmethyl]-amino}-cyclohexyl)-methyl-carbamate (120)

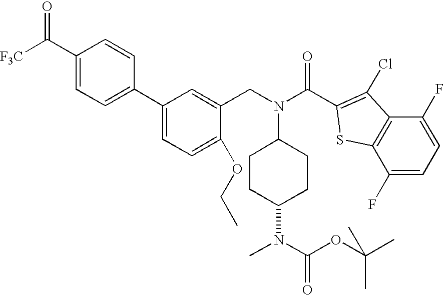

Biaryl amine 119 (487 mg, 0.88 mmol) was treated with acid chloride 8 (235 mg, 0.88 mmol) using Method D to give the title compound.

Yield: 248 mg (36%).

LC/MS tᵣ 2.15 min. The hydrate appears at 1.92 min.

MS (ES+) m/z 785, 783 (M+H₂O+H), 767, 765 (M+H), 729, 727 (M−C(CH₃)₃+H₂O+H), 711, 709 (M−C(CH₃)₃+H).

### 3-Chloro-4,7-difluoro-benzo[b]thiophene-2-carboxylic acid [4-ethoxy-4′-(2,2,2-trifluoro-acetyl)-biphenyl-3-ylmethyl]-(4-methylamino-cyclohexyl)-amide trifluoroacetate (121)

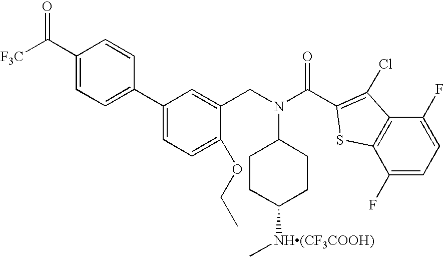

tert-Butyl carbamate 120 (248 mg, 0.32 mmol) was deprotected using Method F then purified by column chromatography (20% MeOH in EtOAc with 0.25% conc. ammonia) followed by preparative HPLC to afford the title compound.

Yield: 113 mg (45%).

LC/MS tᵣ 1.74 min. The hydrate appears at 1.46 min.

MS (ES+) m/z 685, 683 (M+H₂O+H), 667, 665 (M+H), 654, 652 (M−31+H₂O+H), 636, 634 (M−31+H).

### Synthesis of Compound 342

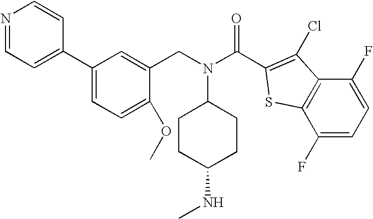

### tert-Butyl {4-[(3-chloro-4,7-difluoro-benzo[b]thiophene-2-carbonyl)-(2-methoxy-5-pyridin-4-yl-benzyl)-amino]-cyclohexyl}-methyl-carbamate (122)

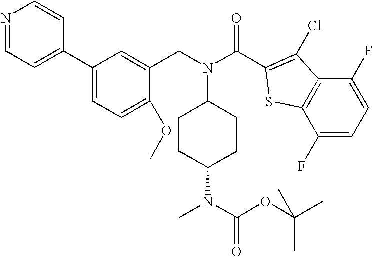

Boronic acid 9 (5.70 g, 9.14 mmol) was coupled to 4-bromopyridine hydrochloride (2.13 g, 11.0 mmol) using Method A to give the title compound.

Yield: 5.71 g (95%). Contains triphenylphosphine oxide (ca. 29%).

LC/MS tᵣ 1.59 min.

MS (ES+) m/z 658, 656 (M+H).

### 3-Chloro-4,7-difluoro-benzo[b]thiophene-2-carboxylic acid (2-methoxy-5-pyridin-4-yl-benzyl)-(4-methylamino-cyclohexyl)-amide dihydrochloride (123)

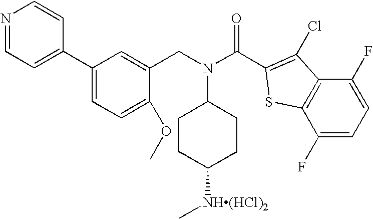

tert-Butyl carbamate 122 (5.71 g, 8.70 mmol) was deprotected using Method F. On removal of the solvents in vacuo, the reaction mixture was dissolved in DCM (250 mL) and extracted into 2 M HCl (4×50 mL). The combined HCl phases were washed with DCM (2×50 mL) then taken to pH 9 by careful addition of solid NaHCO₃. The resultant aqueous suspension was then extracted into DCM (3×100 mL), the combined DCM phases dried (Na₂SO₄) and reduced in vacuo. This residue was purified by chromatography (gradient elution—5-10% MeOH in DCM with 0.5% triethylamine) then converted to the title compound by Method H.

Yield: 4.0 g (78%).

LC/MS tᵣ 1.08 min.

MS (ES+) m/z 558, 556 (M+H), 279, 278 [(M+H)/2].

¹H NMR δ_{H} ppm (400 MHz, D₆-DMSO, 95° C.): 8.92 (2H, br. s), 8.61 (2H, d), 7.85 (2H, d), 7.72 (1H, d), 7.60 (1H, s), 7.26-7.12 (2H, m), 7.04 (1H, d), 4.55 (2H, s), 3.82-3.68 (1H, obsc. m), 3.75 (3H, s), 2.74 (1H, br. s), 2.29 (3H, s), 2.02-1.92 (2H, m), 1.80-1.58 (4H, m), 1.36-1.16 (2H, m).

### Synthesis of Compound 343

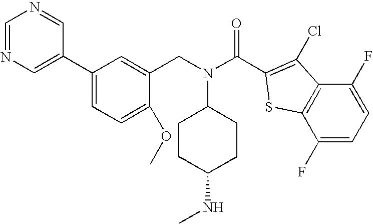

### tert-Butyl {4-[(3-chloro-4,7-difluoro-benzo[b]thiophene-2-carbonyl)-(2-methoxy-5-pyrimidin-5-yl-benzyl)-amino]-cyclohexyl}-methyl-carbamate (124)

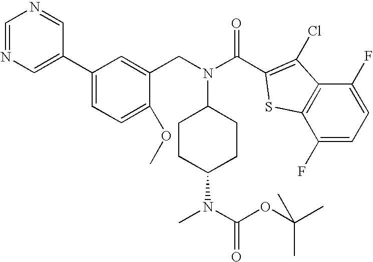

Boronic acid 9 (275 mg, 0.44 mmol) was coupled to 5-bromopyrimidine (79 mg, 0.48 mmol) using Method B to give the title compound.

Yield: 178 mg (61%).

LC/MS tᵣ 1.73 min.

MS (ES+) m/z 559, 557 (M−CO₂C(CH₃)₃+H).

### 3-Chloro-4,7-difluoro-benzo[b]thiophene-2-carboxylic acid (2-methoxy-5-pyrimidin-5-yl-benzyl)-(4-methylamino-cyclohexyl)-amide dihydrochloride (125)

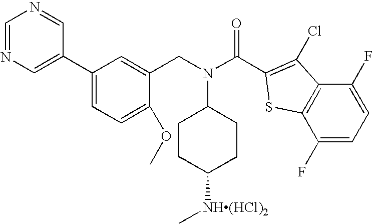

tert-Butyl carbamate 124 (178 mg, 0.27 mmol) was deprotected using Method E to give the title compound.

Yield: 146 mg (86%).

LC/MS tᵣ 1.23 min.

MS (ES+) m/z 559, 557 (M+H).

### Synthesis of Compound 344

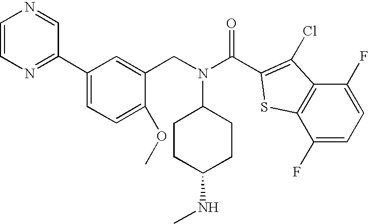

### tert-Butyl {4-[(3-chloro-4,7-difluoro-benzo[b]thiophene-2-carbonyl)-(2-methoxy-5-pyrazin-2-yl-benzyl)-amino]-cyclohexyl}-methyl-carbamate (126)

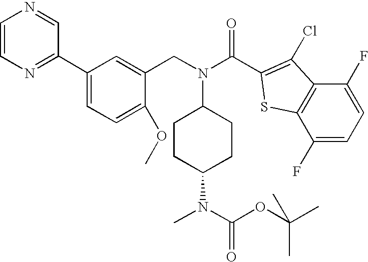

Boronic acid 9 (201 mg, 0.32 mmol) was coupled to 2-chloropyrazine (50 μL, 0.35 mmol) using Method B to give the title compound.

Yield: 129 mg (61%).

LC/MS tᵣ 1.86 min.

MS (ES+) m/z 659, 657 (M+H), 603, 601 (M−C(CH₃)₃+H).

### 3-Chloro-4,7-difluoro-benzo[b]thiophene-2-carboxylic acid (2-methoxy-5-pyrazin-2-yl-benzyl)-(4-methylamino-cyclohexyl)-amide dihydrochloride (127)

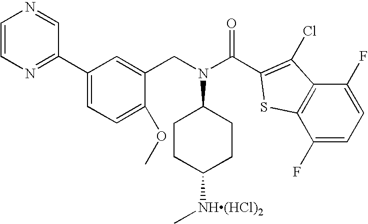

tert-Butyl carbamate 126 (129 mg, 0.19 mmol) was deprotected using Method F. Purification by column chromatography (20% MeOH in EtOAc with 2% triethylamine) followed by formation of the HCl salt by Method H gave the title compound.

Yield: 50 mg (42%).

LC/MS tᵣ 1.30 min.

MS (ES+) m/z 559, 557 (M+H).

### Synthesis of Compound 345

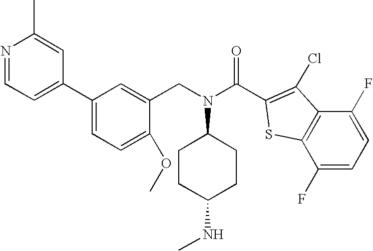

### tert-Butyl (4-{(3-chloro-4,7-difluoro-benzo[b]thiophene-2-carbonyl)-[2-methoxy-5-(2-methyl-pyridin-4-yl)-benzyl]-amino}-cyclohexyl)-methyl-carbamate (128)

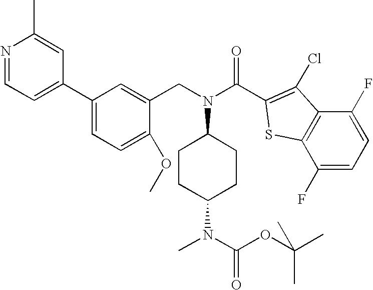

Crude biaryl amine 88 (1.96 g, 4.45 mmol) was treated with acid chloride 8 (1.43 g, 5.35 mmol) using Method D to give the title compound.

Yield: 784 mg (26%).

LC/MS tᵣ 1.62 min.

MS (ES+) m/z 672, 670 (M+H).

### 3-Chloro-4,7-difluoro-benzo[b]thiophene-2-carboxylic acid [2-methoxy-5-(2-methyl-pyridin-4-yl)-benzyl]-(4-methylamino-cyclohexyl)-amide dihydrochloride (129)

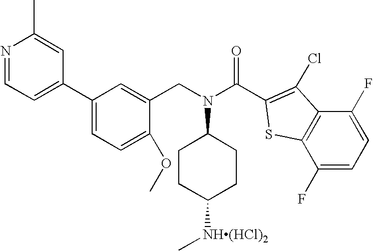

tert-Butyl carbamate 128 (784 mg, 1.17 mmol) was deprotected using Method F. On removal of the solvents in vacuo, the residue was dissolved in water (50 mL) and washed with EtOAc (3×25 mL). The aqueous phase was then reduced in vacuo to afford the title compound.

Yield: 694 mg (92%).

LC/MS tᵣ 1.14 min.

MS (ES+) m/z 572, 570 (M+H).

### Synthesis of Compound 346

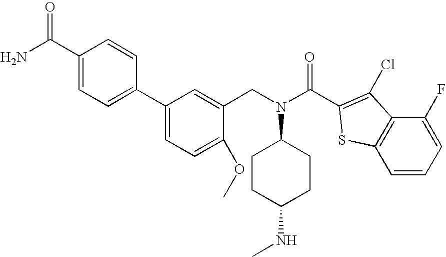

### tert-Butyl {4-[(4′-carbamoyl-4-methoxy-biphenyl-3-ylmethyl)-(3-chloro-4-fluoro-benzo[b]thiophene-2-carbonyl)-amino]-cyclohexyl}-methyl-carbamate (130)

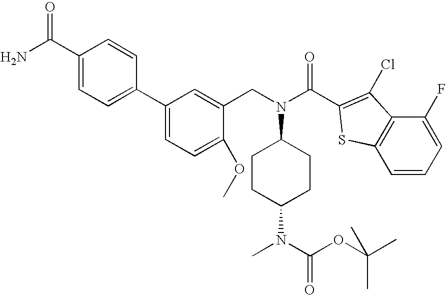

Biaryl amine 55 (216 mg, 0.46 mmol) was treated with acid chloride 6 (137 mg, 0.55 mmol) using Method D to give the title compound.

Yield: 105 mg (34%).

LC/MS tᵣ 1.72 min.

MS (ES+) m/z 626, 624 (M−C(CH₃)₃+H), 582, 580 (M−CO₂C(CH₃)₃+H).

### 3-Chloro-4-fluoro-benzo[b]thiophene-2-carboxylic acid (4′-carbamoyl-4-methoxy-biphenyl-3-ylmethyl)-(4-methylamino-cyclohexyl)-amide hydrochloride (131)

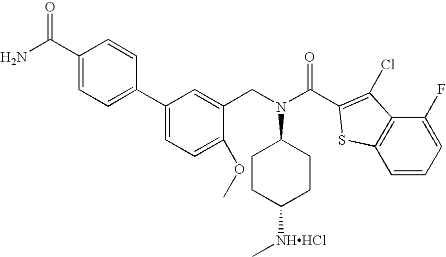

tert-Butyl carbamate 130 (105 mg, 0.15 mmol) was deprotected using Method F to give the title compound.

Yield: 49 mg (55%).

LC/MS tᵣ 1.21 min.

MS (ES+) m/z 582, 580 (M+H), 551, 549 (M−31+H).

### Synthesis of Compound 347

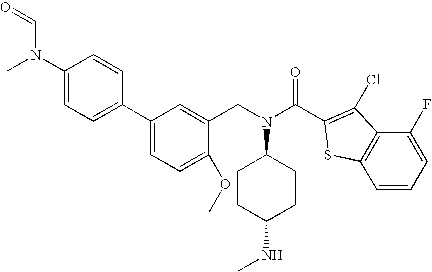

### tert-Butyl (4-{(3-chloro-4-fluoro-benzo[b]thiophene-2-carbonyl)-[4′-(formyl-methyl-amino)-4-methoxy-biphenyl-3-ylmethyl]-amino}-cyclohexyl)-methyl-carbamate (132)

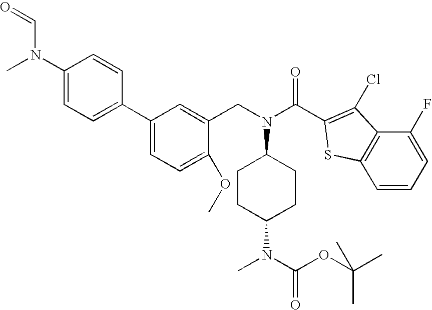

Biaryl amine 52 (200 mg, 0.42 mmol) was treated with acid chloride 6 (120 mg, 0.49 mmol) using Method D to afford the title compound.

Yield: 190 mg (66%).

LC/MS tᵣ 1.88 min.

MS (ES+) m/z 640, 638 (M−C(CH₃)₃+H).

### 3-Chloro-4-fluoro-benzo[b]thiophene-2-carboxylic acid [4′-(formyl-methyl-amino)-4-methoxy-biphenyl-3-ylmethyl]-(4-methylamino-cyclohexyl)-amide trifluoroacetate (133)

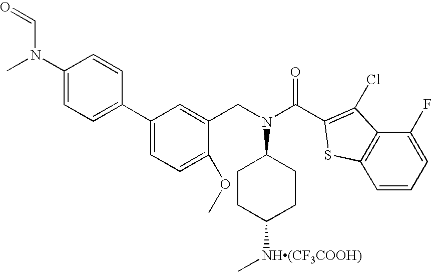

tert-Butyl carbamate 132 (190 mg, 0.27 mmol) was deprotected using Method G. The reaction mixture was then diluted with water (10 mL) and the DCM removed in vacuo to give a precipitate in the water layer. Acetonitrile (5 mL) was added to dissolve the solid and the resulting solution was lyophilised to give the title compound.

Yield: 180 mg (quant.).

LC/MS tᵣ 1.36 min.

MS (ES+) m/z 596, 594 (M+H).

### Synthesis of Compound 348

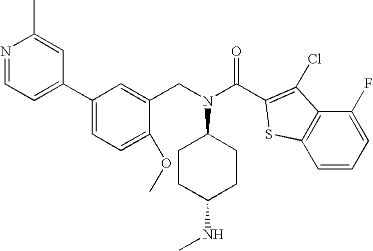

### tert-Butyl (4-{(3-chloro-4-fluoro-benzo[b]thiophene-2-carbonyl)-[2-methoxy-5-(2-methyl-pyridin-4-yl)-benzyl]-amino}-cyclohexyl)-methyl-carbamate (134)

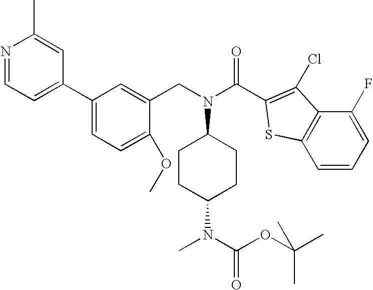

Biaryl amine 88 (160 mg, 0.36 mmol) was treated with acid chloride 6 (99 mg, 0.40 mmol) using Method D to give the title compound.

Yield: 140 mg (61%).

LC/MS tᵣ 1.59 min.

MS (ES+) m/z 654, 652 (M+H).

### 3-Chloro-4-fluoro-benzo[b]thiophene-2-carboxylic acid [2-methoxy-5-(2-methyl-pyridin-4-yl)-benzyl]-(4-methylamino-cyclohexyl)-amide bis(trifluoroacetate) (135)

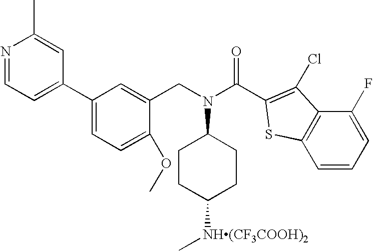

tert-Butyl carbamate 134 (140 mg, 0.22 mmol) was deprotected using Method G to give the title compound.

Yield: 167 mg (quant.).

LC/MS tᵣ 1.12 min.

MS (ES+) m/z 554, 552 (M+H).

### Synthesis of Compound 349

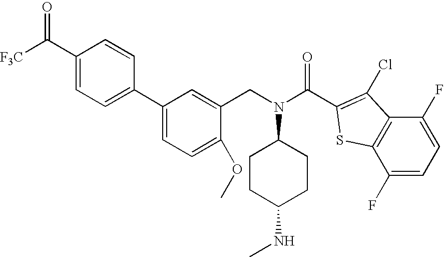

### tert-Butyl (4-{(3-chloro-4,7-difluoro-benzo[b]thiophene-2-carbonyl)-[4-methoxy-4′-(2,2,2-trifluoro-acetyl)-biphenyl-3-ylmethyl]-amino}-cyclohexyl)-methyl-carbamate (136)

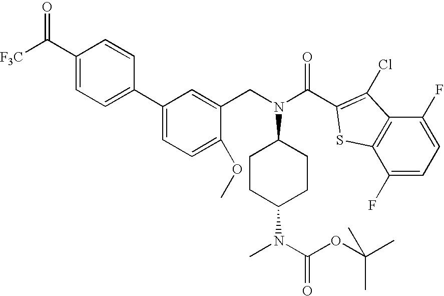

Boronic acid 9 (250 mg, 0.40 mmol) was coupled to 4′-bromo-2,2,2-trifluoroacetophenone (120 mg, 0.48 mmol) using Method B to give the title compound.

Yield: 230 mg (76%).

LC/MS tᵣ 1.88 min.

MS (ES+) m/z 771, 769 (M+H₂O+H), 697, 695 (M−C(CH₃)₃+H).

### 3-Chloro-4,7-difluoro-benzo[b]thiophene-2-carboxylic acid [4-methoxy-4′-(2,2,2-trifluoro-acetyl)-biphenyl-3-ylmethyl]-(4-methylamino-cyclohexyl)-amide trifluoroacetate (137)

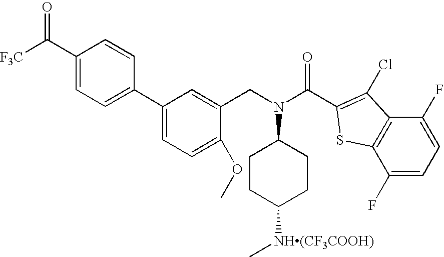

tert-Butyl carbamate 136 (170 mg, 0.23 mmol) was deprotected using Method E. Purification by preparative HPLC then gave the title compound as the TFA salt.

Yield: 92 mg (62%).

LC/MS tᵣ 1.39 min.

MS (ES+) m/z 671, 669 (M+H₂O+H), 653, 651 (M+H).

### Synthesis of Compound 350

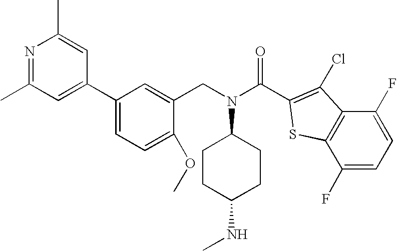

### tert-Butyl (4-{(3-chloro-4,7-difluoro-benzo[b]thiophene-2-carbonyl)-[5-(2,6-dimethyl-pyridin-4-yl)-2-methoxy-benzyl]-amino}-cyclohexyl)-methyl-carbamate (138)

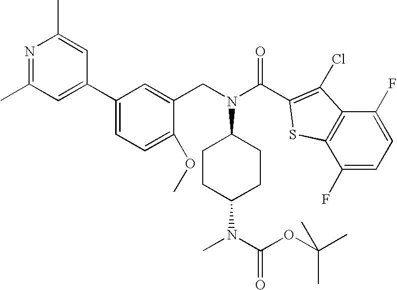

Biaryl amine 92 (268 mg, 0.59 mmol) was treated with acid chloride 8 (205 mg, 0.77 mmol) using Method D to afford the title compound.

Yield: 101 mg (25%).

LC/MS tᵣ 1.68 min.

MS (ES+) m/z 686, 684 (M+H).

### 3-Chloro-4,7-difluoro-benzo[b]thiophene-2-carboxylic acid [5-(2,6-dimethyl-pyridin-4-yl)-2-methoxy-benzyl]-(4-methylamino-cyclohexyl)-amide dihydrochloride (139)

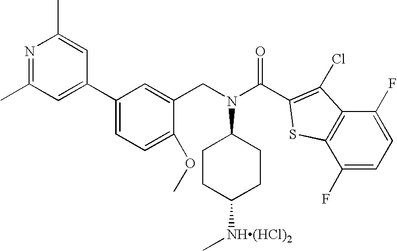

tert-Butyl carbamate 138 (101 mg, 0.15 mmol) was deprotected using Method F to give the title compound.

Yield: 63 mg (73%).

LC/MS tᵣ 1.12 min.

MS (ES+) m/z 586, 584 (M+H).

### Synthesis of Compound 351

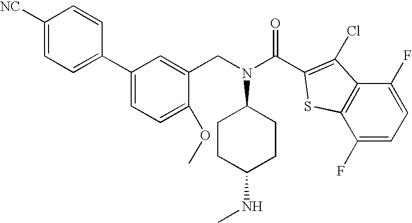

### tert-Butyl {4-[(3-chloro-4,7-difluoro-benzo[b]thiophene-2-carbonyl)-(4′-cyano-4-methoxy-biphenyl-3-ylmethyl)-amino]-cyclohexyl}-methyl-carbamate (140)

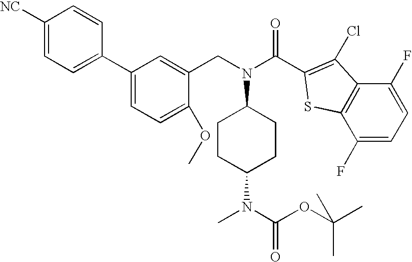

Biaryl amine 13 (300 mg, 0.70 mmol) was treated with acid chloride 8 (214 mg, 0.80 mmol) using Method D to afford the title compound.

Yield: 355 mg (75%).

LC/MS tᵣ 1.94 min.

MS (ES+) m/z 626, 624 (M−C(CH₃)₃+H).

### 3-Chloro-4,7-difluoro-benzo[b]thiophene-2-carboxylic acid (4′-cyano-4-methoxy-biphenyl-3-ylmethyl)-(4-methylamino-cyclohexyl)-amide hydrochloride (141)

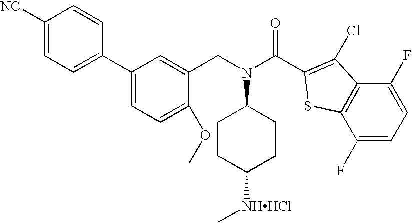

tert-Butyl carbamate 140 (350 mg, 0.50 mmol) was deprotected using Method F to give the title compound.

Yield: 308 mg (quant.).

LC/MS (10 min) tᵣ 5.79 min.

MS (ES+) m/z 623, 621 (M+CH₃CN+H), 582, 580 (M+H).

### Synthesis of Compound 352

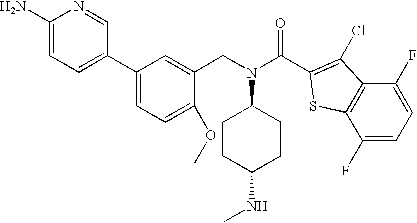

### tert-Butyl {4-[5-(6-amino-pyridin-3-yl)-2-methoxy-benzylamino]-cyclohexyl}-methyl-carbamate (142)

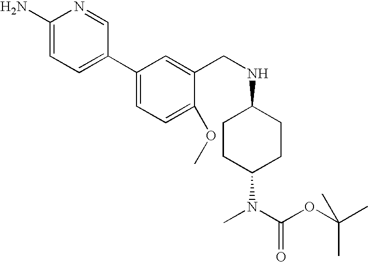

Boronic acid 4 (900 mg, 2.29 mmol) was coupled to 2-amino-5-bromopyridine (475 mg, 2.74 mmol) using Method A. On completion, the cooled reaction mixture was diluted with 1 M HCl (10 mL) and extracted with TBME (3×10 mL). The aqueous phase was then basified to pH 9 by careful addition of solid NaHCO₃ and extracted into EtOAc (50 mL) and DCM (3×50 mL). The combined organic phases were dried over Na₂SO₄ and reduced in vacuo to yield the crude title product.

Yield: 470 mg (47%).

### tert-Butyl {4-[[5-(6-amino-pyridin-3-yl)-2-methoxy-benzyl]-(3-chloro-4,7-difluorobenzo[b]thiophene-2-carbonyl)-amino]-cyclohexyl}-methyl-carbamate (143)

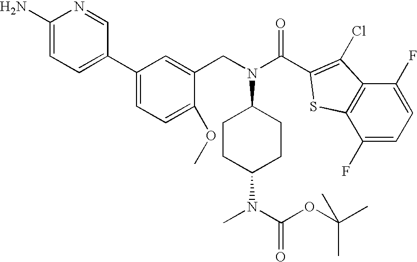

Crude biaryl amine 142 (300 mg, 0.68 mmol) was treated with acid chloride 8 (182 mg, 0.68 mmol) using Method D to give the title compound.

Yield: 120 mg (26%).

### 3-Chloro-4,7-difluoro-benzo[b]thiophene-2-carboxylic acid [5-(6-amino-pyridin-3-yl)-2-methoxy-benzyl]-(4-methylamino-cyclohexyl)-amide dihydrochloride (144)

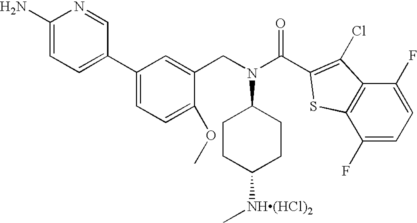

tert-Butyl carbamate 143 (120 mg, 0.18 mmol) was deprotected using Method F. On removal of the solvents in vacuo, the residue was dissolved in water (15 mL) and washed with TBME (3×10 mL). Reduction of the aqueous phase in vacuo afforded the title product.

Yield: 72 mg (62%).

LC/MS tᵣ 1.16 min.

MS (ES+) m/z 573, 571 (M+H).

### Synthesis of Compound 353

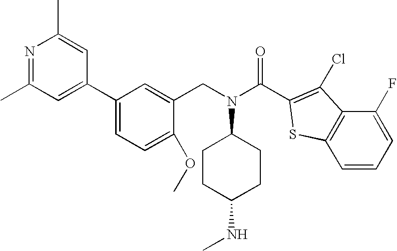

### tert-Butyl (4-{(3-chloro-4-fluoro-benzo[b]thiophene-2-carbonyl)-[5-(2,6-dimethyl-pyridin-4-yl)-2-methoxy-benzyl]-amino}-cyclohexyl)-methyl-carbamate (145)

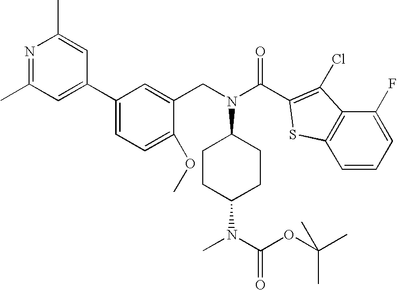

Biaryl amine 92 (62 mg, 0.14 mmol) was treated with acid chloride 6 (51 mg, 0.21 mmol) using Method D to afford the title compound.

Yield: 30 mg (33%).

LC/MS tᵣ 1.58 min.

MS (ES+) m/z 668, 666 (M+H).

### 3-Chloro-4-fluoro-benzo[b]thiophene-2-carboxylic acid [5-(2,6-dimethyl-pyridin-4-yl)-2-methoxy-benzyl]-(4-methylamino-cyclohexyl)-amide dihydrochloride (146)

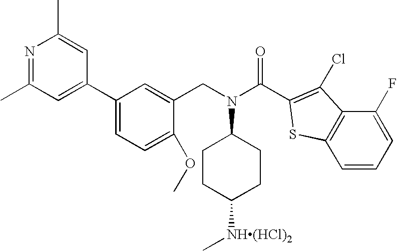

tert-Butyl carbamate 145 (10 mg, 0.02 mmol) was deprotected using Method F to give the title compound.

Yield: 10 mg (quant.).

LC/MS t, 1.08 min.

MS (ES+) m/z 558, 556 (M+H).

### Synthesis of Compound R5

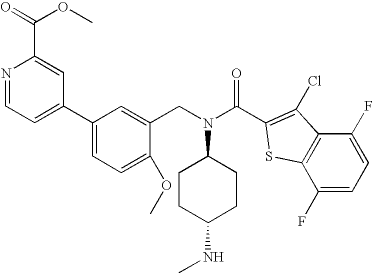

### Methyl 4-bromopyridine-2-carboxylate (147)

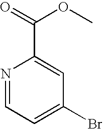

A solution of 4-bromopyridine hydrochloride (595 mg, 3.06 mmol) in DCM (20 mL) was washed with aqueous NaHCO₃ (2×20 mL), dried (MgSO₄) and filtered. The filtrate was made up to 45 mL by the addition of more DCM, then water (3 mL) was added, followed by iron(II) sulphate heptahydrate (8.51 g, 30.6 mmol) and conc. H₂SO₄ (0.95 mL, 9.18 mmol). In a separate flask, methyl pyruvate (4.15 mL, 46 mmol) was treated with hydrogen peroxide (3.5 mL, 30.6 mmol, 30% solution in water) at −10° C., then this solution was added to the DCM/water mixture at −10° C. with vigorous stirring. After 15 minutes, the reaction was diluted with iced water (100 mL) and extracted into DCM (4×20 mL). The combined DCM phases were dried (MgSO₄) and removed in vacuo. The title compound was obtained after sequential column chromatography (gradient elution—10-40% EtOAc in heptane with 0.5% triethylamine, then repeating with 0-20% EtOAc in heptane with 0.5% triethylamine).

Yield: 211 mg (32%).

LC/MS tᵣ 0.98 min.

MS (ES+) m/z 218, 216 (M+H).

### Methyl 4-(3-{[[4-(tert-butoxycarbonyl-methyl-amino)-cyclohexyl]-(3-chloro-4,7-difluoro-benzo[b]thiophene-2-carbonyl)-amino]-methyl}-4-methoxy-phenyl)-pyridine-2-carboxylate (148)

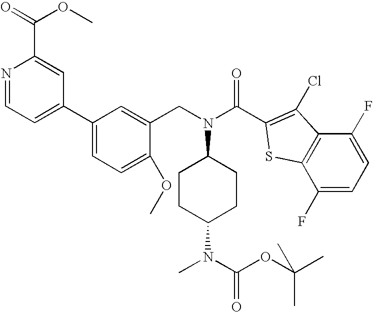

Boronic acid 9 (748 mg, 1.20 mmol) was coupled to pyridyl bromide 147 (258 mg, 1.20 mmol) using Method A. However, the reaction temperature was lowered to 87° C. to avoid hydrolysis of the methyl ester. In addition, EtOH was replaced by MeOH and was used in a 1:30 ratio to toluene.

Yield: 247 mg (29%), containing ca. 37% triphenylphosphine oxide.

LC/MS tᵣ 1.77 min.

MS (ES+) m/z 716, 714 (M+H).

### Methyl 4-(3-{[(3-chloro-4,7-difluoro-benzo[b]thiophene-2-carbonyl)-(4-methylamino-cyclohexyl)-amino]-methyl}-4-methoxy-phenyl)-pyridine-2-carboxylate (149)

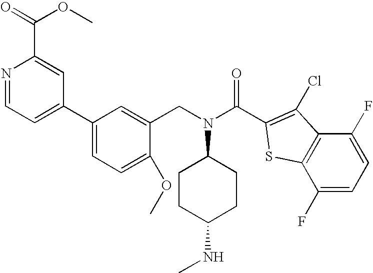

Acetyl chloride (5 mL) was added to anhydrous MeOH (10 mL) at 0° C. tert-Butyl carbamate 148 (247 mg, 0.35 mmol) was then treated with this solution and stirred 2 h. On removal of the solvents in vacuo, the reaction mixture was dissolved in DCM (25 mL) and extracted into 2 M HCl (4×10 mL). The combined HCl phases were washed with DCM (2×20 mL) then taken to pH 9 by careful addition of solid NaHCO₃. The resultant aqueous suspension was then extracted into DCM (3×20 mL), the combined DCM phases dried (Na₂SO₄) and reduced in vacuo to afford the title compound.

Yield: 110 mg (51%).

LC/MS tᵣ 1.29 min.

MS (ES+) m/z 616, 614 (M+H).

### Synthesis of Compound 354

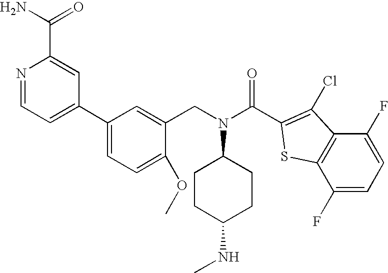

### tert-Butyl {4-[[5-(2-carbamoyl-pyridin-4-yl)-2-methoxy-benzyl]-(3-chloro-4,7-difluoro-benzo[b]thiophene-2-carbonyl)-amino]-cyclohexyl}-methyl-carbamate (150)

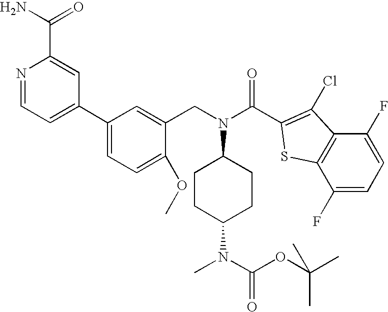

A solution of methyl ester 148 (50 mg, 0.07 mmol) in 1,4-dioxane (2 mL) was treated with conc. ammonia (1 mL) and the reaction stirred at RT 6 h. LC/MS at this juncture showed some residual starting material so more conc. ammonia (1 mL) was added and the reaction mixture heated 2 h at 40° C. LC/MS at this juncture showed the reaction was complete. The solvents were removed in vacuo to give the title compound.

Yield: 45 mg (93%).

LC/MS tᵣ 1.74 min.

MS (ES+) m/z 701, 699 (M+H), 645, 643 (M−C(CH₃)₃+H).

### 4-(3-{[(3-Chloro-4,7-difluoro-benzo[b]thiophene-2-carbonyl)-(4-methylamino-cyclohexyl)-amino]-methyl}-4-methoxy-phenyl)-pyridine-2-carboxylic acid amide dihydrochloride (151)

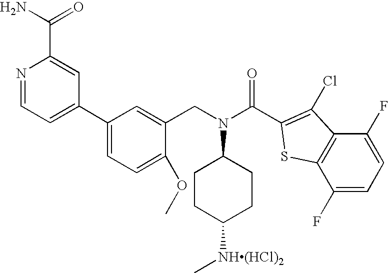

tert-Butyl carbamate 150 (45 mg, 0.06 mmol) was deprotected using Method F. Purification by column chromatography followed by formation of the HCl salt by Method H gave the title compound.

Yield: 27 mg (70%).

LC/MS tᵣ 1.23 min.

MS (ES+) m/z 601, 599 (M+H).

### Synthesis of Compound 355

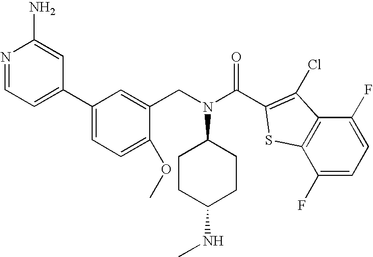

### 4-Amino-2-methylpyridine (152)

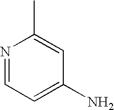

A stirred solution of 4-nitro-2-methylpyridine-N-oxide (20 g, 0.13 mol) in AcOH (300 mL) was treated with iron powder (40 g, 0.72 mol) in one portion at RT. This grey suspension was then gently heated to 100° C. and stirred 2 h. The reaction mixture was then filtered through celite, and the solids collected washed with acetonitrile (1 L). The dark brown filtrate was then reduced in vacuo, diluted with 6 M NaOH (500 mL) and extracted into TBME (4×200 mL). The TBME phases were combined, dried (MgSO₄) and reduced in vacuo to afford the title compound.

Yield: 10.1 g (72%).

LC/MS tᵣ 0.52 min.

MS (ES+) m/z 216 (2M+H).

### 4-Bromo-2-methylpyridine (153)

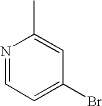

A stirred solution of amino-pyridine 152 (10.1 g, 93.4 mmol) in 48% aqueous HBr (165 mL) at −10° C. was treated with a pre-cooled (0° C.) solution of sodium nitrite (7.04 g, 0.102 mol) in water (165 mL) dropwise over 0.5 h. The solution was then warmed to RT and stirred 16 h. It was then diluted with 4 M NaOH (400 mL) and extracted into TBME (4×150 mL). The TBME phases were combined, dried (MgSO₄) and reduced in vacuo to afford the title compound.

Yield: 14.8 g (92%).

LC/MS tᵣ 0.57 min.

MS (ES+) m/z 174, 172 (M+H).

### 4-Bromopyridine-2-carboxylic acid (154)

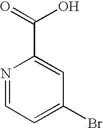

A stirred solution of pyridyl bromide 153 (5.03 g, 29.0 mmol) in water (130 mL) was treated at RT with KMnO₄ (4.72 g, 29.5 mmol) in one portion then heated to reflux 1.5 h. At this juncture more KMnO₄ was added (4.72 g, 29.5 mmol) and the solution heated at reflux a further 2 h. Another portion of KMnO₄ was then added (9.43 g, 59 mmol), the reaction heated at reflux a further 3 h then filtered whilst still hot, washing the isolated solids with boiling water (200 mL). The aqueous filtrate was then concentrated in vacuo to approximately 40 mL, acidified to pH 4 by careful addition of 1 M HCl and the resultant white precipitate isolated by filtration. The filtrate was then reduced in vacuo to approximately 10 mL and the forthcoming precipitate also isolated by filtration. Combining these two isolated precipitates gave the title compound.

Yield: 1.28 g (22%).

LC/MS tᵣ 0.67 min.

MS (ES+) m/z 204, 202 (M+H).

### tert-Butyl (4-bromo-pyridin-2-yl)-carbamate (155)

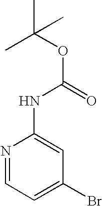

This compound was prepared from 4-bromopyridine-2-carboxylic acid (154, 600 mg, 2.97 mmol) in accordance with the procedure of Deady, Korytsky and Rowe (Deady, L. W.; Korytsky, O. L.; Rowe, J. E. Aust. J. Chem., 1982, 35, 2025-2034).

Yield: 750 mg (93%).

### tert-Butyl [4-(3-{[[4-(BOC-methyl-amino)-cyclohexyl]-(3-chloro-4,7-difluoro-benzo[b]thiophene-2-carbonyl)-amino]-methyl}-4-methoxy-phenyl)-pyridin-2-yl]-carbamate (156)

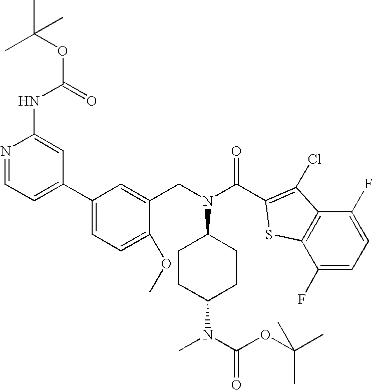

Boronic acid 9 (686 mg, 1.10 mmol) was coupled to pyridyl bromide 155 (300 mg, 1.10 mmol) using Method A to give the title compound.

Yield: 50 mg (6%).

LC/MS tᵣ 1.80 min.

MS (ES+) m/z 773, 771 (M+H), 717, 715 (M−C(CH₃)₃+H).

### 3-Chloro-4,7-difluoro-benzo[b]thiophene-2-carboxylic acid [5-(2-amino-pyridin-4-yl)-2-methoxy-benzyl]-(4-methylamino-cyclohexyl)-amide dihydrochloride (157)

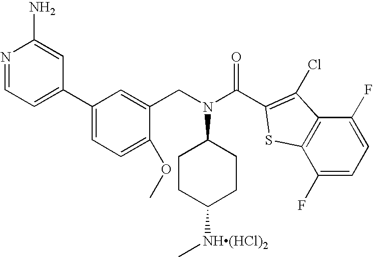

tert-Butyl carbamate 156 (50 mg, 0.06 mmol) was deprotected using Method F. Purification by column chromatography (10% MeOH in EtOAc with 2% triethylamine) followed by formation of the HCl salt using Method H gave the title compound.

Yield: 11 mg (30%).

LC/MS tᵣ 1.12 min.

MS (ES+) m/z 573, 571 (M+H).

### Synthesis of Compound 356

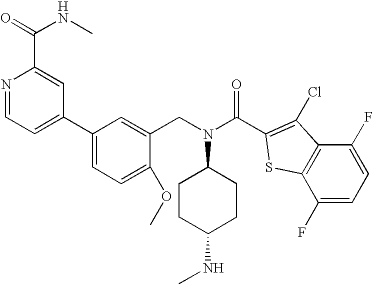

### tert-Butyl (4-{(3-chloro-4,7-difluoro-benzo[b]thiophene-2-carbonyl)-[2-methoxy-5-(2-methylcarbamoyl-pyridin-4-yl)-benzyl]-amino}-cyclohexyl)-methyl-carbamate (158)

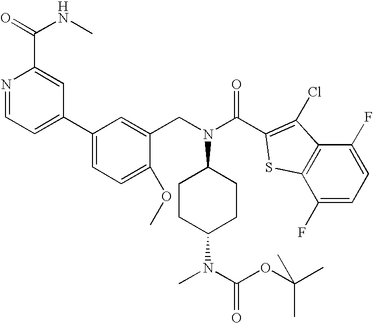

A stirred solution of methyl ester 148 (30 mg, 0.05 mmol) in MeOH (2 mL) was treated with methylamine (1 mL, 40% solution in water) at RT. After 1 h, the reaction mixture was reduced in vacuo and the residue purified by column chromatography (75% EtOAc in heptane) to give the title compound.

Yield: 25 mg (83%).

LC/MS tᵣ 1.82 min.

MS (ES+) m/z 715, 713 (M+H), 659, 657 (M−C(CH₃)₃+H), 615, 613 (M−CO₂C(CH₃)₃+H).

### 4-(3-{[Chloro-4,7-difluoro-benzo[b]thiophene-2-carbonyl)-(4-methylamino-cyclohexyl)-amino]-methyl}-4-methoxy-phenyl)-pyridine-2-carboxylic acid methylamide dihydrochloride (159)

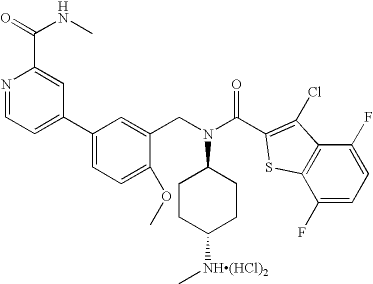

tert-Butyl carbamate 158 (25 mg, 0.04 mmol) was deprotected using Method F to give the title compound.

Yield: 16 mg (75%).

LC/MS tᵣ 1.28 min.

MS (ES+) m/z 615, 613 (M+H).

### Synthesis of Compound 357

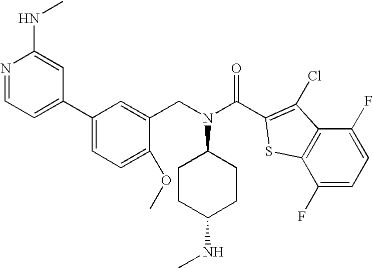

### tert-Butyl (4-bromo-pyridin-2-yl)-methyl-carbamate (160)

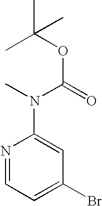

To a solution of tert-butyl carbamate 155 (300 mg, 1.10 mmol) in THF (5 mL) at 0° C. was added sodium hydride (53 mg, 1.32 mmol, 60% dispersion in mineral oil) in one portion. After stirring 15 minutes, iodomethane was added (82 μL, 1.32 mmol) and the reaction mixture warmed to RT and stirred 16 h. The reaction was then quenched with 5% citric acid (10 mL) and extracted into EtOAc (2×10 mL). The combined organic phases were dried over Na₂SO₄ and purified by column chromatography (70% EtOAc in heptane) to give the title compound.

Yield: 240 mg (76%).

LC/MS tᵣ 1.62 min.

MS (ES+) m/z 289, 287 (M+H), 233, 231 (M−C(CH₃)₃+H).

### tert-Butyl [4-(3-{[[4-(BOC-methyl-amino)-cyclohexyl]-(3-chloro-4,7-difluoro-benzo[b]thiophene-2-carbonyl)-amino]-methyl}-4-methoxy-phenyl)-pyridin-2-yl]-methyl-carbamate (161)

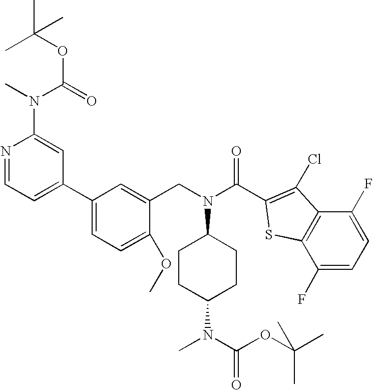

Boronic acid 9 (520 mg, 0.83 mmol) was coupled to pyridyl bromide 160 (240 mg, 0.83 mmol) using Method A to give the title compound.

Yield: 230 mg (35%).

LC/MS tᵣ 1.89 min.

MS (ES+) m/z 787, 785 (M+H), 731, 729 (M−C(CH₃)₃+H).

### 3-Chloro-4,7-difluoro-benzo[b]thiophene-2-carboxylic acid [2-methoxy-5-(2-methylamino-pyridin-4-yl)-benzyl]-(4-methylamino-cyclohexyl)-amide bis(trifluoroacetate) (162)

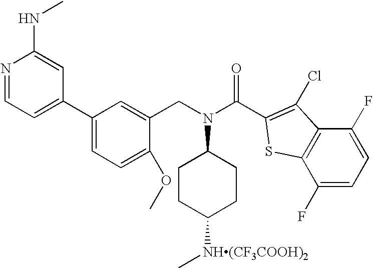

tert-Butyl carbamate 161 (130 mg, 0.17 mmol) was deprotected using Method F. Purification by column chromatography (10% MeOH in EtOAc with 2% triethylamine) followed by preparative HPLC gave the title compound as the TFA salt.

Yield: 45 mg (33%).

LC/MS tᵣ 1.15 min.

MS (ES+) m/z 587, 585 (M+H).

### Synthesis of Compound R9

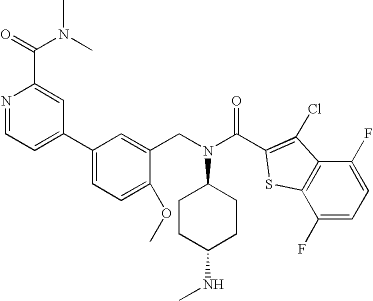

### tert-Butyl (4-{(3-chloro-4,7-difluoro-benzo[b]thiophene-2-carbonyl)-[5-(2-dimethylcarbamoyl-pyridin-4-yl)-2-methoxy-benzyl]-amino}-cyclohexyl)-methyl-carbamate (163)

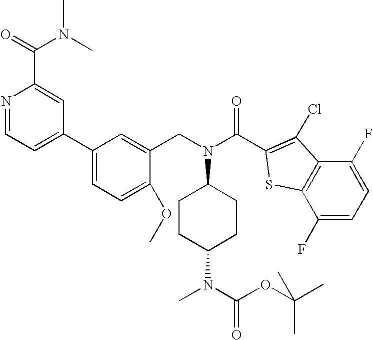

A stirred solution of amide 158 (125 mg, 0.175 mmol) in THF (5 mL) was treated with sodium hydride (11 mg, 0.26 mmol, 60% dispersion in mineral oil) at 0° C. After warming to RT and stirring 0.5 h, iodomethane (0.13 mL, 0.26 mmol, 2 M solution in TBME) was added via syringe and the reaction stirred 1 h. Analysis by LC/MS at this juncture revealed the reaction was only 75% complete. Hence more sodium hydride (11 mg, 0.26 mmol, 60% dispersion in mineral oil) was added, followed after 0.5 h by iodomethane (0.13 mL, 0.26 mmol, 2 M solution in TBME). After a further hour at RT, the reaction was diluted with water (10 mL) and extracted into EtOAc (3×20 mL). The combined EtOAc phases were dried (Na₂SO₄) and the solvent removed in vacuo. The title compound was obtained after chromatography (gradient elution—50-80% EtOAc in heptane with 0.5% triethylamine).

Yield: 105 mg (83%).

LC/MS tᵣ 1.67 min.

MS (ES+) m/z 729, 727 (M+H).

### 4-(3-{[(3-Chloro-4,7-difluoro-benzo[b]thiophene-2-carbonyl)-(4-methylamino-cyclohexyl)-amino]-methyl}-4-methoxy-phenyl)-pyridine-2-carboxylic acid dimethylamide dihydrochloride (164)

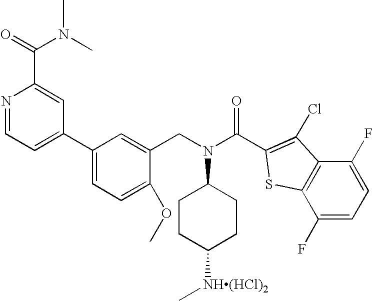

tert-Butyl carbamate 163 (105 mg, 0.14 mmol) was deprotected using Method F. On removal of the solvents in vacuo, the reaction mixture was dissolved in DCM (20 mL) and extracted into 2 M HCl (4×10 mL). The combined HCl phases were washed with DCM (2×20 mL) then taken to pH 9 by careful addition of solid NaHCO₃. The resultant aqueous suspension was then extracted into DCM (3×20 mL), the combined DCM phases dried (Na₂SO₄) and reduced in vacuo. The title compound was then obtained by Method H.

Yield: 67 mg (72%).

LC/MS tᵣ 1.20 min.

MS (ES+) m/z 629, 627 (M+H), 315, 314 [(M+H)/2].

### Synthesis of Compound R6

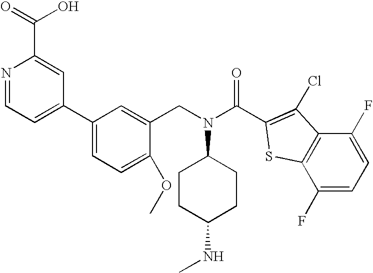

### 4-(3-{[(3-Chloro-4,7-difluoro-benzo[b]thiophene-2-carbonyl)-(4-methylamino-cyclohexyl)-amino]-methyl}-4-methoxy-phenyl)-pyridine-2-carboxylic acid bis(trifluoroacetate) (165)

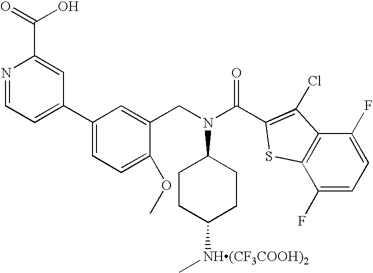

Methyl ester 149 (69 mg, 0.09 mmol) was dissolved in MeOH (2 mL) and treated with 6 M sodium hydroxide (500 μL). The reaction mixture was stirred 2 h during which time a precipitate formed. The precipitate was filtered off, suspended in DCM (2 mL) and TFA (300 μL) added dropwise. The solvents were then removed in vacuo to give a yellow oil. Purification by preparative HPLC gave the product as the TFA salt.

Yield: 49 mg (83%).

LC/MS tᵣ 1.71 min.

MS (ES+) m/z 602, 600 (M+H).

### Synthesis of Compound 358

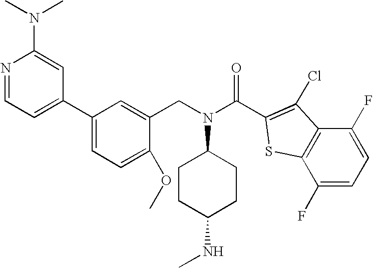

### 2-Amino-4-bromopyridine (166)

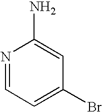

tert-Butyl carbamate 155 (200 mg, 0.73 mol) was suspended in water (1 mL) and treated with HBr (1 mL, 48 wt. % in water). After stirring 16 h, the reaction mixture was poured onto aqueous NaHCO₃ (25 mL) and extracted into EtOAc (3×25 mL). The combined EtOAc phases were dried (MgSO₄) and reduced in vacuo. The title compound was obtained after chromatography of this residue (neat EtOAc).

Yield: 73 mg (58%).

LC/MS tᵣ 0.69 min.

MS (ES+) m/z 175, 173 (M+H).

### 4-Bromo-2-(dimethylamino)pyridine (167)

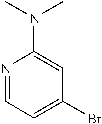

A stirred solution of amino-pyridine 166 (73 mg, 0.42 mmol) in THF (5 mL) was treated with sodium hydride (50 mg, 1.26 mmol, 60% dispersion in mineral oil) at 0° C. After warming to RT and stirring 10 minutes, iodomethane (58 μL, 0.93 mmol) was added via syringe and the reaction stirred 16 h. The reaction was then diluted with water (10 mL) and extracted into EtOAc (3×20 mL). The combined EtOAc phases were dried (Na₂SO₄) and the solvent removed in vacuo to afford the title compound.

Yield: 92 mg (quant.).

### 3-Chloro-4,7-difluoro-benzo[b]thiophene-2-carboxylic acid [5-(2-dimethylamino-pyridin-4-yl)-2-methoxy-benzyl]-(4-methylamino-cyclohexyl)-amide dihydrochloride (168)

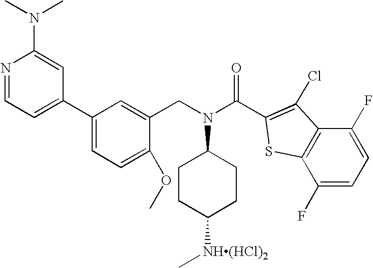

Boronic acid 9 (200 mg, 0.32 mmol) was coupled to pyridyl bromide 167 (71 mg, 0.35 mmol) using Method A. The tert-butyl carbamate (180 mg, 0.26 mmol) isolated after chromatography was then directly deprotected using Method F to afford the title compound.

Yield: 183 mg (85% over two steps).

LC/MS tᵣ 1.16 min.

MS (ES+) m/z 601, 599 (M+H).

### Synthesis of Compound 359

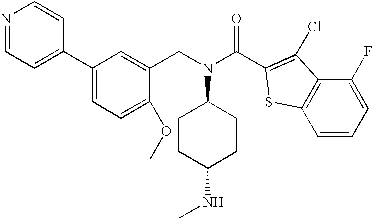

### tert-Butyl {4-[(3-chloro-4-fluoro-benzo[b]thiophene-2-carbonyl)-(2-methoxy-5-pyridin-4-yl-benzyl)-amino]-cyclohexyl}-methyl-carbamate (169)

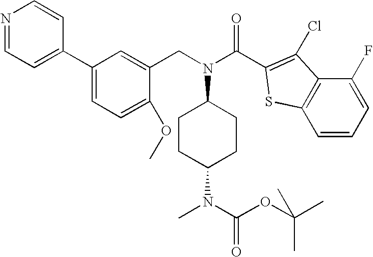

Boronic acid 7 (150 mg, 0.25 mmol) was coupled to 4-bromopyridine hydrochloride (61 mg, 0.31 mmol) using Method B to give the title compound.

Yield: 45 mg (28%). Contains ca. 47% triphenylphosphine oxide.

LC/MS tᵣ 1.53 min.

MS (ES+) m/z 640, 638 (M+H).

### 3-Chloro-4-fluoro-benzo[b]thiophene-2-carboxylic acid (2-methoxy-5-pyridin-4-yl-benzyl)-(4-methylamino-cyclohexyl)-amide dihydrochloride (170)

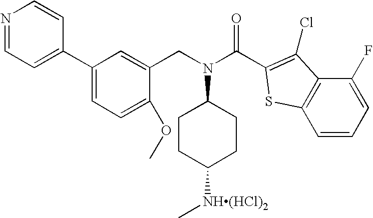

tert-Butyl carbamate 169 (45 mg, 71 μmol) was deprotected using Method F. On removal of the solvents in vacuo, the residue was dissolved in water (3 mL) and washed with TBME (3×1 mL). Reduction of the aqueous phase in vacuo afforded the title product.

Yield: 34 mg (78%).

LC/MS tᵣ 1.08 min.

MS (ES+) m/z 540, 538 (M+H).

### Synthesis of Compound 360

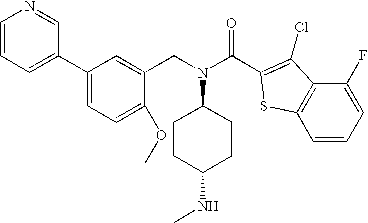

### tert-Butyl {4-[(3-chloro-4-fluoro-benzo[b]thiophene-2-carbonyl)-(2-methoxy-5-pyridin-3-yl-benzyl)-amino]-cyclohexyl}-methyl-carbamate (171)

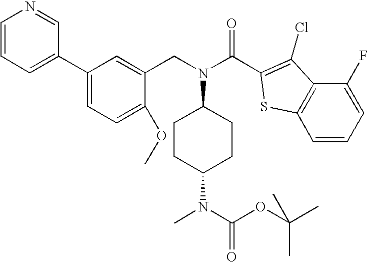

Boronic acid 7 (50 mg, 83 μmol) was coupled to 3-bromopyridine (16 mg, 0.10 mmol) using Method B to give the title compound.

Yield: 34 mg (64%).

LC/MS tᵣ 1.62 min.

MS (ES+) m/z 640, 638 (M+H).

### 3-Chloro-4-fluoro-benzo[b]thiophene-2-carboxylic acid (2-methoxy-5-pyridin-3-yl-benzyl)-(4-methylamino-cyclohexyl)-amide dihydrochloride (172)

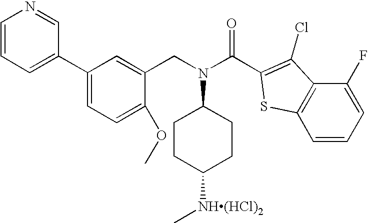

tert-Butyl carbamate 171 (34 mg, 53 μmol) was deprotected using Method F. On removal of the solvents in vacuo, the residue was dissolved in water (3 mL) and washed with TBME (3×1 mL). Reduction of the aqueous phase in vacuo afforded the title product.

Yield: 24 mg (74%).

LC/MS tᵣ 1.05 min.

MS (ES+) m/z 540, 538 (M+H).

### Synthesis of Compound 361

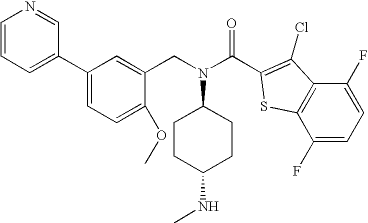

### tert-Butyl [4-(2-methoxy-5-pyridin-3-yl-benzylamino)-cyclohexyl]-methyl-carbamate (173)

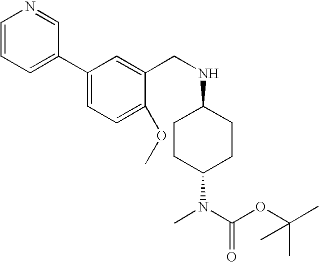

Boronic acid 4 (500 mg, 1.27 mmol) was coupled to 3-bromopyridine (202 mg, 1.27 mmol) using Method A to afford the title compound.

Yield: 320 mg (59%).

LC/MS tᵣ 1.04 min.

MS (ES+) m/z 851 (2M+H), 426 (M+H).

### tert-Butyl {4-[(3-chloro-4,7-difluoro-benzo[b]thiophene-2-carbonyl)-(2-methoxy-5-pyridin-3-yl-benzyl)-amino]-cyclohexyl}-methyl-carbamate (174)

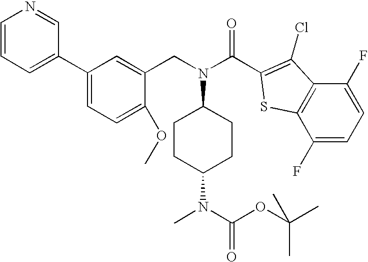

Biaryl amine 173 (320 mg, 0.75 mmol) was treated with acid chloride 8 (221 mg, 0.83 mmol) using Method D to give the title compound.

Yield: 170 mg (35%).

LC/MS tᵣ 1.60 min.

MS (ES+) m/z 658, 656 (M+H).

### 3-Chloro-4,7-difluoro-benzo[b]thiophene-2-carboxylic acid (2-methoxy-5-pyridin-3-yl-benzyl)-(4-methylamino-cyclohexyl)-amide dihydrochloride (175)

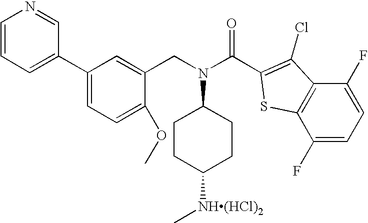

tert-Butyl carbamate 174 (170 mg, 0.26 mmol) was deprotected using Method F to afford the title compound.

Yield: 107 mg (66%).

LC/MS tᵣ 1.08 min.

MS (ES+) m/z 558, 556 (M+H).

### Synthesis of Compound 362

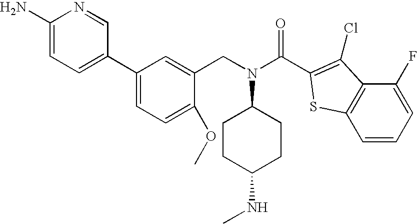

### tert-Butyl {4-[[5-(6-amino-pyridin-3-yl)-2-methoxy-benzyl]-(3-chloro-4-fluoro-benzo[b]thiophene-2-carbonyl)-amino]-cyclohexyl}-methyl-carbamate (176)

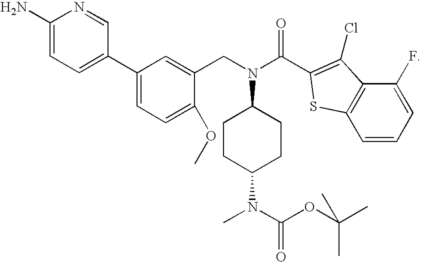

Boronic acid 7 (50 mg, 0.08 mmol) was coupled to 2-amino-5-bromopyridine (17 mg, 0.10 mmol) using Method A to give the title compound.

Yield: 29 mg (55%).

LC/MS tᵣ 1.64 min.

MS (ES+) m/z 655, 653 (M+H).

### 3-Chloro-4-fluoro-benzo[b]thiophene-2-carboxylic acid [5-(6-amino-pyridin-3-yl)-2-methoxy-benzyl]-(4-methylamino-cyclohexyl)-amide dihydrochloride (177)

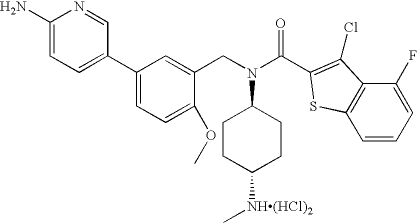

tert-Butyl carbamate 176 (29 mg, 0.04 mmol) was deprotected using Method F to give the title compound.

Yield: 20 mg (72%).

LC/MS tᵣ 1.12 min.

MS (ES+) m/z 555, 553 (M+H).

### Synthesis of Compound 363

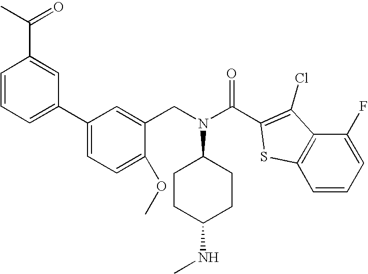

### tert-Butyl {4-[(3′-acetyl-4-methoxy-biphenyl-3-ylmethyl)-amino]-cyclohexyl}-methyl-carbamate (178)

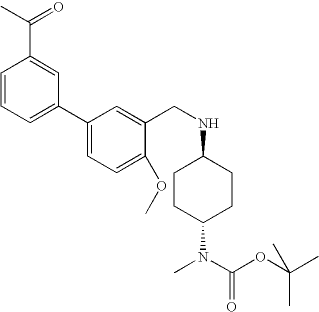

Boronic acid 4 (140 mg, 0.36 mmol) was coupled to 3′-bromoacetophenone (85 mg, 0.43 mmol) using Method B to give the title compound.

Yield: 160 mg (96%).

LC/MS tᵣ 1.39 min.

MS (ES+) m/z 467 (M+H).

### tert-Butyl {4-[(3′-acetyl-4-methoxy-biphenyl-3-ylmethyl)-(3-chloro-4-fluoro-benzo[b]thiophene-2-carbonyl)-amino]-cyclohexyl}-methyl-carbamate (179)

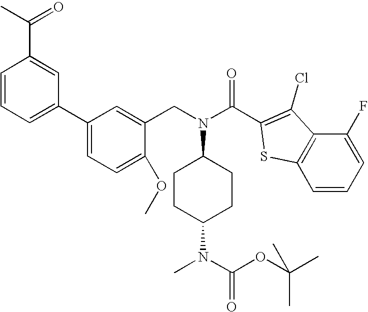

Biaryl amine 178 (80 mg, 0.15 mmol) was treated with acid chloride 6 (53 mg, 0.21 mmol) using Method D to give the title compound.

Yield: 62 mg (92%).

LC/MS tᵣ 1.92 min.

MS (ES+) m/z 625, 623 (M−C(CH₃)₃+H).

### 3-Chloro-4-fluoro-benzo[b]thiophene-2-carboxylic acid (3′-acetyl-4-methoxy-biphenyl-3-ylmethyl)-(4-methylamino-cyclohexyl)-amide hydrochloride (180)

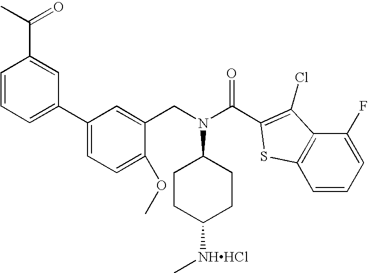

tert-Butyl carbamate 179 (32 mg, 0.05 mmol) was deprotected using Method F to give the title compound.

Yield: 28 mg (quant.).

LC/MS tᵣ 1.46 min.

MS (ES+) m/z 581, 579 (M+H).

### Synthesis of Compound 364

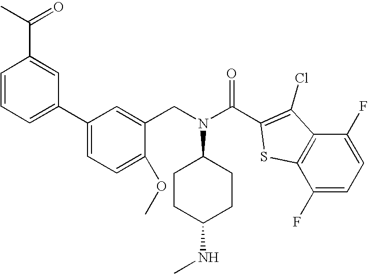

### tert-Butyl {4-[(3′-acetyl-4-methoxy-biphenyl-3-ylmethyl)-(3-chloro-4,7-difluoro-benzo[b]thiophene-2-carbonyl)-amino]-cyclohexyl}-methyl-carbamate (181)

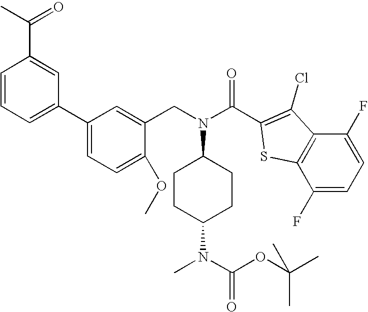

Biaryl amine 178 (80 mg, 0.15 mmol) was treated with acid chloride 8 (57 mg, 0.21 mmol) using Method D to give the title compound.

Yield: 70 mg (57%).

LC/MS t, 1.95 min.

MS (ES+) m/z 643, 641-(M−C(CH₃)₃+H).

### 3-Chloro-4,7-difluoro-benzo[b]thiophene-2-carboxylic acid (3′-acetyl-4-methoxy-biphenyl-3-ylmethyl)-(4-methylamino-cyclohexyl)-amide hydrochloride (182)

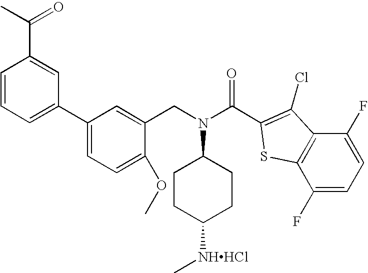

tert-Butyl carbamate 181 (70 mg, 0.10 mmol) was deprotected using Method F to give the title compound.

Yield: 69 mg (quant.).

LC/MS tᵣ 1.45 min.

MS (ES+) m/z 599, 597 (M+H).

### Synthesis of Compound 365

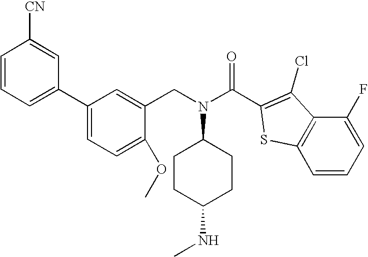

### tert-Butyl {4-[(3-chloro-4-fluoro-benzo[b]thiophene-2-carbonyl)-(3′-cyano-4-methoxy-biphenyl-3-ylmethyl)-amino]-cyclohexyl}-methyl-carbamate (183)

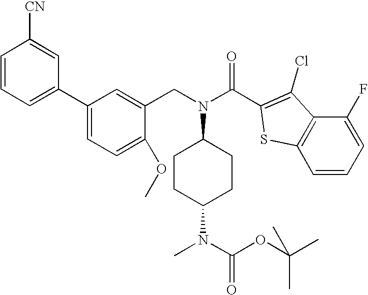

Biaryl amine 22 (57 mg, 0.13 mmol) was treated with acid chloride 6 (38 mg, 0.15 mmol) using Method D to give the title compound.

Yield: 50 mg (17%).

LC/MS tᵣ 1.92 min.

MS (ES+) m/z 608, 606 (M−C(CH₃)₃+H).

### 3-Chloro-4-fluoro-benzo[b]thiophene-2-carboxylic acid (3′-cyano-4-methoxy-biphenyl-3-ylmethyl)-(4-methylamino-cyclohexyl)-amide hydrochloride (184)

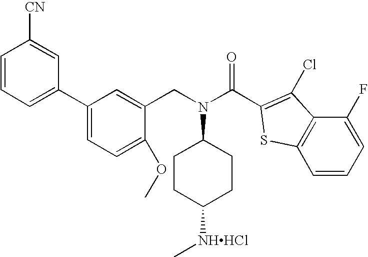

tert-Butyl carbamate 183 (50 mg, 0.08 mmol) was deprotected using Method F. Purification by preparative HPLC followed by formation of the HCl salt using Method H gave the title compound.

Yield: 31 mg (74%).

LC/MS tᵣ 1.44 min.

MS (ES+) m/z 564, 562 (M+H).

### Synthesis of Compound 366

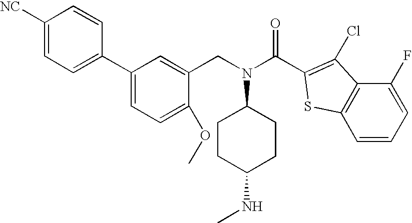

### tert-Butyl {4-[(3-chloro-4-fluoro-benzo[b]thiophene-2-carbonyl)-(4′-cyano-4-methoxy-biphenyl-3-ylmethyl)-amino]-cyclohexyl}-methyl-carbamate (185)

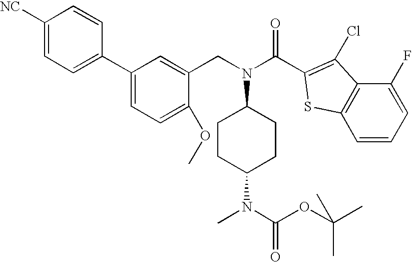

Biaryl amine 13 (87 mg, 0.19 mmol) was treated with acid chloride 6 (58 mg, 0.23 mmol) using Method D to give the title compound.

Yield: 63 mg (49%).

LC/MS tᵣ 1.90 min.

MS (ES+) m/z 608, 606 (M−C(CH₃)₃+H).

### 3-Chloro-4-fluoro-benzo[b]thiophene-2-carboxylic acid (4′-cyano-4-methoxy-biphenyl-3-ylmethyl)-(4-methylamino-cyclohexyl)-amide hydrochloride (186)

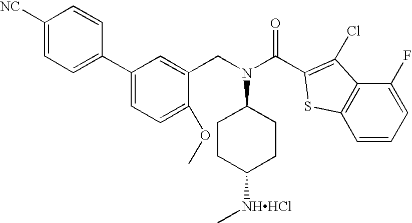

tert-Butyl carbamate 185 (60 mg, 0.09 mmol) was deprotected using Method F to give the title compound.

Yield: 53 mg (quant.).

LC/MS tᵣ 1.09 min.

MS (ES+) m/z 564, 562 (M+H).

### Synthesis of Compound 367

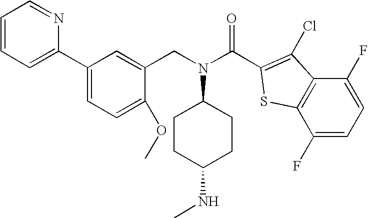

### tert-Butyl {4-[(3-chloro-4,7-difluoro-benzo[b]thiophene-2-carbonyl)-(2-methoxy-5-pyridin-2-yl-benzyl)-amino]-cyclohexyl}-methyl-carbamate (187)

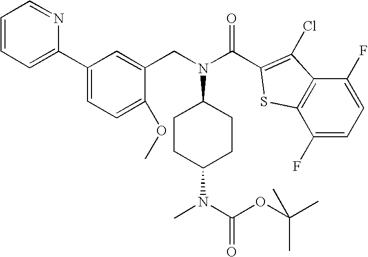

Boronic acid 9 (500 mg, 0.80 mmol) was coupled to 2-bromopyridine (77 μL, 0.80 mmol) using Method B to give the title compound.

Yield: 236 mg (45%).

LC/MS tᵣ 1.59 min.

MS (ES+) m/z 658, 656 (M+H).

### 3-Chloro-4,7-difluoro-benzo[b]thiophene-2-carboxylic acid (2-methoxy-5-pyridin-2-yl-benzyl)-(4-methylamino-cyclohexyl)-amide dihydrochloride (188)

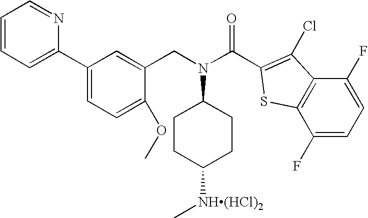

tert-Butyl carbamate 187 (236 mg, 0.36 mmol) was deprotected using Method F to afford the title compound.

Yield: 210 mg (98%).

LC/MS tᵣ 1.09 min.

MS (ES+) m/z 558, 556 (M+H).

### Synthesis of Compound R10

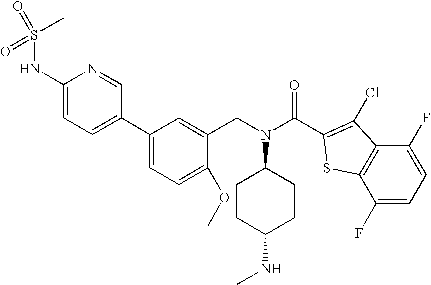

### tert-Butyl {4-[[5-(6-amino-pyridin-3-yl)-2-methoxy-benzyl]-(3-chloro-4,7-difluoro-benzo[b]thiophene-2-carbonyl)-amino]-cyclohexyl}-methyl-carbamate (189)

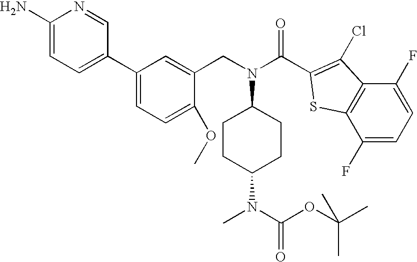

Boronic acid 9 (1.10 g, 1.76 mmol) was coupled to 2-amino-5-bromopyridine (365 mg, 2.11 mmol) using Method A to give the title compound.

Yield: 483 mg (41%).

LC/MS tᵣ 1.59 min.

MS (ES+) m/z 673, 671 (M+H).

### tert-Butyl (4-{(3-chloro-4,7-difluoro-benzo[b]thiophene-2-carbonyl)-[5-(6-methanesulfonylamino-pyridin-3-yl)-2-methoxy-benzyl]-amino}-cyclohexyl)-methyl-carbamate (190)

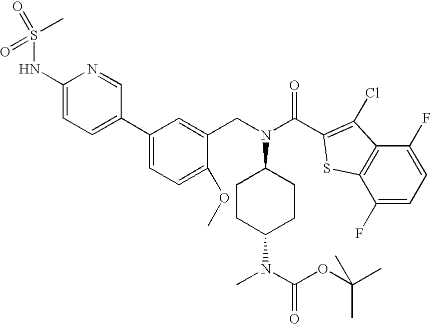

A stirred solution of amino-pyridine 189 (483 mg, 0.72 mmol) and triethylamine (1.20 mL, 8.64 mmol) in DCM (50 mL) was treated with methanesulfonyl chloride (0.62 mL, 8.0 mmol) at 0° C. The reaction was then warmed to RT and stirred 16 h. The solvents were removed in vacuo, the residue treated with aqueous NaHCO₃ (50 mL) and the resultant suspension extracted into EtOAc (3×50 mL). The EtOAc phases were combined, dried (MgSO₄) and reduced in vacuo to give the crude bis-sulfonamide. The residue was then dissolved in MeOH (17 mL) and THF (33 mL) and treated with conc. ammonia solution (17 mL) at RT. After stirring 1.5 h, analysis by LC/MS indicated that only 37% of the bis-sulfonamide had been converted to the title compound. Hence more conc. ammonia solution (17 mL) was added and the reaction stirred a further 2 h. The solvents were then removed in vacuo and the residue purified by chromatography (gradient elution—60% EtOAc in heptane with 0.5% triethylamine increasing to neat EtOAc with 0.5% triethylamine, then 0-10% MeOH in EtOAc with 0.5% triethylamine) to afford the title compound.

Yield: 384 mg (71%).

LC/MS tᵣ 1.75 min.

MS (ES+) m/z 751, 749 (M+H).

### 3-Chloro-4,7-difluoro-benzo[b]thiophene-2-carboxylic acid [5-(6-methanesulfonylamino-pyridin-3-yl)-2-methoxy-benzyl]-(4-methylamino-cyclohexyl)-amide dihydrochloride (191)

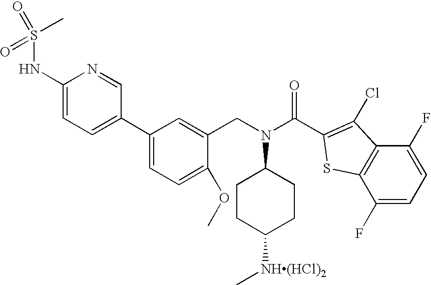

tert-Butyl carbamate 190 (384 mg, 0.51 mmol) was deprotected using Method F. On removal of the solvents in vacuo, the reaction mixture was dissolved in DCM (50 mL) and extracted into 2 M HCl (4×10 mL). The combined HCl phases were washed with DCM (2×25 mL) then taken to pH 9 by careful addition of solid NaHCO₃. The resultant aqueous suspension was then extracted into DCM (3×50 mL), these combined DCM phases dried (Na₂SO₄) and reduced in vacuo to afford the title compound. The DCM phases remaining were also combined, dried (MgSO₄) and the solvent removed in vacuo. Treating this residue according to Method H gave more of the title compound at greater purity.

Yield: 246 mg (66%).

LC/MS tᵣ 1.26 min.

MS (ES+) m/z 651, 649 (M+H).

### Synthesis of Compound 368

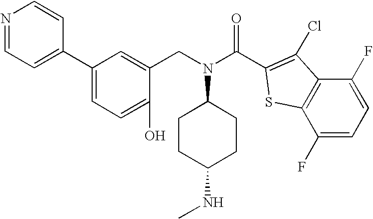

### 2-Methoxy-5-pyridin-4-yl-benzaldehyde (192)

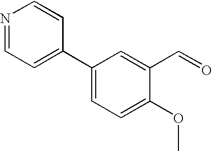

3-formyl-4-methoxybenzeneboronic acid (393 mg, 2.19 mmol) was coupled to 4-bromopyridine hydrochloride (400 mg, 2.19 mmol) using Method A to give the title compound.

Yield: 240 mg (52%).

LC/MS tᵣ 0.85 min.

MS (ES+) m/z 214 (M+H).

### 2-Hydroxy-5-pyridin-4-yl-benzaldehyde (193)

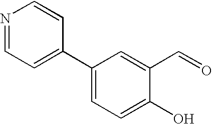

A solution of aldehyde 192 (700 mg, 3.29 mmol) in DCM (8 mL) at −78° C. was treated with boron tribromide (0.93 mL, 9.85 mmol) dropwise over 5 minutes. After 1 h at −78° C., the reaction was warmed to RT and stirred 2 h. The reaction mixture was then cooled (0° C.), quenched with water (5 mL), basified with aqueous NaHCO₃ (10 mL) and extracted into DCM (3×20 mL). The combined organic phases were dried over Na₂SO₄ and the solvent removed in vacuo to give the title compound.

Yield: 330 mg (50%).

LC/MS tᵣ 0.78 min.

MS (ES+) m/z 200 (M+H).

### tert-Butyl [4-(2-hydroxy-5-pyridin-4-yl-benzylamino)-cyclohexyl]-methyl-carbamate (194)

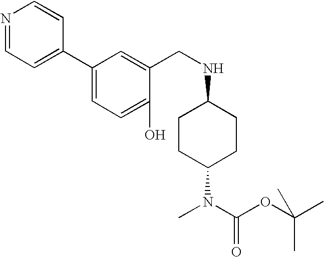

Amine 3 (453 mg, 1.98 mmol) was treated with aldehyde 193 (330 mg, 1.65 mmol) in accordance with Method C. Purification by column chromatography (60% EtOAc in heptane) gave the title compound.

Yield: 240 mg (35%).

LC/MS tᵣ 1.00 min.

MS (ES+) m/z 412 (M+H), 356 (M−C(CH₃)₃+H).

### tert-Butyl {4-[(3-chloro-4,7-difluoro-benzo[b]thiophene-2-carbonyl)-(2-hydroxy-5-pyridin-4-yl-benzyl)-amino]-cyclohexyl}-methyl-carbamate (195)

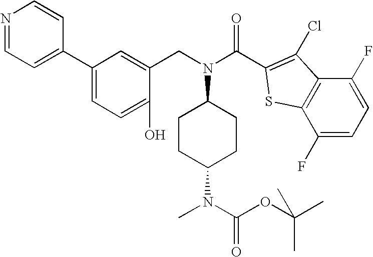

Biaryl amine 194 (240 mg, 0.58 mmol) was treated with acid chloride 8 (389 mg, 1.46 mmol) using Method D, resulting in the functionalisation of both amine and phenol. The resulting amide-ester (310 mg, 0.35 mmol) was dissolved in EtOH (3 mL), treated with 4 M NaOH (1 mL) and stirred 0.5 h at RT. The reaction mixture was then diluted with water (10 mL) and extracted into EtOAc (3×15 mL). The combined organic phases were dried over Na₂SO₄ then reduced in vacuo. Purification by column chromatography gave the title compound.

Yield: 210 mg (56%).

LC/MS tᵣ 1.48 min.

MS (ES+) m/z 644, 642 (M+H), 588, 586 (M−C(CH₃)₃+H).

### 3-Chloro-4,7-difluoro-benzo[b]thiophene-2-carboxylic acid (2-hydroxy-5-pyridin-4-yl-benzyl)-(4-methylamino-cyclohexyl)-amide dihydrochloride (196)

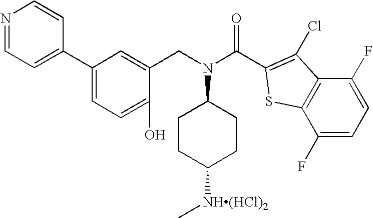

tert-Butyl carbamate 195 (210 mg, 0.33 mmol) was deprotected using Method F to give the title compound.

Yield: 171 mg (96%).

LC/MS tᵣ 1.04 min.

MS (ES+) m/z 544, 542 (M+H).

### Synthesis of Compound 369

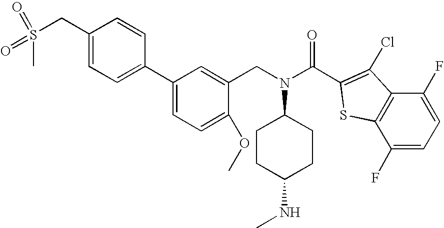

### 1-Bromo-4-methanesulfonylmethylbenzene (197)

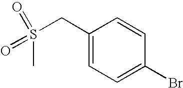

A stirred suspension of 4-bromobenzyl bromide (1.0 g, 4.0 mmol) and sodium methanesulfinate (2.04 g, 20 mmol) in DMF (10 mL) was heated 1 h at 60° C. The reaction mixture was then cooled to RT, diluted with water (200 mL) and extracted into EtOAc (3×25 mL). The combined EtOAc phases were washed with water (2×50 mL) and brine (25 mL) then dried (MgSO₄) and reduced in vacuo to afford the title compound.

Yield: 850 mg (85%).

LC/MS tᵣ 1.10 min

MS (ES+) mass ion not detected.

### tert-Butyl {4-[(4′-methanesulfonylmethyl-4-methoxy-biphenyl-3-ylmethyl)-amino]-cyclohexyl}-methyl-carbamate (198)

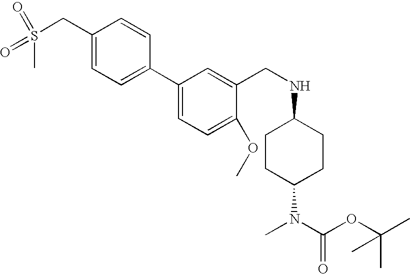

Boronic acid 4 (200 mg, 0.51 mmol) was coupled to aryl bromide 197 (127 mg, 0.51 mmol) using Method A. On filtration of the cooled reaction mixture through celite and removal of the solvents in vacuo, the crude residue obtained was used in the next synthetic step without further purification.

Yield: 362 mg.

LC/MS tᵣ 1.31 min.

MS (ES+) m/z 517 (M+H).

### tert-Butyl {4-[(3-chloro-4,7-difluoro-benzo[b]thiophene-2-carbonyl)-(4′-methanesulfonylmethyl-4-methoxy-biphenyl-3-ylmethyl)-amino]-cyclohexyl}-methyl-carbamate (199)

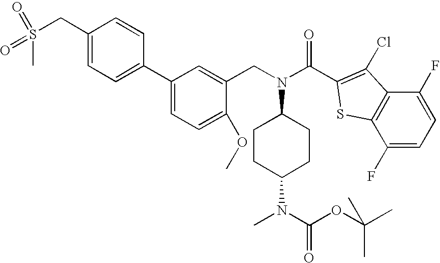

Crude biaryl amine 198 (362 mg) was treated with acid chloride 8 (224 mg, 0.84 mmol) using Method D to give the title compound.

Yield: 224 mg (59% over two steps).

LC/MS tᵣ 1.88 min.

MS (ES+) m/z 749, 747 (M+H), 693, 691 (M−C(CH₃)₃+H).

### 3-Chloro-4,7-difluoro-benzo[b]thiophene-2-carboxylic acid (4′-methanesulfonylmethyl-4-methoxy-biphenyl-3-ylmethyl)-(4-methylamino-cyclohexyl)-amide hydrochloride (200)

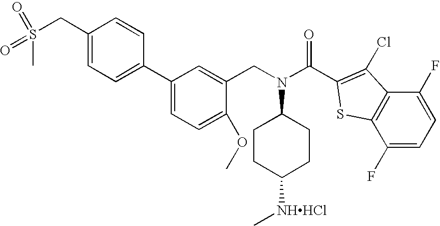

tert-Butyl carbamate 199 (224 mg, 0.30 mmol) was deprotected using Method F. On removal of the solvents in vacuo, the residue was suspended in aqueous NaHCO₃ (20 mL) and extracted into DCM (3×20 mL). The combined DCM phases were washed with water (2×20 mL) and brine (20 mL) then dried (Na₂SO₄) and reduced in vacuo. The free base thus obtained was converted to the HCl salt by Method H. The isolated salt was then dissolved in the minimum amount of DCM and added dropwise to cold (0° C.) TBME (25 mL). The resultant pale yellow precipitate was isolated by filtration, washed with TBME (10 mL) and diethyl ether (10 mL) and dried to afford the title compound.

Yield: 174 mg (90%).

LC/MS tᵣ 1.34 min.

MS (ES+) m/z 649, 647 (M+H).

### Synthesis of Compound 370

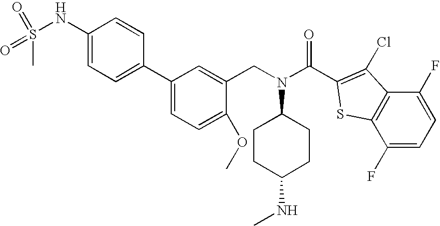

### N-(4-Bromophenyl)-methanesulfonamide (201)

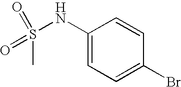

A stirred solution of 4-bromoaniline (1.0 g, 5.81 mmol) and triethylamine (2.43 mL, 17.4 mmol) in DCM (10 mL) at 0° C. was treated with methanesulfonyl chloride (0.47 mL, 6.10 mmol) over 5 minutes via syringe. The reaction was then warmed to RT and stirred 1 h. Analysis by LC/MS at this juncture indicated the reaction mixture contained unreacted starting material, the desired mono-sulfonamide and undesired bis-sulfonamide in a 1:1:1 ratio. Thus more triethylamine (5.0 mL, 35.9 mmol) and methanesulfonyl chloride (1.0 mL, 12.9 mmol) was added and the reaction stirred at RT a further 2 h. On confirmation by LC/MS that all the starting aniline had been converted to the mono- or bis-sulfonamide, the reaction was diluted with water (15 mL) and extracted into EtOAc (3×25 mL). The combined EtOAc phases were then dried (Na₂SO₄) and reduced in vacuo. The residue thus obtained was dissolved in THF (50 mL) and MeOH (50 mL) then treated with conc. ammonia solution (75 mL) at RT and stirred 16 h. The reaction mixture was then concentrated in vacuo, diluted with water (50 mL) and extracted into EtOAc (3×25 mL). The combined EtOAc phases were dried (MgSO₄) and reduced in vacuo to afford the title compound.

Yield: 1.57 g (quant.).

LC/MS tᵣ 1.16 min

MS (ES+) m/z 252, 250 (M+H)

### tert-Butyl {4-[(4′-methanesulfonylamino-4-methoxy-biphenyl-3-ylmethyl)-amino]-cyclohexyl}-methyl-carbamate (202)

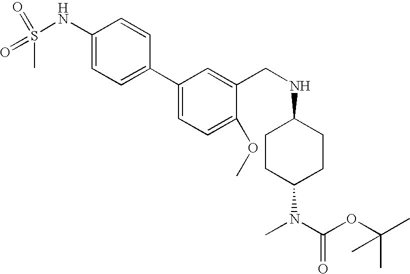

Boronic acid 4 (200 mg, 0.51 mmol) was coupled to aryl bromide 201 (134 mg, 0.54 mmol) using Method A to give the title compound.

Yield: 42 mg (15%).

LC/MS tᵣ 1.34 min.

MS (ES+) m/z 518 (M+H).

### tert-Butyl {4-[(3-chloro-4,7-difluoro-benzo[b]thiophene-2-carbonyl)-(4′-methanesulfonylamino-4-methoxy-biphenyl-3-ylmethyl)-amino]-cyclohexyl}-methyl-carbamate (203)

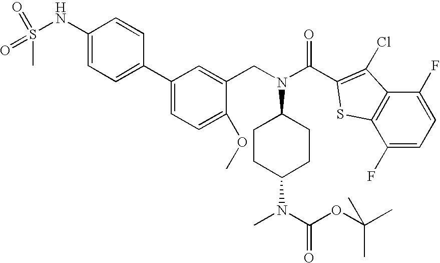

Biaryl amine 202 (42 mg, 81 μmol) was treated with acid chloride 8 (24 mg, 89 μmol) using Method D to give the title compound.

Yield: 45 mg (74%).

LC/MS tᵣ 1.85 min.

MS (ES+) m/z 750, 748 (M+H), 694, 692 (M−C(CH₃)₃+H).

### 3-Chloro-4,7-difluoro-benzo[b]thiophene-2-carboxylic acid (4′-methanesulfonylamino-4-methoxy-biphenyl-3-ylmethyl)-(4-methylamino-cyclohexyl)-amide hydrochloride (204)

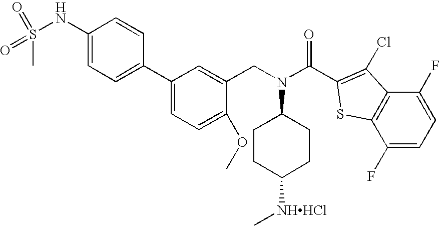

tert-Butyl carbamate 203 (18 mg, 24 μmol) was deprotected using Method F. On removal of the solvents in vacuo, the residue was suspended in aqueous NaHCO₃ (10 mL) and extracted into DCM (3×10 mL). The combined DCM phases were washed with water (2×10 mL) and brine (10 mL) then dried (Na₂SO₄) and reduced in vacuo. The free base thus obtained was then converted to the title compound using Method H.

Yield: 18 mg (quant.).

LC/MS tᵣ 1.36 min.

MS (ES+) m/z 672, 670 (M+Na), 650, 648 (M+H).

### Synthesis of Compound 371

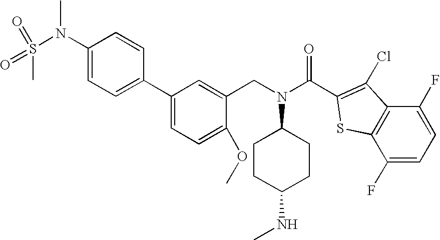

### tert-Butyl (4-{(3-chloro-4,7-difluoro-benzo[b]thiophene-2-carbonyl)-[4′-(methanesulfonyl-methyl-amino)-4-methoxy-biphenyl-3-ylmethyl]-amino}-cyclohexyl)-methyl-carbamate (205)

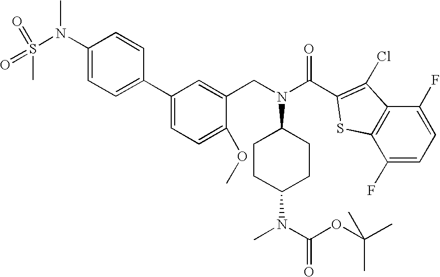

A stirred solution of sulfonamide 203 (23 mg, 31 μmol) in THF (2 mL) was treated with sodium hydride (2 mg, 46 μmol, 60% dispersion in mineral oil) at 0° C. After warming to RT and stirring 0.5 h, iodomethane (10 μL, 0.15 mmol) was added via syringe and the reaction stirred 2 h. Analysis by LC/MS at this juncture revealed the reaction was only 40% complete. Hence more sodium hydride (6 mg, 0.15 mmol, 60% dispersion in mineral oil) was added, followed after 0.5 h by iodomethane (30 μL, 0.46 mmol). After a further 16 h at RT, the reaction was diluted with water (10 mL) and extracted into EtOAc (3×15 mL). The combined EtOAc phases were dried (Na₂SO₄) and reduced in vacuo to afford the title compound.

Yield: 23 mg (quant.).

LC/MS tᵣ 1.92 min.

MS (ES+) m/z 708, 706 (M−C(CH₃)₃+H).

### 3-Chloro-4,7-difluoro-benzo[b]thiophene-2-carboxylic acid [4′-(methanesulfonyl-methyl-amino)-4-methoxy-biphenyl-3-ylmethyl]-(4-methylamino-cyclohexyl)-amide hydrochloride (206)

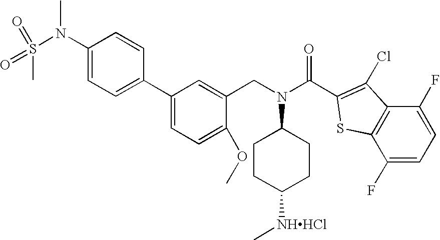

tert-Butyl carbamate 205 (23 mg, 31 μmol) was deprotected using Method F. The isolated salt was then dissolved in the minimum amount of DCM and added dropwise to cold (0° C.) hexane (10 mL). The resultant cream precipitate was isolated by filtration and dried to afford the title compound.

Yield: 19 mg (89%).

LC/MS tᵣ 1.50 min.

MS (ES+) m/z 664, 662 (M+H).

### Synthesis of Compound 372

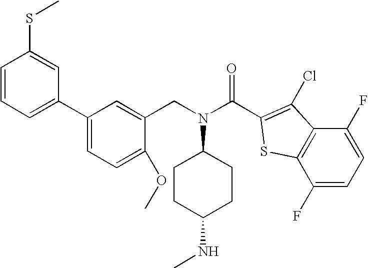

### tert-Butyl {4-[(3-chloro-4,7-difluoro-benzo[b]thiophene-2-carbonyl)-(4-methoxy-3′-methylsulfanyl-biphenyl-3-ylmethyl)-amino]-cyclohexyl}-methyl-carbamate (207)

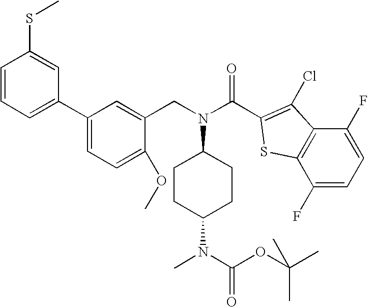

Boronic acid 4 (300 mg, 0.77 mmol) was coupled to 3-bromothioanisole (156 mg, 0.77 mmol) using Method A. On filtration of the cooled reaction mixture through celite and removal of the solvents in vacuo, the crude biaryl amine was treated with acid chloride 8 (246 mg, 0.92 mmol) using Method D to give the title compound.

Yield: 229 mg (42%).

LC/MS tᵣ 2.06 min.

MS (ES+) m/z 647, 645 (M−C(CH₃)₃+H).

### 3-Chloro-4,7-difluoro-benzo[b]thiophene-2-carboxylic acid (4-methoxy-3′-methylsulfanyl-biphenyl-3-ylmethyl)-(4-methylamino-cyclohexyl)-amide hydrochloride (208)

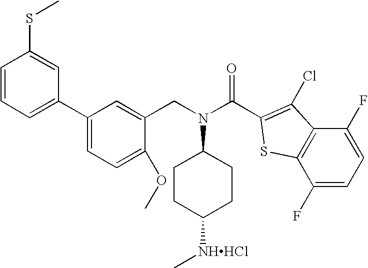

tert-Butyl carbamate 207 (25 mg, 0.04 mmol) was deprotected using Method F to give the title compound.

Yield: 23 mg (quant.).

LC/MS tᵣ 2.20 min.

MS (ES+) m/z 603, 601 (M+H).

### Synthesis of Compound 373

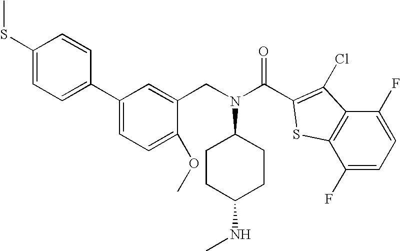

### tert-Butyl {4-[(3-chloro-4,7-difluoro-benzo[b]thiophene-2-carbonyl)-(4-methoxy-4′-methylsulfanyl-biphenyl-3-ylmethyl)-amino]-cyclohexyl}-methyl-carbamate (209)

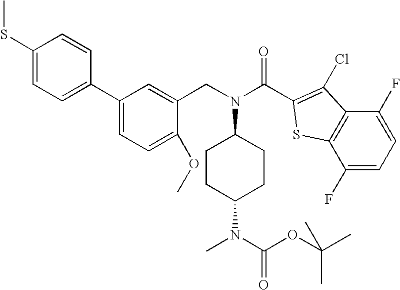

Boronic acid 4 (200 mg, 0.51 mmol) was coupled to 4-bromothioanisole (104 mg, 0.51 mmol) using Method A. On filtration of the cooled reaction mixture through celite and removal of the solvents in vacuo, the crude biaryl amine was treated with acid chloride 8 (163 mg, 0.61 mmol) using Method D to give the title compound.

Yield: 232 mg (65%).

LC/MS tᵣ 2.06 min.

MS (ES+) m/z 647, 645 (M−C(CH₃)₃+H).

### 3-Chloro-4,7-difluoro-benzo[b]thiophene-2-carboxylic acid (4-methoxy-4′-methylsulfanyl-biphenyl-3-ylmethyl)-(4-methylamino-cyclohexyl)-amide hydrochloride (210)

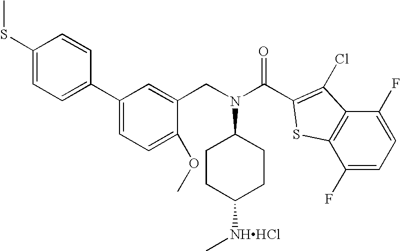

tert-Butyl carbamate 209 (25 mg, 0.04 mmol) was deprotected using Method F to give the title compound.

Yield: 23 mg (quant.).

LC/MS tᵣ 2.21 min.

MS (ES+) m/z 603, 601 (M+H).

### Synthesis of Compound 376

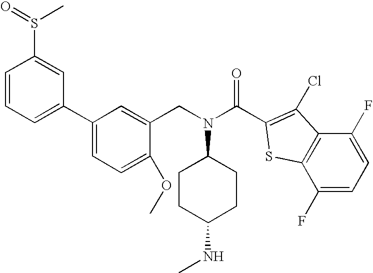

### tert-Butyl {4-[(3-chloro-4,7-difluoro-benzo[b]thiophene-2-carbonyl)-(3′-methanesulfinyl-4-methoxy-biphenyl-3-ylmethyl)-amino]-cyclohexyl}-methyl-carbamate (211) and tert-Butyl {4-[(3-chloro-4,7-difluoro-benzo[b]thiophene-2-carbonyl)-(3′-methanesulfonyl-4-methoxy-biphenyl-3-ylmethyl)-amino]-cyclohexyl}-methyl-carbamate (212)

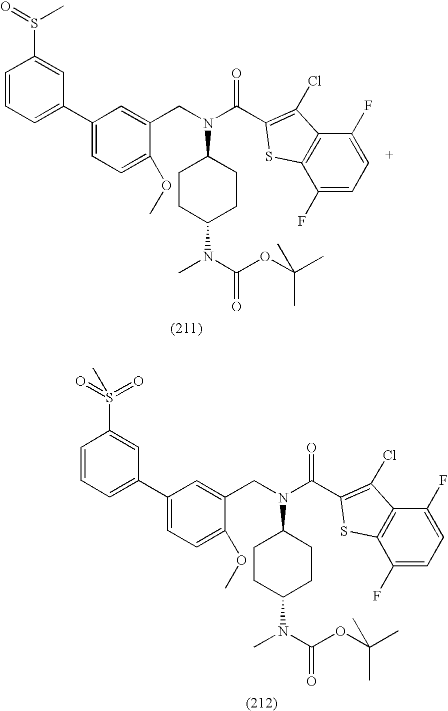

A stirred suspension of methyl sulfide 207 (75 mg, 0.11 mmol) and NaHCO₃ (6 mg, 0.55 mmol) in DCM (3 mL) at 0° C. was treated with a solution of m-chloroperbenzoic acid (34 mg, 0.20 mmol) in DCM (1 mL) dropwise over 2 minutes. After stirring 2 h at RT, the reaction was diluted with aqueous Na₂SO₃ (10 mL) and extracted into DCM (3×10 mL). The combined DCM phases were then dried (Na₂SO₄) and reduced in vacuo. The residue was purified by column chromatography (gradient elution—10-90% EtOAc in heptane with 0.5% triethylamine) to give both the desired sulfoxide 211 and the analogous sulfone 212.

Yield (sulfoxide 211): 17 mg (22%).

LC/MS tᵣ 1.82 min.

MS (ES+) m/z 719, 717 (M+H).

Yield (sulfone 212): 24 mg (30%).

LC/MS tᵣ 1.88 min.

MS (ES+) m/z 735, 733 (M+H), 679, 677 (M−C(CH₃)₃+H).

### 3-Chloro-4,7-difluoro-benzo[b]thiophene-2-carboxylic acid (3′-methanesulfinyl-4-methoxy-biphenyl-3-ylmethyl)-(4-methylamino-cyclohexyl)-amide trifluoroacetate (213)

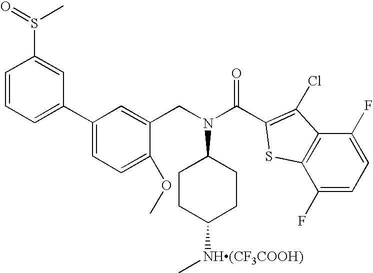

tert-Butyl carbamate 211 (17 mg, 24 μmol) was deprotected using Method G to afford the title compound.

Yield: 23 mg (quant.).

LC/MS tᵣ 1.40 min.

MS (ES+) m/z 619, 617 (M+H).

### Synthesis of Compound 377

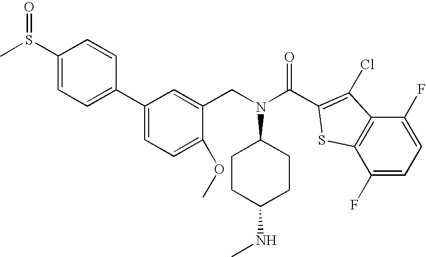

### tert-Butyl {4-[(3-chloro-4,7-difluoro-benzo[b]thiophene-2-carbonyl)-(4′-methanesulfinyl-4-methoxy-biphenyl-3-ylmethyl)-amino]-cyclohexyl}-methyl-carbamate (214)

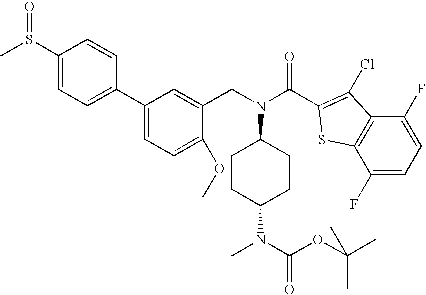

A stirred suspension of methyl sulfide 209 (75 mg, 0.11 mmol) and NaHCO₃ (46 mg, 0.55 mmol) in DCM (3 mL) at 0° C. was treated with a solution of m-chloroperbenzoic acid (20 mg, 0.12 mmol) in DCM (1 mL) dropwise over 2 minutes. After stirring 2 h at RT, the reaction was diluted with aqueous Na₂SO₃ (10 mL) and extracted into DCM (3×10 mL). The combined DCM phases were then dried (Na₂SO₄) and reduced in vacuo. The residue was purified by column chromatography (gradient elution—10-90% EtOAc in heptane with 0.5% triethylamine) to give the title compound.

Yield: 43 mg (54%).

LC/MS tᵣ 1.80 min.

MS (ES+) m/z 719, 717 (M+H), 663, 661 (M−C(CH₃)₃+H).

### 3-Chloro-4,7-difluoro-benzo[b]thiophene-2-carboxylic acid (4′-methanesulfinyl-4-methoxy-biphenyl-3-ylmethyl)-(4-methylamino-cyclohexyl)-amide trifluoroacetate (215)

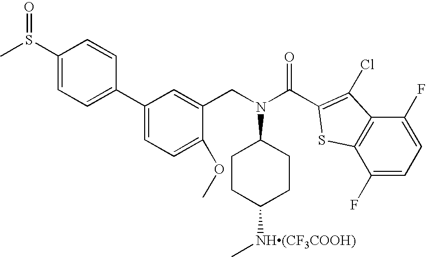

tert-Butyl carbamate 214 (43 mg, 60 μmol) was deprotected using Method G to afford the title compound.

Yield: 54 mg (quant.).

LC/MS tᵣ 1.40 min.

MS (ES+) m/z 619, 617 (M+H).

### Synthesis of Compound 388

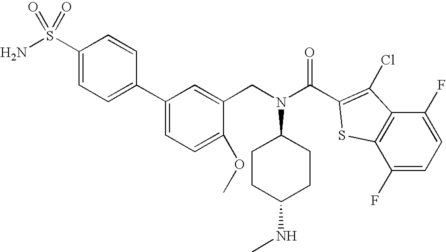

### tert-Butyl {4-[(4-methoxy-4′-sulfamoyl-biphenyl-3-ylmethyl)-amino]-cyclohexyl}-methyl-carbamate (216)

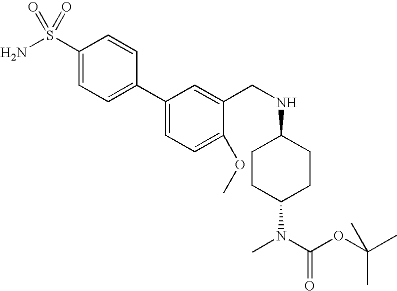

Boronic acid 4 (400 mg, 1.02 mmol) was coupled to 4-bromobenzene-sulfonamide (241 mg, 1.02 mmol) using Method A. On filtration of the cooled reaction mixture through celite and removal of the solvents in vacuo, the crude residue obtained was used in the next synthetic step without further purification.

Yield: 514 mg.

LC/MS tᵣ 1.24 min.

MS (ES+) m/z 504 (M+H).

### tert-Butyl {4-[(3-chloro-4,7-difluoro-benzo[b]thiophene-2-carbonyl)-(4-methoxy-4′-sulfamoyl-biphenyl-3-ylmethyl)-amino]-cyclohexyl}-methyl-carbamate (217)

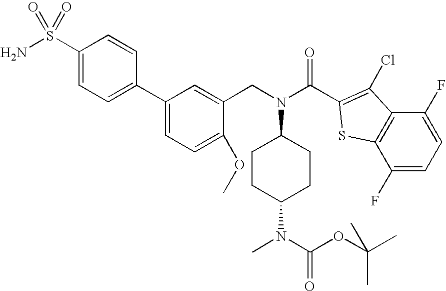

Crude biaryl amine 216 (514 mg) was treated with acid chloride 8 (327 mg, 1.22 mmol) using Method D to give the title compound.

Yield: 324 mg (43% over two steps) containing triphenylphosphine oxide (ca. 14%).

LC/MS tᵣ 1.79 min.

MS (ES+) m/z 736, 734 (M+H), 680, 678 (M−C(CH₃)₃+H).

### 3-Chloro-4,7-difluoro-benzo[b]thiophene-2-carboxylic acid (4-methoxy-4′-sulfamoyl-biphenyl-3-ylmethyl)-(4-methylamino-cyclohexyl)-amide hydrochloride (218)

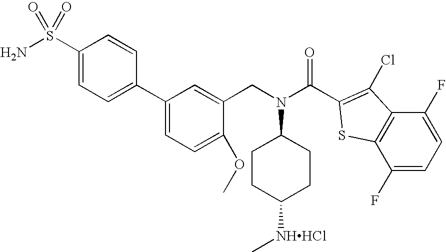

tert-Butyl carbamate 217 (100 mg, 0.14 mmol) was deprotected using Method F. The isolated salt was then dissolved in the minimum amount of DCM and added dropwise to cold (0° C.) TBME (25 mL). The resultant pale yellow precipitate was isolated by filtration, washed with TBME (10 mL) and diethyl ether (10 mL) and dried to afford the title compound.

Yield: 65 mg (69%).

LC/MS tᵣ 1.33 min.

MS (ES+) m/z 636, 634 (M+H).

### Synthesis of Compound 389

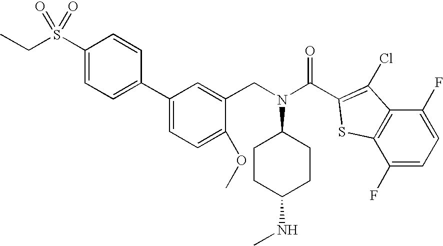

### tert-Butyl [4-(5-bromo-2-methoxy-benzylamino)-cyclohexyl]-methyl-carbamate (219)

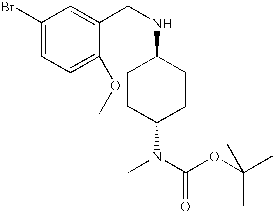

Amine 3 (1.0 g, 4.38 mmol) was treated with 5-bromo-2-methoxy-benzaldehyde (943 mg, 4.38 mmol) in accordance with Method C. Purification by column chromatography (2:1 EtOAc to heptane then 1:1 MeOH and DCM with 2% triethylamine) gave the title compound.

Yield: 1.27 g (68%).

LC/MS tᵣ 1.30 min.

MS (ES+) m/z 429, 427 (M+H).

### tert-Butyl {4-[(5-bromo-2-methoxy-benzyl)-(3-chloro-4,7-difluoro-benzo[b]thiophene-2-carbonyl)-amino]-cyclohexyl}-methyl-carbamate (220)

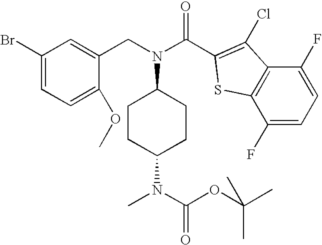

Biaryl amine 219 (0.50 g, 1.17 mmol) was treated with acid chloride 8 (344 mg, 1.29 mmol) using Method D to give the title compound.

Yield: 520 mg (67%).

LC/MS tᵣ 2.01 min.

MS (ES+) m/z 605, 603, 601 (M−C(CH₃)₃+H).

### tert-Butyl {4-[(3-chloro-4,7-difluoro-benzo[b]thiophene-2-carbonyl)-(4′-ethanesulfonyl-4-methoxy-biphenyl-3-ylmethyl)-amino]-cyclohexyl}-methyl-carbamate (221)

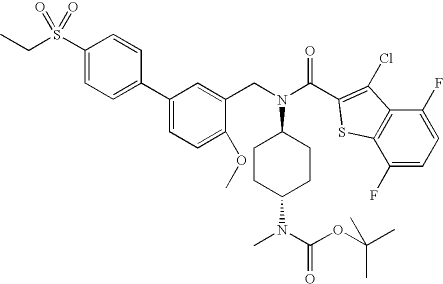

Aryl bromide 220 (200 mg, 0.30 mmol) was coupled to 4-(ethanesulfonyl)-benzene boronic acid (65 mg, 0.30 mmol) using Method A to afford the title compound.

Yield: 94 mg (42%).

LC/MS tᵣ 1.92 min.

MS (ES+) m/z 693, 691 (M−C(CH₃)₃+H).

### 3-Chloro-4,7-difluoro-benzo[b]thiophene-2-carboxylic acid (4′-ethanesulfonyl-4-methoxy-biphenyl-3-ylmethyl)-(4-methylamino-cyclohexyl)-amide hydrochloride (222)

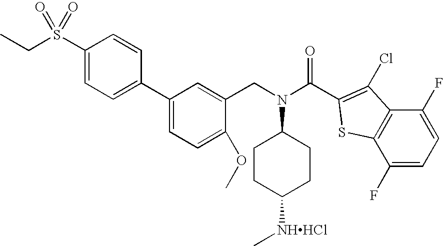

tert-Butyl carbamate 221 (94 mg, 0.13 mmol) was deprotected using Method F to afford the title compound.

Yield: 88 mg (quant.).

LC/MS tᵣ 1.39 min.

MS (ES+) m/z 649, 647 (M+H).

### Synthesis of Compound 390

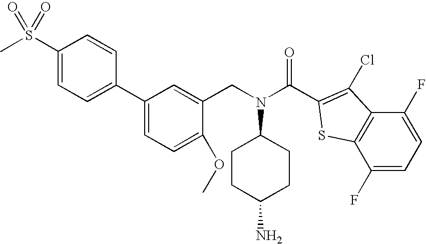

### tert-Butyl (4-amino-cyclohexyl)-carbamate (223)

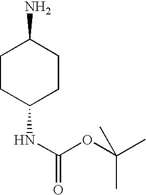

A solution of trans-1,4-diaminocyclohexane (1 kg, 8.76 mol) in THF (14 L) at 0° C. was treated with a solution of di-tert-butyl dicarbonate (238 g, 1.09 mol) in THF (500 mL) over 30 minutes. The reaction mixture was then warmed to RT, stirred 16 h and filtered. The isolated solids were washed with THF (3×2 L) and then the filtrate was reduced in vacuo. The residue thus obtained was suspended in water (8 L) and filtered once more. The filtrate was extracted with DCM (3×2 L) and the combined organic phases dried over Na₂SO₄ and reduced in vacuo. The residue was taken up in TBME (2.4 L), washed with water (3×300 mL) then concentrated to ˜400 mL. Heptane (1 L) was added to induce precipitation of the desired amine; the resulting suspension was stirred 1 h at 0° C., filtered and washed with heptane (2×100 mL) to afford the title compound.

Yield: 100.5 g (43%).

### 3-[(4-BOC-cyclohexylamino)-methyl]-4-methoxy-benzene boronic acid (224)

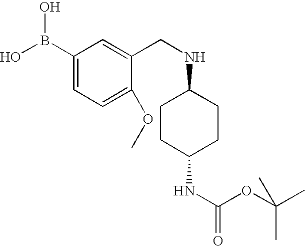

Amine 223 (598 mg, 2.79 mmol) was treated with 3-formyl-4-methoxyphenylboronic acid (500 mg, 2.79 mmol) in accordance with Method C to give the crude title compound.

Yield: 968 mg (quant.).

LC/MS tᵣ 1.10 min.

MS (ES+) m/z 379 (M+H), 323 (M−C(CH₃)₃+H).

### tert-Butyl {4-[(4′-methanesulfonyl-4-methoxy-biphenyl-3-ylmethyl)-amino]-cyclohexyl}-carbamate (225)

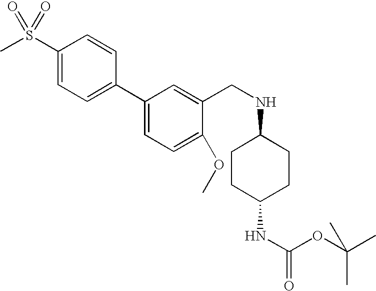

Boronic acid 224 (500 mg, 1.32 mmol) was coupled to 4-bromophenyl methyl sulfone (311 mg, 1.32 mmol) using Method A to give the title compound.

Yield: 502 mg (77%).

LC/MS tᵣ 1.28 min.

MS (ES+) m/z 489 (M+H).

### tert-Butyl {4-[(3-chloro-4,7-difluoro-benzo[b]thiophene-2-carbonyl)-(4′-methanesulfonyl-4-methoxy-biphenyl-3-ylmethyl)-amino]-cyclohexyl}-carbamate (226)

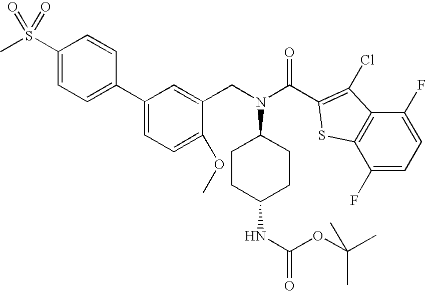

Biaryl amine 225 (502 mg, 1.02 mmol) was treated with acid chloride 8 (301 mg, 1.13 mmol) using Method D to give the title compound.

Yield: 452 mg (62%).

LC/MS tᵣ 1.79 min.

MS (ES+) m/z 721, 719 (M+H), 665, 663 (M−C(CH₃)₃+H).

### 3-Chloro-4,7-difluoro-benzo[b]thiophene-2-carboxylic acid (4-amino-cyclohexyl)-(4′-methanesulfonyl-4-methoxy-biphenyl-3-ylmethyl)-amide hydrochloride (227)

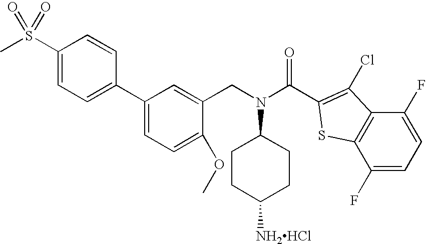

tert-Butyl carbamate 226 (452 mg, 0.62 mmol) was deprotected using Method F to give the title compound.

Yield: 332 mg (85%).

LC/MS tᵣ 1.35 min.

MS (ES+) m/z 621, 619 (M+H).

### Synthesis of Compound 391

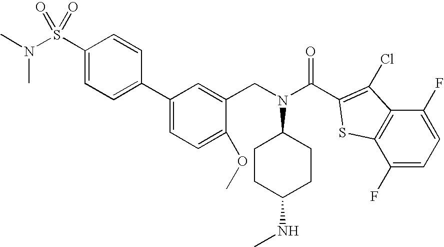

### tert-Butyl {4-[(3-chloro-4,7-difluoro-benzo[b]thiophene-2-carbonyl)-(4′-dimethylsulfamoyl-4-methoxy-biphenyl-3-ylmethyl)-amino]-cyclohexyl}-methyl-carbamate (228)

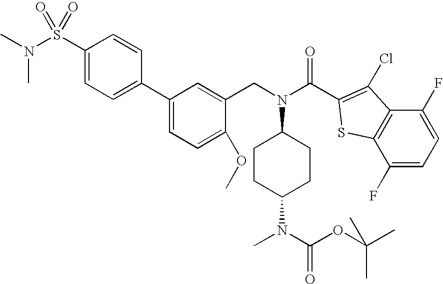

A stirred suspension of hexane-washed sodium hydride (27 mg, 0.68 mmol, 60% dispersion in mineral oil) in THF (10 mL) at 0° C. was treated with sulfonamide 217 (100 mg, 0.14 mmol) in one portion. After stirring 0.5 h, the reaction mixture was treated with iodomethane (90 μL, 1.40 mmol) via syringe and stirred 16 h at RT. The reaction was then diluted with water (10 mL) and extracted into EtOAc (3×20 mL). The combined EtOAc phases were dried (Na₂SO₄) and reduced in vacuo to afford the title compound.

Yield: 107 mg (quant.).

LC/MS tᵣ 1.92 min.

MS (ES+) m/z 708, 706 (M−C(CH₃)₃+H).

### 3-Chloro-4,7-difluoro-benzo[b]thiophene-2-carboxylic acid (4′-dimethylsulfamoyl-4-methoxy-biphenyl-3-ylmethyl)-(4-methylamino-cyclohexyl)-amide trifluoroacetate (229)

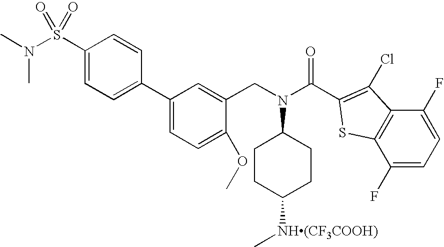

tert-Butyl carbamate 228 (107 mg, 0.14 mmol) was deprotected using Method G. The isolated salt was then dissolved in the minimum amount of DCM and added dropwise to cold (0° C.) TBME (10 mL), but the product failed to precipitate out cleanly. Thus the TBME was removed in vacuo and the residue purified by column chromatography (gradient elution—30-100% EtOAc in heptane with 0.5% triethylamine) followed by preparative HPLC to afford the title compound.

Yield: 23 mg (21%).

LC/MS tᵣ 1.46 min.

MS (ES+) m/z 664, 662 (M+H).

### Synthesis of Compound 392

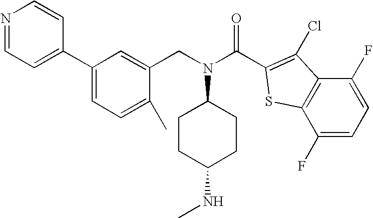

### 5-Bromo-2-methylbenzaldehyde (230)

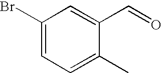

This compound was prepared from o-tolualdehyde (1.0 g, 8.32 mmol) in accordance with the procedure of Kelly et al. (Kelly, T. R.; Silva, R. A.; De Silva, H.; Jasmin, S.; Zhao, Y. J. Am. Chem. Soc., 2000, 122, 6935-6949).

Yield: 19 mg (1.1%).

### 2-Methyl-5-pyridin-4-ylbenzaldehyde (231)

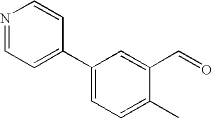

Pyridine-4-boronic acid (14 mg, 96 μmol) was coupled to aryl bromide 230 (19 mg, 96 μmol) using Method A to give the title compound.

Yield: 16 mg (84%).

LC/MS tᵣ 0.89 min.

MS (ES+) m/z 198 (M+H).

### tert-Butyl methyl-[4-(2-methyl-5-pyridin-4-yl-benzylamino)-cyclohexyl]-carbamate (232)

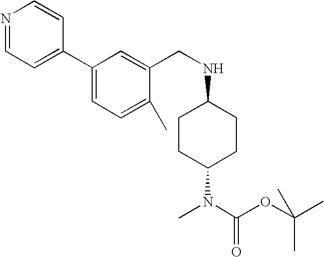

Amine 3 (19 mg, 81 μmol) was treated with aldehyde 231 (16 mg, 81 μmol) in accordance with Method C to give the crude title compound.

Yield: 36 mg.

LC/MS tᵣ 0.73 min.

MS (ES+) m/z 310 (M−CO₂C(CH₃)₃+H).

### tert-Butyl {4-[(3-chloro-4,7-difluoro-benzo[b]thiophene-2-carbonyl)-(2-methyl-5-pyridin-4-yl-benzyl)-amino]-cyclohexyl}-methyl-carbamate (233)

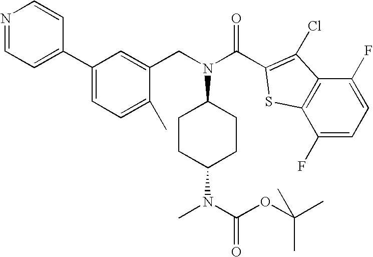

Crude biaryl amine 232 (36 mg) was treated with acid chloride 8 (29 mg, 0.11 mmol) using Method D to afford the title compound.

Yield: 27 mg (52% over two steps).

LC/MS tᵣ 1.65 min.

MS (ES+) m/z 642, 640 (M+H).

### 3-Chloro-4,7-difluoro-benzo[b]thiophene-2-carboxylic acid (4-methylamino-cyclohexyl)-(2-methyl-5-pyridin-4-yl-benzyl)-amide bis(trifluoroacetate) (234)

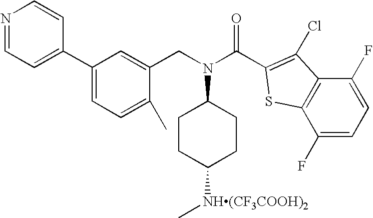

tert-Butyl carbamate 233 (27 mg, 42 μmol) was deprotected using Method G. The isolated salt was then dissolved in the minimum amount of DCM and added dropwise to cold (0° C.) TBME (5 mL). The resultant cream precipitate was isolated by filtration and dried to afford the title compound.

Yield: 13 mg (41%).

LC/MS tᵣ 1.16 min.

MS (ES+) m/z 542, 540 (M+H).

### Synthesis of Compound 393

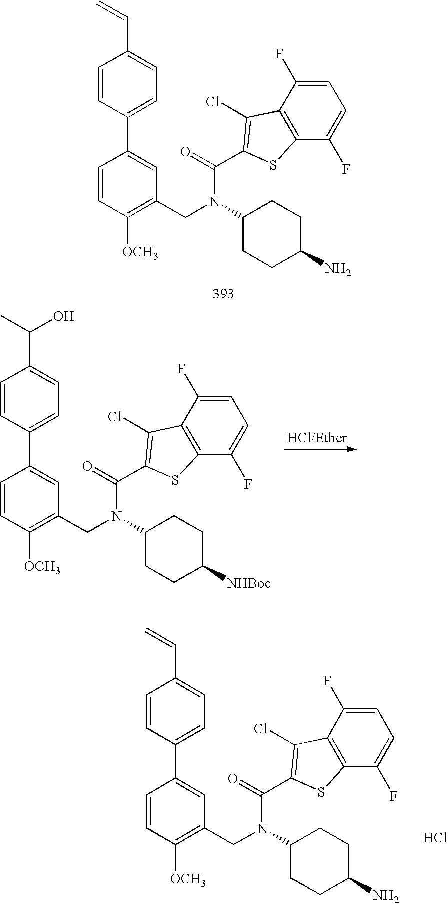

Treatment of tert-butyl (1r,4r)-4-(3-chloro-4,7-difluoro-N-((4′-(1-hydroxyethyl)-4-methoxybiphenyl-3-yl)methyl)benzo[b]thiophene-2-carboxamido)cyclohexylcarbamate (prepared by methods described herein) with ethereal hydrochloride led to the quantitative formation of N-((1r,4r)-4-aminocyclohexyl)-3-chloro-4,7-difluoro-N-((4-methoxy-4′-vinylbiphenyl-3-yl)methyl)benzo[b]thiophene-2-carboxamide as the HCl salt.

¹H NMR (400 MHz, DMSO-d₆, 60° C.) δ ppm 0.51-0.69 (m, 1H) 0.63-0.85 (m, 2H) 1.05-1.47 (m, 6H) 1.69-1.87 (m, 2H) 2.16-2.43 (m, 3H) 2.91-3.26 (m, 3H) 3.78-3.89 (m, 1H) 3.97 (s, 2H) 6.20-6.31 (m, 1H) 6.31-6.46 (m, 1H) 6.58-6.94 (m, 4H) 7.10 (1H) 7.21 (d, J=8.24 Hz, 1H)

ESI MS m/z 567 [C₃₁H₂₉ClF₂N₂O₂S+H]⁺.

### Synthesis of Compound 395

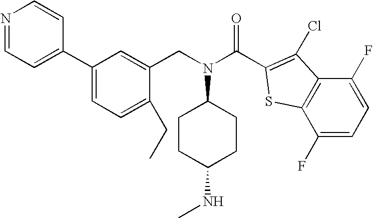

### 5-Bromo-2-ethyl-benzaldehyde (235)

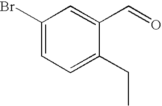

A stirred suspension of 2-ethylbenzaldehyde (0.98 mL, 7.45 mmol) and AlCl₃ (1.74 g, 13 mmol) in DCM (4.5 mL) at 0° C. was treated with a solution of Br₂ (0.38 mL, 7.45 mmol) in DCM (4.5 mL) dropwise over 6 h then stirred 16 h at RT. After this time, the reaction mixture was poured over ice-water (50 μL), the layers separated and the aqueous layer extracted with DCM (3×50 mL). The organic phases were combined and washed with 2M HCl (100 mL), aqueous NaHCO₃ (100 mL) and brine (100 mL), dried over Na₂SO₄ then the solvents removed in vacuo. Purification by column chromatography (gradient elution—100% heptane increasing to 10% EtOAc in heptane) gave the title compound.

Yield: 925 mg (58%).

LC/MS tᵣ 1.55 min.

MS (ES+) m/z mass ion not detected.

### 2-Ethyl-5-pyridin-4-yl-benzaldehyde (236)

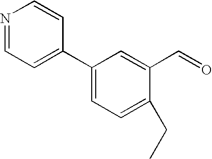

Pyridine-4-boronic acid (75 mg, 0.52 mmol) was coupled to aryl bromide 235 (111 mg, 0.52 mmol) using Method A to give the title compound.

Yield: 59 mg (54%).

LC/MS tᵣ 1.01 min.

MS (ES+) m/z 212 (M+H).

### tert-Butyl [4-(2-ethyl-5-pyridin-4-yl-benzylamino)-cyclohexyl]-methyl-carbamate (237)

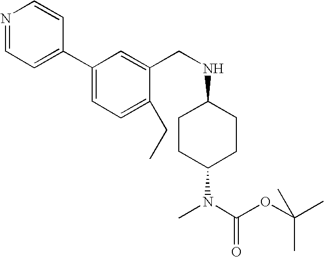

Amine 3 (54 mg, 0.23 mmol) was treated with aldehyde 236 (50 mg, 0.23 mmol) in accordance with Method C. Purification by column chromatography (50% EtOAc in heptane with 2% triethylamine) gave the title compound.

Yield: 45 mg (45%).

LC/MS tᵣ 1.14 min.

MS (ES+) m/z 424 (M+H), 368 (M−C(CH₃)₃+H).

### tert-Butyl {4-[(3-chloro-4,7-difluoro-benzo[b]thiophene-2-carbonyl)-(2-ethyl-5-pyridin-4-yl-benzyl)-amino]-cyclohexyl}-methyl-carbamate (238)

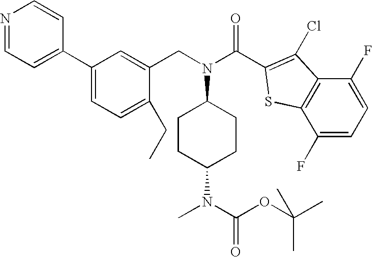

Biaryl amine 237 (45 mg, 0.10 mmol) was treated with acid chloride 8 (31 mg, 0.11 mmol) using Method D to afford the title compound.

Yield: 40 mg (58%).

LC/MS tᵣ 1.72 min.

MS (ES+) m/z 656, 654 (M+H), 600, 598 (M−C(CH₃)₃+H).

### 3-Chloro-4,7-difluoro-benzo[b]thiophene-2-carboxylic acid (2-ethyl-5-pyridin-4-yl-benzyl)-(4-methylamino-cyclohexyl)-amide dihydrochloride (239)

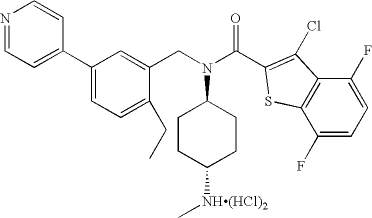

tert-Butyl carbamate 238 (40 mg, 0.06 mmol) was deprotected using Method F. The isolated salt was then dissolved in the minimum amount of DCM and added dropwise to cold (0° C.) TBME (5 mL). The resultant precipitate was isolated by filtration and dried to afford the title compound.

Yield: 6 mg (18%).

LC/MS tᵣ 1.55 min.

MS (ES+) m/z 556, 554 (M+H).

### Synthesis of Compound 394

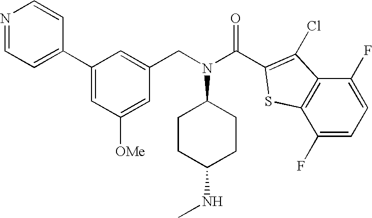

### 3-Hydroxy-5-methoxybenzaldehyde (240)

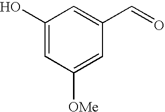

A stirred solution of 3,5-dihydroxybenzaldehyde (1.0 g, 7.24 mmol) in DMF (20 mL) was treated with sodium hydride (319 mg, 8.0 mmol, 60% dispersion in mineral oil) at 0° C. After warming to RT and stirring 0.5 h, the reaction was treated with iodomethane (0.50 mL, 8.0 mmol) via syringe and stirred 16 h. It was then diluted with 1 M HCl (100 mL) and extracted into EtOAc (50 mL). The EtOAc phase was washed with 1 M HCl (50 mL), water (2×50 mL) and brine (50 mL), dried over Na₂SO₄ then the EtOAc removed in vacuo. The title compound was obtained after chromatography (gradient elution—0-50% EtOAc in heptane).

Yield: 315 mg (29%).

### 3-Formyl-5-methoxyphenyl trifluoromethanesulfonate (241)

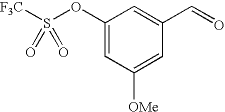

A stirred solution of phenol 240 (315 mg, 2.07 mmol) and pyridine (0.33 mL, 4.14 mmol) in DCM (8 mL) and THF (8 mL) at 0° C. was treated with trifluoromethanesulfonic anhydride (0.40 mL, 2.38 mmol) dropwise via syringe over 5 minutes. After warming to RT and stirring 16 h, the reaction was diluted with water (25 mL) and extracted into DCM (3×50 mL). The combined DCM phases were then dried over Na₂SO₄ and reduced in vacuo. The title compound was obtained after chromatography (gradient elution—0-40% EtOAc in heptane).

Yield: 387 mg (66%).

LC/MS tᵣ 1.54 min.

MS (ES+) mass ion not detected.

### 3-Methoxy-5-pyridin-4-ylbenzaldehyde (242)

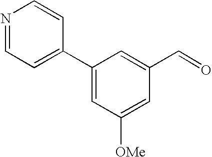

Pyridine-4-boronic acid (50 mg, 0.35 mmol) was coupled to aryl triflate 241 (66 mg, 0.23 mmol) using Method A to give the title compound.

Yield: 22 mg (45%).

LC/MS tᵣ 0.91 min.

MS (ES+) m/z 214 (M+H).

### tert-Butyl [4-(3-methoxy-5-pyridin-4-yl-benzylamino)-cyclohexyl]-methyl-carbamate (243)

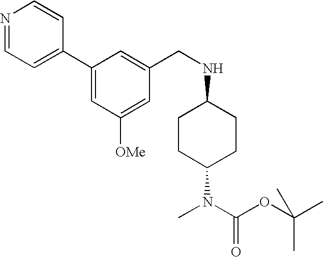

Amine 3 (17 mg, 75 μmol) was treated with aldehyde 242 (16 mg, 75 μmol) in accordance with Method C. The title compound was obtained after chromatography (gradient elution—40-100% EtOAc in heptane with 0.5% triethylamine).

Yield: 18 mg (56%).

LC/MS tᵣ 1.08 min.

MS (ES+) m/z 426 (M+H).

### tert-Butyl {4-[(3-chloro-4,7-difluoro-benzo[b]thiophene-2-carbonyl)-(3-methoxy-5-pyridin-4-yl-benzyl)-amino]-cyclohexyl}-methyl-carbamate (244)

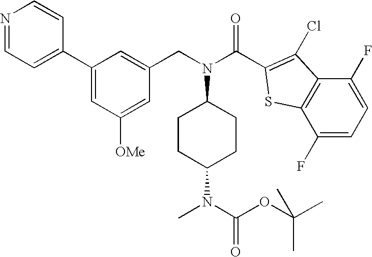

Biaryl amine 243 (18 mg, 42 μmol) was treated with acid chloride 8 (14 mg, 51 μmol) using Method D to afford the title compound.

Yield: 15 mg (54%).

LC/MS tᵣ 1.60 min.

MS (ES+) m/z 658, 656 (M+H).

### 3-Chloro-4,7-difluoro-benzo[b]thiophene-2-carboxylic acid (3-methoxy-5-pyridin-4-yl-benzyl)-(4-methylamino-cyclohexyl)-amide bis(trifluoroacetate) (245)

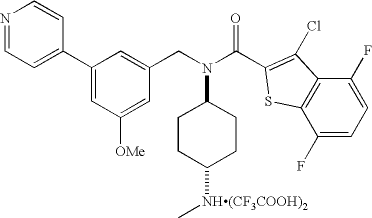

tert-Butyl carbamate 244 (15 mg, 23 mmol) was deprotected using Method G to afford the title compound.

Yield: 18 mg (quant.).

LC/MS tᵣ 1.50 min.

MS (ES+) m/z 558, 556 (M+H).

¹H NMR δ_{H} ppm (400 MHz, D₆-DMSO, 95° C.): 8.58 (2H, d), 8.15 (2H, br. s), 7.57 (2H, d), 7.36-7.20 (2H, m), 7.17 (1H, br. s), 7.13 (1H, dd), 6.91 (1H, br. s), 4.66 (2H, s), 3.92-3.73 (1H, obsc. m), 3.75 (3H, s), 2.93-2.78 (1H, m), 2.42 (3H, obsc. s), 2.06-1.92 (2H, m), 1.87-1.63 (4H, m), 1.36-1.16 (2H, m).

### Synthesis of Compound 310

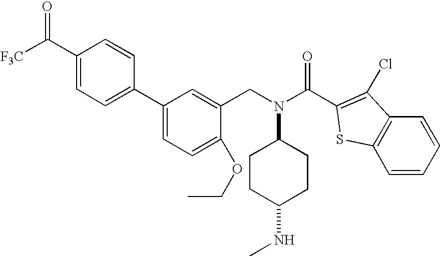

### 3-Chloro-benzo[b]thiophene-2-carboxylic acid [4-ethoxy-4′-(2,2,2-trifluoro-acetyl)-biphenyl-3-ylmethyl]-(4-methylamino-cyclohexyl)-amide hydrochloride (246)

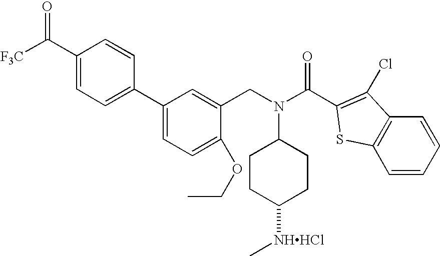

The title compound was prepared from boronic acid 11 (20 mg, 49 μmol) and 4′-bromo-2,2,2-trifluoroacetophenone (10.4 mg, 41 μmol) in accordance with Method L1.

Yield: 17.3 mg (63%).

LC/MS tᵣ 1.49 min.

MS (ES+) m/z 649, 647 (M+H₂O+H), 618, 616 (M−31+H₂O+H).

### Synthesis of Compound 378

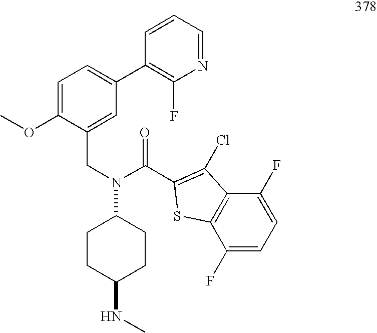

### Synthesis of 5-(2-Fluoro-pyridin-3-yl)-2-methoxy-benzaldehyde

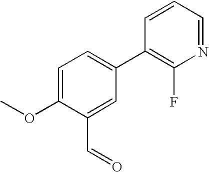

Following the synthetic protocol described in Method A′; 5-(2-Fluoro-pyridin-3-yl)-2-methoxy-benzaldehyde was prepared starting from 2-Fluoro-3-boronic acid pyridine and 5-Bromo-2-methoxy-benzaldehyde. The desired product was isolated in 72%.

ESI MS m/z 232 [C₁₃H₁₀FNO₂+H]⁺.

### Synthesis of {4-[5-(2-Fluoro-pyridin-3-yl)-2-methoxy-benzylamino]-cyclohexyl}-methyl-carbamic acid tert-butyl ester

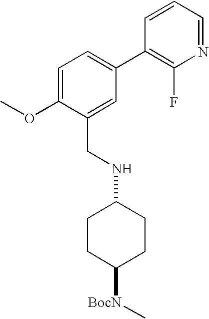

Following the synthetic protocol described in Method C′; {4-[5-(2-Fluoro-pyridin-3-yl)-2-methoxy-benzylamino]-cyclohexyl}-methyl-carbamic acid tert-butyl ester was prepared starting from 5-(2-Fluoro-pyridin-3-yl)-2-methoxy-benzaldehyde and (4-Amino-cyclohexyl)-methyl-carbamic acid tert-butyl ester. The desired product was isolated in 70%.

ESI MS m/z 444 [C₂₅H₃₄FN₃O₃+H]⁺.

1H NMR (400 MHz, CHLOROFORM-d) δ ppm 1.42 (s, 4H) 1.46 (s, 9H) 1.68-1.76 (m, J=1.47 Hz, 2H) 1.93-2.01 (m, 1H) 2.01-2.13 (m, J=12.20 Hz, 2H) 2.42-2.56 (m, 1H) 2.70 (s, 3H) 3.40 (br. s., 1H) 3.80-3.98 (m, 5H) 6.94-6.99 (m, J=9.03 Hz, 1H) 7.23-7.28 (m, 1H) 7.47-7.53 (m, 2H) 7.87 (ddd, J=9.76, 7.56, 1.95 Hz, 1H) 8.15 (dt, J=3.17, 1.71 Hz, 1H

### Synthesis of 3-Chloro-4,7-difluoro-benzo[b]thiophene-2-carboxylic acid [5-(2-fluoro-pyridin-3-yl)-2-methoxy-benzyl]-(4-methylamino-cyclohexyl)-amide

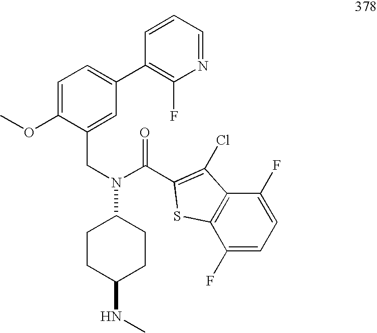

Following the synthetic protocol described in Method D′; 3-Chloro-4,7-difluoro-benzo[b]thiophene-2-carboxylic acid [5-(2-fluoro-pyridin-3-yl)-2-methoxy-benzyl]-(4-methylamino-cyclohexyl)-amide was prepared starting from {4-[5-(2-Fluoro-pyridin-3-yl)-2-methoxy-benzylamino]-cyclohexyl}-methyl-carbamic acid tert-butyl ester and 3-Chloro-4,7-difluoro-benzo[b]thiophene-2-carbonyl chloride. The desired product was isolated in 45%.

¹H NMR (400 MHz, DMSO-d₆, 60° C.) δ ppm 1.12-1.44 (m, 2H) 1.56-2.14 (m, 6H) 2.40-2.48 (m, 3H) 2.82-3.01 (m, 1H) 3.64-4.14 (m, 4H) 4.56-4.70 (m, 2H) 6.98-7.26 (m, 1H) 7.31-7.60 (m, 5H) 7.99-8.08 (m, 1H) 8.14-8.41 (m, 3H)

ESI MS m/z 574 [C₂₉H₂₇ClF₃N₃O₂S+H]⁺.

### Synthesis of Compound 379

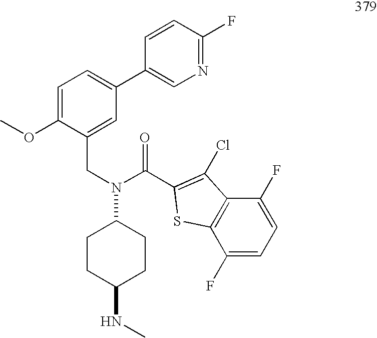

### 5-(6-Fluoro-pyridin-3-yl)-2-methoxy-benzaldehyde

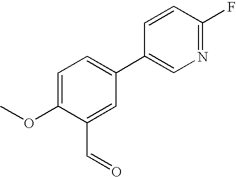

Following the synthetic protocol described in Method A′; 5-(6-Fluoro-pyridin-3-yl)-2-methoxy-benzaldehyde was prepared starting from 6-Fluoro-2-boronic acid pyridine and 5-Bromo-2-methoxy-benzaldehyde. The desired product was isolated in 88%.

ESI MS m/z 232 [C₁₃H₁₀FNO₂+H]⁺.

### {4-[5-(6-Fluoro-pyridin-3-yl)-2-methoxy-benzylamino]-cyclohexyl}-methyl-carbamic acid tert-butyl ester

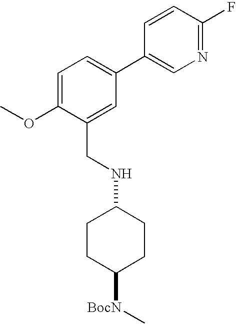

Following the synthetic protocol described in Method C′; {4-[5-(6-Fluoro-pyridin-3-yl)-2-methoxy-benzylamino]-cyclohexyl}-methyl-carbamic acid tert-butyl ester was prepared starting from 5-(6-Fluoro-pyridin-3-yl)-2-methoxy-benzaldehyde and (4-Amino-cyclohexyl)-methyl-1-carbamic acid tert-butyl ester. The desired product was isolated in 84%.

ESI MS m/z 444 [C₂₅H₃₄FN₃O₃+H]⁺.

### 3-Chloro-4,7-difluoro-benzo[b]thiophene-2-carboxylic acid [5-(6-fluoro-pyridin-3-yl)-2-methoxy-benzyl]-(4-methylamino-cyclohexyl)-amide

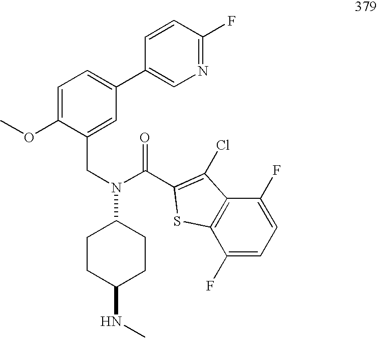

Following the synthetic protocol described in Method D′; 3-Chloro-4,7-difluoro-benzo[b]thiophene-2-carboxylic acid [5-(6-fluoro-pyridin-3-yl)-2-methoxy-benzyl]-(4-methylamino-cyclohexyl)-amide was prepared starting from {4-[5-(6-Fluoro-pyridin-3-yl)-2-methoxy-benzylamino]-cyclohexyl}-methyl-carbamic acid tert-butyl ester and 3-Chloro-4,7-difluoro-benzo[b]thiophene-2-carbonyl chloride. The desired product was isolated in 57%.

¹H NMR (400 MHz, DMSO-d₆, 60° C.) δ ppm 1.12-1.46 (m, 2H) 1.52-2.14 (m, 6H) 2.39-2.50 (m, 3H) 2.79-2.99 (m, 1H) 3.59-4.12 (m, 4H) 4.56-4.70 (m, 2H) 6.96-7.22 (m, 1H) 7.22-7.58 (m, 4H) 7.63 (dd, J=8.54, 2.20 Hz, 1H) 8.07-8.25 (m, 2H) 8.25-8.37 (m, 1H) 8.39-8.49 (m, 1H)

ESI MS m/z 574 [C₂₉H₂₇ClF₃N₃O₂S+H]⁺.

### Synthesis of Compound 380

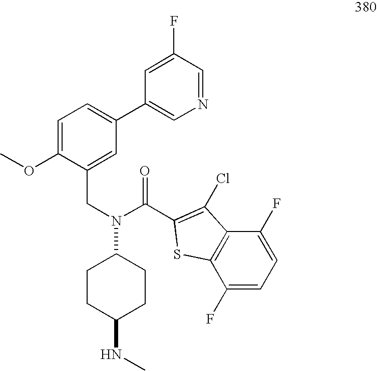

### 5-(5-Fluoro-pyridin-3-yl)-2-methoxy-benzaldehyde

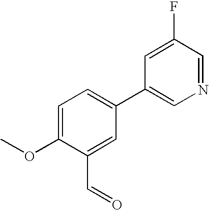

Following the synthetic protocol described in Method A′; 5-(5-Fluoro-pyridin-3-yl)-2-methoxy-benzaldehyde was prepared starting from 5-Fluoro-2-boronic acid pyridine and 5-Bromo-2-methoxy-benzaldehyde. The desired product was isolated in 44%.

ESI MS m/z 232 [C₁₃H₁₀FNO₂+H]⁺.

### {4-[5-(5-Fluoro-pyridin-3-yl)-2-methoxy-benzylamino]-cyclohexyl}-methyl-carbamic acid tert-butyl ester

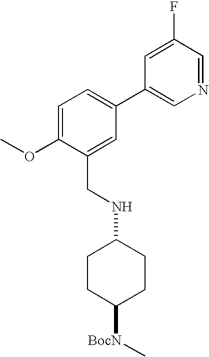

Following the synthetic protocol described in Method C′; {4-[5-(5-Fluoro-pyridin-3-yl)-2-methoxy-benzylamino]-cyclohexyl}-methyl-carbamic acid tert-butyl ester was prepared starting from 5-(5-Fluoro-pyridin-3-yl)-2-methoxy-benzaldehyde and (4-Amino-cyclohexyl)-methyl-carbamic acid tert-butyl ester. The desired product was isolated in 64%.

ESI MS m/z 444 [C₂₅H₃₄FN₃O₃+H]⁺.

1H NMR (400 MHz, CHLOROFORM-d) δ ppm 1.37-1.54 (m, 12H) 1.67-1.81 (m, 2H) 1.99 (s, 2H) 2.07-2.20 (m, 2H) 2.52-2.63 (m, 1H) 2.70 (s, 3H) 3.92 (s, 3H) 3.96 (s, 2H) 5.32 (s, 1H) 6.98 (d, J=8.54 Hz, 1H) 7.50 (dd, J=2.44 Hz, 1H) 7.57 (t, J=2.32 Hz, 1H) 7.58-7.62 (m, 1H) 8.41 (d, J=2.68 Hz, 1H) 8.63 (t, J=1.71 Hz, 1H).

### 3-Chloro-4,7-difluoro-benzo[b]thiophene-2-carboxylic acid [5-(5-fluoro-pyridin-3-yl)-2-methoxy-benzyl]-(4-methylamino-cyclohexyl)-amide

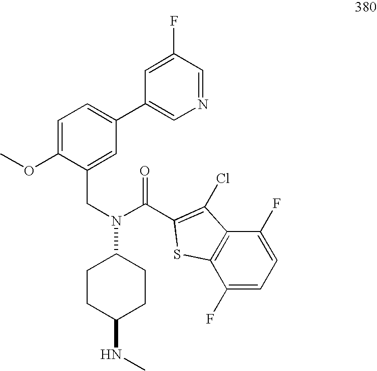

Following the synthetic protocol described in Method D′; 3-Chloro-4,7-difluoro-benzo[b]thiophene-2-carboxylic acid [5-(5-fluoro-pyridin-3-yl)-2-methoxy-benzyl]-(4-methylamino-cyclohexyl)-amide was prepared starting from {4-[5-(5-Fluoro-pyridin-3-yl)-2-methoxy-benzylamino]-cyclohexyl}-methyl-carbamic acid tert-butyl ester and 3-Chloro-4,7-difluoro-benzo[b]thiophene-2-carbonyl chloride. The desired product was isolated in 52%.

¹H NMR (300 MHz, DMSO-d₆, 60° C.) δ ppm 1.24-1.49 (m, 2H) 1.63-1.97 (m, 4H) 2.08 (d, J=10.98 Hz, 2H) 2.45 (t, J=4.85 Hz, 3H) 2.81-2.98 (m, 1H) 4.59-4.71 (m, 3H) 7.13 (d, J=7.87 Hz, 2H) 7.28-7.53 (m, 2H) 7.60 (d, J=2.20 Hz, 1H) 7.70 (dd, J=8.60, 2.38 Hz, 1H) 7.89 (dt, J=10.43, 2.20 Hz, 1H) 8.53 (d, J=2.38 Hz, 1H) 8.71 (s, 1H) 8.80 (s, 2H)

ESI MS m/z 574 [C₂₉H₂₇ClF₃N₃O₂S+H]⁺.

### Synthesis of Compound 381

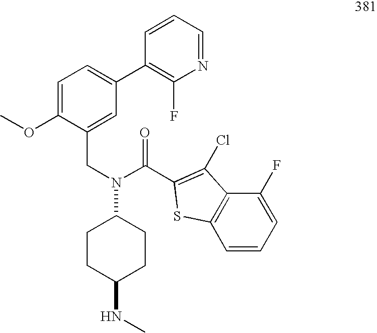

### 3-Chloro-4,7-difluoro-benzo[b]thiophene-2-carboxylic acid [5-(2-fluoro-pyridin-3-yl)-2-methoxy-benzyl]-(4-methylamino-cyclohexyl)-amide

Following the synthetic protocol described in Method D′; 3-Chloro-4,7-difluoro-benzo[b]thiophene-2-carboxylic acid [5-(2-fluoro-pyridin-3-yl)-2-methoxy-benzyl]-(4-methylamino-cyclohexyl)-amide was prepared starting from {4-[5-(2-Fluoro-pyridin-3-yl)-2-methoxy-benzylamino]-cyclohexyl}-methyl-carbamic acid tert-butyl ester and 3-Chloro-4-fluoro-benzo[b]thiophene-2-carbonyl chloride. The desired product was isolated in 18%.

¹H NMR (400 MHz, DMSO-d₆, 60° C.) δ ppm 1.12-1.45 (m, 2H) 1.58-1.91 (m, 4H) 1.91-2.12 (m, 2H) 2.35-2.47 (m, 3H) 2.77-3.02 (m, 1H) 3.58-4.17 (m, 4H) 4.50-4.74 (m, 2H) 6.95-7.22 (m, 1H) 7.24-7.42 (m, 1H) 7.42-7.63 (m, 4H) 7.77-8.09 (m, 2H) 8.09-8.26 (m, 2H) 8.27-8.43 (m, 1H)

ESI MS m/z 556 [C₂₉H₂₈ClF₂N₃O₂S+H]⁺.

### Synthesis of Compound 382

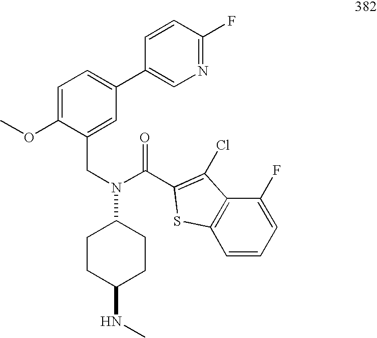

### 3-Chloro-4-fluoro-benzo[b]thiophene-2-carboxylic acid [5-(6-fluoro-pyridin-3-yl)-2-methoxy-benzyl]-(4-methylamino-cyclohexyl)-amide

Following the synthetic protocol described in Method D′; 3-Chloro-4,7-difluoro-benzo[b]thiophene-2-carboxylic acid [5-(6-fluoro-pyridin-3-yl)-2-methoxy-benzyl]-(4-methylamino-cyclohexyl)-amide was prepared starting from {4-[5-(6-Fluoro-pyridin-3-yl)-2-methoxy-benzylamino]-cyclohexyl}-methyl-carbamic acid tert-butyl ester and 3-Chloro-4-fluoro-benzo[b]thiophene-2-carbonyl chloride. The desired product was isolated in 50%.

¹H NMR (400 MHz, DMSO-d₆, 60° C.) δ ppm 1.09-1.45 (m, 2H) 1.55-2.16 (m, 6H) 2.33-2.49 (m, 2H) 2.80-3.02 (m, 1H) 3.59-4.15 (m, 4H) 4.51-4.72 (m, 2H) 6.97-7.22 (m, 1H) 7.23-7.69 (m, 5H) 7.76-8.07 (m, 1H) 8.08-8.55 (m, 4H)

ESI MS m/z 556 [C₂₉H₂₈ClF₂N₃O₂S+H]⁺.

### Synthesis of Compound 383

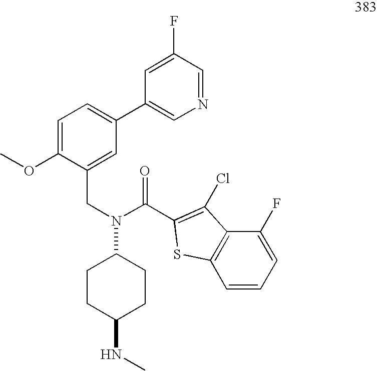

### 3-chloro-4-fluoro-N-(5-(5-fluoropyridin-3-yl)-2-methoxybenzyl)-N-((1s,4s)-4-(methylamino)cyclohexyl)benzo[b]thiophene-2-carboxamide

Following the synthetic protocol described in Method D′; 3-Chloro-4,7-difluoro-benzo[b]thiophene-2-carboxylic acid [5-(5-fluoro-pyridin-3-yl)-2-methoxy-benzyl]-(4-methylamino-cyclohexyl)-amide was prepared starting from {4-[5-(5-Fluoro-pyridin-3-yl)-2-methoxy-benzylamino]-cyclohexyl}-methyl-carbamic acid tert-butyl ester and 3-Chloro-4,7-difluoro-benzo[b]thiophene-2-carbonyl chloride. The desired product was isolated in 46%.

¹H NMR (400 MHz, DMSO-d₆, 60° C.) δ ppm 1.22-1.46 (m, 2H) 1.63-1.96 (m, 4H) 1.99-2.14 (m, 2H) 2.40-2.48 (m, 3H) 2.81-2.97 (m, 1H) 3.76-3.98 (m, 4H) 4.62-4.71 (m, 2H) 7.06-7.21 (m, 1H) 7.23-7.38 (m, 1H) 7.45-7.57 (m, 1H) 7.57-7.62 (m, 1H) 7.64-7.73 (m, 1H) 7.82-7.95 (m, 2H) 8.50-8.54 (m, 1H) 8.58-8.77 (m, 3H)

ESI MS m/z 556 [C₂₉H₂₈ClF₂N₃O₂S+H]⁺.

### Synthesis of Compound 384

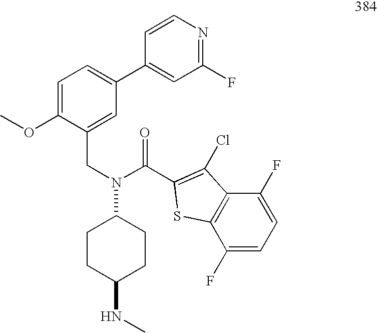

### 5-(3-Fluoro-pyridin-4-yl)-2-methoxy-benzaldehyde

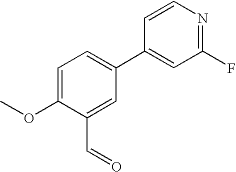

Following the synthetic protocol described in Method A′; 5-(3-Fluoro-pyridin-4-yl)-2-methoxy-benzaldehyde was prepared starting from 2-Fluoro-4-boronic acid pyridine and 5-Bromo-2-methoxy-benzaldehyde. The desired product was isolated in 82%.

ESI MS m/z 232 [C₁₃H₁₀FNO₂+H]⁺.

### {4-[5-(2-Fluoro-pyridin-4-yl)-2-methoxy-benzylamino]-cyclohexyl}-methyl-carbamic acid tert-butyl ester

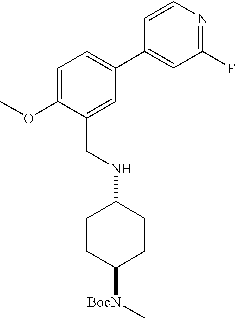

Following the synthetic protocol described in Method C′; {4-[5-(2-Fluoro-pyridin-4-yl)-2-methoxy-benzylamino]-cyclohexyl}-methyl-carbamic acid tert-butyl ester was prepared starting from 5-(3-Fluoro-pyridin-4-yl)-2-methoxy-benzaldehyde and (4-Amino-cyclohexyl)-methyl-carbamic acid tert-butyl ester. The desired product was isolated in 58%.

ESI MS m/z 444 [C₂₅H₃₄FN₃O₃+H]⁺.

### 3-Chloro-4,7-difluoro-benzo[b]thiophene-2-carboxylic acid [5-(2-fluoro-pyridin-4-yl)-2-methoxy-benzyl]-(4-methylamino-cyclohexyl)-amide

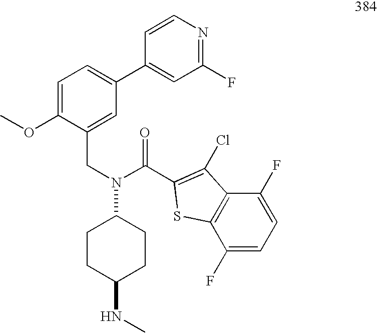

Following the synthetic protocol described in Method D′; 3-Chloro-4,7-difluoro-benzo[b]thiophene-2-carboxylic acid [5-(2-fluoro-pyridin-4-yl)-2-methoxy-benzyl]-(4-methylamino-cyclohexyl)-amide was prepared starting from {4-[5-(2-Fluoro-pyridin-4-yl)-2-methoxy-benzylamino]-cyclohexyl}-methyl-carbamic acid tert-butyl ester and 3-Chloro-4,7-difluoro-benzo[b]thiophene-2-carbonyl chloride. The desired product was isolated in 52%.

¹H NMR (400 MHz, DMSO-d₆, 60° C.) δ ppm 1.21-1.43 (m, 2H) 1.58-2.14 (m, 6H) 2.81-3.02 (m, 1H) 4.62-4.70 (m, 4H) 7.09-7.20 (m, 1H) 7.30-7.50 (m, 3H) 7.56 (dt, J=3.57, 1.65 Hz, 1H) 7.66 (d, J=2.20 Hz, 1H) 7.79 (dd, J=8.60, 2.38 Hz, 1H) 8.15-8.30 (m, J=5.31 Hz, 2H) 8.27 (d, J=5.31 Hz, 1H)

ESI MS m/z 574 [C₂₉H₂₇ClF₃N₃O₂S+H]⁺.

### Synthesis of Compound 385

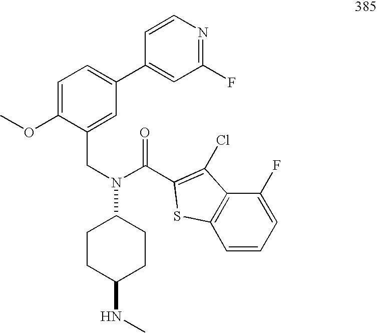

### 3-Chloro-4-fluoro-benzo[b]thiophene-2-carboxylic acid [5-(2-fluoro-pyridin-4-yl)-2-methoxy-benzyl]-(4-methylamino-cyclohexyl)-amide

Following the synthetic protocol described in Method D′; 3-Chloro-4-fluoro-benzo[b]thiophene-2-carboxylic acid [5-(2-fluoro-pyridin-4-yl)-2-methoxy-benzyl]-(4-methylamino-cyclohexyl)-amide was prepared starting from {4-[5-(2-Fluoro-pyridin-4-yl)-2-methoxy-benzylamino]-cyclohexyl}-methyl-carbamic acid tert-butyl ester and 3-Chloro-4-fluoro-benzo[b]thiophene-2-carbonyl chloride. The desired product was isolated in 49%.

¹H NMR (400 MHz, DMSO-d₆, 60° C.) δ ppm 1.17-1.41 (m, 2H) 1.61-1.93 (m, 4H) 1.93-2.10 (m, 2H) 2.45-2.48 (m, 1H) 2.81-2.99 (m, 1H) 3.76-4.01 (m, 4H) 4.60-4.71 (m, 2H) 7.07-7.23 (m, 1H) 7.24-7.41 (m, 2H) 7.52 (br. s., 1H) 7.56 (dt, J=3.52, 1.74 Hz, 1H) 7.66 (d, J=2.20 Hz, 1H) 7.79 (dd, J=8.51, 2.29 Hz, 1H) 7.91 (br. s., 1H) 8.19 (m, 2H) 8.28 (d, J=5.49 Hz, 1H)

ESI MS m/z 556 [C₂₉H₂₈ClF₂N₃O₂S+H]⁺.

### Synthesis of Compound 386

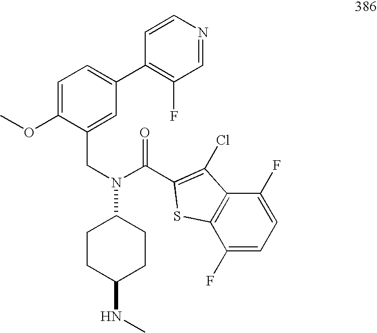

### Synthesis of 5-(3-Fluoro-pyridin-4-yl)-2-methoxy-benzaldehyde

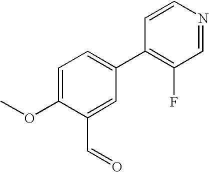

Following the synthetic protocol described in Method A′; 5-(3-Fluoro-pyridin-4-yl)-2-methoxy-benzaldehyde was prepared starting from 3-Fluoro-4-boronic acid pyridine and 5-Bromo-2-methoxy-benzaldehyde. The desired product was isolated in 49%.

ESI MS m/z 232 [C₁₃H₁₀FNO₂+H]⁺.

### {4-[5-(3-Fluoro-pyridin-4-yl)-2-methoxy-benzylamino]-cyclohexyl}-methyl-carbamic acid tert-butyl ester

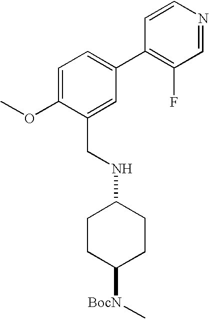

Following the synthetic protocol described in Method C′; {4-[5-(3-Fluoro-pyridin-4-yl)-2-methoxy-benzylamino]-cyclohexyl}-methyl-carbamic acid tert-butyl ester was prepared starting from 5-(3-Fluoro-pyridin-4-yl)-2-methoxy-benzaldehyde and (4-Amino-cyclohexyl)-methyl-carbamic acid tert-butyl ester. The desired product was isolated in 67%.

ESI MS m/z 444 [C₂₅H₃₄FN₃O₃+H]⁺.

### 3-Chloro-4,7-difluoro-benzo[b]thiophene-2-carboxylic acid [5-(3-fluoro-pyridin-4-yl)-2-methoxy-benzyl]-(4-methylamino-cyclohexyl)-amide

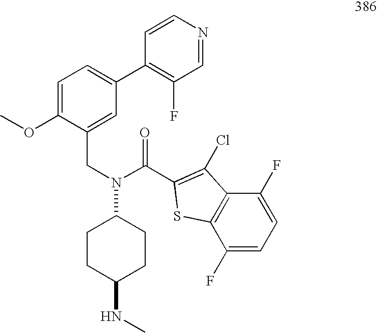

Following the synthetic protocol described in Method D′; 3-Chloro-4,7-difluoro-benzo[b]thiophene-2-carboxylic acid [5-(3-fluoro-pyridin-4-yl)-2-methoxy-benzyl]-(4-methylamino-cyclohexyl)-amide was prepared starting from {4-[5-(3-Fluoro-pyridin-4-yl)-2-methoxy-benzylamino]-cyclohexyl}-methyl-carbamic acid tert-butyl ester and 3-Chloro-4,7-difluoro-benzo[b]thiophene-2-carbonyl chloride. The desired product was isolated in 54%.

¹H NMR (400 MHz, DMSO-d₆, 60° C.) δ ppm 1.25-1.51 (m, 2H) 1.63-1.94 (m, 4H) 2.00-2.14 (m, 2H) 2.41-2.47 (m, 3H) 2.82-2.96 (m, 1H) 3.71-4.06 (m, 4H) 4.67 (s, 2H) 7.51-7.65 (m, 3H) 8.49 (d, J=4.94 Hz, 1H) 8.63 (d, J=2.93 Hz, 1H) 8.85 (m, 2H)

ESI MS m/z 574 [C₂₉H₂₇ClF₃N₃O₂S+H]⁺.

### Synthesis of Compound 387

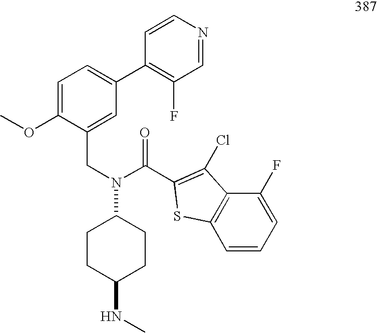

### 3-Chloro-4-fluoro-benzo[b]thiophene-2-carboxylic acid [5-(3-fluoro-pyridin-4-yl)-2-methoxy-benzyl]-(4-methylamino-cyclohexyl)-amide

Following the synthetic protocol described in Method D′; 3-Chloro-4-fluoro-benzo[b]thiophene-2-carboxylic acid [5-(3-fluoro-pyridin-4-yl)-2-methoxy-benzyl]-(4-methylamino-cyclohexyl)-amide was prepared starting from {4-[5-(2-Fluoro-pyridin-4-yl)-2-methoxy-benzylamino]-cyclohexyl}-methyl-carbamic acid tert-butyl ester and 3-Chloro-4-fluoro-benzo[b]thiophene-2-carbonyl chloride. The desired product was isolated in 61%.

¹H NMR (400 MHz, DMSO-d₆, 60° C.) δ ppm 1.26-1.46 (m, 2H) 1.64-1.95 (m, 4H) 1.97-2.18 (m, 2H) 2.37-2.48 (m, 3H) 2.77-3.00 (m, 1H) 3.70-4.05 (m, 4H) 4.66 (s, 2H) 7.47-7.66 (m, 4H) 7.81-8.02 (m, 1H) 8.49 (d, J=4.94 Hz, 1H) 8.63 (d, J=2.75 Hz, 1H) 8.73-9.00 (m, 2H)

ESI MS m/z 556 [C₂₉H₂₈ClF₂N₃O₂S+H]⁺.

### Synthesis of Compound 374

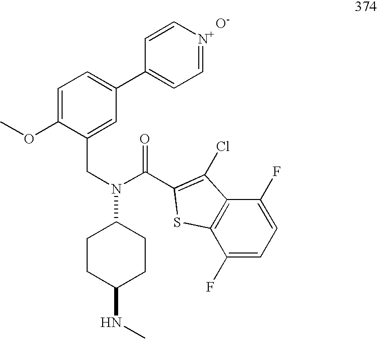

### 4-(3-((3-chloro-4,7-difluoro-N-((1s,4s)-4-(methylamino)cyclohexyl)benzo[b]thiophene-2-carboxamido)methyl)-4-methoxyphenyl)pyridine 1-oxide

Following the synthetic protocol described in Method X; 4-(3-((3-chloro-4,7-difluoro-N-((1s,4s) -4-(methylamino)cyclohexyl)benzo[b]thiophene-2-carboxamido)methyl)-4-methoxyphenyl)pyridine 1-oxide was prepared starting from tert-butyl (1s,4s)-4-(3-chloro-4,7-difluoro-N-(2-methoxy-5-(pyridin-4-yl)benzyl)benzo[b]thiophene-2-carboxamido)cyclohexyl(methyl)carbamate. The desired product was isolated in 65%.

H NMR (300 MHz, DMSO-d₆, 60° C.) δ ppm 1.18-1.48 (m, 2H) 1.59-1.95 (m, 4H) 1.97-2.14 (m, 2H) 2.41-2.48 (m, 3H) 2.77-3.02 (m, 1H) 3.73-3.95 (m, 4H) 4.65 (s, 2H) 7.05-7.20 (m, 1H) 7.31-7.49 (m, 2H) 7.60 (d, J=2.38 Hz, 1H) 7.65-7.76 (m, 3H) 8.34 (d, J=6.95 Hz, 2H) 8.58-8.77 (m, 2H)

ESI MS m/z 572 [C₂₉H₂₈ClF₂N₃O₃S+H]⁺.

### Synthesis of Compound 375

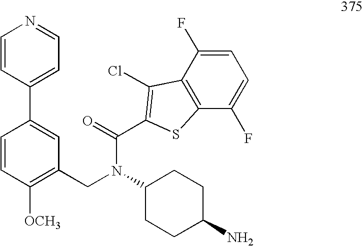

### tert-Butyl (1r,4r)-4-(2-methoxy-5-(pyridin-4-yl)benzylamino)cyclohexylcarbamate

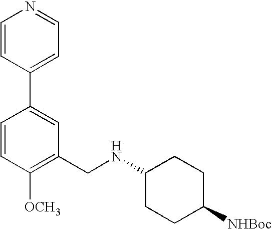

Following the synthetic protocol described in Method C′; tert-butyl (1r,4r)-4-(2-methoxy-5-(pyridin-4-yl)benzylamino)cyclohexylcarbamate was prepared starting from 2-methoxy-5-(pyridin-4-yl)benzaldehyde and tert-butyl (1r,4r)-4-aminocyclohexylcarbamate. The desired product was isolated in 58%.

ESI MS m/z 412 [C₂₄H₃₃N₃O₃+H]⁺.

### N-((1r,4r)-4-aminocyclohexyl)-3-chloro-4,7-difluoro-N-(2-methoxy-5-(pyridin-4-yl)benzyl)benzo[b]thiophene-2-carboxamide hydrochloride

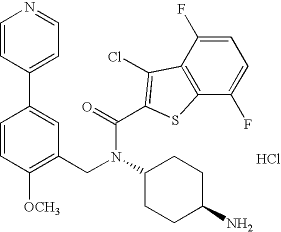

Following the synthetic protocol described in Method D′; N-((1r,4r)-4-aminocyclohexyl)-3-chloro-4,7-difluoro-N-(2-methoxy-5-(pyridin-4-yl)benzyl)benzo[b]thiophene-2-carboxamide hydrochloride was prepared starting from tert-butyl (1r,4r)-4-(2-methoxy-5-(pyridin-4-yl)benzylamino)cyclohexylcarbamate and 3-Chloro-4,7-difluoro-benzo[b]thiophene-2-carbonyl chloride. The desired product was isolated in 49% as the HCl salt.

¹H NMR (400 MHz, DMSO-d₆, 60° C.) δ ppm 1.14-1.57 (m, 2H) 1.61-2.20 (m, 6H) 2.65-3.08 (m, 2H) 4.68 (s, 2H) 7.00-7.59 (m, 5H) 7.68-7.85 (m, 1H) 7.86-8.24 (m, 6H) 8.59-8.96 (m, 3H)

ESI MS m/z 556 [C₂₉H₂₈ClF₂N₃O₂S+H]⁺.

ESI MS m/z 542 [C₂₈H₂₆ClF₂N₃O₂S+H]⁺.

### Process Exemplification

A. Overview of an Improved Synthetic Scheme:

An improved synthetic scheme for the preparation of P8a was developed (Scheme PI).

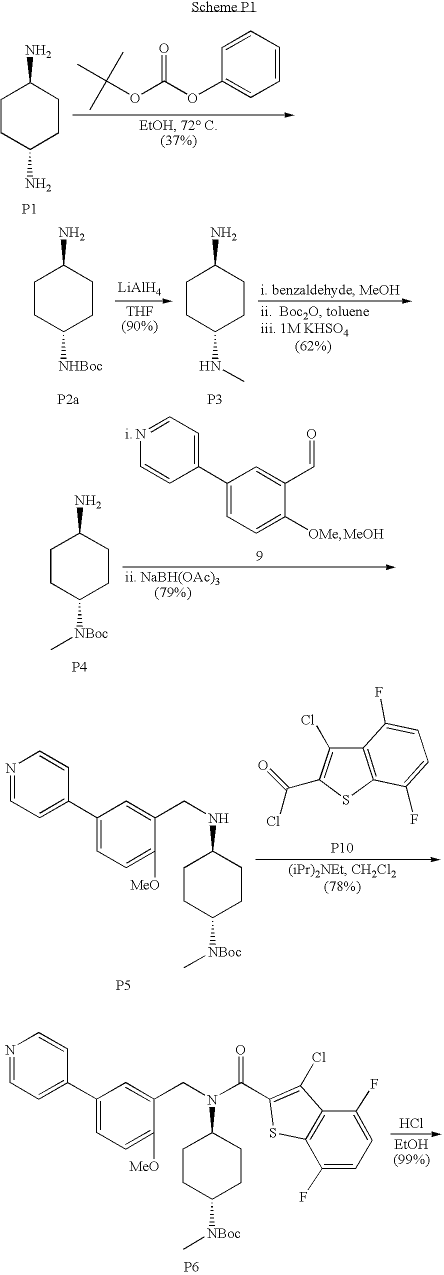

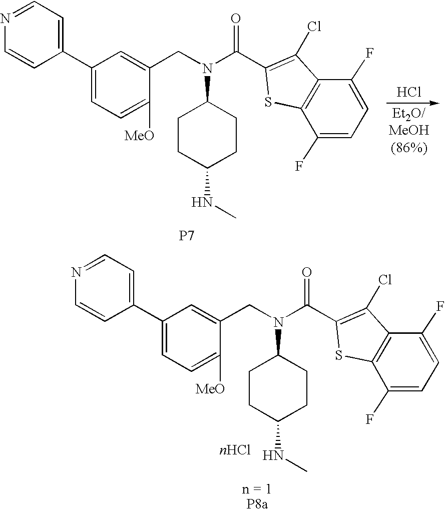

TABLE P1
Overall Summary of quantities of intermediates prepared.

| | P2 | P3 | P4 | P5 | P6 | P7 | P8 |
|---|---|---|---|---|---|---|---|
| starting | 288.2 g | 198.2 g | 102.9 g | 110.7 g | 134.6 g | 133.8 g | 111.8 g |
| material used | | | | | | | |
| amount obtained | 199.0 g | 107.2 g | 115.7 g | 151.3 g | 162.5 g | 112.6 g | 102.0 g |
| | (37%) | (90%) | (62%) | (79%) | (78%) | (99%) | (86%) |

In summary, 101.7 g of P8a (monohydrochloride salt) was prepared via a 9-step synthesis (11% overall yield) in two batches (11.14 g; 90.6 g).

B. Discussion

B.1 First Synthetic Scheme

In the first synthetic route, target compound P8b was prepared in 10 steps with an overall yield of 3% (Scheme P2). A sequence in which the order of acylation and Suzuki coupling was reversed was also investigated and gave compound P8b in a comparable overall yield (4%, scheme not shown).

Preparation of the first key intermediate, functionalized diamine P4, was accomplished via a 5-step (3-pot) synthetic sequence. The yield for the monofunctionalization of diamine was calculated based on the amount of amine used in all instances.

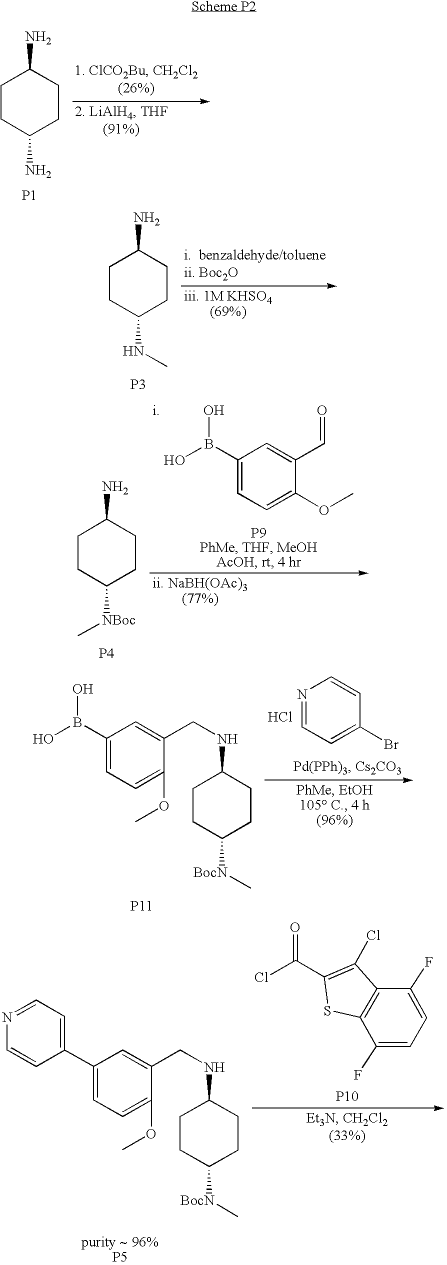

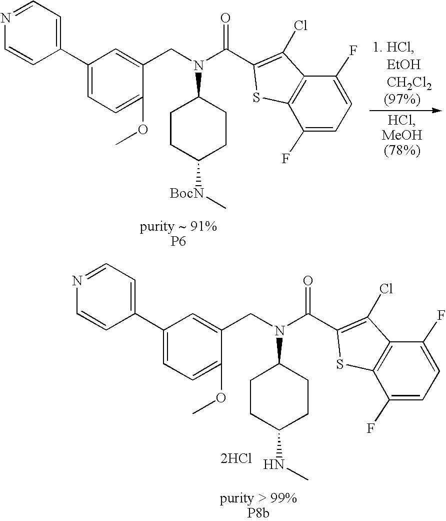

B.2 Modification of the First Synthetic Scheme with a Biaryl Aldehyde

The first synthetic route was modified by incorporating the biaryl aldehyde in one piece as (Scheme P3). Monoacylation of 1,4-cyclohexanediamine produced the desired monocarbamate in 17% yield. The acylated intermediate P6 was isolated in 61% yield.

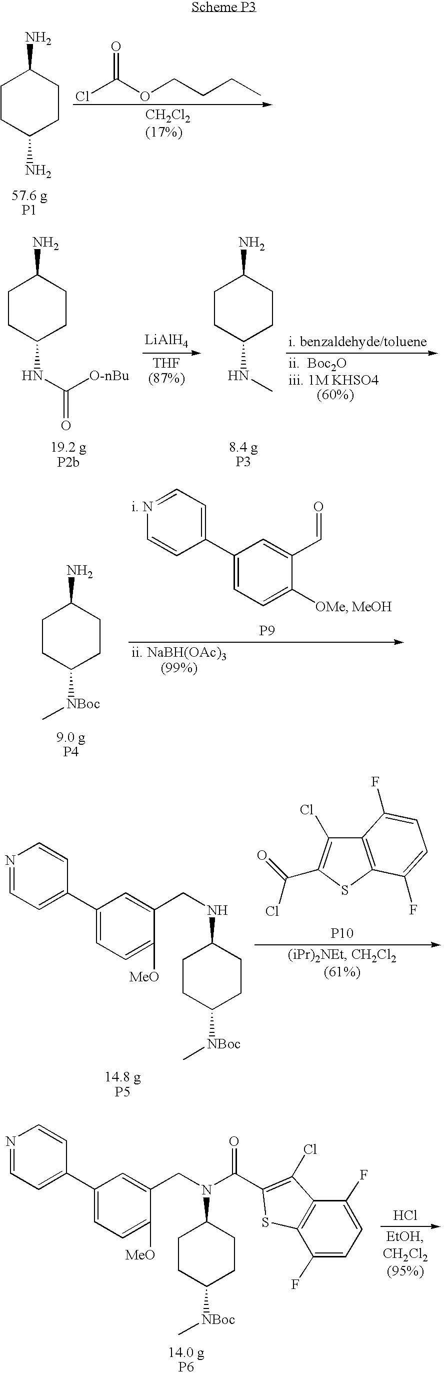

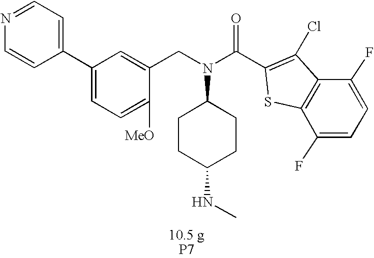

B.3 Early Impurity Profile Analysis

Two main impurities P12 and P13 were identified by LC/MS analysis during the synthesis of P7:

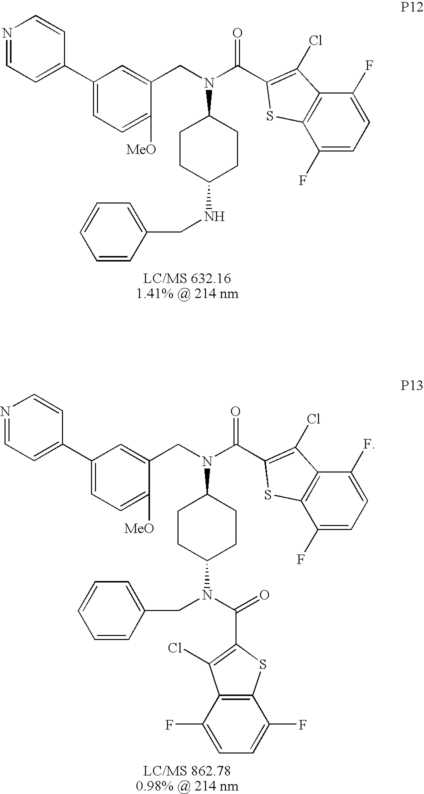

Both impurities were structural analogs of P7 differing by the presence of a benzyl group in place of the methyl group on the cyclohexane diamine fragment. Notably, the second impurity was an acylated derivative of the first. Not surprisingly, the acylated impurity P13 was eliminated by formation of a HCl salt, however the benzylated impurity P12 remained in appreciable amounts (˜1%). Notably, due to the structural interrelation of the two impurities impeding benzylation would eliminate or reduce both impurities. In order to identify the origin of the impurity further LC/MS analysis of upstream intermediates was conducted and revealed that the impurity arose during the reinstallation of the Boc group onto the secondary amine

(Scheme P4).

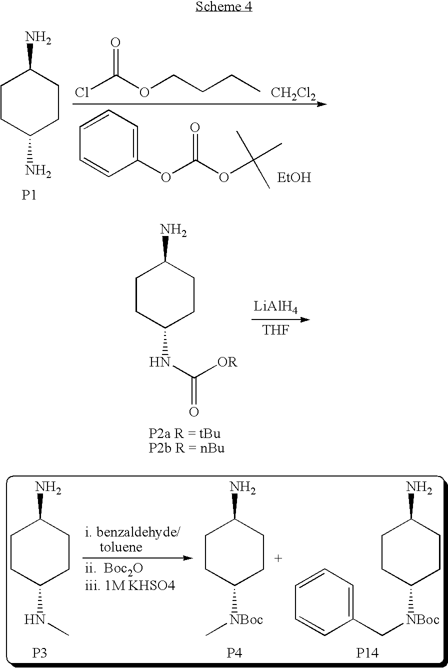

Likely the impurity was generated during the preparation of the imine in refluxing toluene. Initially alternative solvent systems were investigated in which the imine formation was studied at room temperature (Table P2) While imine formation was observed in all three solvent systems investigated, Boc protection in THF and CH₂Cl₂ was sluggish presumably due to solubility problems, which resulted in a heterogeneous reaction mixture. In methanol the imine formed smoothly at room temperature and the solvent was readily exchanged for toluene for subsequent Boc protection.

TABLE P2
investigation of alternative solvents for
imine formation and Boc protection.

| en- | | | |
|---|---|---|---|
| try | scale | reaction conditions | reaction outcome |
| 1 | 513.9 | i. benzaldehyde, MeOH | 677 mg (74%) |
| | mg | ii. Boc₂O, toluene | |
| | | iii. KHSO₄ | |
| 2 | 500 | i. benzaldehyde, THF | 430 mg (48%) |
| | mg | ii. Boc₂O iii. KHSO₄ | ppt formation as Boc₂O |
| | | | is added, difficult work-up |
| 3 | 500 | i. benzaldehyde, | 170 mg (19%) |
| | mg | CH₂Cl₂ ii. Boc₂O | slow imine formation. ppt |
| | | iii. KHSO₄ | formation as Boc₂O is added |

B.4 Preparation of a Single Crystal

The crystal form of the di- and monohydrochloride salts of P7 were also investigated. While the monohydrochloride had been previously prepared, the salt had not been examined for crystallinity by DSC (differential scanning calorimetry) and the dihydrochloride had been previously shown to be amorphous. The dihydrochloride also proved to be amorphous even after recrystallization, while the monohydrochloride produced a stable single crystal. The DSC data for P8a and P8b are shown below:

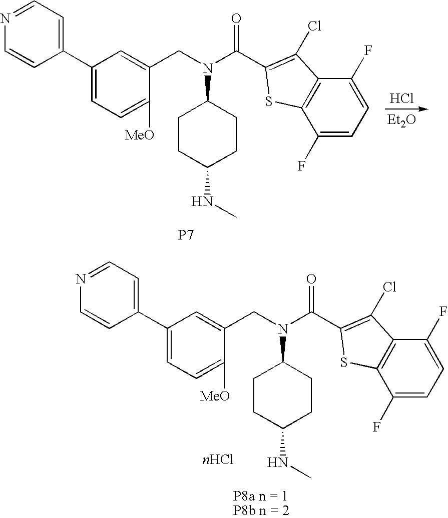

[image]

B.5 Execution of the Improved Synthetic Scheme

Monofunctionalization of 1,4-cyclohexanediamine

A study to monofuctionalize 1,4-cyclohexanediamine was conducted (Table P3).

### Preparation of the Monocarbamate, Monoamide as Well as Monosulfonaminde was Studied and ultimately the use of t-butyl phenyl carbonate in ethanol (see Pittlekow, M.; Lewinsky, R.; Christensen, J. B. Synthesis 2002, 2195-2202) provided a scalable method yielding the mono-Boc protected diamine P8a in an improved 41% yield on a 50 g scale (Table P4, entry 7). The reagents were initially heated to reflux for several hours and once cooled the heterogeneous reaction mixture was vacuum filtered to remove the insoluble bis-carbamate. The reaction mixture was subsequently concentrated to remove the majority of ethanol, and dichloromethane and HCl were added. This resulted in precipitation of the reaction product as the HCl salt, which could be isolated by filtration. The HCl salt was free-based via a basic extractive work-up. The free-base intermediate required no further purification.

TABLE P3
Monofunctionalization of 1,4-cyclohexanediamine

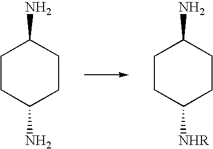

| entry | scale | rxn conditions | rxn outcome |
|---|---|---|---|
| 1 | 1.06 g | i. 9BBN, THF¹ | Bis-sulfonylation |
| | | ii. 2,4-dinitrobenzenesulfonyl | Confirmed by LC/MS |
| | | Cl | |
| 2 | 0.99 g | TsCl, CsOH H₂O, DMF, 4Å | 1.991 g Bis-Ts |
| | | MS² | confirmed by LCMS |
| 3 | 1.10 g | i. 9-BBN, THF | Bis-amide |
| | | ii. Cl₃CCOCl | LC/MS (Meiyi) |
| 4 | 0.5 g | i. 9-BBN, THF | 225.8 mg bis-Boc |
| | | ii. Boc₂O | 1.13 g messy mixture |
| | | | by NMR |
| 5 | 2.16 g | Boc₂O (0.5 eq), | 0.94 g (48%, based on |
| | | THF, 0 to 22° C. | Boc₂O) |
| 6 | 1.03 g | t-butyl phenyl carbonate | 0.49 g (26%) also |
| | | (3 equiv), THF, 86° C. | bis-Boc (1.5 g) |
| 7 | 50.07 g | t-butyl phenyl carbonate | 38.2 g (41%) |
| | | (1.0 equiv), EtOH, 85° C. | |

¹Zhang, Z.; Yin, Z.; Meanwell, N. A.; Kadow, J. F.; Wang, T. Org. Lett. 2003, 5, 3399-3402.

Reduction

Reduction of the carbamate P2a was performed analogously to the above procedure (Scheme P5). For scale-up, a 1M solution of LAH in THF purchased from Aldrich was employed. The reduction was scaled up to 50 g and gave the desired monomethyl diamine in excellent yield. Following filtration the reaction mixture was concentrated and this intermediate was used without further purification

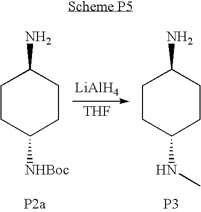

Boc Protection of the Secondary Amine

During the preparation of initial batches of P7a benzylated impurity was identified (see section B.3). After a solvent screen, toluene was replaced by methanol in the first part of the ²Salvatore, R. N.; Schmidt, S. E.; Shin, S.; Nagle, A. S.; Worrell, J. H.; Jung, K. W. Tetrahedron Lett. 200, 41, 9705-9708. reaction sequence, which not only suppressed formation of the undesired impurity but also allowed for imine formation at room temperature and reduced reaction time (Scheme P6). Progress of imine formation was monitored by ¹H NMR and once complete methanol was removed in vacuo. The residue was azeotroped twice with toluene to remove any residual methanol prior to the subsequent Boc-protection. The remainder of the procedure was performed as described above. Following an aqueous work-up the intermediate was used without further purification.

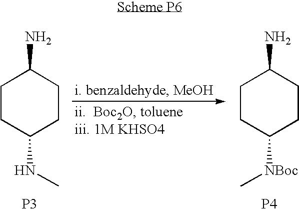

Suzuki Coupling

The reaction conditions for the Suzuki coupling to prepare P9 were modified from the first procedure (Scheme P7). For scale-up, cost was reduced by with Pd(OAc)₂ replacing Pd(PPh₃)₄ and by utilizing the less expensive 4-chloropyridine hydrochloride. The yield utilizing the modified reaction conditions (93%) compared favorably with the original yield (67%).

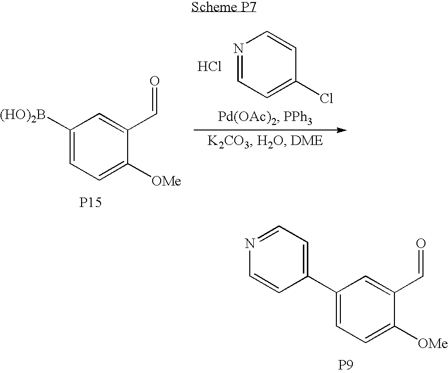

While the aldehyde was purified by flash chromatography, residual metal analysis revealed quantities of residual palladium ranging from 4519-7310 ppm. The palladium content was decreased 41-181 ppm via treatment with N-acetylcysteine. The residual metal content was further reduced during the remaining reaction sequence and the final product was found to contain 2 ppm palladium.

Reductive Amination

The reaction conditions for the reductive amination were modified from the first experimental procedure, which called for the use of solvent mixtures and acid additives. Stirring the aldehyde and amine in methanol at room temperature smoothly provided the imine intermediate, which was reduced via treatment with NaBH(OAc)₃ (Scheme P8). Interestingly, when using the HCl salt of functionalized diamine P4 the reaction had a less favorable outcome as the imine formation did not go to completion. The intermediate amine P5 was purified by flash chromatography. Notably, intermediate amine P5 was prone to undergo dealkylation (cleavage at the benzylic position leading to loss of the biaryl moiety) in the presence of acid.

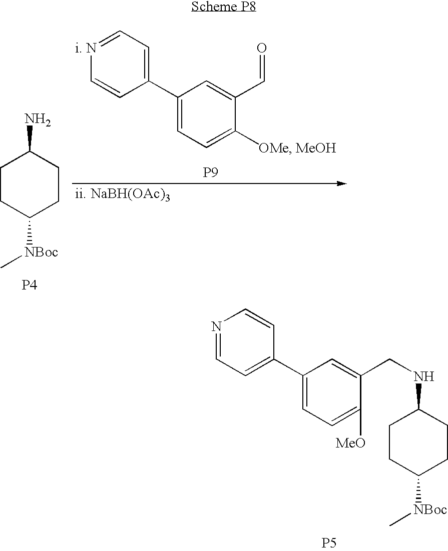

Preparation of 3-chloro-4,7-difluorobenzothiophene-2-carbonyl chloride

The preparation of benzothiophene intermediate P10 was performed in accordance with literature procedures (Scheme P9). The product was purified either via recrystallization or trituration with heptane and ranged in color from a yellow to brown solid. The reaction product retained varying amounts of HCl, which may have contributed to the formation of a dealkylated by-product during the acylation step (see next paragraph).

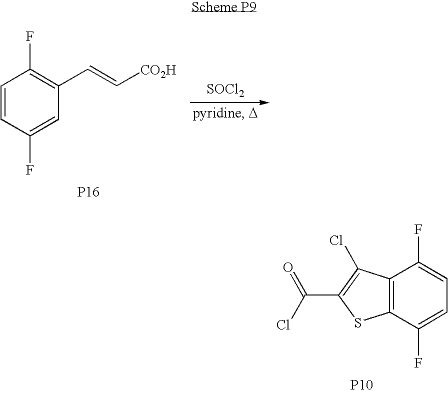

Acylation

The acylation was carried out in analogy to the first procedure (Scheme P10). However, slow addition of a dichloromethane solution of acid chloride reduced the amount of dealkylation observed. While 2.1 equivalents of base were used, dealkylation could be observed by HPLC while monitoring the reaction. Reduction or elimination of the impurity was achieved via careful washing and drying of the acid chloride under vacuum prior to use, alternatively the amount of base used could be increased to compensate for any excess hydrochloric acid.

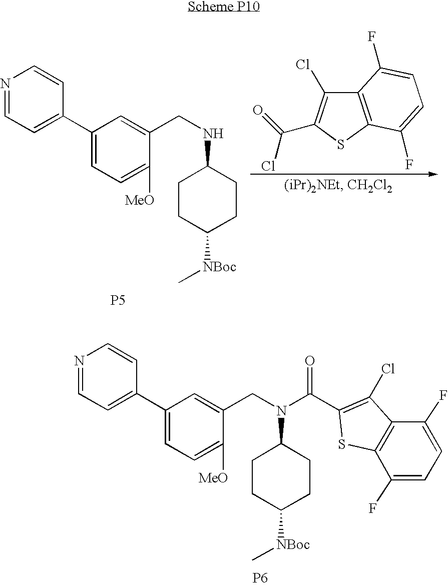

Deprotection

Removal of the Boc protecting group was carried out in ethanol at room temperature in the absence of a co-solvent (Scheme P11, CH₂Cl₂ was used in the first procedure). In addition the amount of solvent used was reduced by >50% during scale-up. Once the reaction was complete as indicated by HPLC analysis (2% starting material remained) the solvent was removed in vacuo and the residue was diluted with water and extracted with dichloromethane, which eliminated some of the less polar impurities present. Subsequently, the aqueous layer was cooled and the pH was adjusted to pH 13 with 50% NaOH, which allowed product isolation via extraction.

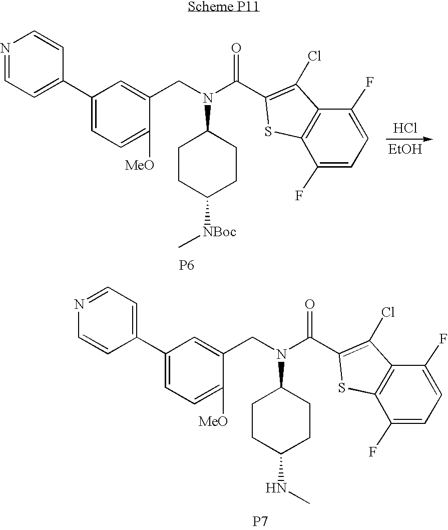

Preparation of the Monohydrochloride Salt

The monohydrochloride P8a was prepared via slow addition of hydrochloric acid (1M in ether) to a solution of P7 in ether/methanol (Scheme P12). In one run, the substrate (11.32 g) was suspended in 600 mL ether, and methanol (160 mL) was added until a homogeneous solution was obtained. Addition of HCl in this case resulted in the formation of initially a milky suspension and then a white slurry, which was stirred for 40 minutes and filtered. On larger scale (100.5 g) the amount of solvent was reduced by >50% resulting initially in formation of a gum-like sticky residue on the flask wall. The residue solidified after scraping the flask wall with a spatula and prolonged stirring. The reduced amount of solvent employed in the large-scale reaction did not adversely affect the crystal form of the hydrochloride salt. The monohydrochloride thus obtained required no additional purification or recrystallization.

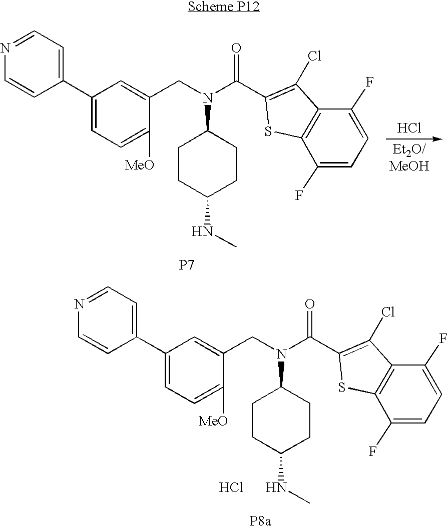

B.6 Investigation of Two Alternative Synthetic Routes

Two alternative reaction routes toward 7 were studied. In the first route, a four step approach to the free base was investigated (Scheme P13). Aminoalcohol P17 was considered as a starting material. Reductive amination installing the biaryl moiety would be followed by oxidation of the alcohol to give aminoketone P19. Acylation would be followed by reductive amination to furnish P7 as the free base.

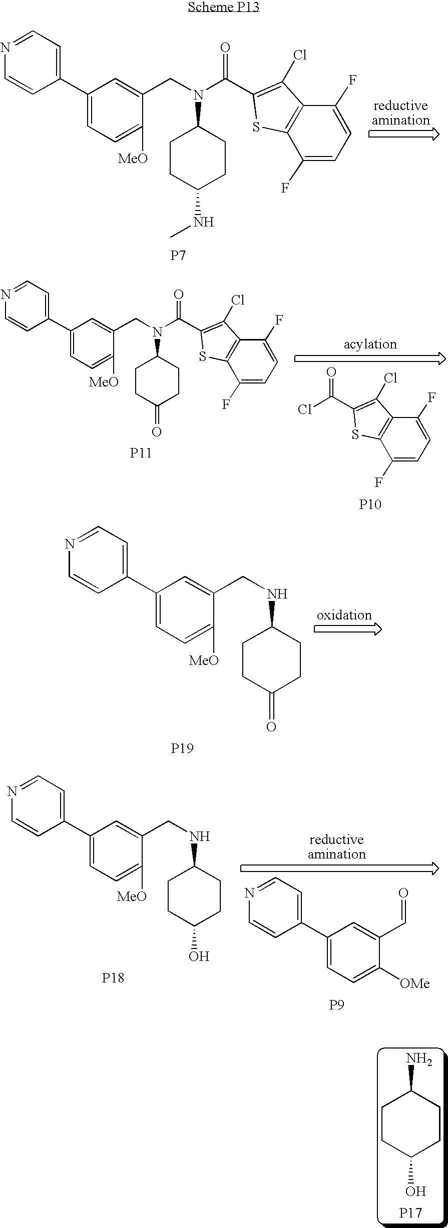

In the forward direction reductive amination produced the desired aminoalcohol P18 in good yield (Scheme P14). However, substrate P18 proved to undergo decomposition (dealkylation) under the oxidation conditions investigated. Swern oxidation gave an unidentified reaction product that was inconsistent with the desired aminoketone P19.

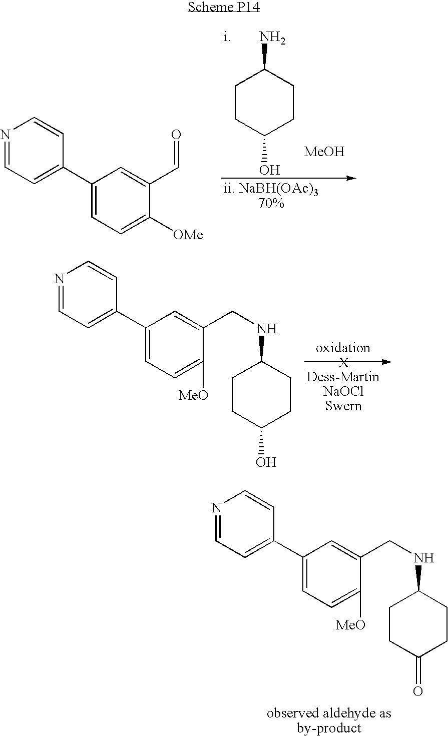

A second alternative route was then considered (Scheme P15). This five-step route would still make use of monofunctionalizing diamine P1 but the N-methyl substituent would be incorporated at a later stage in the synthesis, circumventing five-step sequence used to prepare functionalized diamine P4.

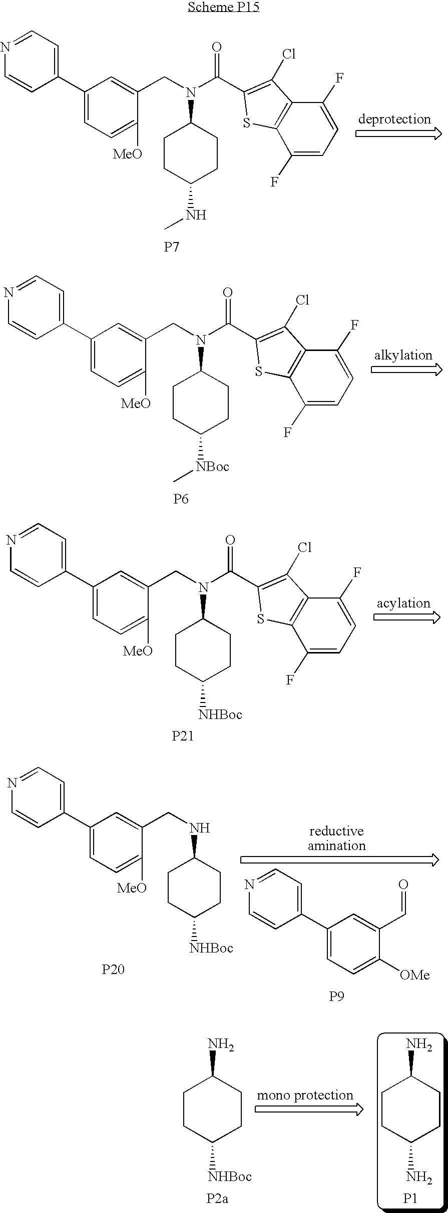

This approach was reduced to practice P7 was prepared via this route (Scheme P16). Notably, reductive amination utilizing the non-methylated diamine proceeded much slower, with reduction of the imine being the rate-determining step. Acylation gave the desired amide P21 in 63% yield after chromatography. The carbamate nitrogen was successfully methylated with methyl iodide in the presence of sodium hydride and the crude product was deprotected to yield P7 (purity 98%). While treatment with sodium hydride and methyl iodide did produce the desired alkylated product, the reaction did not go to completion during a second run.

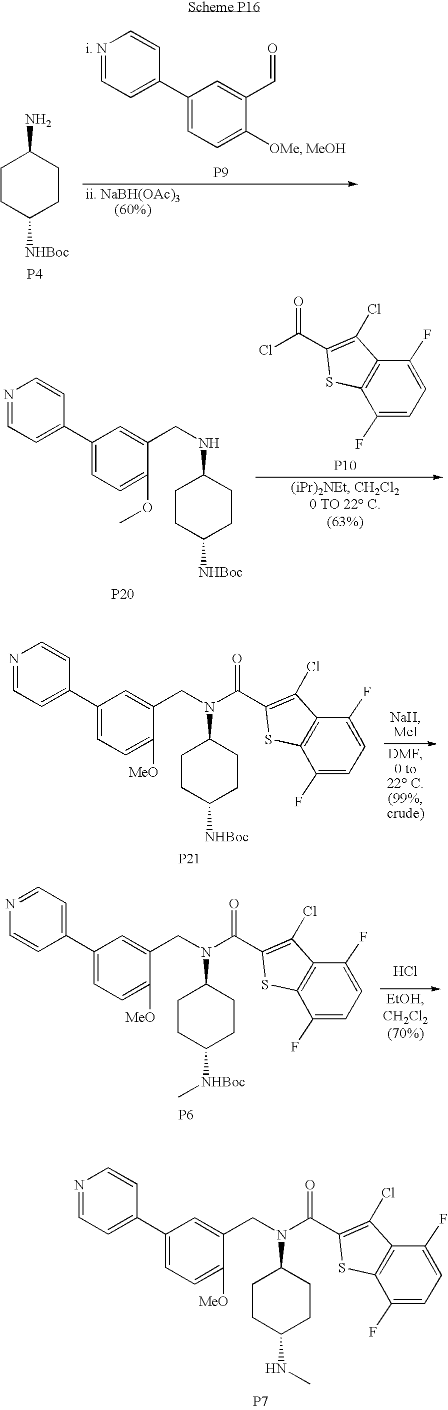

A route to assemble methylated carbamate P4 in two steps from 1,4-cyclohexanediamine was also studied by treating the monocarbamate P2a with methyl iodide in the presence of base (Table P4). Using potassium t-butoxide gave the most promising result and while residual starting material and bis-alkylation were detected by LC/MS, the impurities were not quantified.

TABLE P4
Direct methylation of carbamate P2a.

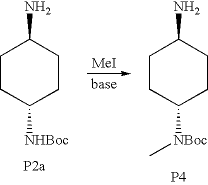

| | | reaction | reaction |
|---|---|---|---|
| entry | scale | conditions | outcome |
| 1 | 0.2 g | NaH (1.6 equiv), | 0.1 g (45%), mixture |
| | | MeI (1.03 equiv), | of desired product, |
| | | DMF, 0° C. | bis-alkylation and |
| | | | starting material by |
| | | | LC/MS |
| 2 | 0.5 g | KOtBu (1.03 equiv), | 0.3 g (53%) desired |
| | | MeI (1.03 equiv), | product (major), sm |
| | | THF, 0° C. | and bis-alkylated |
| | | | product present by |
| | | | LC/MS |

C. Summary

To increase convergence and in an effort to facilitate isolation and purification of intermediates the synthetic scheme ultimately used during the scale-up was slightly modified (Scheme P1). In addition, reaction conditions for some of the synthetic steps were altered to improve the overall yield and impurity profile. Overall of monohydrochloride P8a was prepared as a stable single crystal form and 102 g P8a was submitted in two batches (11.14 g; 90.6 g). To make most efficient use of 1,4-cyclohexanediamine, an alternative method using t-butyl phenyl carbonate in ethanol was employed to produce monocarbamate P2. Impurity profile analysis resulted in an investigation of the reaction conditions used during the reinstallation of the Boc group onto the secondary amine. Ultimately, a solvent switch from toluene to methanol gave the desired reaction product with an improved impurity profile and in a shorter reaction time. The reaction yield for the acylation was increased by ensuring purity of the starting material as well as eliminating chromatographic purification.

D. Experimental Procedures for Improved Synthetic Scheme

Experimental procedures are described below. For some transformations, experimental procedures for different reaction scales are reported.

### Monofunctionalization of Diamine

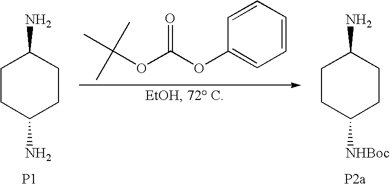

| reagents | MW | Equiv. | mmol | g | d/M | V | S |
|---|---|---|---|---|---|---|---|
| 1,4- | 114.19 | 1 | 1332.9 | 152.2 | | | TCI |
| diamino- | | | | | | | |
| cyclohexane | | | | | | | |
| t-butyl | 194.23 | 1.01 | 1351.5 | | 1.05 | 250 | Aldrich |
| phenyl | | | | | | mL | |
| carbonate | | | | | | | |
| absolute | | | | | | 1 L | EMD |
| EtOH | | | | | | | |

Procedure: To a 3 L 3-neck RB flask equipped with a mechanical stirrer, condenser with N₂ inlet and temperature probe was added 1,4-diaminocylcohexane P1 (152.2 g, 1332.9 mmol), EtOH (1 L) and t-butyl phenyl carbonate (250 mL, 1351.5 mmol). The reaction mixture was wrapped with Al foil and heated using a heating mantle (JKem temperature controller >2 L setting, Tₛₑₜ=100° C.). A brown solution was obtained and an internal temperature of 73.8° C. was recorded after 2.5 h. After heating for 18 h the now heterogeneous reaction mixture was allowed too cool. The mixture was filtered to remove bis-Boc protected amine, which precipitated as a white solid. The filtrate was concentrated in vacuo to a thick peach colored syrup. The mixture was transferred to an Erlenmeyer flask and the transfer was completed with CH₂Cl₂ (50 mL). To the mixture H₂O (100 mL) was added (pH=9) followed by concentrated HCl (180 mL to pH=2) resulting in the precipitation of the (4-aminocyclohexyl)carbamic acid tert-butyl ester as a hydrochloride salt. The mixture was swirled vigorously until a thick slurry was obtained. Additional CH₂Cl₂ was added to maximize the amount of precipitate and the slurry was filtered (additional product was obtained from this filtrate # 1, as described below). The solid was washed with Et₂O (1.4 L). To free-base the HCl salt H₂O (500 mL) was added followed by NaOH (2.5N, to pH=12). The mixture was extracted with CH₂Cl₂ (3×). The combined organic extracts were washed with H₂O (3×400 mL), dried over anhydrous Na₂SO₄ and concentrated in vacuo to give (4-aminocyclohexyl)carbamic acid tert-butyl ester P2a (92.89 g) as a white solid.

Additional product was recovered via acid base extraction of filtrate # 1: The filtrate was transferred to a separatory funnel and the layers were separated. The CH₂Cl₂ layer was washed with H₂O (2×500 mL) and the combined aqueous layers were back-extracted with CH₂Cl₂. The pH of the aqueous layer was adjusted to pH=12 with NaOH (50%). The milky suspension was extracted with CH₂Cl₂. The combined organic extracts were washed with H₂O, dried over anhydrous Na₂SO₄ and concentrated in vacuo to give additional (4-aminocyclohexyl)carbamic acid tert-butyl ester P2a (4.6 g). Combined yield 97.2 g, 34%: ¹H NMR (400 MHz, cdcl₃) δ ppm 4.39 (s, 1H), 3.31 (s, 1H), 2.53-2.59 (m, 1H), 1.96-1.91 (m, 2H), 1.75-1.85 (m, 2H), 1.37 (s, 9H), 0.96-1.25 (m, 5H); ¹³C NMR (101 MHz, cdcl₃) δ ppm 155.20, 78.99, 49.87, 49.19, 35.33, 32.12, 28.35; LRMS (ESI) Calcd for C₁₁H₂₂N₂O₂ [M]:214.1682. Found [M+H]: 215.2.

| reagents | MW | Equiv. | mmol | g | d/M | V | S |
|---|---|---|---|---|---|---|---|
| 1,4- | 114.19 | 1 | 438.5 | 50.07 | | | TCI |
| diamino- | | | | | | | |
| cyclo- | | | | | | | |
| hexane | | | | | | | |
| t-butyl | 194.23 | 1.01 | 443.3 | | 1.05 | 82.0 mL | Aldrich |
| phenyl | | | | | | | |
| carbonate | | | | | | | |
| absolute | | | | | | 330 mL | EMD |
| EtOH | | | | | | | |

Procedure: A mixture of 1,4-diaminocylcohexane P1 (50.07 g, 438.5 mmol) and t-butyl phenyl carbonate (82.0 mL, 443.3 mmol) in EtOH (330 mL) was heated to reflux for 16 h. The heterogeneous reaction mixture was allowed to cool and the precipitated bis-Boc diamine was removed by filtration. The filtrate was concentrated to a thick slurry and CH₂Cl₂ was added followed by concentrated HCl to pH=2. The mixture was swirled vigorously and filtered to give t-butyl-4-aminocyclohexylcarbamate hydrochloride (64.07 g). Water (100 mL) and NaOH (2.5N, 280 mL) was added and the slurry was extracted with CH₂Cl₂ (2×250 mL). The combined organic extracts were washed with H₂O (2×200 mL) dried over anhydrous Na₂SO₄ and concentrated in vacuo to give (4-aminocyclohexyl)carbamic acid tert-butyl ester P2a (38.2 g, 41%) as a white solid.

### Reduction

### N-methylcyclohexane-1,4-diamine

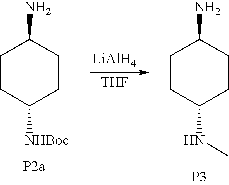

| reagents | MW | Equiv. | mmol | g | d/M | V | S |
|---|---|---|---|---|---|---|---|
| (4-amino- | 214.30 | 1 | 225.59 | 48.34 | | | L31584- |
| cyclo- | | | | | | | 20-3,4 |
| hexyl) | | | | | | | |
| carbamic | | | | | | | |
| acid tert- | | | | | | | |
| butyl ester | | | | | | | |
| LAH | | 3.3 | 750.0 | | 1 M | 750 | Aldrich |
| | | | | | in | mL | |
| | | | | | THF | | |
| THF | | | | | | 300 | EMD |
| | | | | | | mL | |

Procedure: To a 3 L 3-neck-RB flask equipped with a mechanical stirrer, condenser with N₂ inlet and addition funnel was added LAH (750.0 mL, 1M in THF, 750.0 mmol). The addition funnel was rinsed with 20 mL THF and the solution was cooled in an ice bath for 20 minutes. (4-aminocyclohexyl)carbamic acid tert-butyl ester P2a (48.34 g, 225.59 mmol) was dissolved in THF (250 mL) and insoluble material was removed via filtration to give a light yellow solution, which was added to the cooled LAH solution over 1 h 45 minutes via addition funnel. The transfer was completed by rinsing with THF (2×10 mL). After 20 minutes the cold bath was removed and the pink solution was allowed to warm over 45 minutes. The flask was wrapped with Al foil for insulation and the opaque pink mixture was heated (JKem controller 300 mL-2 L setting, T_{set external}=55° C.). The external temperature setting was gradually increased to 85° C. until the internal temperature reached 64° C. and the solvent started to reflux. Reflux was continued for 3 h and TLC analysis showed no remaining starting material. The mixture was allowed to cool and stirred overnight at 22° C. The resulting slurry was cooled in an ice bath and H₂O (40 mL) was added carefully via addition funnel (1 drop every 10 seconds) over 2 h while maintaining an internal temperature of 10° C. or below. Subsequently, NaOH (16% wt., 40 mL) was added dropwise over 15 minutes followed by H₂O (100 mL) over 10 minutes. The cold bath was removed and the slurry was stirred for 1 h and filtered through a medium fritted filter funnel. The transfer was completed with THF (2×100 mL) and the filter cake was washed with TBME (2×200 mL) and CH₂Cl₂ (4×250 mL). The light yellow filtrate was concentrated in vacuo to give N-methylcyclohexane-1,4-diamine P3 (28.1 g, 97%) as an off-white crystalline solid: ¹H NMR (400 MHz, cdcl₃) δ ppm 2.64-2.56 (m, 1H), 2.36 (s, 3H), 2.33-2.19 (m, 2H), 1.92-1.85 (m, 2H), 1.85-1.78 (m, 2H), 1.12-0.98 (m, 4H); ¹³C NMR (101 MHz, cdcl₃) δ ppm 58.05, 50.43, 35.33, 35.23, 35.16, 30.25, 33.83, 31.78, 31.73, 30.25; LRMS (ESI) Calcd for C₇H₁₆N₂ [M]:128.217. Found [M+H]: 129.

### Selective Boc-Protection of Secondary Amine

### (4-aminocyclohexyl)methylcarbamic acid tert-butyl ester

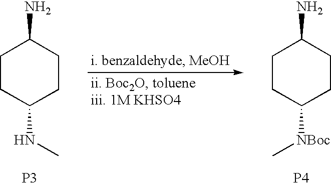

| reagents | MW | Equiv. | mol | g | d/M | V | S |
|---|---|---|---|---|---|---|---|
| N-methyl- | 128.22 | 1 | 213.69 | 27.4 | | | L31584- |
| cylco- | | | | | | | 27-1 |
| hexane- | | | | | | | |
| 1,4- | | | | | | | |
| diamine | | | | | | | |
| benzalde- | 106.12 | 1 | 214.47 | | 1.044 | 21.8 | |
| hyde | | | | | | mL | |
| MeOH | | | | | | 800 | Aldrich |
| | | | | | | mL | |
| Boc₂O | 218.25 | 1.01 | 216.27 | 47.2 | | | Aldrich |
| | | | | g | | | |
| toluene | | | | | | 300 | Aldrich |
| | | | | | | mL | |
| KHSO₄ | | | | | 1 M | 900 | Aldrich |
| | | | | | aq | mL | |

Two reactions of similar scale 27.4 g and 29.6 g were run side by side and were combined during the work-up.

Procedure: To a 2 L 3-neck RB flask equipped with a magnetic stirrer, N₂ inlet and stopper was added N-methylcyclohexane-1,4-diamine P3 (27.4 g, 213.69 mmol), MeOH (800 mL) and benzaldehyde (21.8 mL, 214.47 mmol). After stirring the mixture for 1.5 h ¹HMNR analysis of a small aliquot showed that imine formation was complete (aliquot was concentrated and diluted with dr-dmso). The reaction mixture was concentrated and azeotroped twice with toluene (200 mL and 100 mL). The residue was diluted with toluene (300 mL) and cooled in an ice bath. Di-tert-butyldicarboxylate (47.2 g, 216 mmol) was added over 15 minutes and the mixture was stirred for 2 h. The ice bath was removed and the mixture was concentrated in vacuo. To the residue was added an aqueous solution of KHSO₄ (1M, 900 mL) and the mixture was stirred at room temperature for 2.5 h. At this stage the reaction mixture was combined with a second run (29.6 g scale). The combined mixture was extracted with methyl tert-butyl ether (3×700 mL) until no UV active material could be extracted. The pH of the aqueous mixture was adjusted to pH 13 with 25% aqueous NaOH and extracted with dichloromethane (3×600 mL). The combined organic layers were dried over anhydrous Na₂SO₄, filtered and concentrated in vacuo to give (4-aminocyclohexyl)methylcarbamic acid tert-butyl ester P4 (64.6 g, 64%) as a yellow oil, which was used without further purification.

| reagents | MW | Equiv. | mol | g | d/M | V | S |
|---|---|---|---|---|---|---|---|
| N-methylcylcohexane- | 128.22 | 1 | 79.55 | 10.2 | | | L31584- |
| 1,4-diamine | | | | | | | 27-1 |
| benzaldehyde | 106.12 | 1.01 | 80.67 | | 1.044 | 8.2 mL | |
| MeOH | | | | | | 320 mL | Aldrich |
| Boc₂O | 218.25 | 1.01 | 80.64 | 17.6 g | | | Aldrich |
| toluene | | | | | | 100 mL | Aldrich |
| KHSO₄ | | | | | 1M aq | 300 mL | Aldrich |

Procedure: To a 2 L RB flask equipped with a magnetic stirrer, N₂ inlet and stopper was added N-methylcyclohexane-1,4-diamine (10.2 g, 79.55 mmol), MeOH (320 mL) and benzaldehyde (8.2 mL, 80.67 mmol). After stirring the mixture for 1.5 h ¹HMNR analysis of a small aliquot showed that imine formation was complete (aliquot was concentrated and diluted with d₆-dmso). The reaction mixture was concentrated and azeotroped twice with toluene (2×50 mL). The residue was diluted with toluene (100 mL) and cooled in an ice bath. Di-tert-butyldicarboxylate (17.6 g, 80.64 mmol) was added over 5 minutes and the mixture was stirred for 2 h. The ice bath was removed and the mixture was concentrated in vacuo. To the residue was added an aqueous solution of KHSO₄ (1M, 300 mL) and the mixture was stirred at room temperature for 2 h. The mixture was extracted with methyl tert-butyl ether (3×250 mL). The pH of the aqueous mixture was adjusted to pH 13 with 25% aqueous NaOH and extracted with dichloromethane (3×250 mL). The combined organic layers were dried over anhydrous Na₂SO₄, filtered and concentrated in vacuo to a yellow oil, which was used without further purification (12.0 g, 66%). ¹H NMR (400 MHz, cdcl₃) δ ppm 2.65 (s, 3H), 2.60-2.50 (m, 1H), 1.89-1.78 (appd, J=12.6 Hz, 2H), 1365-1.59 (appd, J=11.5, 2H), 1.50-1.40 (m, 1H), 1.40 (s, 9H), 1.25-1.10 (m, 4H); LRMS (ESI) Calcd for C₁₂H₂₄N₂O₂ [M]: 228.184. Found [M+H]: 229.2.

### Suzuki Coupling

### 2-methoxy-5-(pyridin-4-yl)benzaldehyde

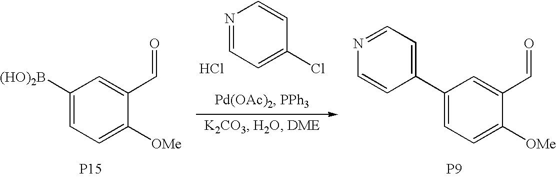

| reagents | MW | equiv. | mmol | g | d/M | V | S |
|---|---|---|---|---|---|---|---|
| 3-formyl-4-methoxy | 179.97 | 1 | 276.90 | 49.83 | | | |
| phenylboronic acid | | | | | | | |
| 4-chloropyridine | 150.01 | 1.4 | 396.33 | 59.45 | | | |
| hydrochloride | | | | | | | |
| Pd(OAc)₂ | 224.49 | 0.037 | 10.16 | 2.28 | | | Ald. |
| PPh₃ | 262.29 | 0.15 | 42.13 | 11.05 | | | Ald. |
| K₂CO₃ | | | | | 2 M aq | 290 mL | |
| DME | | | | | | 280 mL | Ald. |

Procedure: To a 2-L 3-neck RB flask equipped with a mechanical stirrer, condenser with N₂ inlet and temperature probe was added 3-formyl-4-methoxyphenylboronic acid P15 (49.83 g, 276.90 mmol), 4-chloropyridine hydrochloride (46.63 g, 310.84 mmol), triphenylphosphine (7.42 g, 28.28 mmol) and dimethoxyethane (280 mL). To the resulting white slurry was added 2M aqueous K₂CO₃ (290 mL). The resulting mixture was degassed for 20 minutes with N₂ and Pd(OAc)₂ (1.65 g, 7.36 mmol) was added. Under N₂ atmosphere the mixture was heated to 65° C. After 47 h additional 4-chloropyridine hydrochloride (12.82 g, 85.50 mmol), triphenylphosphine (3.63 g, 13.83 mmol) and Pd(OAc)₂ (632.3 mg, 2.82 mmol) was added and heating was resumed for an additional 24 h. The reaction mixture was partitioned between EtOAc (300 mL) and H₂O (300 mL). The aqueous layer was extracted with EtOAc (4×400 mL) and the combined organic extracts were concentrated to a yellow-orange viscous oil. The crude reaction product was purified by filter chromatography on SiO₂ eluting with EtOAc/hexanes (30 to 100%) followed by 3% MeOH/EtOAc. The yellow solid was triturated with 10% Et₂O/hexanes and filtered to give 2-methoxy-5-(pyridin-4-yl)benzaldehyde P9 (53.0 g, 90%) as a yellow solid: ¹H NMR (400 MHz, DMSO-d₆) δ ppm 10.39 (s, 1H), 8.61 (d, J=6.10 Hz, 2H), 8.11 (d, J=8.54 Hz, 1H), 8.06 (d, J=2.20 Hz, 1H), 7.70 (d, J=5.12 Hz, 2H), 7.38 (d, J=8.78 Hz, 1H), 3.98 (s, 3H) ¹³C NMR (101 MHz, DMSO-d₆) δ ppm 189.63, 162.77, 150.92, 146.15, 135.17, 130.03, 126.71, 125.13, 121.34, 114.37, 57.01; LRMS (ESI) Calcd for C₁₃H₁₁NO₂ [M]: 213.079. Found [M+H]: 214.

| reagents | MW | equiv. | mmol | g | d/M | V | S |
|---|---|---|---|---|---|---|---|
| 3-formyl-4- | 179.97 | 1 | 142.38 | 25.62 | | | |
| methoxyphenylboronic | | | | | | | |
| acid | | | | | | | |
| 4-chloropyridine | 150.01 | 1.4 | 157.66 | 23.65 | | | |
| hydrochloride | | | | | | | |
| Pd(OAc)₂ | 224.49 | 0.026 | 3.74 | 0.8409 | | | Ald. |
| PPh₃ | 262.29 | 0.1 | 14.37 | 3.77 | | | Ald. |
| K₂CO₃ | | | | | 2M aq | 150 mL | |
| DME | | | | | | 140 mL | Ald. |

Procedure: To a 1 L 3-neck RB flask equipped with a N₂ inlet, condenser and temperature probe was added 3-formyl-4-methoxyphenylboronic acid P15 (25.62 g, 142.38 mmol), 4-chloropyridine hydrochloride (23.65 g, 157.66 mmol), triphenylphosphine (3.77 g, 14.37 mmol), diethoxymethane (140 mL), and a solution of K₂CO₃ (2.7M, 150 mL). The yellow slurry was degassed with N₂ for 10 minutes and Pd(OAc)₂ (840.9 mg, 3.74 mmol) was added. The mixture was heated to 80° C. for 19 h. The layers were separated and the aqueous layer was extracted with EtOAc. The combined organic extracts were concentrated in vacuo and purified by filter chromatography on SiO₂ eluting with EtOAc/hexanes (30 to 100%) followed by 3% MeOH/EtOAc, to give 2-methoxy-5-(pyridin-4-yl)benzaldehyde P9 (29.97 g, 99%) as a yellow solid. (L30464-208, 266146). To remove excess palladium 2-methoxy-5-(pyridin-4-yl)benzaldehyde P9 (28.26 g, 132.53 mmol) in EtOAc (500 mL) was treated with N-acetylcysteine (2.22 g, 13.58 mmol) in H₂O (100 mL). The mixture was stirred for 4 h diluted with H₂O and the layers were separated. The aqueous layer was extracted with EtOAc (5×200 mL) and the combined organic extracts were concentrated in vacuo and filtered through a Magnesol pad to give 2-methoxy-5-(pyridin-4-yl)benzaldehyde P9 (25.33 g, 90%) as a yellow solid.

### Reductive Amination

### [4-(2-methoxy-5-pyridin-4-yl-benzylamino)cyclohexyl]methylcarbamic acid t-butyl ester

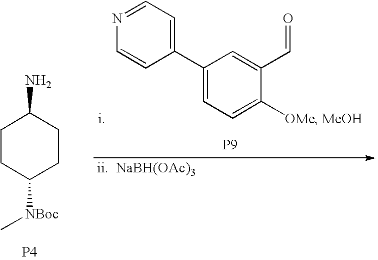

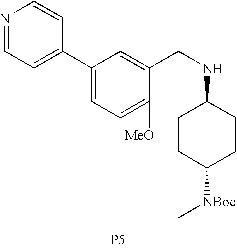

| reagents | MW | equiv. | mmol | g | d/M | V | S |
|---|---|---|---|---|---|---|---|
| (4-aminocylcohexyl) | 228.33 | 1.08 | 162.48 | 37.1 | | | |
| methylcarbamic acid | | | | | | | |
| tert-butyl ester | | | | | | | |
| 2-methoxy-5-(pyridin- | 213.23 | 1 | 150.12 | 32.01 | | | |
| 4-yl)benzaldehyde | | | | | | | |
| NaBH(OAc)₃ | 211.94 | 2.7 | 404.08 | 85.64 | | | Aldrich |
| MeOH | | | | | | 600 mL | |

Procedure: To a 2 L 3-neck RB flask was added 4-aminocyclohexyl)methylcarbamic acid t-butyl ester P4 (37.1 g, 162.5 mmol) and MeOH (550 mL). To the resulting light yellow solution was added 2-methoxy-5-(pyridin-4-yl)benzaldehyde P9 (32.01 g, 150.1 mmol) as a solid, and transfer was completed with MeOH (50 mL). After stirring the reaction mixture at room temperature for 1 h, the mixture was cooled in ice and NaBH(OAc) (82.83 g, 390.8 mmol) was added in portions over 1 h. After stirring the yellow slurry for 40 minutes the cold bath was removed and stirring was continued for 1 h at room temperature. Additional NaBH(OAc)₃ (2.81 g, 18.0 mmol) was added and stirring was continued for 30 minutes. The reaction mixture was concentrated in vacuo and the residue was diluted with H₂O (300 mL) and NaOH (2N and 25%) was added to pH=13. The milky suspension was extracted with dichloromethane (3×400 mL) and the combined organic extracts were dried over anhydrous Na₂SO₄ and concentrated in vacuo. The residue was purified via filter chromatography on SiO₂ eluting with 2M NH₃ in MeOH/EtOAc (5-10%) to give [4-(2-methoxy-5-pyridin-4-yl-benzylamino)cyclohexyl]methylcarbamic acid t-butyl ester P5 (45.3 g, 71%) as a light yellow foamy residue: ¹H NMR (400 MHz, DMSO-d₆) δ ppm 8.58 (d, J=8 Hz, 2H), 7.80 (d, J=4 Hz, 1H), 7.68-7.64 (m, 3H), 7.08 (d, J=8 Hz, 1H), 3.84 (s, 3H), 3.75 (s, 2H), 2.62 (s, 3H), 2.32-2.20 (m, 1H), 1.98-1.89 (m, 1H), 1.53-1.38 (m, 4H) 1.38 (s, 9H), 1.25-1.00 (m, 4H); ¹³C NMR (101 MHz, DMSO-d₆) δ ppm 170.94, 158.67, 155.21, 150.75, 147.53, 130.60, 129.38, 127.62, 126.67, 121.20, 111.69, 78.92, 60.39, 56.21, 55.69, 45.18, 32.83, 28.76; LRMS (ESI) Calcd for C₂₅H₃₅N₃O₃ [M]: 425.571. Found [M+H]: 426.2.

### Preparation of 3-chloro-4,7-difluorobenzothiophene-2-carbonyl chloride

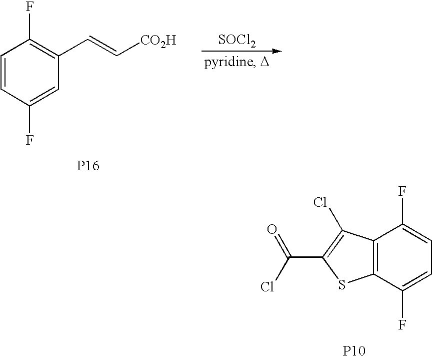

| reagents | MW | equiv. | mmol | g | d/M | V | S |
|---|---|---|---|---|---|---|---|
| 2,5- | 184.14 | 1 | 237.86 | 43.8 | | | |
| difluoro- | | | | | | | |
| cinnamic | | | | | | | |
| acid | | | | | | | |
| thionyl | 118.97 | 3.49 | 830.78 | | 1.631 | 60.6 | Aldrich |
| chloride | | | | | | mL | |
| pyridine | 79.10 | 0.28 | 66.39 | | 0.978 | 5.37 | Aldrich |
| | | | | | | mL | |

Procedure: To 2,5-difluorocinnamic acid P16 (43.8 g, 237.86 mmol) in thionyl chloride (60.6 mL, 830.78 mmol) was slowly added pyridine (5.37 mL, 66.39 mmol) over 50 minutes and the resulting yellow solution was heated gradually until the internal temperature reached 140° C. After 21 h the reaction mixture was allowed to cool briefly and 300 mL heptane was added. Heating was resumed for 10-15 minutes and the reaction mixture was filtered while hot to remove insoluble impurities. The filtrate was allowed to cool under vacuum for 30 minutes until formation of a precipitate was observed. The precipitate was isolated by filtration and washed with cold heptane. The filtrate was concentrated to a brown solid, which after trituration with hot heptane yielded additional crops of the desired product. This was repeated twice and the solids were combined to give 3-chloro-4,7-difluorobenzothiophene-2-carbonyl chloride P10 (39.04 g, 61%) as a yellow-brown solid.

### Acylation

### {4-[(3-chloro-4,7-difluoro-benzo[b]thiophene-2-carbonyl)-(2-methoxy-5-pyridin-4-yl-benzyl)-amino]-cyclohexyl}-methyl-carbamic acid tert-butyl ester

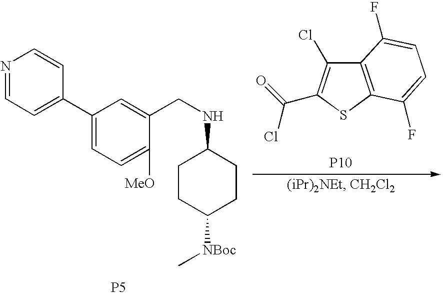

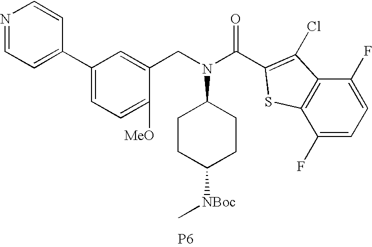

| reagents | MW | equiv. | mmol | g | d/M | V | S |
|---|---|---|---|---|---|---|---|
| [4-(2- | 425.56 | 1 | 118.20 | 50.3 | | | |
| methoxy- | | | | | | | |
| 5- | | | | | | | |
| pyridin-4- | | | | | | | |
| yl-benzyl- | | | | | | | |
| amino)- | | | | | | | |
| cyclo- | | | | | | | |
| hexyl] | | | | | | | |
| methyl- | | | | | | | |
| carbamic | | | | | | | |
| acid | | | | | | | |
| t-butyl | | | | | | | |
| ester | | | | | | | |
| 3-chloro- | 267.08 | 1.1 | 129.92 | 34.7 | | | |
| 4,7-di- | | | | | | | |
| fluoro- | | | | | | | |
| benzo- | | | | | | | |
| thiophene- | | | | | | | |
| 2-carbonyl | | | | | | | |
| chloride | | | | | | | |
| (iPr)₂NEt | 129.25 | 2 | 248.0 | | 0.742 | 43.2 mL | A |
| CH₂Cl₂ | | | | | | 1 L | A |

Procedure: To a 3 L 3-neck RB flask equipped with a mechanical stirrer, N₂ inlet and addition funnel was added [4-(2-methoxy-5-pyridin-4-yl-benzylamino)cyclohexyl]methylcarbamic acid t-butyl ester P5 (50.3 g, 118.20 mmol) and CH₂Cl₂ (750 mL). The resulting mixture was cooled in an ice bath and 3-chloro-4,7-difluorobenzothiophene-2-carbonyl chloride P10 (34.7 g, 129.92 mmol) in CH₂Cl₂ (250 mL) was added over 4 h. The reaction mixture was diluted with CH₂Cl₂ and washed with 1N HCl (800 mL), 1N NaOH (800 mL), H₂O (500 mL) and brine (500 mL). The organic layer was dried over anhydrous Na₂SO₄ and concentrated in vacuo to an orange-brown solid. The crude product was ground to a fine solid, diluted with EtOAc (450 mL) and the resulting slurry was stirred for 1 h. The mixture was filtered and the filter cake was washed with EtOAc (100 mL), and dried to give {4-[(3-chloro-4,7-difluorobenzothiophene-2-carbonyl)-(2-methoxy-5-pyridin-4-ylbenzyl)amino]cyclohexyl}methylcarbamic acid tert-butyl ester P6 (66.0 g, 85%) as an off-white solid.

### Deprotection

### 3-chloro-4,7-difluoro-benzo[b]thiophene-2-carboxylic acid (2-methoxy-5-pyridin-4-yl-benzyl)-(4-methylaminocyclohexyl)amide

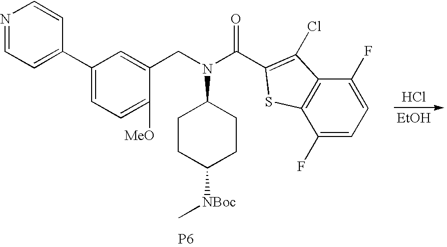

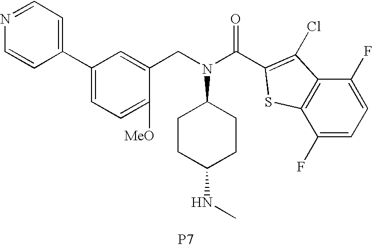

| reagents | MW | equiv. | mmol | g | d/M | V | S |
|---|---|---|---|---|---|---|---|
| {4-[(3-chloro-4,7- | 556.07 | 1 | 83.70 | 54.92 | | | |
| difluorobenzo[b] | | | | | | | |
| thiophene-2- | | | | | | | |
| carbonyl)-(2- | | | | | | | |
| methoxy-5-pyridin- | | | | | | | |
| 4-ylbenzyl)amino]- | | | | | | | |
| cyclohexyl} | | | | | | | |
| methylcarbamic | | | | | | | |
| acid tert-butyl ester | | | | | | | |
| HCl | | | | | 12 | 800 | |
| | | | | | | mL | |
| EtOH | | | | | | 1.5 | |
| | | | | | | L | |

Procedure: To a 3 L 3-neck RB flask with a mechanical stirrer, addition funnel, temperature probe and N₂ inlet was added {4-[(3-chloro-4,7-difluorobenzothiophene-2-carbonyl)-(2-methoxy-5-pyridin-4-ylbenzyl)amino]cyclohexyl}methylcarbamic acid tert-butyl ester P6 (54.92 g, 83.70 mmol) and EtOH (1.5 L). The resulting beige slurry was cooled to 5° C. and concentrated HCl (800 mL) was added over 1 h. The cold bath was removed and the solution was stirred for 4.5 h at room temperature. The reaction mixture was concentrated by removing 1.5 L solvent in vacuo and H₂O (500 mL) was added. The mixture was extracted with CH₂Cl₂ (2×350 mL). The aqueous layer was cooled to 2° C. and 50% NaOH (300 mL) was added to pH=13. The mixture was extracted with CH₂Cl₂ (3×600 mL) and the combined organic layers were washed with brine, dried over anhydrous Na₂SO₄, and concentrated in vacuo to give 3-chloro-4,7-difluoro-benzo[b]thiophene-2-carboxylic acid (2-methoxy-5-pyridin-4-ylbenzyl)-(4-methyl-aminocyclohexyl)amide P7 (46.5 g, 99%) as a tan foam.

### Preparation of HCl Salt

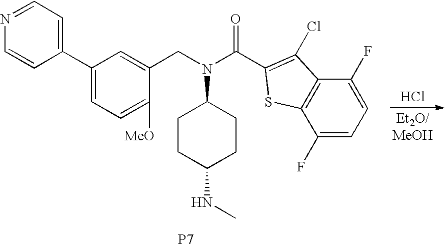

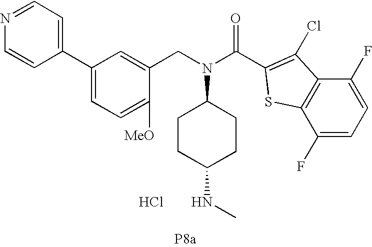

| reagents | MW | equiv. | mmol | g | d/M | V | S |
|---|---|---|---|---|---|---|---|
| 3-chloro-4,7- | 556.07 | 1 | 180.73 | 100.5 | | | |
| difluoro- | | | | | | | |
| benzo- | | | | | | | |
| thiophene-2- | | | | | | | |
| carboxylic | | | | | | | |
| acid (2- | | | | | | | |
| methoxy-5- | | | | | | | |
| pyridin-4- | | | | | | | |
| ylbenzyl)- | | | | | | | |
| (4-methyl- | | | | | | | |
| aminocyclo- | | | | | | | |
| hexyl)amide | | | | | | | |
| HCl | | 0.95 | 172.0 | | 1 M | 172 | |
| (1 M in | | | | | | mL | |
| Et₂O) | | | | | | | |
| Et₂O | | | | | | 2.4 | EMD |
| | | | | | | L | |
| MeOH | | | | | | 450 | Aldrich |
| | | | | | mL | | |

Procedure: To a 5 L 3-neck RB flask was added 3-chloro-4,7-difluoro-benzothiophene-2-carboxylic acid (2-methoxy-5-pyridin-4-ylbenzyl)-(4-methyl-aminocyclohexyl)amide P7 (100.5 g, 180.73 mmol) methanol (450 mL) and Et₂O (1.7 L). A 1.0 M solution of HCl (172 mL, 172 mmol) in Et₂O was added over 20 minutes. The HCl salt initially precipitated as a gum, which solidified after additional Et₂O (700 mL) was added and the flask wall was scratched with a spatula. Stirring was continued for 1 h and the mixture was filtered. The solid was dried under vacuum and N₂ overnight to give 3-chloro-4,7-difluoro-N-[2-methoxy-5-(pyridin-4-yl)benzyl]-N-(4-(methylamino)cyclohexyl)benzothiophene-2-carboxamide hydrochloride P8a (90.6 g, 85%) as an off-white solid.

| reagents | MW | equiv. | mmol | g | d/M | V | S |
|---|---|---|---|---|---|---|---|
| 3-chloro-4,7-difluoro- | 556.07 | 1 | 20.36 | 11.32 | | | |
| benzothiophene-2- | | | | | | | |
| carboxylic acid (2- | | | | | | | |
| methoxy-5-pyridin-4- | | | | | | | |
| ylbenzyl)-(4-methyl- | | | | | | | |
| aminocyclohexyl)amide | | | | | | | |
| HCl (1M in Et₂O) | | 1.02 | 20.8 | | 1M | 20.8 mL | |
| Et₂O | | | | | | 600 mL | EMD |
| MeOH | | | | | | 160 mL | Aldrich |

Procedure: To a 2 L RB flask containing 3-chloro-4,7-difluoro-benzothiophene-2-carboxylic acid (2-methoxy-5-pyridin-4-ylbenzyl)-(4-methyl-aminocyclohexyl)amide P7 (11.32 g, 20.36 mmol) was added 600 mL Et₂O. To the white suspension was added MeOH (160 mL) until a homogeneous solution was obtained. A solution of HCl (1M in Et₂O, 20.3 mL) was added dropwise over 17 minutes and the mixture was stirred for 30 minutes. The slurry was filtered through a medium fritted filter funnel and the filter cake was washed with Et₂O (500 mL) and dried under vacuum and N₂ to give 3-chloro-4,7-difluoro-N-[2-methoxy-5-(pyridin-4-yl)benzyl]-N-(4-(methylamino)cyclohexyl)benzothiophene-2-carboxamide hydrochloride P8a (11.41 g, 95%) as an off-white solid.

E. Overview of an Alternate Improved Synthetic Scheme

An alternate improved synthetic scheme for the preparation of P8a was developed (Scheme P17). 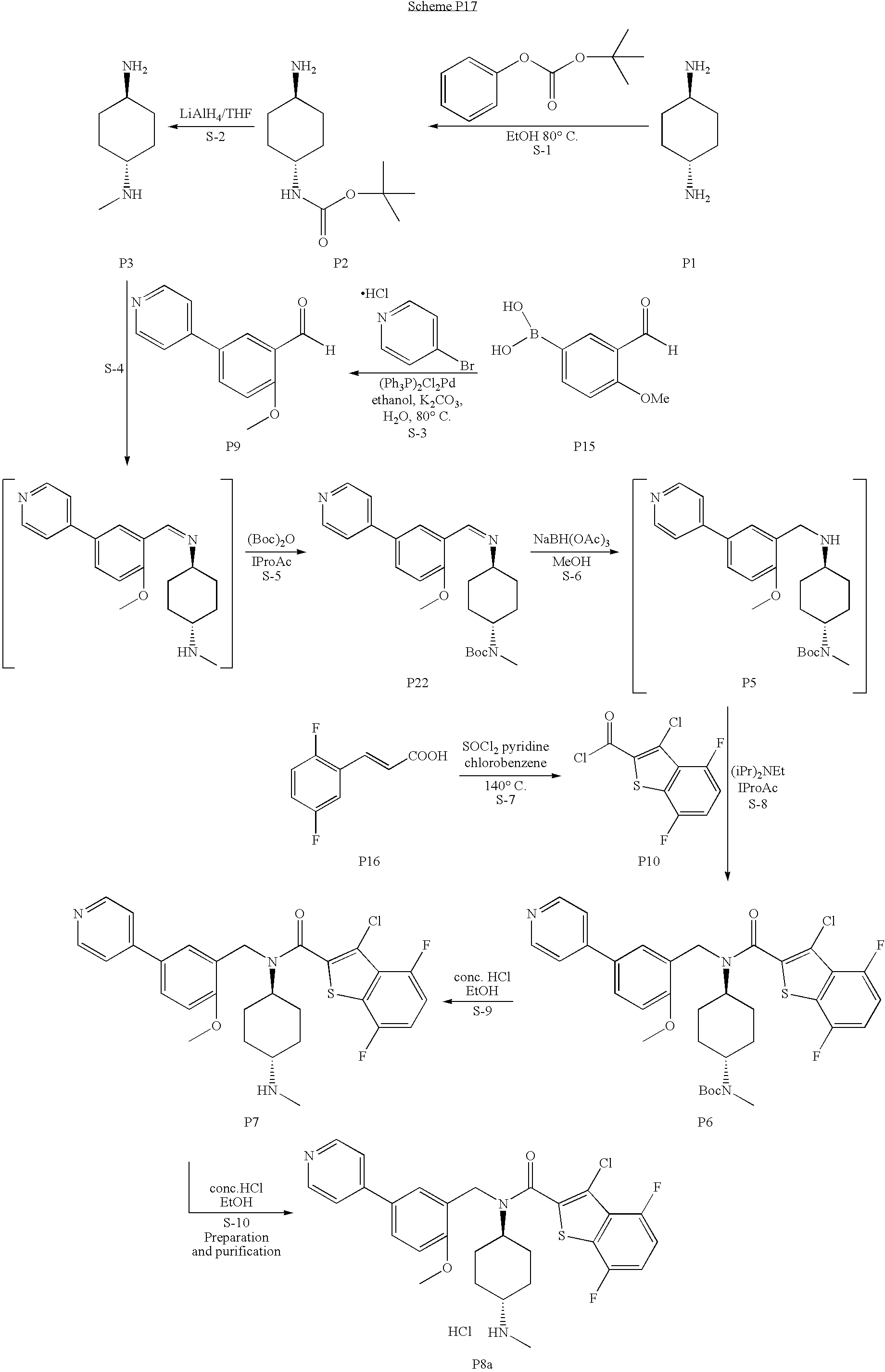
F. Experimental Procedures for Alternate Improved Synthetic Scheme

### Synthesis of P2a

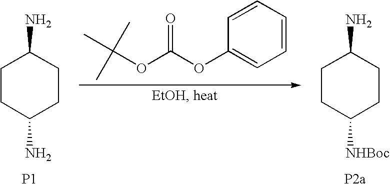

| reagents | MW | equiv | mmol | g | d/M | V |
|---|---|---|---|---|---|---|
| trans-1,4 | 114.19 | 1 | 307.18 | 342 | | |
| diamino- | | | | | | |
| cyclohexane | | | | | | |
| t-butyl- | 194.23 | 1 | 308.14 | 550.8 | 1.05 | |
| phenyl | | | | | | |
| carbonate | | | | | | |
| EtOH | | | | | | 1620 ml |

Procedure: To a 5 L RB flask was added trans-1,4-diaminocyclohexane P1 (342 g, 2.99 mol), EtOH (1620 ml) and t-butyl phenyl carbonate (550.8 g, 2.84 mol). The resulting clear solution was heated for 15-18 h at 85° C. (reflux). Reaction was monitored by GC for the disappearance of t-butyl phenyl carbonate (reaction is complete if all the t-butyl phenyl carbonate is consumed or is ≧1%). The reaction mixture becomes heterogeneous due to the precipitation of bis bocamine. When the reaction is complete by GC, the reaction mixture was allowed to cool to room temperature and the precipitated bis-Boc protected diamine was removed by filtration. The solids were washed with 2×200 ml EtOH (total filtrate 1.75 L). The filtrate was transferred to 5 L RB and EtOH was distilled off atmospherically. Total approx. 1300 ml of Ethanol was distilled off. Heating was discontinued. While hot, 3.5 L of water was added to the residue (pH˜10). Cool to room temperature with stirring and then to ˜10° C. using an ice water bath. PH was adjusted to ˜13 by adding 5N NaOH. (precipitation of light pink solids is observed). The resultant slurry was stirred at room temperature for 2-3 h. (may need longer stirring time to remove major by product phenol, un-reacted diamine also is water soluble). Light pink solids were filtered off through a buchner funnel fitted with polypropylene filter cloth and washed with 2×250 ml water followed by heptane 2×250 ml. The solids were first dried on house vacuum under nitrogen for overnight and then in vacuum oven at 50° C. under nitrogen bleed till the weight is constant. The solids, desired mono-boc carbonate P2a, weighed 226 g, Yield: 35%, GC 94%, phenol 2.8%

### Synthesis of P3

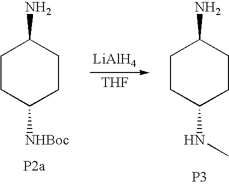

| reagents | MW | Equv. | mmol | g | |
|---|---|---|---|---|---|
| P2a | 214 | 1 | 701 | 150 | |
| LAH, 1 M | | 3 | 2100 | 2100 | mL |
| THF | | | | | |
| THF | | | | 1500 | ML |
| 15% NaOH | | | | 240 | mL |
| Toluene | | | | 800 | mL |
| CH₂Cl₂ | | | | 6000 | mL |

Procedure: To slurry of P2a in THF (1500 mL) at 30-40° C. was added 1M LAH (2100 mL) over 1 h. The reaction is exothermic and gas (CO₂) evolved. The mixture was heated to reflux and stirred for 3 h. The mixture was cooled to 10-15° C. Then, water (80 mL), 15% NaOH (80 mL), water (240 mL) were added respectively. (Exothermic) The mixture was stirred for 3 h after quench. Then, the solid was filtered off and washed and stirred with THF (3×500 mL). The solid was washed and stirred again with CH₂Cl₂ (6×1000 mL). (Only recover ˜8% product by this washes. The product has a very limited solubility in organic solvent and soluble in water). The combined organic phases were distillated and chased with toluene (2×400 mL) to dryness to give 66.8 g of P3 as a solid. 74%. GC: 95%

### Synthesis of P9

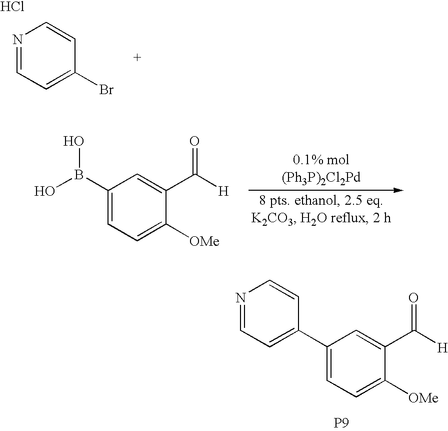

| reagents | MW | equiv | mol | g |
|---|---|---|---|---|
| P15 | 179.97 | 1 | 0.556 | 100 |
| 4-Bromopyridine | 194 | 1 | 0.556 | 108 |
| HCl | | | | |
| Pd(PPh₃)₂Cl₂ | 702 | 0.001 | 0.000556 | 0.39 |
| | | mol | | |
| K₂CO₃(4M) | | 2.5 | 1.39 | 192 |
| EtOH (2B) | | 8 pts | | |
| EtOAc | | | | |
| Conc. HCl | | | | |
| 50% NaOH | | | | 53 g |

Procedure: To a 2 L 3-neck RB flask equipped with a mechanical stirrer, condenser with N2 inlet and temperature probe was added 4-Bromopyridine hydrochloride (108 g, 0.556 mol), Then, a solution of K₂CO₃ (4M, 348 mL) was added slowly. Then, EtOH (2B) (800 mL), 3-formyl-4-methoxyphenylboronic acid P15 (10 g, 0.556 mol) and Pd(PPh₃)₂Cl₂ (0.39 g) were added. The reaction mixture was heated to 80° C. for 2 h (reflux). The reaction mixture was allowed to cool to 40-50° C. and EtOAc (1200 mL) was added. The solid was filtered. Then, conc. HCl (75 mL) was added to the filtrate to pH 2-3. The solid was stirred for 30 min and filtered and washed with EtOAc (2×200 mL). The solid was dried by air for 2 h. The crude solid was taken into water (1500 mL) and a mixture of 53 g of 50% NaOH and water (70 mL) was added slowly to pH=12-13. The mixture was stirred for 1 h and the solid was filtered and washed with water (2×500 ml) and dried at 40 oC for 24 h to give 113 g of P9 as a solid. (Yield: 96%, Pd: 31 ppm, HPLC: SLI: <0.5%, 98%)

### Synthesis of P5

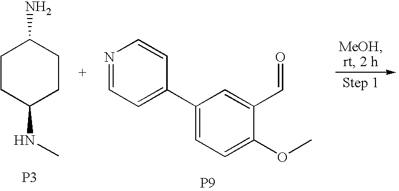

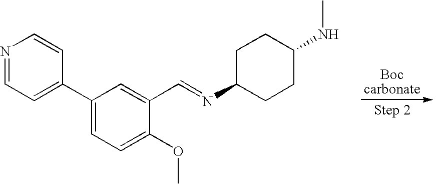

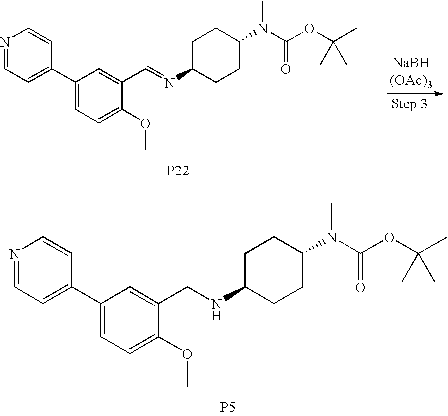

| reagents | MW | Equv. | mol | g |
|---|---|---|---|---|
| amine | 128.3 | 1.00 | 0.5 | 64 |
| aldehyde | 213.23 | 0.93 | 0.47 | 100 |
| Boc-carbonate | 218.2 | 1 | 0.5 | 109 |
| NaBH(OAc)₃ | 211.94 | 1.2 | 0.59 | 125 |
| Isopropyl acetate | | | | |

Procedure: Step 1: Imine Formation: To a 2-L 4-neck RB flask fitted with a mechanical stirrer and thermocouple, was charged 150 mL of Isopropyl acetate. To this was added the N-methyl trans1,4-diaminocyclohexane P3 (64 g, 0.5 mole). It was warmed to 28-30 C to get a clear solution. An amber solution was obtained. The aldehyde (100 g, 0.47 mole) was added to 350 mL of Isopropyl acetate and warmed 50 C. The clear pale yellow solution was filtered through a carbon pad (Norit, 90 mm diameter) and the pad was rinsed with 2×100 mL of Isopropyl acetate. The aldehyde solution was added to the amine solution in the 2-L flask maintaining temperature between 30-35 C. It was stirred for 2.0-2.5 h at 20-24 C when solids precipitated out. An aliquot was withdrawn, concentrated to dryness and analyzed by NMR. NMR indicated that the reaction was complete. An additional 250 mL of isopropyl acetate was added and the slurry was cooled to 10 C.

Step 2: Boc carbonate (109 g, 0.5 mole) was added drop wise maintaining temperature between 10 to 25 C (Highly exothermic) with stirring. The slurry became thick initially and thinned out upon further addition of the reagent. The reaction mixture was stirred for 16 h (reaction is complete in ˜2 h by NMR). The slurry was concentrated to ˜450 mL volume in a rotary evaporator at 45 C and 100 torr. 300 mL of n-heptane was added and the slurry was cooled to 10-15 C and stirred for 2 h at 20-22 C. It was filtered through a Buchner funnel and washed with 2×100 mL of n-Heptane. The pale yellow solid (180 g wet, was dried in a vacuum at 40 C for 2 h to give 138 g of product (86% yield). NMR of the product corresponded to the structure of the imine P22.

Step 3: Reduction To a 2 L 3 neck RB flask equipped with mechanical stirrer, N2 inlet and temperature probe, was added P22 (110 g, 0.26 mol), and methanol (850 mL). The reaction mixture was cooled to 2 C. Sodium triacetoxyborohydride (125 g, 0.59 mol) was added portion wise maintaining the temperature between 2-5 C over 1 h. The mixture was stirred for 1 h at 5-15 C. An aliquot was withdrawn and analyzed by HPLC. It was concentrated and analyzed by NMR. The reaction mixture was diluted with cold water (500 mL and 5N NaOH (500 mL, 3-5 C, pH 11-12). It was stirred for 0.5 h and the mixture was extracted with 2×700 mL of isopropyl acetate. The combined organic phase was washed with 2×500 mL of water. The isopropyl acetate extract was concentrated to a volume of 300 mL and this solution was used for the coupling with P10.

### Synthesis of P10

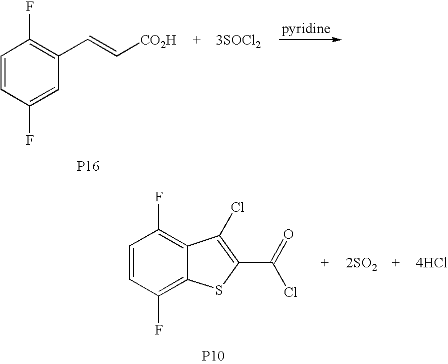

| reagents | MW | Equv. | mmol | g |
|---|---|---|---|---|
| P16 | 184.14 | 1 | 815 | 150 |
| Thionyl chloride | 118.97 | 6 | 82 | 587 |
| Pyridine | 79.1 | 0.1 | 80.4 | 6.36 |
| Chlorobenzene | 112.56 | | | 1160 |

Procedure: To a 3-L flask under nitrogen and connected to a sodium hydroxide scrubber was charged 150.0 g (0.815 mol) of trans-2,5-difluorocinnamic acid, 500 mL of chlorobenzene and 6.5 mL of pyridine, (0.1 eq.) The mixture was heated to 80° C. and 360 mL (6 eq) of thionyl chloride was added over 2 hours at 75-80° C. The mixture was heated to reflux (117-125° C.) and held for 23 hours. Excess thionyl chloride was atmospherically distilled out with chlorobenzene (125-145° C.), to a residual volume of 300 ml. The residue was cooled to 90° and 750 mL of heptane was added. The mixture was reheated to 95° C. and filtered to remove some yellow solid, 13.6 g. The cake was washed with 150 mL of heptane. The mixture cooled to −6° C., solids filtered, and washed with 500 mL of cold heptane. The wet cake (138 g) was dried under nitrogen at room temperature. Yield=104.7 g, 48.1% HPLC 98%

### Synthesis of P6

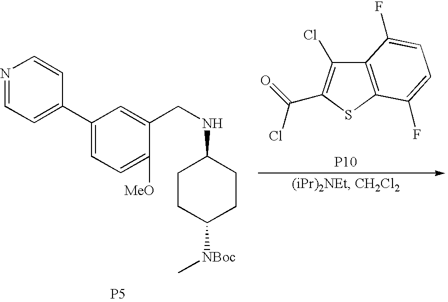

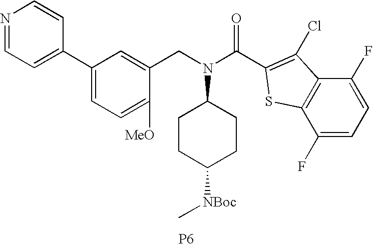

| reagents | MW | equiv. | mol | g |
|---|---|---|---|---|
| P5 | 425.56 | 1 | 0.22 | 95.5 |
| P10 | 267.08 | 1.1 | 0.22 | 59.6 |
| (iPr)₂NEt | 129.25 | 3.0 | 0.67 | 86.8 |
| Isopropyl aetate | | | | |

Procedure: To a 3-L. 4-neck RB flask fitted with a mechanical stirrer and thermocouple, was charged a solution containing 95.5 g (0.22 mole) of P5 in 600 mL of isopropy acetate. Diidopropylethylamine (91.8 g, 0.67 mole was added to it. The solution was cooled to −5 C and 59.6 g of P10 (0.22 mol) in 800 mL of isopropyl acetate was added over 2 h maintaining the temperature between −2 to +5° C. with stirring. The reaction mixture was stirred for an additional 0.5 h when solids precipitated out. HPLC indicated that the reaction was complete. The solids were filtered through a buchner lined with polypropylene and washed with 400 mL of 2B EtOH, 400 mL of water and finally 400 mL of isopropyl acetate. It was dried in vacuum at 50° C. for 16 h to give 119.6 g of P6. Yield 81.3%, HPLC 95% by area

### Synthesis of P7

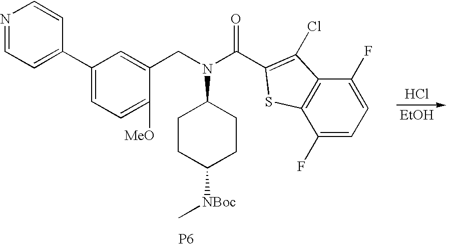

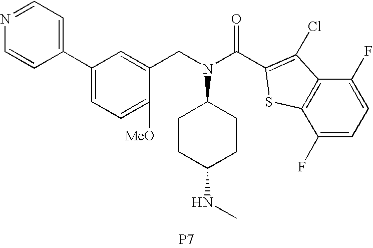

| reagents | MW | equiv. | mol | g | |
|---|---|---|---|---|---|
| P6 | 656.18 | 1 | 0.15 | 100 | g |
| HCl (conc) | 36.5 | 3.3 | 0.5 | 50.4 | g |
| EtOH (2B) | | | | | |

Procedure: To a 3-L 4-neck RB flask fitted with a mechanical stirrer and thermocouple, was charged P6 (100 g, 0.15 mol) followed by 2B-EtOH (750 mL). To the white suspension was added concentrated HCl (42 mL, 0.51 mol) at ambient temperature. The mixture was heated to 70° C. for 2 h when HPLC indicated that the starting material had disappeared. The reaction mixture was cooled to 22-25° C. and water, (750 mL) was added. The solution was extracted with 2×300 mL of methylene chloride and the aq. phase separated. The methylene chloride extract was washed with 300 mL of water containing 15 mL of 37% HCl. The organic phase was discarded and the combined aq. phase basified with 10N NaOH to pH˜13. The basic aq. phase was extracted with 2×400 mL of TBME. The phases were separated and the organic phase was washed with 2×400 mL of water. The layers were separated and the organic layer was concentrated to a voll of ˜150 mL under vacuum. TBME (20050 mL) was added to the residue and stirred for 2 h at room temperature when solids crystallized out. The solids were filtered and washed with 150 mL of TBME. The cake was dried in vacuum at 40° C. for 24 h to give 70.2 g of P7 85%) as an off-white solid. HPLC 98.2%.

### Synthesis of P8a

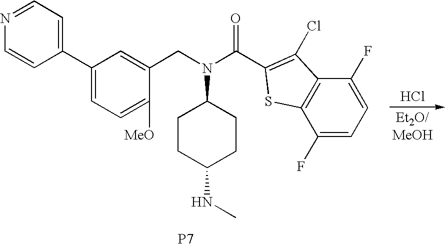

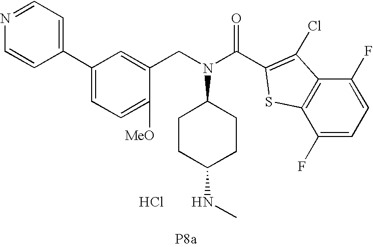

| reagents | MW | equiv. | mol | g | |
|---|---|---|---|---|---|
| P7 | 556.07 | 1 | 0.44 | 246.4 | |
| Conc. HCl (37%) | 36.5 | 0.997 | | 42.9 | g |
| EtOH (2B) | | | | | |

Procedure: To a 3-L 4-neck RB flask fitted with a mechanical stirrer and thermocouple, was charged P7 (246.41 g, 0.44 mol) followed by Ethanol (2B 1800 mL). It was stirred for 30 min at 22-24 C to effect complete dissolution. The solution was clarified by filtering through a polypropylene filter cloth. The filtrate was transferred to a 3-L 4-neck RB flask fitted with a mechanical stirrer and thermocouple. To the solution was added conc. HCl (42.9 g, 0.44 mol) dropwise over 30 minutes and the clear solution was stirred for 1.5 h at 22-24 C. (The product started precipitating after ˜45 min after HCl addition). The slurry was cool to 0-4 C and stirred for 1 h. It was filtered through a Buchner funnel lined with polypropylene. The collected solid was washed with cold EtOH (2B, 100 mL, 4-8 C) and dried under vacuum for 24 h at 45 C (N₂ flow) to give P8a (225.7 g, Yield: 86%) as a white solid. HPLC purity: 99.62%, single largest=0.11%.

### EQUIVALENTS

Those skilled in the art will recognize, or be able to ascertain using no more than routine experimentation, numerous equivalents to the compounds and methods of use thereof described herein. Such equivalents are considered to be within the scope of this invention and are covered by the following claims.

The contents of all references, patents and published patent applications cited throughout this Application, as well as their associated figures are hereby incorporated by reference in entirety.

1-115. (canceled)

116. A method for
A) the preparation of a compound of Formula D, or a salt thereof: 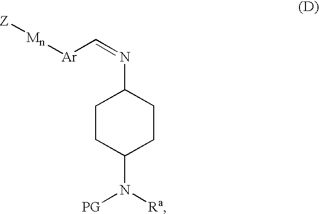
comprising treating a compound of Formula A, or a salt thereof, with a compound of Formula B, or a salt thereof: 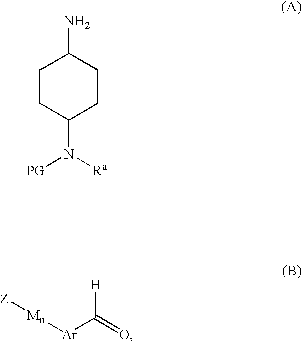
under imine formation conditions,
wherein, as valence and stability permit,
Ar is a substituted or unsubstituted aryl or heteroaryl;
M, independently for each occurrence, represents a substituted or unsubstituted methylene group, NR⁴ (wherein R⁴ is H, substituted or unsubstituted alkyl, or an amine protecting group), O, S, S(O), or S(O)₂, selected such that no two heteroatoms are adjacent to each other;
n is an integer from 0-4;
Z is H, a substituted or unsubstituted aryl, carbocyclyl, heterocyclyl, or heteroaryl, or a nitro, cyano, or halogen;
R^{a} is H, substituted or unsubstituted alkyl or an amine protecting group; and
PG is H or an amine protecting group; or
B) the preparation of a compound of Formula D, or a salt thereof: 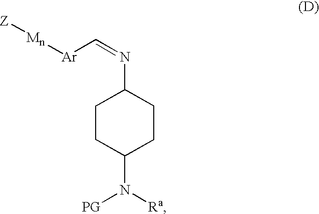
comprising treating a compound of Formula C, or a salt thereof: 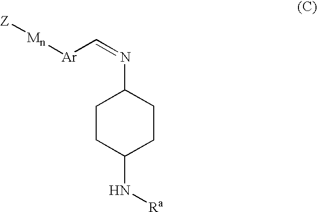
with an amine protecting group source under amine protection conditions,
wherein, as valence and stability permit,
M, R⁴, n, Ar, Z, and R^{a} are as defined in section A); and
PG is an amine protecting group; or
C) the preparation of a compound of Formula E, or a salt thereof: 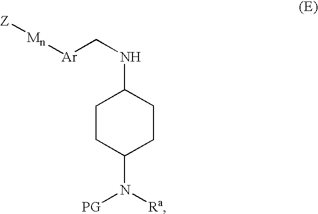
comprising treating a compound of Formula D, or a salt thereof: 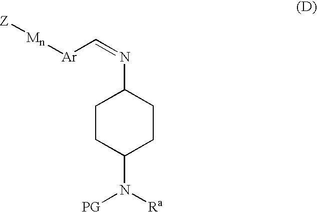
with a reducing agent under imine reduction conditions,
wherein, as valence and stability permit,
M, R⁴, n, Ar, Z, PG, and R^{a} are as defined in section B); or
D) the preparation of a compound of Formula G, or a salt thereof: 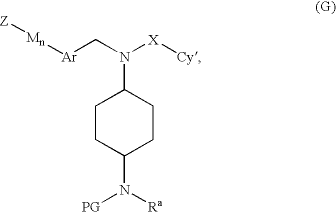
comprising treating a compound of Formula E, or a salt thereof, with a compound of Formula F, or a salt thereof: 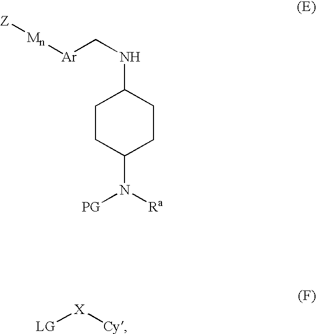
under acylation conditions,
wherein, as valence and stability permit,
M, R⁴, n, Ar, Z, PG, and R^{a} are as defined in section B);
LG is a leaving group;
X is —C(═O)—, —C(═S)—, —S(O₂)—, —S(O)—, —C(═NCN)—, or —P(═O)(OR)—; and
Cy′ is a substituted or unsubstituted benzothiophene; or
E) the preparation of a compound of Formula I, or a pharmaceutically acceptable salt thereof: 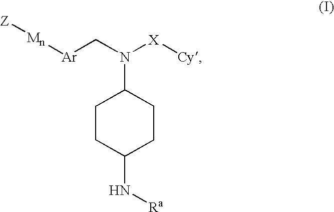
comprising subjecting a compound of Formula G, or a salt thereof: 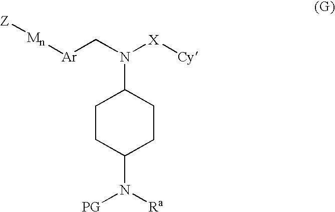
to amine deprotection conditions,
wherein, as valence and stability permit,
M, R⁴, n, Ar, Z, PG, R^{a}, X, and Cy′ are as defined in section D); or
F) the preparation of a compound of Formula B, or a salt thereof, wherein n is 0: 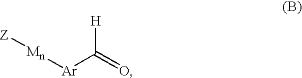
comprising treating a compound of Formula B1, or a salt thereof, with a compound of Formula B2, or a salt thereof: 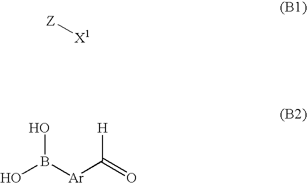
under carbon-carbon bond-forming conditions,
wherein, as valence and stability permit,
Ar is as defined in section A);
Z is a substituted or unsubstituted aryl or heteroaryl;
X¹ is a halogen, triflate, or mesylate; and
the carbon bond-forming conditions comprise a catalytic amount of Pd(OAc)₂ or (Ph₃P)₂PdCl₂, a metal carbonate, ethanol or dimethoxyethane solvent, and water; or
G) the preparation of a compound according to Formula A3, or a salt thereof: 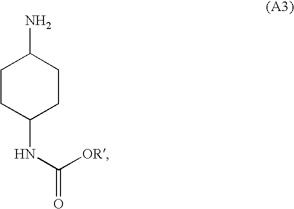
comprising treating a compound of Formula A1, or a salt thereof, with a compound of Formula A2: 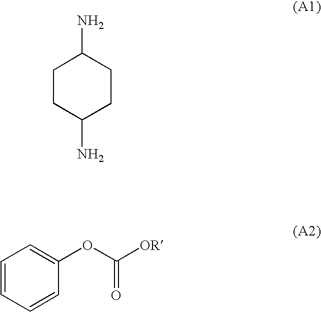
under carbamate forming conditions,
wherein, as valence and stability permit,
R′ is a substituted or unsubstituted alkyl; or
H) the preparation of a compound of Formula A, or a salt thereof: 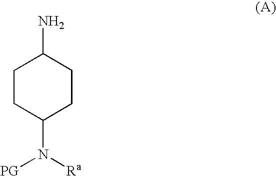
comprising treating a compound of Formula A4, or a salt thereof: 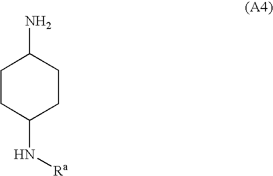
with an aldehyde under imine forming conditions followed by treating with an amine protecting group source under amine protecting conditions followed by treating with imine cleavage conditions,
wherein, as valence and stability permit,
R^{a} is substituted or unsubstituted alkyl, or an amine protecting group;
PG is as defined in section B); and
the imine formation conditions comprise an alkyl alcohol solvent; or
I) the preparation of a compound of Formula C, or a salt thereof: 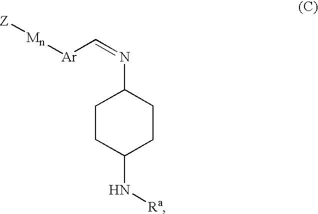
comprising treating a compound of Formula A4, or a salt thereof, with a compound of Formula B, or a salt thereof: 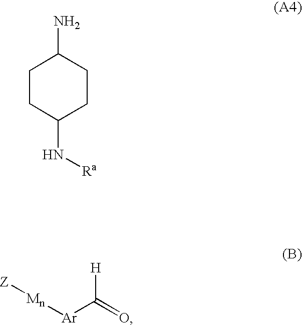
under imine formation conditions,
wherein, as valence and stability permit,
M, R⁴, n, Ar, and Z are as defined in section A); and
R^{a} is as defined in section H); or
J) the preparation of a compound according to Formula E, or a salt thereof: 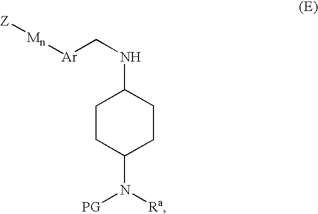
comprising treating a compound of Formula A, or a salt thereof, with a compound of Formula B, or a salt thereof: 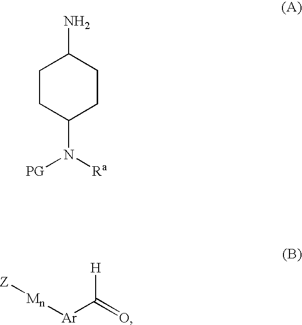
under imine formation conditions followed by treating with a reducing agent under imine reduction conditions,
wherein, as valence and stability permit,
M, R⁴, n, Ar, Z, PG, and R^{a} are as defined in section B); and
the imine formation conditions comprise an alkyl alcohol solvent; or
K) the preparation of a compound according to Formula I, or a pharmaceutically acceptable salt thereof: 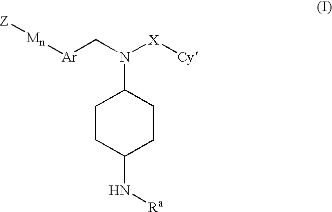
comprising:
a) treating a compound of Formula A4, or a salt thereof, with a compound of Formula B, or a salt thereof: 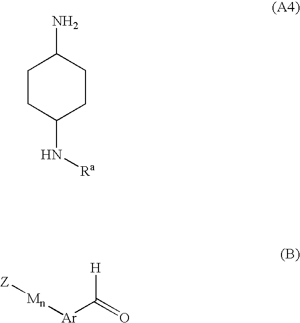
under imine formation conditions to generate a compound of Formula C, or a salt thereof: 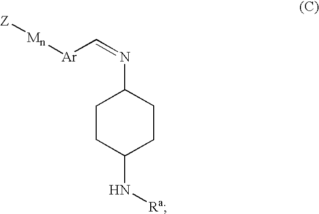
b) treating the compound of Formula C, or a salt thereof, with an amine protecting group source under amine protecting conditions to generate a compound of Formula D, or a salt thereof: 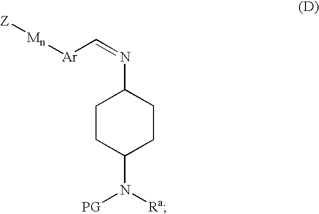
c) treating the compound of Formula D, or a salt thereof, with a reducing agent under imine reduction conditions to generate a compound of Formula E, or a salt thereof: 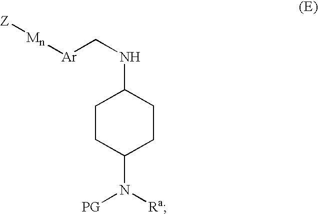
d) treating the compound of Formula E, or a salt thereof, with a compound of Formula F, or a salt thereof: 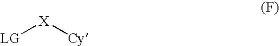
under acylation conditions to generate a compound of Formula G, or a salt thereof: 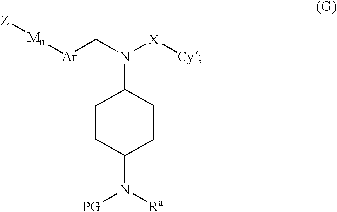
e) treating the compound of Formula G, or a salt thereof, under conditions that remove PG to prepare a compound of Formula I, or a pharmaceutically acceptable salt thereof,
wherein, as valence and stability permit,
M, R⁴, n, Ar, Z, X, PG, R^{a}, Cy′, and LG are as defined in section D); or
L) the preparation of a compound according to Formula I, or a pharmaceutically acceptable salt thereof: 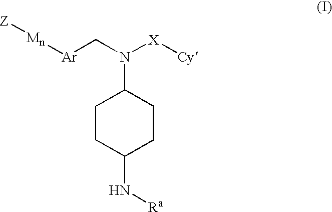
comprising:
a) treating a compound of Formula A, or a salt thereof, with a compound of Formula B, or a salt thereof: 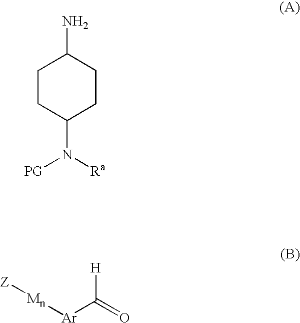
under imine formation conditions to generate a compound of Formula D, or a salt thereof: 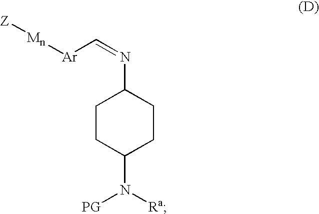
b) treating the compound of Formula D, or a salt thereof, with a reducing agent under imine reduction conditions to generate a compound of Formula E, or a salt thereof: 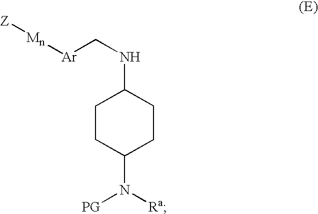
c) treating the compound of Formula E, or a salt thereof, with a compound of Formula F, or a salt thereof: 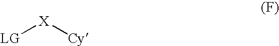
under acylation conditions to generate a compound of Formula G, or a salt thereof: 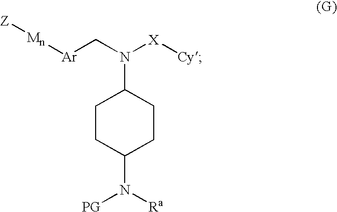
d) treating the compound of Formula G, or a salt thereof, under conditions that remove PG to prepare a compound of Formula I, or a pharmaceutically acceptable salt thereof,
wherein, as valence and stability permit,
M, R⁴, n, Ar, Z, X, PG, R^{a}, Cy′, and LG are as defined in section D).
